(19) United States
(12) Patent Application Publication
(10) Pub. No.: US 2011/0039758 A1
(43) Pub. Date: Feb. 17, 2011

(54) Molecular Interactions in Neurons

(75) Inventors: Peter S. Lu, Mountain View, CA (US); Jonathan David Garman, San Jose, CA (US); Michael P. Belmares, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arbor Vita Corporation

(21) Appl. No.: 12/779,501

(22) Filed: May 13, 2010

Related U.S. Application Data
(63) Continuation of application No. 10/714,537, filed on Nov. 14, 2003, now abandoned.
Application No. 10/714,537 is a continuation-in-part of application No. PCT/US02/24655, filed on Aug. 2, 2002.
Application No. 10/714,537 is a continuation-in-part of application No. 09/724,553, filed on Nov. 28, 2000, now abandoned.
Application No. 09/724,553 is a continuation-in-part of application No. 09/547,276, filed on Apr. 11, 2000, now abandoned.
(60) Provisional application No. 60/426,212, filed on Nov. 14, 2002.
(60) Provisional application No. 60/426,213, filed on Nov. 14, 2002.
(60) Provisional application No. 60/309,841, filed on Aug. 3, 2001.
(60) Provisional application No. 60/360,061, filed on Feb. 25, 2002.
(60) Provisional application No. 60/134,117, filed on May 14, 1999.

(51) Int. Cl. A61K 38/02; C07K 19/00

(52) U.S. Cl. 514/1.2; 530/300; 530/328; 530/324; 530/325; 530/326; 530/327; 530/329

(57) ABSTRACT

Inhibitors that disrupt binding between a PDZ protein and cognate ligands such as N-methyl-D-aspartate (NMDA) receptors that are involved in various neurological disorders are provided. Pharmaceutical compositions containing such inhibitors and their use in treating neurological diseases such as stroke and ischemia are also disclosed. Screening methods to identify additional inhibitors of specific protein ligand interactions with PDZ proteins are also described.

### CROSS-REFERENCES TO RELATED APPLICATIONS

This application claims the benefit of U.S. Provisional Application No. 60/426,212, filed Nov. 14, 2002, and U.S. Provisional Application No. 60/426,213, filed Nov. 14, 2002. This application is also (a) a continuation-in-part of PCT Application No. US02/24655, filed Aug. 2, 2002, which claims the benefit of U.S. Provisional Application No. 60/309,841, filed Aug. 3, 2001, and U.S. Provisional Application No. 60/360,061, filed Feb. 25, 2002, and (b) a continuation-in-part of U.S. application Ser. No. 09/724,553, filed Nov. 28, 2000, which is a continuation-in-part of U.S. application Ser. No. 09/547,276, filed Apr. 11, 2000, which claims the benefit of U.S. Provisional Application No. 60/134,117, filed May 14, 1999. All of the foregoing applications are are incorporated herein by reference in their entirety for all purposes.

### FIELD OF THE INVENTION

The present invention relates to the prevention and treatment of neurological disorders, including cellular damage following stroke episodes or ischemia. The invention discloses methods of treating these disorders by administering inhibitors that disrupt protein-protein interactions involved in these disorders, screening methods to identify such inhibitors and specific compositions useful for treating these disorders.

### BACKGROUND

Stroke is predicted to affect more than 600,000 people in the United States this year. In a 1999 report, over 167,000 people died from strokes, with a total mortality of 278,000. In 1998, 3.6 billion was paid to just those Medicare beneficiaries that were discharged from short-stay hospitals, not including the long term care for >1,000,000 people that reportedly have functional limitations or difficulty with activities of daily living resulting from stroke (Heart and Stroke Statistical update, American Heart Association, 2002). At this time, no therapeutics are available to reduce brain damage resulting from stroke.

Stroke is characterized by neuronal cell death in areas of ischemia, brain hemorrhage or trauma. Many lines of evidence have demonstrated that this cell death is triggered by glutamate over-excitation of neurons, leading to increased intracellular Ca²⁺ and increased nitric oxide due to an increase in nNOS (neuronal nitric oxide synthase) activity.

Glutamate is the main excitatory neurotransmitter in the central nervous system (CNS) and mediates neurotransmission across most excitatory synapses. Three classes of glutamate-gated ion channel receptors (N-methyl-D-aspartate (NMDA), alpha-amino-3-hydroxy-5-methylisoxazole-4-propionic acid (AMPA) and Kainate) transduce the postsynaptic signal. Of these, NMDA receptors (NMDAR) have been shown to be responsible for a significant portion of the excitotoxicity of glutamate. NMDA receptors are complex, being composed of an NR1 subunit and one or more NR2 subunits (2A, 2B, 2C or 2D) (see, e.g., McDain, C. and Mayer, M. (1994) Physiol. Rev. 74:723-760), and less commonly, an NR3 subunit (Chatterton et al. (2002) Nature 415:793-798). The NR1 subunits have been shown to bind glycine, while NR2 subunits bind glutamate. Both glycine and glutamate binding are required to open the ion channel and allow calcium entry into the cell. The four NR2 receptor subunits appear to determine the pharmacology and properties of NMDA receptors, with further contributions from alternative splicing of the NR1 subunit (Kornau et al. (1995) Science 269:1737-40). Whereas NR1 and NR2A subunits are ubiquitously expressed in the brain, NR2B expression is restricted to the forebrain, NR2C to the cerebellum, and NR2D is rare compared to the other types.

Because of the key role these two proteins have in the excitotoxicity response, various approaches have been utilized to target these proteins. For example, the NMDA receptor contains a large number of modulatory sites and has been targeted by many therapeutics since the 1970's. Drugs have been developed that target the ion channel (ketamine, phencyclidine, PCP, MK801, amantadine), the outer channel (magnesium), the glycine binding site on NR1 subunits, the glutamate binding site on NR2 subunits, and specific sites on NR2 subunits (Zinc—NR2A; Ifenprodil, Traxoprodil—NR2B). Among these, the non-selective antagonists of NMDA receptor have been the most neuroprotective agents in animal models of stroke. However, clinical trials with these drugs in stroke and traumatic brain injury have so far failed, generally as a result of severe side effects such as hallucination and even coma. Pharmaceutical companies have focused on subunit selective antagonists in hopes of obtaining neuroprotection without the negative side effects that limit the clinical utility of the compounds studied to date. These, however, have also been unsuccessful in the clinic thus far.

These failures have underscored the need to unravel the mechanisms of neurotoxicity downstream from the NMDA receptors as alternative drug targets. The goal in developing drugs to such targets is to identify drugs that inhibit the glutamate excitotoxcity response associated with glutamate activity, while not inhibiting the ability of NMDA receptors to function as ion channels.

### SUMMARY

The present invention relates to the treatment of neuronal disorders such as brain damage resulting from stroke, ischemia or related trauma by modulating specific protein:protein interactions between PDZ and PL proteins that are involved in these diseases. Methods for identifying specific therapeutics that modulate the specific protein:protein interactions involved in these disorders are also provided. Compounds for treating these neuronal disorders are also disclosed.

Methods of identifying the cellular PDZ proteins that are bound by the 5 main subunits of the NMDA receptor complex (R1, R2A, R2B, R2C, and R2D) are provided. Methods are also provided to identify inhibitors that are both high affinity for specific subunits. Other methods are provided to determine selectivity of inhibition, both against the different NMDA receptor subunits and the PDZs that can bind them. Methods for delivering peptide inhibitors to cells such as neuron cells are also disclosed. One class of inhibitors of PDZ:PL interactions that are disclosed are isolated, recombinant or synthetic polypeptides that inhibit binding between a N-methyl-D-aspartate (NMDA) receptor and a PDZ protein. Some inhibitors of this type include at least the C-terminal 4 amino acids of the NMDA receptor. The inhibitors can be relatively short polypeptides (e.g., less than 40, such as 3-8 or 4-20 amino acids). The polypeptide inhibitors in this class can optionally be fusion polypeptides that comprise a PL sequence and a segment of a transmembrane transporter sequence that is effective to transport the polypeptide into neuron cells.

Another class of inhibitors are polypeptides that also inhibit the binding between a NMDA receptor protein and a PDZ protein and have a C-terminal amino acid sequence of the polypeptide of X-T-X-V/L/A. Exemplary C-terminal sequences with this motif include, but are not limited to, ETEV, ETQL, QTQV, ETAL, QTEV, ETVA or FTDV. Inhibitors in this class can vary in size but are in some instances less than 40 amino acids in length, such as 3-20 amino acids in length or 3-8 amino acids in length. These inhibitors can also be fusion polypeptides that include a segment of a transmembrane transporter sequence that is effective to transport the polypeptide into neuron cells.

Another class of inhibitors capable of disrupting NMDA receptor protein:PDZ interactions are only three amino acids in length. Specific examples of such inhibitors include TEV or SDV.

Other inhibitors are isolated, recombinant or synthetic polypeptides that inhibit binding between PSD-95 and NMDAR2A, NMDAR2C and/or NMDAR2D, but not NMDAR2B. Certain inhibitors in this particular class inhibit binding of PSD-95 to all three of these NMDAR2 proteins. Other inhibitors block binding to one or more of NMDAR2A, NMDAR2C and/or NMDAR2D but still allow PSD-95 to bind to one or more of these proteins. Thus, the inhibitors can be used to selectively inhibit certain interactions.

Some inhibitors of the foregoing types disrupt interactions between NMDA receptor proteins and a PDZ that is selected from the group consisting of DLG1, DLG2, KIAA0973, NeDLG, Outermembrane protein, PSD-95, Syntrophin alpha 1, TIP1, TIP2, INADL, KIAA0807, KIAA1634, Lim-Mystique, LIM-RIL, MAGI1, MAGI2, Syntrophin beta-1 and Syntrophin gamma-1. Other inhibitors inhibit binding between NMDA receptor proteins and a PDZ protein selected from the group consisting of DLG1, DLG2, KIAA0973, NeDLG, Outermembrane protein, PSD-95, Syntrophin alpha 1, TIP1 and TIP2. Still other inhibitors interfere with binding between NMDA receptor proteins and DLG1, INADL, KIAA0807, KIAA1634, Lim-Mystique, LIM-RIL, MAGI1, MAGI2, PSD-95, Syntrophin beta-1 or Syntrophin gamma-1.

Also provided are inhibitors that can selectively disrupt binding between a PDZ protein and specific NMDA receptor subunits. For instance, some inhibitors inhibit binding between a PDZ protein and NMDAR2A, NMDAR2B, NMDAR2C and/or NMDAR2D.

Any of the inhibitors of the types just described can be formulated as a pharmaceutical composition. Thus the use of the foregoing inhibitors in the manufacture of a medicament are also provided. Such compositions typically include an inhibitor and a physiologically acceptable carrier, diluent or excipient. As a specific example, certain pharmaceutical compositions comprise a fusion polypeptide that inhibits binding between a N-methyl-D-aspartate (NMDA) receptor and a PDZ protein and a physiologically acceptable carrier, diluent or excipient. The fusion polypeptide in certain compositions is a fusion of (i) a 9 amino acid segment whose C-terminal sequence is selected from the group of amino acid sequences consisting of ETEV, ETQL, QTQV, ETAL, QTEV, ETVA and FTDV and (ii) an amino acid segment of a transmembrane transporter that is effective to transport the polypeptide into a neuron. The transmembrane transporter fused to the polypeptide is selected from the group consisting of HIV tat, Drosophila antennapedia, herpes simplex virus VP22 and anti-DNA CDR2 and anti-DNA CDR3. The transporter segment can be of varying lengths, such as 10-40 amino acids long. Other segments are 11 amino acids long. So, for instance, the inhibitor can include the the C-terminal sequence of the polypeptide is ETEV, and the transmembrane transporter sequence is YGRKKRRQRRR.

A variety of methods for inhibiting binding between a N-methyl-D-aspartate (NMDA) receptor and a PDZ protein in a neuron cell are provided. Some methods of this type involve introducing into the cell a polypeptide that inhibits binding between the NMDA receptor and a PDZ protein and comprises a C-terminal amino acid sequence of X-T-X-V/L/A. The C-terminal sequence thus can be , for example, ETEV, ETQL, QTQV, ETAL, QTEV, ETVA or FTDV.

In certain methods, the polypeptide inhibits binding between the NMDA Receptor 2 subunit and domain 1 of PSD-95. Certain polypeptides causing such inhibition have a C-terminal amino acid sequence of ETVA or FTDV. Other inhibitors that are disclosed inhibit binding between the NMDA receptor and domain 2 of PSD-95. Inhibitors with this type of specificity have a C-terminal amino acid sequence of the polypeptide is ETEV, ETQL, QTQV, ETAL, QTEV.

Other methods for inhibiting binding between a N-methyl-D-aspartate (NMDA) receptor and a PDZ protein in a neuron cell involve introducing into the cell a polypeptide that inhibits binding of the NMDA receptor and a particular PDZ protein. The PDZ protein, for example, can be selected from the group consisting of DLG1, DLG2, KIAA0973, NeDLG, Outermembrane protein, Syntrophin alpha 1, TIP1, TIP2, INADL, KIAA0807, KIAA1634, Lim-Mystique, LIM-RIL, MAGI1, MAGI2, Syntrophin beta-1 and Syntrophin gamma-1. In other methods, the PDZ protein is selected from the group consisting of DLG1, DLG2, KIAA0973, NeDLG, Outermembrane protein, Syntrophin alpha 1, TIP1 and TIP2. With other methods, the PDZ protein is selected from the group consisting of DLG1, INADL, KIAA0807, KIAA1634, Lim-Mystique, LIM-RIL, MAGI1, MAGI2, Syntrophin beta-1 and Syntrophin gamma-1.

Other methods for inhibiting binding between a N-methyl-D-aspartate (NMDA) receptor and a PDZ protein in a neuron cell involve introducing into the cell a polypeptide that inhibits binding of the NMDA receptor and a PDZ protein and is 3 amino acids in length. For example, the sequence can be TEV or SDV.

Still other methods involve introducing into the cell a polypeptide that inhibits binding between the NMDA receptor and the PDZ protein, wherein the polypeptide is a fusion of (i) a 9 amino acid segment whose C-terminal sequence is selected from the group of amino acid sequences consisting of ETEV, ETQL, QTQV, ETAL, QTEV, ETVA and FTDV and (ii) an amino acid segment of a transmembrane transporter that is effective to transport the polypeptide into a neuron.

All of these inhibitory methods can be conducted in vitro or in vivo.

A number of different screening methods are also provided. For instance, some methods are for determining whether a test compound inhibits binding between a PDZ protein and a N-methyl-D-aspartate (NMDA) receptor. These methods generally involve contacting a PDZ-domain polypeptide comprising a PDZ domain from the PDZ protein and a PL peptide that comprises at least the C-terminal 3 amino acids of the NMDA receptor in the presence of the test compound, wherein the PDZ protein is selected from the group listed in TABLE 7. In some screening methods the PDZ is a protein other than PSD-95. The concentration of complex formed between the PDZ-domain polypeptide and the PL peptide is then determined. The test compound is identified as a potential inhibitor of binding between the PDZ protein and the NMDA receptor if a lower concentration of the complex is detected in the presence of the test compound relative to the concentration of the complex in the absence of the test compound.

Some screening methods are conducted with PDZ proteins that are selected from the group consisting of DLG1, DLG2, KIAA0973, NeDLG, Outermembrane protein, Syntrophin alpha 1, TIP1, TIP2, INADL, KIAA0807, KIAA1634, Lim-Mystique, LIM-RIL, MAGI1, MAGI2, Syntrophin beta-1 and Syntrophin gamma-1. The PDZ protein in other screening methods is PSD-95.

The screening methods can optionally involve assaying a compound identified in during the initial screening method to have inhibitory activity to determine whether the identified compound mitigates against a condition associated with a neuronal disorder. Examples of such assays include, but are not limited to, apoptosis assays, caspase assays, cytochrome c assays and cell lysis assays.

The inhibitors and pharmaceutical compositions that are provided or that can be identified using the screening methods disclosed herein can be utilized to treat a variety of neurological diseases. In general, these methods involve administering an effective amount of an inhibitor or pharmaceutical composition as described herein to an individual having the neuronal injury or at risk of obtaining the neuronal injury. The individual can be a non-human mammal (e.g., as in an animal model system) or a human. Various types of neurological diseases can be treated, including diseases associated with stroke and ischemia.

### BRIEF DESCRIPTION OF THE DRAWINGS

FIG. 1 shows the interaction of NMDAR2A with PSD95, TIP2, DLG1, and LIM. Light gray bars represent the background binding of NMDAR2A when 2% BSA is substituted for PDZ protein in the assay. Standard deviation is presented for all data points. Absorbance (y-axis) is measured at 450 nm.

FIG. 2 shows the PDZ binding profile for each NMDA receptor 2 subunit to each of 238 PDZ proteins. Y axis indicates the A₄₅₀ nm reading using the ‘G’ assay described herein; higher vertical bars are stronger interactions. The X axis indicates individually cloned and expressed human PDZ domains, numbered from 1 to 238.

FIG. 3 demonstrates that NMDA Receptor subunits 2A, 2B and 2C can bind PDZ domains 1 and 2 of PSD-95 (and a construct containing all three domains of PSD-95), but do not interact significantly with PSD-95 PDZ domain 3.

FIG. 4 shows titrations of NMDA Receptor 2 subunits (A=R2A, B=R2B, C=R2C, D=R2D) against the individual domains of PSD-95 and a construct containing all three domains. GST is a negative control, and PTPL/PBK is a weak positive control for the ELISA assay. The legend indicates the concentration in uM of the NMDA Receptor peptide that was added.

FIG. 5 shows that binding of NMDA R2A to PSD95 domain 1 or domain 2 can be competed off by the addition of 3 amino acid peptides TEV (labeled TAT) or SDV (labeled 2B).

FIG. 6 demonstrates that 19 amino acid peptides corresponding to the C-termini of TAX or HPV E6 type 16 can compete for binding of NMDA Receptor 2C to PSD95 domain 2 but not to domain 1 in these concentration ranges.

FIG. 7 demonstrates that 3 amino acid peptides corresponding to the C-termini of TAX or HPV E6 type 16 can compete for binding of NMDA Receptor 2C to PSD95 domain 2 but not to domain 1 in these concentration ranges.

FIG. 8 demonstrates that 4 amino acid peptides corresponding to the C-termini of TAX or HPV E6 type 16 can compete for binding of NMDA Receptor 2C to PSD95 domain 2 but not to domain 1 in these concentration ranges.

FIG. 9 shows that binding of NMDA R2A to PSD95 domain 1 or domain 2 can be competed off by the addition of 19 amino acid peptides corresponding to the C-termini of TAX or HPV E6 type 16 in these concentration ranges.

FIG. 10 demonstrates that when a TAT transporter sequence is coupled to the C-terminal 9 amino acids of Tax binding is still mediated through the C-terminal PDZ Ligand motif (PL). TatTAXAA is a construct that changes the binding specificity of TAT by alanine substitution at the critical positions 0 and −2 of the PL. This figure shows that the TATTAX peptide can inhibit NMDA R2A and R2B binding to the second PDZ of PSD95 but that the mutated PL version (TATTAXAA) cannot.

FIG. 11 demonstrates that the internal PL motif of nNOS specifically binds PDZ domain 2 of PSD95 but does not bind PDZ domain 1.

FIG. 12 demonstrates that 20 amino acid and 3 amino acid peptide inhibitors can selectively inhibit binding of one PL to PSD-95 PDZ domain 1 but not inhibit a second PL binding to the same PDZ domain.

### DETAILED DESCRIPTION

### I. Definitions

“Polypeptide,” “protein” and “peptide” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

A “fusion protein” or “fusion polypeptide” as used herein refers to a composite protein, i.e., a single contiguous amino acid sequence, made up of two (or more) distinct, heterologous polypeptides which are not normally fused together in a single amino acid sequence. Thus, a fusion protein can include a single amino acid sequence that contains two entirely distinct amino acid sequences or two similar or identical polypeptide sequences, provided that these sequences are not normally found together in the same configuration in a single amino acid sequence found in nature. Fusion proteins can generally be prepared using either recombinant nucleic acid methods, i.e., as a result of transcription and translation of a recombinant gene fusion product, which fusion comprises a segment encoding a polypeptide of the invention and a segment encoding a heterologous protein, or by chemical synthesis methods well known in the art.

A “fusion protein construct” as used herein is a polynucleotide encoding a fusion protein.

As used herein, the term “PDZ domain” refers to protein sequence (i.e., modular protein domain) of approximately 90 amino acids, characterized by homology to the brain synaptic protein PSD-95, the Drosophila septate junction protein Discs-Large (DLG), and the epithelial tight junction protein ZO1 (ZO1). PDZ domains are also known as Discs-Large homology repeats (“DHRs”) and GLGF repeats. PDZ domains generally appear to maintain a core consensus sequence (Doyle, D. A., 1996, Cell 85: 1067-76).

PDZ domains are found in diverse membrane-associated proteins including members of the MAGUK family of guanylate kinase homologs, several protein phosphatases and kinases, neuronal nitric oxide synthase, and several dystrophin-associated proteins, collectively known as syntrophins.

Exemplary PDZ domain-containing proteins and PDZ domain sequences are shown in TABLE 4. The term “PDZ domain” also encompasses variants (e.g., naturally occurring variants) of the sequences of TABLE 4 (e.g., polymorphic variants, variants with conservative substitutions, and the like). Typically, PDZ domains are substantially identical to those shown in TABLE 4, e.g., at least about 70%, at least about 80%, or at least about 90% amino acid residue identity when compared and aligned for maximum correspondence.

As used herein, the term “PDZ protein” refers to a naturally occurring protein containing a PDZ domain. Exemplary PDZ proteins include CASK, MPP1, DLG1, PSD95, NeDLG, TIP33, SYN1a, TIP43, LDP, LIM, LIMK1, LIMK2, MPP2, NOS1, AF6, PTN-4, prIL16, 41.8 kD, KIAA0559, RGS12, KIAA0316, DVL1, TIP40, TIAM1, MINT1, KIAA0303, CBP, MINT3, TIP2, KIAA0561, and those listed in TABLE 4.

As used herein, the term “PDZ-domain polypeptide” refers to a polypeptide containing a PDZ domain, such as a fusion protein including a PDZ domain sequence, a naturally occurring PDZ protein, or an isolated PDZ domain peptide.

As used herein, the term “PL protein” or “PDZ Ligand protein” refers to a naturally occurring protein that forms a molecular complex with a PDZ-domain, or to a protein whose carboxy-terminus, when expressed separately from the full length protein (e.g., as a peptide fragment of 4-25 residues, e.g., 8, 10, 12, 14 or 16 residues), forms such a molecular complex. The molecular complex can be observed in vitro using the “A assay” or “G assay” described infra, or in vivo. Exemplary NMDA receptor PL proteins listed in TABLE 2 are demonstrated to bind specific PDZ proteins. This definition is not intended to include anti-PDZ antibodies and the like.

As used herein, the terms “NMDA receptor,” “NMDAR,” or “NMDA receptor protein” refer to a membrane associated protein that is known to interact with NMDA. The term thus includes the various subunit forms, including for example, those listed in TABLE 2. The receptor can be a non-human mammalian NMDAR (e.g., mouse, rat, rabbit, monkey) or a human NMDAR, for example.

As used herein, the term “NMDAR-PL” or “NMDA receptor-PL” refers to a NMDA receptor that forms a molecular complex with a PDZ domain or to a NMDAR protein whose carboxy-terminus, when expressed separately from the full length protein (e.g., as a peptide fragment of 4-25 residues, e.g., 8, 10, 12, 14 or 16 residues), forms such a molecular complex.

As used herein, a “PL sequence” refers to the amino acid sequence of the C-terminus of a PL protein (e.g., the C-terminal 2, 3, 4, 5, 6, 7, 8, 9, 10, 12, 14, 16, 20 or 25 residues) (“C-terminal PL sequence”) or to an internal sequence known to bind a PDZ domain (“internal PL sequence”).

As used herein, a “PL peptide” is a peptide of having a sequence from, or based on, the sequence of the C-terminus of a PL protein. Exemplary PL peptides (biotinylated) are listed in TABLE 2.

As used herein, a “PL fusion protein” is a fusion protein that has a PL sequence as one domain, typically as the C-terminal domain of the fusion protein. An exemplary PL fusion protein is a tat-PL sequence fusion.

As used herein, the term “PL inhibitor peptide sequence” refers to PL peptide amino acid sequence that (in the form of a peptide or PL fusion protein) inhibits the interaction between a PDZ domain polypeptide and a PL peptide (e.g., in an A assay or a G assay).

As used herein, a “PDZ-domain encoding sequence” means a segment of a polynucleotide encoding a PDZ domain. In various embodiments, the polynucleotide is DNA, RNA, single stranded or double stranded.

As used herein, the terms “antagonist” and “inhibitor,” when used in the context of modulating a binding interaction (such as the binding of a PDZ domain sequence to a PL sequence), are used interchangeably and refer to an agent that reduces the binding of the, e.g., PL sequence (e.g., PL peptide) and the, e.g., PDZ domain sequence (e.g., PDZ protein, PDZ domain peptide).

As used herein, the terms “agonist” and “enhancer,” when used in the context of modulating a binding interaction (such as the binding of a PDZ domain sequence to a PL sequence), are used interchangeably and refer to an agent that increases the binding of the, e.g., PL sequence (e.g., PL peptide) and the, e.g., PDZ domain sequence (e.g., PDZ protein, PDZ domain peptide).

The terms “isolated” or “purified” means that the object species (e.g., a polypeptide) has been purified from contaminants that are present in a sample, such as a sample obtained from natural sources that contain the object species. If an object species is isolated or purified it is the predominant macromolecular (e.g., polypeptide) species present in a sample (i.e., on a molar basis it is more abundant than any other individual species in the composition), and preferably the object species comprises at least about 50 percent (on a molar basis) of all macromolecular species present. Generally, an isolated, purified or substantially pure composition comprises more than 80 to 90 percent of all macromolecular species present in a composition. Most preferably, the object species is purified to essential homogeneity (i.e., contaminant species cannot be detected in the composition by conventional detection methods), wherein the composition consists essentially of a single macromolecular species.

The term “recombinant” when used with respect to a polypeptide refers to a polypeptide that has been prepared be expressing a recombinant nucleic acid molecule in which different nucleic acid segments have been joined together using molecular biology techniques.

The term “synthesized” when used with respect to a polypeptide generally means that the polypeptide has been prepared by means other than simply purifying the polypeptide from naturally occurring sources. A synthesized polypeptide can thus be prepared by chemical synthesis, recombinant means, or by a combination of chemical synthesis and recombinant means. Segments of a synthesized polypeptide, however, may be obtained from naturally occurring sources.

The term “biological function” or “biological activity” in the context of a cell, refers to a detectable biological activity normally carried out by the cell, e.g., a phenotypic change such as proliferation, cell activation, excitotoxicity responses, neurotransmitter release, cytokine release, degranulation, tyrosine phosphorylation, ion (e.g., calcium) flux, metabolic activity, apoptosis, changes in gene expression, maintenance of cell structure, cell migration, adherence to a substrate, signal transduction, cell-cell interactions, and others described herein or known in the art.

As used herein, the terms “peptide mimetic,” “peptidomimetic,” and “peptide analog” are used interchangeably and refer to a synthetic chemical compound which has substantially the same structural and/or functional characteristics of an PL inhibitory or PL binding peptide of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or inhibitory or binding activity. As with polypeptides of the invention which are conservative variants, routine experimentation will determine whether a mimetic is within the scope of the invention, i.e., that its structure and/or function is not substantially altered. Thus, a mimetic composition is within the scope of the invention if it is capable of binding to a PDZ domain and/or inhibiting a PL-PDZ interaction.

Polypeptide mimetic compositions can contain any combination of nonnatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N=-dicyclohexylcarbodiimide (DCC) or N,N=-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, A Peptide Backbone Modifications, Marcell Dekker, NY).

A polypeptide can also be characterized as a mimetic by containing all or some non-natural residues in place of naturally occurring amino acid residues. Nonnatural residues are well described in the scientific and patent literature; a few exemplary nonnatural compositions useful as mimetics of natural amino acid residues and guidelines are described below.

Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(tri fluoromethyl)-phenylalanine; D-p-fluorophenylalanine; D- or L-p-biphenylphenylalanine; K- or L-p-methoxybiphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a nonnatural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R═—N—C—N—R═) such as, e.g., 1-cyclohexyl-3(2-morpholinyl-(4-ethyl) carbodiimide or 1-ethyl-3(4-azonia-4,4-dimetholpentyl)carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions.

Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively.

Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole.

Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide.

Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a natural polypeptide (e.g., a PL polypeptide or PDZ polypeptide) can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, generally referred to as the D-amino acid, but which can additionally be referred to as the R— or S— form.

The mimetics of the invention can also include compositions that contain a structural mimetic residue, particularly a residue that induces or mimics secondary structures, such as a beta turn, beta sheet, alpha helix structures, gamma turns, and the like. For example, substitution of natural amino acid residues with D-amino acids; N-alpha-methyl amino acids; C-alpha-methyl amino acids; or dehydroamino acids within a peptide can induce or stabilize beta turns, gamma turns, beta sheets or alpha helix conformations. Beta turn mimetic structures have been described, e.g., by Nagai (1985) Tet. Lett. 26:647-650; Feigl (1986) J. Amer. Chem. Soc. 108:181-182; Kahn (1988) J. Amer. Chem. Soc. 110:1638-1639; Kemp (1988) Tet. Lett. 29:5057-5060; Kahn (1988) J. Molec. Recognition 1:75-79. Beta sheet mimetic structures have been described, e.g., by Smith (1992) J. Amer. Chem. Soc. 114:10672-10674. For example, a type VI beta turn induced by a cis amide surrogate, 1,5-disubstituted tetrazol, is described by Beusen (1995) Biopolymers 36:181-200. Incorporation of achiral omega-amino acid residues to generate polymethylene units as a substitution for amide bonds is described by Banerjee (1996) Biopolymers 39:769-777. Secondary structures of polypeptides can be analyzed by, e.g., high-field 1H NMR or 2D NMR spectroscopy, see, e.g., Higgins (1997) J. Pept. Res. 50:421-435. See also, Hruby (1997) Biopolymers 43:219-266, Balaji, et al., U.S. Pat. No. 5,612,895.

As used herein, “peptide variants” and “conservative amino acid substitutions” refer to peptides that differ from a reference peptide (e.g., a peptide having the sequence of the carboxy-terminus of a specified PL protein) by substitution of an amino acid residue having similar properties (based on size, polarity, hydrophobicity, and the like). Thus, insofar as the compounds that are encompassed within the scope of the invention are partially defined in terms of amino acid residues of designated classes, the amino acids may be generally categorized into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated electron system (aromatic group). The aromatic group may be further substituted with groups such as alkyl, alkenyl, alkynyl, hydroxyl, sulfanyl, nitro and amino groups, as well as others. Examples of genetically encoded aromatic amino acids include Phe, Tyr and Trp. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chloro-phenylalanine, 2-fluorophenyl-alanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include Gly, Pro and Met. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include Asp and Glu.

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include Arg, Lys and His. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include Asx and Glx. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

As will be appreciated by those having skill in the art, the above classification are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a peptide.

Certain commonly encountered amino acids which are not genetically encoded of which the peptides and peptide analogues of the invention may be composed include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). These amino acids also fall conveniently into the categories defined above.

The classifications of the above-described genetically encoded and non-encoded amino acids are summarized in TABLE 1, below. It is to be understood that TABLE 1 is for illustrative purposes only and does not purport to be an exhaustive list of amino acid residues which may comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 1

| | Genetically | |
|---|---|---|
| Classification | Encoded | Genetically Non-Encoded |
| Hydrophobic | | |
| Aromatic | F, Y, W | Phg, Nal, Thi, Tic, Phe(4-Cl), Phe(2-F), |
| | | Phe(3-F), Phe(4-F), Pyridyl Ala, |
| | | Benzothienyl Ala |
| Apolar | M, G, P | |
| Aliphatic | A, V, L, I | t-BuA, t-BuG, MeIle, Nle, MeVal, Cha, |
| | | bAla, MeGly, Aib |
| Hydrophilic | | |
| Acidic | D, E | |
| Basic | H, K, R | Dpr, Orn, hArg, Phe(p-NH₂), DBU, |
| | | A₂BU |
| Polar | Q, N, S, T, Y | Cit, AcLys, MSO, hSer |
| Cysteine-Like | C | Pen, hCys, p-methyl Cys |

As used herein, a “detectable label” has the ordinary meaning in the art and refers to an atom (e.g., radionuclide), molecule (e.g., fluorescein), or complex, that is or can be used to detect (e.g., due to a physical or chemical property), indicate the presence of a molecule or to enable binding of another molecule to which it is covalently bound or otherwise associated. The term “label” also refers to covalently bound or otherwise associated molecules (e.g., a biomolecule such as an enzyme) that act on a substrate to produce a detectable atom, molecule or complex. Detectable labels suitable for use in the present invention include any composition detectable by spectroscopic, photochemical, biochemical, immunochemical, electrical, optical or chemical means. Labels useful in the present invention include biotin for staining with labeled streptavidin conjugate, magnetic beads (e.g., Dynabeads™), fluorescent dyes (e.g., fluorescein, Texas red, rhodamine, green fluorescent protein, enhanced green fluorescent protein, and the like), radiolabels (e.g., ³H, ¹²⁵I, ³⁵S, ¹⁴C, or ³²P), enzymes (e.g., hydrolases, particularly phosphatases such as alkaline phosphatase, esterases and glycosidases, or oxidoreductases, particularly peroxidases such as horse radish peroxidase, and others commonly used in ELISAs), substrates, cofactors, inhibitors, chemiluminescent groups, chromogenic agents, and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels and chemiluminescent labels may be detected using photographic film or scintillation counters, fluorescent markers may be detected using a photodetector to detect emitted light (e.g., as in fluorescence-activated cell sorting). Enzymatic labels are typically detected by providing the enzyme with a substrate and detecting the reaction product produced by the action of the enzyme on the substrate, and colorimetric labels are detected by simply visualizing the colored label. Thus, a label is any composition detectable by spectroscopic, photochemical, biochemical, immunochemical, electrical, optical or chemical means. The label may be coupled directly or indirectly to the desired component of the assay according to methods well known in the art. Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal generating system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody. The molecules can also be conjugated directly to signal generating compounds, e.g., by conjugation with an enzyme or fluorophore. Means of detecting labels are well known to those of skill in the art. Thus, for example, where the label is a radioactive label, means for detection include a scintillation counter, photographic film as in autoradiography, or storage phosphor imaging. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Also, simple colorimetric labels may be detected by observing the color associated with the label. It will be appreciated that when pairs of fluorophores are used in an assay, it is often preferred that they have distinct emission patterns (wavelengths) so that they can be easily distinguished.

As used herein, the term “substantially identical” in the context of comparing amino acid sequences, means that the sequences have at least about 70%, at least about 80%, or at least about 90% amino acid residue identity when compared and aligned for maximum correspondence. An algorithm that is suitable for determining percent sequence identity and sequence similarity is the FASTA algorithm, which is described in Pearson, W. R. & Lipman, D. J., 1988, Proc. Natl. Acad. Sci. U.S.A. 85: 2444. See also W. R. Pearson, 1996, Methods Enzymol. 266: 227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

As used herein, the terms “test compound” or “test agent” are used interchangeably and refer to a candidate agent that may have enhancer/agonist, or inhibitor/antagonist activity, e.g., inhibiting or enhancing an interaction such as PDZ-PL binding. The candidate agents or test compounds may be any of a large variety of compounds, both naturally occurring and synthetic, organic and inorganic, and including polymers (e.g., oligopeptides, polypeptides, oligonucleotides, and polynucleotides), small molecules, antibodies (as broadly defined herein), sugars, fatty acids, nucleotides and nucleotide analogs, analogs of naturally occurring structures (e.g., peptide mimetics, nucleic acid analogs, and the like), and numerous other compounds. In certain embodiment, test agents are prepared from diversity libraries, such as random or combinatorial peptide or nonpeptide libraries. Many libraries are known in the art that can be used, e.g., chemically synthesized libraries, recombinant (e.g., phage display libraries), and in vitro translation-based libraries. Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383. Examples of phage display libraries are described in Scott and Smith, 1990, Science 249:386-390; Devlin et al., 1990, Science, 249:404-406; Christian, R. B., et al., 1992, J. Mol. Biol. 227:711-718); Lenstra, 1992, J. Immunol. Meth. 152:149-157; Kay et al., 1993, Gene 128:59-65; and PCT Publication No. WO 94/18318 dated Aug. 18, 1994. In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026. By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay).

As used herein, a “plurality” of PDZ proteins (or corresponding PDZ domains or PDZ fusion polypeptides) has its usual meaning. In some embodiments, the plurality is at least 5, and often at least 25, at least 40, or at least 60 different PDZ proteins. In some embodiments, the plurality is selected from the list of PDZ polypeptides listed in TABLE 4. In some embodiments, the plurality of different PDZ proteins are from (i.e., expressed in) a particular specified tissue or a particular class or type of cell. In some embodiments, the plurality of different PDZ proteins represents a substantial fraction (e.g., typically at least 50%, more often at least 80%) of all of the PDZ proteins known to be, or suspected of being, expressed in the tissue or cell(s), e.g., all of the PDZ proteins known to be present in neurons. In some embodiments, the plurality is at least 50%, usually at least 80%, at least 90% or all of the PDZ proteins disclosed herein as being expressed in a particular cell.

When referring to PL peptides (or the corresponding proteins, e.g., corresponding to those listed in TABLE 2, or elsewhere herein) a “plurality” may refer to at least 5, at least 10, and often at least 25 PLs such as those specifcally listed herein, or to the classes and percentages set forth supra for PDZ domains.

The term “neurological disorder,” “neurological injury”, “neurological disease” and other related terms generally refers to a disorder correlated with some type neuronal insult or neuronal cell death. Specific examples of such disorders include, but are not limited to, stroke, ischemic stroke, Parkinson's disease, Huntington's disease, Alzheimer's disease, epilepsy, inherited ataxias and motor neuron diseases.

A “stroke” has the meaning normally accepted in the art and generally refers to neurological injury resulting from impaired blood flow regardless of cause. Potential causes include, but are not limited to, embolism, hemorrhage and thrombosis. An “ischemic stroke” refers more specifically to a type of stroke that is of limited extent and caused due to blockage of blood flow.

A difference is in general is typically considered to be “statistically significant” if the difference is less than experimental error. Thus a difference is considered statistically significant if the probability of the observed difference occurring by chance (the p-value) is less than some predetermined level. As used herein a “statistically significant difference” can refer to a p-value that is <0.05, preferably <0.01 and most preferably <0.001.

### II. General

The present inventors have identified a large number of interactions between PDZ proteins and proteins that contain a PL motif that are involved in various biological functions in different types of cells. Some of these interactions involve PDZ:PL protein interactions between proteins that have important roles in neuronal cells. As such, modulation of these interactions have direct implications for the treatment of various neurological disorders, including stroke and ischemia.

Based upon the PDZ:PL interactions that have been detected, the inventors have identified a number of distinct strategies for treating various neurological disorders. One strategy is based upon the finding that the interaction between NMDAR proteins (which contain a PL sequence) and PSD-95 (a PDZ protein) is an important factor in triggering an excitotoxicity response in neuron cells. The inventors have determined common structural features of a class of polypeptides that are effective in disrupting the interaction between NMDAR proteins and PSD-95; polypeptides with these features are thus useful in treating neurological disorders associated with excitotoxicity. The second strategy is based upon the recognition that nNOS also has an important role in excitotoxicity responses. nNOS has an interesting structure in that it includes a PDZ domain, as well as an internal PL sequence. The current inventors determined that the internal PL sequence in nNOS binds to PSD-95. Thus, the second strategy involves the use of inhibitors to interfere with this interaction as a means to modulate biological activity in neurons. The third strategy is based upon the identification of specific PL proteins that bind to the PDZ domain of nNOS. Inhibitors can also be utilized to disrupt interactions between these protein binding combinations to affect biological activity in neurons.

The current inventors have thus identified compounds that inhibit the interactions between these different proteins, as well as developed methods for designing additional compounds. One general class of inhibitors are those that mimic the carboxy terminus of a PL protein and thus interfere with the ability of the carboxy terminus of the PL protein to bind its cognate PDZ protein. Another general class of inhibitors include the PDZ domain from a PDZ protein that is involved in an interaction that is to be disrupted. These inhibitors bind the PL protein that is the cognate ligand for the PDZ protein of interest and thus prevent binding between the PL protein and PDZ protein. Because the PDZ:PL protein interactions that are described herein are involved in the biological activity of neuronal cells, the inhibitors that are provided can be used to inhibit PDZ:PL protein interactions for the treatment of neurological disorders such as stroke, ischemia, Parkinson's disease, Huntington's disease, Alzheimer's disease, epilepsy, inherited ataxias and motor neuron diseases. Methods for determining whether a test compound acts a modulator of a particular PDZ protein and PL protein binding pair are also described.

For those PDZ proteins containing multiple PDZ domains, the methods that are provided can be utilized to determine to which specific domain(s) a particular PL protein of interest binds. The methods can thus be utilized to identify or design inhibitors that have increased selectivity for a particular PDZ domain. For instance, as described in greater detail below, the inventors have found that inhibitors with certain structural motifs preferentially inhibit binding between NMDR2 and the second PDZ domain of PSD-95, whereas inhibitors with different structural motifs preferentially inhibit binding between NMDR2 and the first PDZ domain of PSD-95. The methods that are disclosed can also be used to identify inhibitors with high binding affinity.

Because NMDAR proteins play a key regulatory role in neurons, an initial set of studies were undertaken to determine what PDZ proteins bind to the various NMDAR subunits (there are eight different isoforms of the NMDAR1 subunits, four different NMDAR2 subunit forms and several different NMDAR3 subunits). These analyses were conducted using the “A” and “G” assays described in detail below. The PDZ proteins identified as binding at least one NMDAR subunit protein are listed in TABLE 3. PDZ proteins found to bind all four NMDAR2 subunits are listed on the left-hand side of TABLE 7. Those PDZ proteins that bound at least one, but not all, of the NMDAR2 subunits are listed separately in TABLE 7.

The C-terminal sequences of the various NMDAR subunits that contain a PL sequence are listed in TABLE 2. Because the C-terminal region of the PL protein is the region that binds to PDZ proteins, agents that include similar amino acid motifs can be used to inhibit binding between NMDAR proteins and the PDZ proteins that bind to them (see TABLE 3). As described in greater detail below, for example, certain classes of peptide inhibitors typically include at least 2 contiguous amino acids from the C-terminus of the NMDAR proteins listed in TABLE 2, but can include 3-20 or more contiguous amino acids from the C-terminus.

One of the PDZ proteins identified in the initial investigation as interacting with NMDAR proteins was PSD-95 (see TABLE 7). Additional studies were subsequently undertaken to identify the structural motifs that were common to the polypeptides capable of inhibiting the interaction between NMDAR and PSD-95 (see Example 5). One class of compounds are polypeptides that have the following characteristics: 1) a length of about 3-20 amino acids (although somewhat longer polypeptides can be used), and 2) a C-terminal consensus sequence of X-T-X-V/L/A (the slash separates different amino acids that can appear at a given position). These polypeptides also typically had IC₅₀ values of less than 50 uM.

As alluded to above, in addition to the studies with respect to the PDZ proteins that bind to NMDAR proteins, the current inventors have also identified proteins having PL sequences that can bind to the PDZ domain of nNOS. Identification of these interactions also provides insight into excitotoxicity in neurons because of the key role that nNOS also plays in this process. Inhibitors having sequences that mimic the C-terminal motifs of these proteins can be used to inhibit the interaction of these proteins with nNOS.

In another set of experiments (see Example 9 and TABLE 9) PL sequences in addition to NMDAR2 sequences were identified as capable of binding to the PDZ domain of PSD-95. Thus, inhibitors incorporating these PL sequences can also be used to disrupt interactions between PL proteins and PDZ.

The inventors have also found that the C-terminus of PSD-95 is itself a PL sequence (RERL) and thus can bind PDZ proteins. Accordingly, another class of inhibitors are those that disrupt binding between the PL sequence of PSD-95 and its PDZ binding partners. Interactions of this type thus provide another therapeutic target for treatment of various neurological diseases.

Although the foregoing classes of inhibitors are based upon the C-terminal sequences of PL proteins that bind a PDZ protein, as alluded to above, another class of inhibitors includes polypeptides that include all or a part of a PDZ domain that binds to the PL sequence of a NMDAR protein or the internal PL sequence of nNOS. Because inhibitors in this class typically include most or the entire PDZ domain, polypeptide inhibitors in this class typically are at least 50-70 amino acids in length.

The various classes of polypeptide inhibitors just described can also be fusion proteins. These generally include a PL inhibitor peptide sequence such as those just listed that is fused to another sequence that encodes a separate protein domain. One specific example of an inhibitory fusion protein is one in which a PL sequence (e.g., those listed above) are coupled to a transmembrane transporter peptide. As described in greater detail infra and in Example 6, a variety of different transmembrane transporter peptides can be utilized.

Although certain classes of inhibitors such as those just described are polypeptides, other inhibitors are peptide mimetics or variants of these polypeptides as described in greater detail infra. Regardless of type, the inhibitors typically had IC₅₀ values less than 50 uM, 25 uM, 10 uM, 0.1 uM or 0.01 uM. In general the inhibitors typically have an IC₅₀ value of between 0.1-1 uM. These inhibitors can be formulated as pharmaceutical compositions and then used in the treatment of various neurological disorders such as those listed above.

The following sections provide additional details regarding the identification of PDZ:PL interactions in neuron cells, the structural characteristics of inhibitors that disrupt these interactions and treatment methods utilizing such inhibitors.

### III. Identification of Candidate PL Proteins and Synthesis of Peptides

A PL protein (short for PDZ Ligand protein), such as the NMDAR proteins described herein, is a protein (or a C-terminal fragment thereof) that can bind PDZ proteins via its carboxy terminus. PDZ proteins, in turn, are proteins with PDZ domains, which are domains common to three prototypical proteins: post synaptic density protein-95 (PSD-95), Drosophila large disc protein and Zonula Occludin 1 protein (see, e.g., Gomperts et al., 1996, Cell 84:659-662; see also, Songyang et al., 1997, Science 275:73; and Doyle et al., 1996, Cell 88:1067-1076). Certain classes of PDZ proteins contain three PDZ domains, one SH3 domain and one guanylate kinase domain. As described in greater detail herein, PL proteins have certain carboxy terminal motifs that enable these proteins to functions as ligands to PDZ proteins. When these carboxy terminal regions are referred to, the positioning of the carboxy terminal residues are sometimes referred to herein by a numbered position, which is illustrated in the following scheme:
Position: −3 −2 −1 0 (C-terminal)

Certain PDZ domains are bound by the C-terminal residues of PDZ-binding proteins. To identify NMDA receptors containing a PL motif, the C-terminal residues of sequences were visually inspected to identify sequences that bind to PDZ-domain containing proteins (see, e.g., Doyle et al., 1996, Cell 85, 1067; Songyang et al., 1997, Science 275, 73). TABLE 2 lists these proteins, and provides corresponding C-terminal sequences and GenBank accession numbers. Another investigation was conducted to identify PL motifs that bind to the PDZ domain of nNOS. The PL C-terminal motifs of the PL proteins binding to the PDZ domain are listed in TABLE 8.

A. Preparation of Peptides

1) Chemical Synthesis

The peptides of the invention or analogues thereof, may be prepared using virtually any art-known technique for the preparation of peptides and peptide analogues. For example, the peptides may be prepared in linear form using conventional solution or solid phase peptide syntheses and cleaved from the resin followed by purification procedures (Creighton, 1983, Protein Structures And Molecular Principles, W.H. Freeman and Co., N.Y.). Suitable procedures for synthesizing the peptides described herein are well known in the art. The composition of the synthetic peptides may be confirmed by amino acid analysis or sequencing (e.g., the Edman degradation procedure and mass spectroscopy).

In addition, analogues and derivatives of the peptides can be chemically synthesized. The linkage between each amino acid of the peptides of the invention may be an amide, a substituted amide or an isostere of amide. Nonclassical amino acids or chemical amino acid analogues can be introduced as a substitution or addition into the sequence. Nonclassical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general. Furthermore, the amino acid can be D (dextrorotary) or L (levorotary).

Synthetic peptides of defined sequence (e.g., corresponding to the carboxyl-termini of the indicated proteins) can be synthesized by any standard resin-based method (see, e.g., U.S. Pat. No. 4,108,846; see also, Caruthers et al., 1980, Nucleic Acids Res. Symp. Ser., 215-223; Horn et al., 1980, Nucleic Acids Res. Symp. Ser., 225-232; Roberge, et al., 1995, Science 269:202). The peptides used in the assays described herein were prepared by the FMOC (see, e.g., Guy and Fields, 1997, Meth. Enz. 289:67-83; Wellings and Atherton, 1997, Meth. Enz. 289:44-67). In some cases (e.g., for use in the A and G assays of the invention), peptides were labeled with biotin at the amino-terminus by reaction with a four-fold excess of biotin methyl ester in dimethylsulfoxide with a catalytic amount of base. The peptides were cleaved from the resin using a halide containing acid (e.g. trifluoroacetic acid) in the presence of appropriate antioxidants (e.g. ethanedithiol) and excess solvent lyophilized.

2) Recombinant Synthesis

If the peptide is composed entirely of gene-encoded amino acids, or a portion of it is so composed, the peptide or the relevant portion may also be synthesized using conventional recombinant genetic engineering techniques. For recombinant production, a polynucleotide sequence encoding a linear form of the peptide is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected into a suitable target cell which will express the peptide. Depending on the expression system used, the expressed peptide is then isolated by procedures well-established in the art. Methods for recombinant protein and peptide production are well known in the art (see, e.g., Maniatis et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y.).

A variety of host-expression vector systems may be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) may be used; when generating cell lines that contain multiple copies of expression product, SV40-, BPV- and EBV-based vectors may be used with an appropriate selectable marker.

In cases where plant expression vectors are used, the expression of sequences encoding the peptides of the invention may be driven by any of a number of promoters. For example, viral promoters such as the 35S RNA and 19S RNA promoters of CaMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680; Broglie et al., 1984, Science 224:838-843) or heat shock promoters, e.g., soybean hsp17.5-E or hsp17.3-B (Gurley et al., 1986, Mol. Cell. Biol. 6:559-565) may be used. These constructs can be introduced into planleukocytes using Ti plasmids, Ri plasmids, plant virus vectors, direct DNA transformation, microinjection, electroporation, etc. For reviews of such techniques see, e.g., Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

In one insect expression system that may be used to produce the peptides of the invention, Autographa californica nuclear polyhidrosis virus (AcNPV) is used as a vector to express the foreign genes. The virus grows in Spodoptera frugiperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (e.g., see Smith et al., 1983, J. Virol. 46:584; Smith, U.S. Pat. No. 4,215,051). Further examples of this expression system may be found in Current Protocols in Molecular Biology, Vol. 2, Ausubel et al., eds., Greene Publish. Assoc. & Wiley Interscience.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts. (e.g., See Logan & Shenk, 1984, Proc. Natl. Acad. Sci. USA 81:3655-3659). Alternatively, the vaccinia 7.5 K promoter may be used, (see, e.g., Mackett et al., 1982, Proc. Natl. Acad. Sci. USA 79:7415-7419; Mackett et al., 1984, J. Virol. 49:857-864; Panicali et al., 1982, Proc. Natl. Acad. Sci. USA 79:4927-4931).

Other expression systems for producing linear peptides of the invention will be apparent to those having skill in the art.

B. Purification of Peptides and Peptide Analogues

The peptides and peptide analogues of the invention can be purified by art-known techniques such as high performance liquid chromatography, ion exchange chromatography, gel electrophoresis, affinity chromatography and the like. The actual conditions used to purify a particular peptide or analogue will depend, in part, on factors such as net charge, hydrophobicity, hydrophilicity, etc., and will be apparent to those having skill in the art. The purified peptides can be identified by assays based on their physical or functional properties, including radioactive labeling followed by gel electrophoresis, radioimmuno-assays, ELISA, bioassays, and the like.

For affinity chromatography purification, any antibody which specifically binds the peptides or peptide analogues may be used. For the production of antibodies, various host animals, including but not limited to rabbits, mice, rats, etc., may be immunized by injection with a peptide. The peptide may be attached to a suitable carrier, such as BSA or KLH, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a peptide may be prepared using any technique which provides for the production of antibody molecules by continuous cell lines in culture. These include but are not limited to the hybridoma technique originally described by Koehler and Milstein, 1975, Nature 256:495-497, the human B-cell hybridoma technique, Kosbor et al., 1983, Immunology Today 4:72; Cote et al., 1983, Proc. Natl. Acad. Sci. U.S.A. 80:2026-2030 and the EBV-hybridoma technique (Cole et al., 1985, Monoclonal Antibodies and Cancer Therapy, Alan R. Liss, Inc., pp. 77-96 (1985)). In addition, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce peptide-specific single chain antibodies.

Antibody fragments which contain deletions of specific binding sites may be generated by known techniques. For example, such fragments include but are not limited to F(ab′)₂ fragments, which can be produced by pepsin digestion of the antibody molecule and Fab fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity for the peptide of interest.

The antibody or antibody fragment specific for the desired peptide can be attached, for example, to agarose, and the antibody-agarose complex is used in immunochromatography to purify peptides of the invention. See, Scopes, 1984, Protein Purification: Principles and Practice, Springer-Verlag New York, Inc., NY, Livingstone, 1974, Methods Enzymology: Immunoaffinity Chromatography of Proteins 34:723-731.

For the peptides used in the present invention, cleavage from resin and lyophilization was followed by peptides being redissolved and purified by reverse phase high performance liquid chromatography (HPLC). One appropriate HPLC solvent system involves a Vydac C-18 semi-preparative column running at 5 mL per minute with increasing quantities of acetonitrile plus 0.1% trifluoroacetic acid in a base solvent of water plus 0.1% trifluoroacetic acid. After HPLC purification, the identities of the peptides are confirmed by MALDI cation-mode mass spectrometry. As noted, exemplary biotinylated peptides are provided in TABLE 2.

### IV. PDZ Protein and PL Protein Interactions

TABLES 3, 7, 8 and 9 (Dave: Don't we also want Table 9) list PDZ proteins and other PL proteins which the current inventors have identified as binding to one another. Each page of TABLE 3 includes seven columns. The columns are numbered from left to right such that the left-most column is column 1 and the right-most column is column 7. Thus, the first column is labeled “internal PL ID” and lists AA numbers that serve as unique internal designations for each PL peptide. These ID numbers correspond to those listed in column 6 of TABLE 2. The second column is labeled “PL Name” and lists the various PL proteins/PDZ-Ligands that were examined. This column lists gene abbreviations, with subtypes included, for peptides corresponding to the carboxyl-terminal 20 amino acids of the protein listed. The third column, labeled “PL 20 mer Sequence,” lists the carboxyl-terminal 20 amino acids of the protein. All ligands are biotinylated at the amino-terminus. Some have been modified to eliminate cysteine amino acids from the 20 mer sequence. In these cases, wildtype sequences are presented in TABLE 2.

The PDZ protein (or proteins) that interact(s) with a PL peptide are listed in the fourth column that is labeled “PDZ Name”. This column provides the gene name for the PDZ portion of the GST-PDZ fusion that interacts with the PDZ-ligand to the left. For PDZ domain-containing proteins with multiple domains, the domain number is listed to the right of the PDZ (i.e., in column 5 labeled “PDZ Domain”), and indicates the PDZ domain number when numbered from the amino-terminus to the carboxy-terminus.

The sixth column labeled “Binding Strength” is a measure of the level of binding. In particular, it provides an absorbence value at 450 nm which indicates the amount of PL peptide bound to the PDZ protein. The following numerical values have the following meanings: ‘1’—A450 nm 0-1; ‘2’—A450 nm 1-2; ‘3’—A450 nm 2-3; ‘4’—A450 nm 3-4; ‘5’-A450 nm of 4. All interactions have been repeated a total of at least 4 times, and all show A450 nm values that are at least two times that of controls. Note that the binding strength has not been indicated for all interactions, and should not be used as a quantitative comparison of avidity between interactions. The last column in TABLE 3, labeled “Assay Used,” indicates whether the interaction was detected using the “A Assay,” the “G Assay,” or both assays (see below).

Further information regarding these PL proteins and PDZ proteins is provided in TABLES 2 and 4. In particular, TABLE 2 provides a list of known NMDA receptors, along with the amino acid sequence of the carboxyl-terminal 20 amino acids. When numbered from left to right, the first column labeled “Name” provides the commonly used abbreviation of the gene name. Genbank GI numbers are listed in column 2, labeled “GI#.” Columns 3 and 4, labeled “C-terminal 20 mer sequence” and “C-terminal 4 mer sequence,” respectively, list the last 20 amino acids, and the last 4 amino acids of each protein. Column 5, labeled “PL?” marks with an “X” those carboxy-terminal sequences that are predicted to display a classic PL amino acid motif. Many of the carboxyl-terminal motifs that are not marked in column 5 may exhibit binding to PDZ proteins, and the designation as a classic PL motif is in no way intended to predict or restrict NMDAR binding patterns to PDZ proteins. The sixth column labeled “internal PL ID” provides the internal designation number used to refer to a particular PL protein and correlates with the designation used in column 1 of TABLE 3.

Many of the genes listed in TABLE 2 express more than one amino acid sequence, depending on alternative exon splicing and single amino acid changes. When the information was available, alternatively spliced and point mutated isoforms of the same gene have been represented separately in TABLE 2. It is understood in the art that many alternatively spliced and point mutated forms of the same gene may exist in nature. As indicated supra, all peptides were biotinylated at the amino terminus and the amino acid sequences correspond to the C-terminal sequence of the gene name listed in column 1.

TABLE 4 lists the sequences of the PDZ domains cloned into a vector (PGEX-3X vector) for production of GST-PDZ fusion proteins (Pharmacia). More specifically, the first column (left to right) entitled “Gene Name” lists the name of the gene containing the PDZ domain. The second column labeled “GI or Acc#” is a unique Genbank identifier for the gene used to design primers for PCR amplification of the listed sequence. The next column labeled “Domain#” indicates the Pfam-predicted PDZ domain number, as numbered from the amino-terminus of the gene to the carboxy-terminus. The last column entitled “Sequence fused to GST Construct” provides the actual amino acid sequence inserted into the GST-PDZ expression vector as determined by DNA sequencing of the constructs.

As discussed in detail herein, the PDZ proteins listed in TABLES 3 and 4 are naturally occurring proteins containing a PDZ domain. Only significant interactions are presented in this TABLE 3. Thus, the present invention is directed to the detection and modulation of interactions between a PDZ protein and PL protein pair listed in TABLE 3. As used herein the phrase “protein pair” or ‘protein binding pair” when used in reference to a PDZ protein and PL protein refers to a PL protein and PDZ protein listed in TABLE 3 which bind to one another. It should be understood that TABLE 3 is set up to show that certain PL proteins bind to a plurality of PDZ proteins. For example, PL protein AA34.2 binds to PDZ proteins PSD95 and DLG1.

The interactions summarized in TABLE 3 can occur in a wide variety of cell types. Examples of such cells include hematopoietic, stem, neuronal, muscle, epidermal, epithelial, endothelial, and cells from essentially any tissue such as liver, lung, placenta, uterus, kidney, ovaries, testes, stomach, colon and intestine. Because the interactions disclosed herein can occur in such a wide variety of cell types, these interactions can also play an important role in a variety of biological functions.

Thus, for example, in certain embodiments of the invention, the PL proteins and/or the PDZ protein to which it binds are expressed in the nervous system (e.g., neurons). In an embodiment, the PL proteins of the invention bind a PDZ protein that is expressed in neurons. In various embodiments, the PL protein is highly expressed in neuronal cells. In still other instances the PL proteins and/or the PDZ protein to which it binds are expressed in non-neural cells (e.g., in hematopoietic cells).

In various embodiments of the invention, the PL protein is expressed or up-regulated upon cell activation (e.g., in stimulated neurons), upon entry into mitosis (e.g., up-regulation in rapidly proliferating cell populations), or in association with apoptosis.

In certain other various embodiments of the invention, the PL protein is (i) a protein that mediates the biological function of a neuronal cell, (ii) a protein that mediates apoptosis in a neural cell, (iii) a protein that is a N-methyl-D-aspartate receptor, or (iv) a protein that is a N-methyl-D-aspartate receptor and is expressed in neural cells.

In certain various embodiments of the invention, the methods disclosed infra are used to block the interaction between (i) NMDAR2A and an intracellular PDZ protein, (ii) NMDAR2B and an intracellular PDZ protein, (iii) NMDAR2C and an intracellular PDZ protein, and/or (iv) NMDAR2D and an intracellular PDZ protein.

In a preferred embodiment of the invention, the methods disclosed infra are used to block an interaction between all type 2 NMDA receptors (NMDAR2) and any intracellular PDZ.

In one embodiment of the invention, the methods disclosed infra are used to block an interaction between any type 2 NMDA receptor (NMDAR2) and any intracellular PDZ.

A. Detection of PDZ Domain-Containing Proteins

As noted supra, the present inventors have identified a number of PDZ protein and NMDAR PL protein interactions that can play a role in modulation of a number of biological functions in a variety of cell types. A comprehensive list of PDZ domain-containing proteins was retrieved from the Sanger Centre database (Pfam) searching for the keyword, “PDZ”. The corresponding cDNA sequences were retrieved from GenBank using the NCBI “entrez” database (hereinafter, “GenBank PDZ protein cDNA sequences”). The DNA portion encoding PDZ domains was identified by alignment of cDNA and protein sequence using CLUSTALW. Based on the DNA/protein alignment information, primers encompassing the PDZ domains were designed. The expression of certain PDZ-containing proteins in cells was detected by polymerase chain reaction (“PCR”) amplification of cDNAs obtained by reverse transcription (“RT”) of cell-derived RNA (i.e., “RT-PCR”). PCR, RT-PCR and other methods for analysis and manipulation of nucleic acids are well known and are described generally in Sambrook et al., (1989) MOLECULAR CLONING: A LABORATORY MANUAL, 2ND ED., VOLS. 1-3, Cold Spring Harbor Laboratory hereinafter, “Sambrook”); and Ausubel et al., CURRENT PROTOCOLS IN MOLECULAR BIOLOGY, Greene Publishing and Wiley-Interscience, New York (1997), as supplemented through January 1999 (hereinafter “Ausubel”).

Samples of cDNA for those sequences identified through the foregoing search were obtained and then amplified. In general a sample of the cDNA (typically, ⅕ of a 20 μl reaction) was used to conduct PCR. PCR was conducted using primers designed to amplify specifically PDZ domain-containing regions of PDZ proteins of interest. Oligonucleotide primers were designed to amplify one or more PDZ-encoding domains. The DNA sequences encoding the various PDZ domains of interest were identified by inspection (i.e., conceptual translation of the PDZ protein cDNA sequences obtained from GenBank, followed by alignment with the PDZ domain amino acid sequence). TABLE 4 shows the PDZ-encoded domains amplified, and the GenBank accession number of the PDZ-domain containing proteins. To facilitate subsequent cloning of PDZ domains, the PCR primers included endonuclease restriction sequences at their ends to allow ligation with pGEX-3X cloning vector (Pharmacia, GenBank XXI13852) in frame with glutathione-S transferase (GST).

TABLE 4 lists the proteins isolated for use in the aforementioned assays.

B. Production of Fusion Proteins Containing PDZ-Domains

GST-PDZ domain fusion proteins were prepared for use in the assays of the invention. PCR products containing PDZ encoding domains (as described supra) were subcloned into an expression vector to permit expression of fusion proteins containing a PDZ domain and a heterologous domain (i.e., a glutathione-S transferase sequence, “GST”). PCR products (i.e., DNA fragments) representing PDZ domain encoding DNA was extracted from agarose gels using the “sephaglas” gel extraction system (Pharmacia) according to the manufacturer's recommendations.

As noted supra, PCR primers were designed to include endonuclease restriction sites to facilitate ligation of PCR fragments into a GST gene fusion vector (pGEX-3X; Pharmacia, GenBank accession no. XXU13852) in-frame with the glutathione-S transferase coding sequence. This vector contains a IPTG inducible lacZ promoter. The pGEX-3X vector was linearized using Bam HI and Eco RI or, in some cases, Eco RI or Sma I, and dephosphorylated. For most cloning approaches, double digestion with Bam HI and Eco RI was performed, so that the ends of the PCR fragments to clone were Bam HI and Eco RI. In some cases, restriction endonuclease combinations used were Bgl II and Eco RI, Bam HI and Mfe I, or Eco RI only, Sma I only, or BamHI only. When more than one PDZ domain was cloned, the DNA portion cloned represents the PDZ domains and the cDNA portion located between individual domains. Precise locations of cloned fragments used in the assays are indicated in TABLE 4. Examples of the primers used to generate fragments for cloning are presented in TABLE 5. DNA linker sequences between the GST portion and the PDZ domain containing DNA portion vary slightly, dependent on which of the above described cloning sites and approaches were used. As a consequence, the amino acid sequence of the GST-PDZ fusion protein varies in the linker region between GST and PDZ domain. Protein linkers sequences corresponding to different cloning sites/approaches are shown below. Linker sequences (vector DNA encoded) are bold, PDZ domain containing gene derived sequences are in italics.

| | |
|---|---|
| 1) | GST-BamHI/BamHI-PDZ domain insert |
| | Gly--Ile-PDZ domain insert |
| 2) | GST-BamHI/BglII-PDZ domain insert |
| | Gly-Ile-PDZ domain insert |
| 3) | GST-EcoRI/EcoI-PDZ domain insert |
| | Gly-Ile-Pro-Gly--Asn-PDZ domain insert |
| 4) | GST--SmaI/SmaI-PDZ domain insert |
| | Gly-Ile-Pro-PDZ domain insert |

The PDZ-encoding PCR fragment and linearized pGEX-3X vector were ethanol precipitated and resuspended in 10 ul standard ligation buffer. Ligation was performed for 4-10 hours at 7° C. using T4 DNA ligase. It will be understood that some of the resulting constructs include very short linker sequences and that, when multiple PDZ domains were cloned, the constructs included some DNA located between individual PDZ domains.

The ligation products were transformed in DH5α or BL-21 E. coli bacteria strains. Colonies were screened for presence and identity of the cloned PDZ domain containing DNA as well as for correct fusion with the glutathione S-transferase encoding DNA portion by PCR and by sequence analysis. Positive clones were tested in a small scale assay for expression of the GST/PDZ domain fusion protein and, if expressing, these clones were subsequently grown up for large scale preparations of GST/PDZ fusion protein.

GST-PDZ domain fusion protein was overexpressed following addition of IPTG to the culture medium and purified. Detailed procedure of small scale and large scale fusion protein expression and purification are described in “GST Gene Fusion System” (second edition, revision 2; published by Pharmacia). In brief, a small culture (3-5 mls) containing a bacterial strain (DH5α, BL21 or JM109) with the fusion protein construct was grown overnight in LB-media at 37° C. with the appropriate antibiotic selection (100 ug/ml ampicillin; a.k.a. LB-amp). The overnight culture was poured into a fresh preparation of LB-amp (typically 250-500 mls) and grown until the optical density (OD) of the culture was between 0.5 and 0.9 (approximately 2.5 hours). IPTG (isopropyl β-D-thiogalactopyranoside) was added to a final concentration of 1.0 mM to induce production of GST fusion protein, and culture was grown an additional 1.5-2.5 hours. Bacteria were collect by centrifugation (4500 g) and resuspended in Buffer A− (50 mM Tris, pH 8.0, 50 mM dextrose, 1 mM EDTA, 200 uM phenylmethylsulfonylfluoride). An equal volume of Buffer A+ (Buffer A−, 4 mg/ml lysozyme) was added and incubated on ice for 3 min to lyse bacteria. An equal volume of Buffer B (10 mM Tris, pH 8.0, 50 mM KCl, 1 mM EDTA. 0.5% Tween-20, 0.5% NP40 (a.k.a. IGEPAL CA-630), 200 uM phenylmethylsulfonylfluoride) was added and incubated for an additional 20 min. The bacterial cell lysate was centrifuged (×20,000 g), and supernatant was added to glutathione Sepharose 4B (Pharmacia, cat no. 17-0765-01) previously swelled (rehydrated) in 1× phosphate-buffered saline (PBS). The supernatant-Sepharose slurry was poured into a column and washed with at least 20 bed volumes of 1×PBS. GST fusion protein was eluted off the glutathione sepharose by applying 0.5-1.0 ml aliquots of 5 mM glutathione and collected as separate fractions. Concentrations of fractions were determined using BioRad Protein Assay (cat no. 500-0006) according to manufacturer's specifications. Those fractions containing the highest concentration of fusion protein were pooled and dialyzed against 1×PBS/35% glycerol. Fusion proteins were assayed for size and quality by SDS gel electrophoresis (PAGE) as described in “Sambrook.” Fusion protein aliquots were stored at minus 80° C. and at minus 20° C.

C. Classification of PDZ Domain-Containing Proteins

The PDZ proteins identified herein as interacting with particular PL proteins can be grouped into a number of different categories. Thus, as described in greater detail below, the methods and reagents that are provided herein can be utilized to modulate PDZ interactions, and thus biological functions, that are regulated or otherwise involve the following classes of proteins. It should be recognized, however, that modulation of the interactions that are identified herein can be utilized to affect biological functions involving other protein classes.

1) Protein Kinases

A number of protein kinases contain PDZ domains. Protein kinases are widely involved in cellular metabolism and regulation of protein activity through phosphorylation of amino acids on proteins. An example of this is the regulation of signal transduction pathways such as T cell activation through the T cell Receptor, where ZAP-70 kinase function is required for transmission of the activation signal to downstream effector molecules. These molecules include, but are not limited to KIAA0303, KIAA0561, KIAA0807, KIAA0973, and CASK.

2) Guanalyte Kinases

A number of guanalyte kinases contain PDZ domains. These molecules include, but are not limited to Atrophin 1, CARD11, CARD14, DLG1, DLG2, DLG5, FLJ12615, MPP1, MPP2, NeDLG, p55T, PSD95, ZO-1, ZO-2, and ZO-3.

3) Guanine Exchange Factors

A number of guanine exchange factors contain PDZ domains. Guanine exchange factors regulate signal transduction pathways and other biological processes through facilitating the exchange of differently phosphorylated guanine residues. These molecules include, but are not limited to GTPase, Guanine Exchange, KIAA0313, KIAA0380, KIAA0382, KIAA1389, KIAA1415, TIAM1, and TAIM2.

4) LIM PDZ's

A number of LIM PDZ's contain PDZ domains. These molecules include, but are not limited to α-Actinin 2, ELFIN1, ENIGMA, HEMBA 1003117, KIAA0613, KIAA0858, KIAA0631, LIM Mystique, LIM protein, LIM-RIL, LIMK1, LIMK2, and LU-1.

5) Proteins Containing Only PDZ Domains

A number of proteins contain PDZ domains without any other predicted functional domains. These include, but are not limited to 26 s subunit p27, AIPC, Cytohesion Binding Protein, EZRIN Binding Protein, FLJ00011, FLJ20075, FLJ21687, GRIP1, HEMBA1000505, KIAA0545, KIAA0967, KIAA1202, KIAA1284, KIAA1526, KIAA1620, KIAA1719, MAGI1, Novel PDZ gene, Outer Membrane, PAR3, PAR6, PAR6γ, PDZ-73, PDZK1, PICK1, PIST, prIL16, Shank1, SIP1, SITAC-18, Syntenin, Syntrophin γ2, TIP1, TIP2, and TIP43.

6) Tyrosine Phosphatases

A number of Tyrosine phosphatases contain PDZ domains. Tyrosine phosphatases regulate biological processes such as signal transduction pathways through removal of phosphate groups required for function of the target protein. Examples of such enzymes include, but are not limited to PTN-3, PTN-4, and PTPL1.

7) Serine Proteases

A number of serine proteases contain PDZ domains. Proteases affect biological molecules by cleaving them to either activate or repress their functional ability. These enzymes have a variety of functions, including roles in digestion, blood coagulation and lysis of blood clots. These include, but are not limited to Novel Serine Protease and Serine Protease.

8) Viral Oncogene Interacting Proteins that Contain PDZ Domains

A number of TAX interacting proteins contain PDZ domains. Many of these also bind to multiple viral oncoproteins such as Adenovirus E4, Papillomavirus E6, and HBV protein X. These include, but are not limited to AIPC, Connector Enhancer, DLG1, DLG2, ERBIN, FLJ00011, FLJ11215, HEMBA1003117, INADL, KIAA0147, KIAA0807, KIAA1526, KIAA1634, LIMK1, LIM Mystique, LIM-RIL, MUPP1, NeDLG, Outer Membrane, PSD95, PTN-4, PTPL-1, Syntrophin γ1, Syntrophin γ2, TAX2-like protein, TIP2, TIP1, TIP33, and TIP43.

A number of proteins containing RA and/or RHA and/or DIL and/or IGFBP and/or WW and/or L27 and/or SAM and/or PH and/or DIX and/or DIP and/or Dishevelled and/or LRR and/or Hormone 3 and/or C2 and/or RPH3A and/or zf-TRAF and/or zf-C3HC4 and/or PID and/or NO_Synthase and/or Flavodoxin and/or FAD binding and/or NAD binding and/or Kazal and/or Trypsin and/or RBD and/or RGS and/or GoLoco and/or HR1 and/or BR01 contain PDZ domains. These include, but are not limited to AF6, APXL-1, MAGI1, DVL1, DVL2, DVL3, KIAA0417, KIAA0316, KIAA0340, KIAA0559, KIAA0751, KIAA0902, KIAA1095, KIAA1222, KIAA1634, MINT1, NOS1, RGS12, Rhophilin-like, Shank 3, Syntrophin 1α, Syntrophin P2, and X11β.

D. Assays for Detection of Interactions Between PDZ-Domain Polypeptides and NMDA Receptor PL Proteins

Two complementary assays, termed “A’ and “G,”” were developed to detect binding between a PDZ-domain polypeptide and candidate PDZ ligand. In each of the two different assays, binding is detected between a peptide having a sequence corresponding to the C-terminus of a protein anticipated to bind to one or more PDZ domains (i.e. a candidate PL peptide) and a PDZ-domain polypeptide (typically a fusion protein containing a PDZ domain). In the “A” assay, the candidate PL peptide is immobilized and binding of a soluble PDZ-domain polypeptide to the immobilized peptide is detected (the “A′” assay is named for the fact that in one embodiment an avidin surface is used to immobilize the peptide). In the “G” assay, the PDZ-domain polypeptide is immobilized and binding of a soluble PL peptide is detected (The “G” assay is named for the fact that in one embodiment a GST-binding surface is used to immobilize the PDZ-domain polypeptide). Preferred embodiments of these assays are described in detail infra. However, it will be appreciated by ordinarily skilled practitioners that these assays can be modified in numerous ways while remaining useful for the purposes of the present invention.

1) “A Assay” Detection of PDZ-Ligand Binding Using Immobilized PL Peptide.

In one aspect, the invention provides an assay in which biotinylated candidate PL peptides are immobilized on an avidin coated surface. The binding of PDZ-domain fusion protein to this surface is then measured. In a preferred embodiment, the PDZ-domain fusion protein is a GST/PDZ fusion protein and the assay is carried out as follows:

(1) Avidin is bound to a surface, e.g. a protein binding surface. In one embodiment, avidin is bound to a polystyrene 96 well plate (e.g., Nunc Polysorb (cat #475094) by addition of 100 μL per well of 20 μg/mL of avidin (Pierce) in phosphate buffered saline without calcium and magnesium, pH 7.4 (“PBS”, GibcoBRL) at 4° C. for 12 hours. The plate is then treated to block nonspecific interactions by addition of 200 μL per well of PBS containing 2 g per 100 mL protease-free bovine serum albumin (“PBS/BSA”) for 2 hours at 4° C. The plate is then washed 3 times with PBS by repeatedly adding 200 μL per well of PBS to each well of the, plate and then dumping the contents of the plate into a waste container and tapping the plate gently on a dry surface.

(2) Biotinylated PL peptides (or candidate PL peptides, e.g. see TABLE 2) are immobilized on the surface of wells of the plate by addition of 50 μL per well of 0.4 μM peptide in PBS/BSA for 30 minutes at 4° C. Usually, each different peptide is added to at least eight different wells so that multiple measurements (e.g. duplicates and also measurements using different (3ST/PDZ-domain fusion proteins and a GST alone negative control) can be made, and also additional negative control wells are prepared in which no peptide is immobilized. Following immobilization of the PL peptide on the surface, the plate is washed 3 times with PBS.

(3) GST/PDZ-domain fusion protein (prepared as described supra) is allowed to react with the surface by addition of 50 μL per well of a solution containing 5 μg/mL GST/PDZ-domain fusion protein in PBS/BSA for 2 hours at 4° C. As a negative control, GST alone (i.e. not a fusion protein) is added to specified wells, generally at least 2 wells (i.e. duplicate measurements) for each immobilized peptide. After the 2 hour reaction, the plate is washed 3 times with PBS to remove unbound fusion protein.

(4) The binding of the GST/PDZ-domain fusion protein to the avidin-biotinylated peptide surface can be detected using a variety of methods, and detectors known in the art. In one embodiment, 50 μL per well of an anti-GST antibody in PBS/BSA (e.g. 2.5 μg/mL of polyclonal goat-anti-GST antibody, Pierce) is added to the plate and allowed to react for 20 minutes at 4° C. The plate is washed 3 times with PBS and a second, detectably labeled antibody is added. In one embodiment, 50 μL per well of 2.5 μg/mL of horseradish peroxidase (HRP)-conjugated polyclonal rabbit anti-goat immunoglobulin antibody is added to the plate and allowed to react for 20 minutes at 4° C. The plate is washed 5 times with 50 mM Tris pH 8.0 containing 0.2% Tween 20, and developed by addition of 100 μL per well of HRP-substrate solution (TMB, Dako) for 20 minutes at room temperature (RT). The reaction of the HRP and its substrate is terminated by the addition of 100 μL per well of 1M sulfuric acid and the optical density (O.D.) of each well of the plate is read at 450 nm.

(5) Specific binding of a PL peptide and a PDZ-domain polypeptide is detected by comparing the signal from the well(s) in which the PL peptide and PDZ domain polypeptide are combined with the background signal(s). The background signal is the signal found in the negative controls. Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with repeated measurements of the background will result in a p-value <0.05, more typically a p-value <0.01, and most typically a p-value <0.001 or less.

As noted, in an embodiment of the “A” assay, the signal from binding of a GST/PDZ-domain fusion protein to an avidin surface not exposed to (i.e. not covered with) the PL peptide is one suitable negative control (sometimes referred to as “B”). The signal from binding of GST polypeptide alone (i.e. not a fusion protein) to an avidin-coated surface that has been exposed to (i.e. covered with) the PL peptide is a second suitable negative control (sometimes referred to as “B2”). Because all measurements are done in multiples (i.e. at least duplicate) the arithmetic mean (or, equivalently, average) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N-1). Thus, in one embodiment, specific binding of the PDZ protein to the plate-bound PL peptide is determined by comparing the mean signal (“mean S”) and standard error of the signal (“SE”) for a particular PL-PDZ combination with the mean B1 and/or mean B2.

2) “G Assay”-Detection of PDZ-Ligand Binding Using Immobilized PDZ-Domain Fusion Polypeptide

In one aspect, the invention provides an assay in which a GST/PDZ fusion protein is immobilized on a surface (“G” assay). The binding of labeled PL peptide (e.g., as listed in TABLE 2) to this surface is then measured. In a preferred embodiment, the assay is carried out as follows:

(1) A PDZ-domain polypeptide is bound to a surface, e.g. a protein binding surface. In a preferred embodiment, a GST/PDZ fusion protein containing one or more PDZ domains is bound to a polystyrene 96-well plate. The GST/PDZ fusion protein can be bound to the plate by any of a variety of standard methods known to one of skill in the art, although some care must be taken that the process of binding the fusion protein to the plate does not alter the ligand-binding properties of the PDZ domain. In one embodiment, the GST/PDZ fusion protein is bound via an anti-GST antibody that is coated onto the 96-well plate. Adequate binding to the plate can be achieved when:
a. 100 μL per well of 5 μg/mL goat anti-GST polyclonal antibody (Pierce) in PBS is added to a polystyrene 96-well plate (e.g., Nunc Polysorb) at 4° C. for 12 hours.
b. The plate is blocked by addition of 200 μL per well of PBS/BSA for 2 hours at 4° C.
c. The plate is washed 3 times with PBS.
d. 50 μL per well of 5 μg/mL GST/PDZ fusion protein) or, as a negative control, GST polypeptide alone (i.e. not a fusion protein) in PBS/BSA is added to the plate for 2 hours at 4° C.
e. the plate is again washed 3 times with PBS.

(2) Biotinylated PL peptides are allowed to react with the surface by addition of 50 μL per well of 20 μM solution of the biotinylated peptide in PBS/BSA for 10 minutes at 4° C., followed by an additional 20 minute incubation at 25° C. The plate is washed 3 times with ice cold PBS.

(3) The binding of the biotinylated peptide to the GST/PDZ fusion protein surface can be detected using a variety of methods and detectors known to one of skill in the art. In one embodiment, 100 μL per well of 0.5 μg/mL streptavidin-horse radish peroxidase (HRP) conjugate dissolved in BSA/PBS is added and allowed to react for 20 minutes at 4° C. The plate is then washed 5 times with 50 mM Tris pH 8.0 containing 0.2% Tween 20, and developed by addition of 100 μL per well of HRP-substrate solution (TMB, Dako) for 20 minutes at room temperature (RT). The reaction of the HRP and its substrate is terminated by addition of 100 μL per well of 1 M sulfuric acid, and the optical density (O.D.) of each well of the plate is read at 450 um.

(4) Specific binding of a PL peptide and a PDZ domain polypeptide is determined by comparing the signal from the well(s) in which the PL peptide and PDZ domain polypeptide are combined, with the background signal(s). The background signal is the signal found in the negative control(s). Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with—repeated measurements of the background will result in a p-value<0.05, more typically a p-value<0.01, and most typically a p-value<0.001 or less. As noted, in an embodiment of the “G” assay, the signal from binding of a given PL peptide to immobilized (surface bound) GST polypeptide alone is one suitable negative control (sometimes referred to as “B 1”). Because all measurement are done in multiples (i.e. at least duplicate) the arithmetic mean (or, equivalently, average.) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N-1). Thus, in one embodiment, specific binding of the PDZ protein to the platebound peptide is determined by comparing the mean signal (“mean S”) and standard error of the signal (“SE”) for a particular PL-PDZ combination with the mean B1.

i) “G′ Assay” and “G″ Assay”

Two specific modifications of the specific conditions described supra for the “G assay” are particularly useful. The modified assays use lesser quantities of labeled PL peptide and have slightly different biochemical requirements for detection of PDZ-ligand binding compared to the specific assay conditions described supra.

For convenience, the assay conditions described in this section are referred to as the “G′ assay” and the “G″ assay,” with the specific conditions described in the preceding section on G assays being referred to as the “G⁰ assay.” The “G′ assay” is identical to the “G⁰ assay” except at step (2) the peptide concentration is 10 uM instead of 20 uM. This results in slightly lower sensitivity for detection of interactions with low affinity and/or rapid dissociation rate. Correspondingly, it slightly increases the certainty that detected interactions are of sufficient affinity and half-life to be of biological importance and useful therapeutic targets.

The “G″ assay” is identical to the “G⁰ assay” except that at step (2) the peptide concentration is 1 μM instead of 20 μM and the incubation is performed for 60 minutes at 25° C. (rather than, e.g., 10 minutes at 4° C. followed by 20 minutes at 25° C.). This results in lower sensitivity for interactions of low affinity, rapid dissociation rate, and/or affinity that is less at 25° C. than at 4° C. Interactions will have lower affinity at 25° C. than at 4° C. if (as we have found to be generally true for PDZ-ligand binding) the reaction entropy is negative (i.e. the entropy of the products is less than the entropy of the reactants). In contrast, the PDZ-PL binding signal may be similar in the “G″ assay” and the “G⁰ assay” for interactions of slow association and dissociation rate, as the PDZ-PL complex will accumulate during the longer incubation of the “G″ assay.” Thus comparison of results of the “G″ assay” and the “G⁰ assay” can be used to estimate the relative entropies, enthalpies, and kinetics of different PDZ-PL interactions. (Entropies and enthalpies are related to binding affinity by the equations delta G=RT ln(Kd)=delta H−T delta S where delta G, H, and S are the reaction free energy, enthalpy, and entropy respectively, T is the temperature in degrees Kelvin, R is the gas constant, and Kd is the equilibrium dissociation constant). In particular, interactions that are detected only or much more strongly in the “G⁰ assay” generally have a rapid dissociation rate at 25° C. (t½<10 minutes) and a negative reaction entropy, while interactions that are detected similarly strongly in the “G″ assay” generally have a slower dissociation rate at 25° C. (t½>10 minutes). Rough estimation of the thermodynamics and kinetics of PDZ-PL interactions (as can be achieved via comparison of results of the “G⁰ assay” versus the “G″ assay” as outlined supra) can be used in the design of efficient inhibitors of the interactions. For example, a small molecule inhibitor based on the chemical structure of a PL that dissociates slowly from a given PDZ domain (as evidenced by similar binding in the “G″ assay” as in the “G⁰ assay”) may itself dissociate slowly and thus be of high affinity.

In this manner, variation of the temperature and duration of step (2) of the “G assay” can be used to provide insight into the kinetics and thermodynamics of the PDZ-ligand binding reaction and into design of inhibitors of the reaction.

3) Assay Variations

As discussed supra, it will be appreciated that many of the steps in the above-described assays can be varied, for example, various substrates can be used for binding the PL and PDZ-containing proteins; different types of PDZ containing fusion proteins can be used; different labels for detecting PDZ/PL interactions can be employed; and different ways of detection can be used.

The PL protein used in the assay is not intended to be limited to a 20 amino acid peptide. Full length or partial protein may be used, either alone or as a fusion protein. For example, a GST-PL protein fusion may be bound to the anti-GST antibody, with PDZ protein added to the bound PL protein or peptide.

The PDZ-PL detection assays can employ a variety of surfaces to bind the PL and PDZ-containing proteins. For example, a surface can be an “assay plate” which is formed from a material (e.g. polystyrene) which optimizes adherence of either the PL protein or PDZ-containing protein thereto. Generally, the individual wells of the assay plate will have a high surface area to volume ratio and therefore a suitable shape is a flat bottom well (where the proteins of the assays are adherent). Other surfaces include, but are not limited to, polystyrene or glass beads, polystyrene or glass slides, and the like.

For example, the assay plate can be a “microtiter” plate. The term “microtiter” plate when used herein refers to a multiwell assay plate, e.g., having between about 30 to 200 individual wells, usually 96 wells. Alternatively, high density arrays can be used. Often, the individual wells of the microtiter plate will hold a maximum volume of about 250 ul. Conveniently, the assay plate is a 96 well polystyrene plate (such as that sold by Becton Dickinson Labware, Lincoln Park, N.J.), which allows for automation and high throughput screening. Other surfaces include polystyrene microtiter ELISA plates such as that sold by Nunc Maxisorp, Inter Med, Denmark. Often, about 50 ul to 300 ul, more preferably 100 ul to 200 ul, of an aqueous sample comprising buffers suspended therein will be added to each well of the assay plate.

The detectable labels of the invention can be any detectable compound or composition which is conjugated directly or indirectly with a molecule (such as described above). The label can be detectable by itself (e.g., radioisotope labels or fluorescent labels) or, in the case of an enzymatic label, can catalyze a chemical alteration of a substrate compound or composition which is detectable. The preferred label is an enzymatic one which catalyzes a color change of a non-radioactive color reagent.

Sometimes, the label is indirectly conjugated with the antibody. One of skill is aware of various techniques for indirect conjugation. For example, the antibody can be conjugated with biotin and any of the categories of labels mentioned above can be conjugated with avidin, or vice versa (see also “A” and “G” assay above). Biotin binds selectively to avidin and thus, the label can be conjugated with the antibody in this indirect manner. See, Ausubel, supra, for a review of techniques involving biotin-avidin conjugation and similar assays. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

Assay variations can include different washing steps. By “washing” is meant exposing the solid phase to an aqueous solution (usually a buffer or cell culture media) in such a way that unbound material (e.g., non-adhering cells, non-adhering capture agent, unbound ligand, receptor, receptor construct, cell lysate, or HRP antibody) is removed therefrom. To reduce background noise, it is convenient to include a detergent (e.g., Triton X) in the washing solution. Usually, the aqueous washing solution is decanted from the wells of the assay plate following washing. Conveniently, washing can be achieved using an automated washing device. Sometimes, several washing steps (e.g., between about 1 to 10 washing steps) can be required.

Various buffers can also be used in PDZ-PL detection assays. For example, various blocking buffers can be used to reduce assay background. The term “blocking buffer” refers to an aqueous, pH buffered solution containing at least one blocking compound which is able to bind to exposed surfaces of the substrate which are not coated with a PL or PDZ-containing protein. The blocking compound is normally a protein such as bovine serum albumin (BSA), gelatin, casein or milk powder and does not cross-react with any of the reagents in the assay. The block buffer is generally provided at a pH between about 7 to 7.5 and suitable buffering agents include phosphate and TRIS.

Various enzyme-substrate combinations can also be utilized in detecting PDZ-PL interactions. Examples of enzyme-substrate combinations include, for example:

(i) Horseradish peroxidase (HRPO) with hydrogen peroxidase as a substrate, wherein the hydrogen peroxidase oxidizes a dye precursor (e.g. orthophenylene diamine [OPD] or 3,3′,5,5′-tetramethyl benzidine hydrochloride [TMB]) (as described above).

(ii) alkaline phosphatase (AP) with para-Nitrophenyl phosphate as chromogenic substrate.

(iii) β-D-galactosidase (β D-Gal) with a chromogenic substrate (e.g. p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980, both of which are herein incorporated by reference.

Further, it will be appreciated that, although, for convenience, the present discussion primarily refers antagonists of PDZ-PL interactions, agonists of PDZ-PL interactions can be identified using the methods disclosed herein or readily apparent variations thereof.

E. Detecting PDZ-PL Interactions

The present inventors were able in part to identify the interactions summarized in TABLE 3 and TABLE 8. by developing new high throughput screening assays which are described supra. Various other assay formats known in the art can be used to select ligands that are specifically reactive with a particular protein. For example, solid-phase ELISA immunoassays, immunoprecipitation, Biacore, Fluorescence Polarization (FP), Fluorescence Resonance Energy Transfer (FRET) and Western blot assays can be used to identify peptides that specifically bind PDZ-domain polypeptides. As discussed supra, two different, complementary assays were developed to detect PDZ-PL interactions. In each, one binding partner of a PDZ-PL pair is immobilized, and the ability of the second binding partner to bind is determined. These assays, which are described supra, can be readily used to screen for hundreds to thousands of potential PDZ-ligand interactions in a few hours. Thus these assays can be used to identify yet more novel PDZ-PL interactions in neuronal cells. In addition, they can be used to identify antagonists of PDZ-PL interactions (see infra).

In various embodiments, fusion protein are used in the assays and devices of the invention. Methods for constructing and expressing fusion proteins are well known. Fusion proteins generally are described in Ausubel et al., supra, Kroll et al., 1993, DNA Cell. Biol. 12:441, and Imai et al., 1997, Cell 91:521-30. Usually, the fusion protein includes a domain to facilitate immobilization of the protein to a solid substrate (“an immobilization domain”). Often, the immobilization domain includes an epitope tag (i.e., a sequence recognized by a antibody, typically a monoclonal antibody) such as polyhistidine (Bush et al, 1991, J. Biol Chem 266:13811-14), SEAP (Berger et al, 1988, Gene 66:1-10), or M1 and M2 flag (see, e.g, U.S. Pat. Nos. 5,011,912; 4,851,341; 4,703,004; 4,782,137). In an embodiment, the immobilization domain is a GST coding region. It will be recognized that, in addition to the PDZ-domain and the particular residues bound by an immobilized antibody, protein A, or otherwise contacted with the surface, the protein (e.g., fusion protein), will contain additional residues. In some embodiments these are residues naturally associated with the PDZ-domain (i.e., in a particular PDZ-protein) but they may include residues of synthetic (e.g., poly(alanine)) or heterologous origin (e.g., spacers of, e.g., between 10 and 300 residues).

PDZ domain-containing polypeptide used in the methods of the invention (e.g., PDZ fusion proteins) of the invention are typically made by (1) constructing a vector (e.g., plasmid, phage or phagemid) comprising a polynucleotide sequence encoding the desired polypeptide, (2) introducing the vector into an suitable expression system (e.g., a prokaryotic, insect, mammalian, or cell free expression system), (3) expressing the fusion protein and (4) optionally purifying the fusion protein.

In one embodiment, expression of the protein comprises inserting the coding sequence into an appropriate expression vector (i.e., a vector that contains the necessary elements for the transcription and translation of the inserted coding sequence required for the expression system employed, e.g., control elements including enhancers, promoters, transcription terminators, origins of replication, a suitable initiation codon (e.g., methionine), open reading frame, and translational regulatory signals (e.g., a ribosome binding site, a termination codon and a polyadenylation sequence. Depending on the vector system and host utilized, any number of suitable transcription and translation elements, including constitutive and inducible promoters, can be used.

The coding sequence of the fusion protein includes a PDZ domain and an immobilization domain as described elsewhere herein. Polynucleotides encoding the amino acid sequence for each domain can be obtained in a variety of ways known in the art; typically the polynucleotides are obtained by PCR amplification of cloned plasmids, cDNA libraries, and cDNA generated by reverse transcription of RNA, using primers designed based on sequences determined by the practitioner or, more often, publicly available (e.g., through GenBank). The primers include linker regions (e.g., sequences including restriction sites) to facilitate cloning and manipulation in production of the fusion construct. The polynucleotides corresponding to the PDZ and immobilization regions are joined in-frame to produce the fusion protein-encoding sequence.

The fusion proteins of the invention may be expressed as secreted proteins (e.g., by including the signal sequence encoding DNA in the fusion gene; see, e.g., Lui et al, 1993, PNAS USA, 90:8957-61) or as nonsecreted proteins.

In some embodiments, the PDZ-containing proteins are immobilized on a solid surface. The substrate to which the polypeptide is bound may in any of a variety of forms, e.g., a microtiter dish, a test tube, a dipstick, a microcentrifuge tube, a bead, a spinnable disk, and the like. Suitable materials include glass, plastic (e.g., polyethylene, PVC, polypropylene, polystyrene, and the like), protein, paper, carbohydrate, lipip monolayer or supported lipid bilayer, and other solid supports. Other materials that may be employed include ceramics, metals, metalloids, semiconductive materials, cements and the like.

In some embodiments, the fusion proteins are organized as an array. The term “array,” as used herein, refers to an ordered arrangement of immobilized fusion proteins, in which particular different fusion proteins (i.e., having different PDZ domains) are located at different predetermined sites on the substrate. Because the location of particular fusion proteins on the array is known, binding at that location can be correlated with binding to the PDZ domain situated at that location. Immobilization of fusion proteins on beads (individually or in groups) is another particularly useful approach. In one embodiment, individual fusion proteins are immobilized on beads. In one embodiment, mixtures of distinguishable beads are used. Distinguishable beads are beads that can be separated from each other on the basis of a property such as size, magnetic property, color (e.g., using FACS) or affinity tag (e.g., a bead coated with protein A can be separated from a bead not coated with protein A by using IgG affinity methods). Binding to particular PDZ domain may be determined; similarly, the effect of test compounds (i.e., agonists and antagonists of binding) may be determined.

Methods for immobilizing proteins are known, and include covalent and non-covalent methods. One suitable immobilization method is antibody-mediated immobilization. According to this method, an antibody specific for the sequence of an “immobilization domain” of the PDZ-domain containing protein is itself immobilized on the substrate (e.g., by adsorption). One advantage of this approach is that a single antibody may be adhered to the substrate and used for immobilization of a number of polypeptides (sharing the same immobilization domain). For example, an immobilization domain consisting of poly-histidine (Bush et al, 1991, J. Biol Chem 266:13811-14) can be bound by an anti-histidine monoclonal antibody (R&D Systems, Minneapolis, Minn.); an immobilization domain consisting of secreted alkaline phosphatase (“SEAP”) (Berger et al, 1988, Gene 66:1-10) can be bound by anti-SEAP (Sigma Chemical Company, St. Louis, Mo.); an immobilization domain consisting of a FLAG epitope can be bound by anti-FLAG. Other ligand-antiligand immobilization methods are also suitable (e.g., an immobilization domain consisting of protein A sequences (Harlow and Lane, 1988, Antibodies A Laboratory Manual, Cold Spring Harbor Laboratory; Sigma Chemical Co., St. Louis, Mo.) can be bound by IgG; and an immobilization domain consisting of streptavidin can be bound by biotin (Harlow & Lane, supra; Sigma Chemical Co., St. Louis, Mo.). In a preferred embodiment, the immobilization domain is a GST moiety, as described herein.

When antibody-mediated immobilization methods are used, glass and plastic are especially useful substrates. The substrates may be printed with a hydrophobic (e.g., Teflon) mask to form wells. Preprinted glass slides with 3, 10 and 21 wells per 14.5 cm² slide “working area” are available from, e.g., SPI Supplies, West Chester, Pa.; also see U.S. Pat. No. 4,011,350). In certain applications, a large format (12.4 cm×8.3 cm) glass slide is printed in a 96 well format is used; this format facilitates the use of automated liquid handling equipment and utilization of 96 well format plate' readers of various types (fluorescent, colorimetric, scintillation). However, higher densities may be used (e.g., more than 10 or 100 polypeptides per cm²). See, e.g., MacBeath et al, 2000, Science 289:1760-63.

Typically, antibodies are bound to substrates (e.g., glass substrates) by adsorption. Suitable adsorption conditions are well known in the art and include incubation of 0.5-50 μg/ml (e.g., 10 μg/ml) mAb in buffer (e.g., PBS, or 50 to 300 mM Tris, MOPS, HEPES, PIPES, acetate buffers, pHs 6.5 to 8, at 4° C.) to 37° C. and from 1 hr to more than 24 hours.

Proteins may be covalently bound or noncovalently attached through nonspecific bonding. If covalent bonding between a the fusion protein and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

F. Results of PDZ-PL Interaction Assays

TABLE 3 shows the results of assays in which specific binding was detected between NMDAR proteins and PDZ proteins using the “G′” assay described herein. TABLE 8 summarizes the results of interactions between a number of PL proteins with nNOS. TABLE 9 lists PL sequences that bind to the PDZ domain of PSD-95.

G. Measurement of PDZ-Ligand Binding Affinity

The “A” and “G” assays of the invention can be used to determine the “apparent affinity” of binding of a PDZ ligand peptide to a PDZ-domain polypeptide. Apparent affinity is determined based on the concentration of one molecule required to saturate the binding of a second molecule (e.g., the binding of a ligand to a receptor). Two particularly useful approaches for quantitation of apparent affinity of PDZ-ligand binding are provided infra.

Approach 1:

(1) A GST/PDZ fusion protein, as well as GST alone as a negative control, are bound to a surface (e.g., a 96-well plate) and the surface blocked and washed as described supra for the “G” assay.

(2) 50 μL per well of a solution of biotinylated PL peptide (e.g. as shown in TABLE 2) is added to the surface in increasing concentrations in PBS/BSA (e.g. at 0.1 μM, 0.33 μM, 1 μM, 3.3 μM, 10 μM, 33 μM, and 100 μM). In one embodiment, the PL peptide is allowed to react with the bound GST/PDZ fusion protein (as well as the GST alone negative control) for 10 minutes at 4° C. followed by 20 minutes at 25° C. The plate is washed 3 times with ice cold PBS to remove unbound labeled peptide.

(3) The binding of the PL peptide to the immobilized PDZ-domain polypeptide is detected as described supra for the “G” assay.

(4) For each concentration of peptide, the net binding signal is determined by subtracting the binding of the peptide to GST alone from the binding of the peptide to the GST/PDZ fusion protein. The net binding signal is then plotted as a function of ligand concentration and the plot is fit (e.g. by using the Kaleidagraph software package curve fitting algorithm) to the following equation, where “Signal_{[ligand]}” is the net binding signal at PL peptide concentration “[ligand],” “Kd” is the apparent affinity of the binding event, and “Saturation Binding” is a constant determined by the curve fitting algorithm to optimize the fit to the experimental data:

Signal_{[ligand]}=Saturation Binding×([ligand]/([ligand]+Kd))

For reliable application of the above equation it is necessary that the highest peptide ligand concentration successfully tested experimentally be greater than, or at least similar to, the calculated Kd (equivalently, the maximum observed binding should be similar to the calculated saturation binding). In cases where satisfying the above criteria proves difficult, an alternative approach (infra) can be used.

Approach 2:

(1) A fixed concentration of a PDZ-domain polypeptide and increasing concentrations of a labeled PL peptide (labeled with, for example, biotin or fluorescein, see TABLE 2 for representative peptide amino acid sequences) are mixed together in solution and allowed to react. In one embodiment, preferred peptide concentrations are 0.1 μM, 1 μM, 10 μM, 100 μM, 1 mM. In various embodiments, appropriate reaction times can range from 10 minutes to 2 days at temperatures ranging from 4° C. to 37° C. In some embodiments, the identical reaction can also be carried out using a non-PDZ domain-containing protein as a control (e.g., if the PDZ-domain polypeptide is fusion protein, the fusion partner can be used).

(2) PDZ-ligand complexes can be separated from unbound labeled peptide using a variety of methods known in the art. For example, the complexes can be separated using high performance size-exclusion chromatography (HPSEC, gel filtration) (Rabinowitz et al., 1998, Immunity 9:699), affinity chromatography (e.g. using glutathione Sepharose beads), and affinity absorption (e.g., by binding to an anti-GST-coated plate as described supra).

(3) The PDZ-ligand complex is detected based on presence of the label on the peptide ligand using a variety of methods and detectors known to one of skill in the art. For example, if the label is fluorescein and the separation is achieved using HPSEC, an in-line fluorescence detector can be used. The binding can also be detected as described supra for the G assay.

(4) The PDZ-ligand binding signal is plotted as a function of ligand concentration and the plot is fit. (e.g., by using the Kaleidagraph software package curve fitting algorithm) to the following equation, where “Signal_{[ligand]}” is the binding signal at PL peptide concentration “[ligand],” “Kd” is the apparent affinity of the binding event, and “Saturation Binding” is a constant determined by the curve fitting algorithm to optimize the fit to the experimental data:

Signal_{[Ligand]}=Saturation Binding×([ligand]/([ligand+Kd])

Measurement of the affinity of a labeled peptide ligand binding to a PDZ-domain polypeptide is useful because knowledge of the affinity (or apparent affinity) of this interaction allows rational design of inhibitors of the interaction with known potency. The potency of inhibitors in inhibition would be similar to (i.e. within one-order of magnitude of) the apparent affinity of the labeled peptide ligand binding to the PDZ-domain.

Thus, in one aspect, the invention provides a method of determining the apparent affinity of binding between a PDZ domain and a ligand by immobilizing a polypeptide comprising the PDZ domain and a non-PDZ domain on a surface, contacting the immobilized polypeptide with a plurality of different concentrations of the ligand, determining the amount of binding of the ligand to the immobilized polypeptide at each of the concentrations of ligand, and calculating the apparent affinity of the binding based on that data. Typically, the polypeptide comprising the PDZ domain and a non-PDZ domain is a fusion protein. In one embodiment, the e.g., fusion protein is GST-PDZ fusion protein, but other polypeptides can also be used (e.g., a fusion protein including a PDZ domain and any of a variety of epitope tags, biotinylation signals and the like) so long as the polypeptide can be immobilized In an orientation that does not abolish the ligand binding properties of the PDZ domain, e.g, by tethering the polypeptide to the surface via the non-PDZ domain via an anti-domain antibody and leaving the PDZ domain as the free end. It was discovered, for example, reacting a PDZ-GST fusion polypeptide directly to a plastic plate provided suboptimal results. The calculation of binding affinity itself can be determined using any suitable equation (e.g., as shown supra; also see Cantor and Schimmel (1980) BIOPHYSICAL CHEMISTRY WH Freeman & Co., San Francisco) or software.

Thus, in a preferred embodiment, the polypeptide is immobilized by binding the polypeptide to an immobilized immunoglobulin that binds the non-PDZ domain (e.g., an anti-GST antibody when a GST-PDZ fusion polypeptide is used). In a preferred embodiment, the step of contacting the ligand and PDZ-domain polypeptide is carried out under the conditions provided supra in the description of the “G” assay. It will be appreciated that binding assays are conveniently carried out in multiwell plates (e.g., 24-well, 96-well plates, or 384 well plates).

The present method has considerable advantages over other methods for measuring binding affinities PDZ-PL affinities, which typically involve contacting varying concentrations of a GST-PDZ fusion protein to a ligand-coated surface. For example, some previously described methods for determining affinity (e.g., using immobilized ligand and GST-PDZ protein in solution) did not account for oligomerization state of the fusion proteins used, resulting in potential errors of more than an order of magnitude.

Although not sufficient for quantitative measurement of PDZ-PL binding affinity, an estimate of the relative strength of binding of different PDZ-PL pairs can be made based on the absolute magnitude of the signals observed in the “G assay.” This estimate will reflect several factors, including biologically relevant aspects of the interaction, including the affinity and the dissociation rate. For comparisons of different ligands binding to a given PDZ domain-containing protein, differences in absolute binding signal likely relate primarily to the affinity and/or dissociation rate of the interactions of interest.

H. Assays to Identify Novel PDZ Domain Binding Moieties and to Identify Modulators of PDZ Protein-PL Protein Binding

Although described supra primarily in terms of identifying interactions between PDZ-domain polypeptides and PL proteins, the assays described supra and other assays can also be used to identify the binding of other molecules (e.g., peptide mimetics, small molecules, and the like) to PDZ domain sequences. For example, using the assays disclosed herein, combinatorial and other libraries of compounds can be screened, e.g., for molecules that specifically bind to PDZ domains. Screening of libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and PCT Publication No. WO 94/18318.

In a specific embodiment, screening can be carried out by contacting the library members with a PDZ-domain polypeptide immobilized on a solid support (e.g. as described supra in the “G” assay) and harvesting those library members that bind to the protein. Examples of such screening methods, termed “panning” techniques are described by way of example in Parmley and Smith, 1988, Gene 73:305-318; Fowlkes et al., 1992, BioTechniques 13:422-427; PCT Publication No. WO 94/18318; and in references cited hereinabove.

In another embodiment, the two-hybrid system for selecting interacting proteins in yeast (Fields and Song, 1989, Nature 340:245-246; Chien et al., 1991, Proc. Natl. Acad. Sci. USA 88:9578-9582) can be used to identify molecules that specifically bind to a PDZ domain-containing protein. Furthermore, the identified molecules are further tested for their ability to inhibit transmembrane receptor interactions with a PDZ domain.

In one aspect of the invention, antagonists of an interaction between a PDZ protein and a PL protein are identified. In one embodiment, a modification of the “A” assay described supra is used to identify antagonists. In one embodiment, a modification of the “G” assay described supra is used to identify antagonists.

In one embodiment, screening assays are used to detect molecules that specifically bind to PDZ domains. Such molecules are useful as agonists or antagonists of PDZ-protein-mediated cell function (e.g., cell activation, e.g., T cell activation, vesicle transport, cytokine release, growth factors, transcriptional changes, cytoskeleton rearrangement, cell movement, chemotaxis, and the like). In one embodiment, such assays are performed to screen for leukocyte activation inhibitors for drug development. The invention thus provides assays to detect molecules that specifically bind to PDZ domain-containing proteins. For example, recombinant cells expressing PDZ domain-encoding nucleic acids can be used to produce PDZ domains in these assays and to screen for molecules that bind to the domains. Molecules are contacted with the PDZ domain (or fragment thereof) under conditions conducive to binding, and then molecules that specifically bind to such domains are identified. Methods that can be used to carry out the foregoing are commonly known in the art.

It will be appreciated by the ordinarily skilled practitioner that, in one embodiment, antagonists are identified by conducting the A or G assays in the presence and absence of a known or candidate antagonist. When decreased binding is observed in the presence of a compound, that compound is identified as an antagonist. Increased binding in the presence of a compound signifies that the compound is an agonist.

For example, in one assay, a test compound can be identified as an inhibitor (antagonist) of binding between a PDZ protein and a PL protein by contacting a PDZ domain polypeptide and a PL peptide or protein in the presence and absence of the test compound, under conditions in which they would (but for the presence of the test compound) form a complex, and detecting the formation of the complex in the presence and absence of the test compound. It will be appreciated that less complex formation in the presence of the test compound than in the absence of the compound indicates that the test compound is an inhibitor of a PDZ protein-PL protein binding.

In one embodiment, the “G” assay is used in the presence or absence of an candidate inhibitor. In one embodiment, the “A” assay is used in the presence or absence of a candidate inhibitor.

In one embodiment (in which a G assay is used), one or more PDZ domain-containing GST-fusion proteins are bound to the surface of wells of a 96-well plate as described supra (with appropriate controls including nonfusion GST protein). All fusion proteins are bound in multiple wells so that appropriate controls and statistical analysis can be done. A test compound in BSA/PBS (typically at multiple different concentrations) is added to wells. Immediately thereafter, 30 uL of a detectably labeled (e.g., biotinylated) PL peptide or protein known to bind to the relevant PDZ domain (see, e.g., TABLE 2) is added in each of the wells at a final concentration of, e.g., between about 2 μM and about 40 μM, typically 5 μM, 15 μM, or 25 μM. This mixture is then allowed to react with the PDZ fusion protein bound to the surface for 10 minutes at 4° C. followed by 20 minutes at 25° C. The surface is washed free of unbound PL polypeptide three times with ice cold PBS and the amount of binding of the polypeptide in the presence and absence of the test compound is determined. Usually, the level of binding is measured for each set of replica wells (e.g. duplicates) by subtracting the mean GST alone background from the mean of the raw measurement of polypeptide binding in these wells.

In an alternative embodiment, the A assay is carried out in the presence or absence of a test candidate to identify inhibitors of PL-PDZ interactions.

If assays are conducted in the presence of test compound and compared against binding in the absence of test compound, then the assay can be conducted to determine if the difference between binding in the presence and absence of the test compound is a statistically significant difference.

In certain screening assays, assays are conducted to identify compounds that can inhibit a binding interaction between a NMDA receptor protein and a PDZ listed in TABLE 7. In other screening assays involve screening to identify an inhibitor that interferes with binding between a NMDA receptor protein (e.g., NMDAR2) and a PDZ listed in TABLE 7 other than PSD-95.

In one embodiment, a test compound is determined to be a specific inhibitor of the binding of the PDZ domain (P) and a PL (L) sequence when, at a test compound concentration of less than or equal to 1 mM (e.g., less than or equal to: 500 μM, 100 μM, 10 μM, 1 μM, 100 nM or 1 nM) the binding of P to L in the presence of the test compound less than about 50% of the binding in the absence of the test compound. (in various embodiments, less than about 25%, less than about 10%, or less than about 1%). Preferably, the net signal of binding of P to L in the presence of the test compound plus six (6) times the standard error of the signal in the presence of the test compound is less than the binding signal in the absence of the test compound.

In one embodiment, assays for an inhibitor are carried out using a single PDZ protein-PL protein pair (e.g., a PDZ domain fusion protein and a PL peptide or protein). In a related embodiment, the assays are carried out using a plurality of pairs, such as a plurality of different pairs listed in TABLES 3, 8 and 9.

In some embodiments, it is desirable to identify compounds that, at a given concentration, inhibit the binding of one PL-PDZ pair, but do not inhibit (or inhibit to a lesser degree) the binding of a specified second PL-PDZ pair. These antagonists can be identified by carrying out a series of assays using a candidate inhibitor and different PL-PDZ pairs (e.g., as shown in TABLES 3, 8 and 9) and comparing the results of the assays. All such pairwise combinations are contemplated by the invention (e.g., test compound inhibits binding of PL₁ to PDZ₁ to a greater degree than it inhibits binding of PL₁ to PDZ₂ or PL₂ to PDZ₂). Importantly, it will be appreciated that, based on the data provided in TABLES 3, 8 and 9 and disclosed herein (and additional data that can be generated using the methods described herein) inhibitors with different specificities can readily be designed.

For example, according to the invention, the Ki (“potency”) of an inhibitor of a PDZ-PL interaction can be determined. Ki is a measure of the concentration of an inhibitor required to have a biological effect. For example, administration of an inhibitor of a PDZ-PL interaction in an amount sufficient to result in an intracellular inhibitor concentration of at least between about 1 and about 100 Ki is expected to inhibit the biological response mediated by the target PDZ-PL interaction. In one aspect of the invention, the Kd measurement of PDZ-PL binding as determined using the methods supra is used in determining Ki.

Thus, in one aspect, the invention provides a method of determining the potency (Ki) of an inhibitor or suspected inhibitor of binding between a PDZ domain and a ligand by immobilizing a polypeptide comprising the PDZ domain and a non-PDZ domain on a surface, contacting the immobilized polypeptide with a plurality of different mixtures of the ligand and inhibitor, wherein the different mixtures comprise a fixed amount of ligand and different concentrations of the inhibitor, determining the amount of ligand bound at the different concentrations of inhibitor, and calculating the Ki of the binding based on the amount of ligand bound in the presence of different concentrations of the inhibitor. In an embodiment, the polypeptide is immobilized by binding the polypeptide to an immobilized immunoglobulin that binds the non-PDZ domain. This method, which is based on the “G” assay described supra, is particularly suited for high-throughput analysis of the Ki for inhibitors of PDZ-ligand interactions. Further, using this method, the inhibition of the PDZ-ligand interaction itself is measured, without distortion of measurements by avidity effects.

Typically, at least a portion of the ligand is detectably labeled to permit easy quantitation of ligand binding.

It will be appreciated that the concentration of ligand and concentrations of inhibitor are selected to allow meaningful detection of inhibition. Thus, the concentration of the ligand whose binding is to be blocked is close to or less than its binding affinity (e.g., preferably less than the 5×Kd of the interaction, more preferably less than 2×Kd, most preferably less than 1×Kd). Thus, the ligand is typically present at a concentration of less than 2 Kd (e.g., between about 0.01 Kd and about 2 Kd) and the concentrations of the test inhibitor typically range from 1 nM to 100 μM (e.g. a 4-fold dilution series with highest concentration 10 μM or 1 mM). In a preferred embodiment, the Kd is determined using the assay disclosed supra.

The Ki of the binding can be calculated by any of a variety of methods routinely used in the art, based on the amount of ligand bound in the presence of different concentrations of the inhibitor. in an illustrative embodiment, for example, a plot of labeled ligand binding versus inhibitor concentration is fit to the equation:

S_{inhibitor}=S₀*Ki/([I]+Ki)

where S_{inhibitor} is the signal of labeled ligand binding to immobilized PDZ domain in the presence of inhibitor at concentration [I] and S₀ is the signal in the absence of inhibitor (i.e., [I]=0). Typically [I] is expressed as a molar concentration.

In another aspect of the invention, an enhancer (sometimes referred to as, augmentor or agonist) of binding between a PDZ domain and a ligand is identified by immobilizing a polypeptide comprising the PDZ domain and a non-PDZ domain on a surface, contacting the immobilized polypeptide with the ligand in the presence of a test agent and determining the amount of ligand bound, and comparing the amount of ligand bound in the presence of the test agent with the amount of ligand bound by the polypeptide in the absence of the test agent. At least two-fold (often at least 5-fold) greater binding in the presence of the test agent compared to the absence of the test agent indicates that the test agent is an agent that enhances the binding of the PDZ domain to the ligand. As noted supra, agents that enhance PDZ-ligand interactions are useful for disruption (dysregulation) of biological events requiring normal PDZ-ligand function (e.g., cancer cell division and metastasis, and activation and migration of immune cells).

The invention also provides methods for determining the “potency” or “K_{enhancer}” of an enhancer of a PDZ-ligand interaction. For example, according to the invention, the K_{enhancer} of an enhancer of a PDZ-PL interaction can be determined, e.g., using the Kd of PDZ-PL binding as determined using the methods described supra. K_{enhancer} is a measure of the concentration of an enhancer expected to have a biological effect. For example, administration of an enhancer of a PDZ-PL interaction in an amount sufficient to result in an intracellular inhibitor concentration of at least between about 0.1 and about 100 K_{enhancer} (e.g., between about 0.5 and about 50 K_{enhancer}) is expected to disrupt the biological response mediated by the target PDZ-PL interaction.

Thus, in one aspect the invention provides a method of determining the potency (K_{enhancer}) of an enhancer or suspected enhancer of binding between a PDZ domain and a ligand by immobilizing a polypeptide comprising the PDZ domain and a non-PDZ domain on a surface, contacting the immobilized polypeptide with a plurality of different mixtures of the ligand and enhancer, wherein the different mixtures comprise a fixed amount of ligand, at least a portion of which is detectably labeled, and different concentrations of the enhancer, determining the amount of ligand bound at the different concentrations of enhancer, and calculating the potency (K_{enhancer})) of the enhancer from the binding based on the amount of ligand bound in the presence of different concentrations of the enhancer. Typically, at least a portion of the ligand is detectably labeled to permit easy quantitation of ligand binding. This method, which is based on the “G” assay described supra, is particularly suited for high-throughput analysis of the K_{enhancer} for enhancers of PDZ-ligand interactions.

It will be appreciated that the concentration of ligand and concentrations of enhancer are selected to allow meaningful detection of enhanced binding. Thus, the ligand is typically present at a concentration of between about 0.01 Kd and about 0.5 Kd and the concentrations of the test agent/enhancer typically range from 1 nM to 1 mM (e.g. a 4-fold dilution series with highest concentration 10 μM or 1 mM). In a preferred embodiment, the Kd is determined using the assay disclosed supra.

The potency of the binding can be determined by a variety of standard methods based on the amount of ligand bound in the presence of different concentrations of the enhancer or augmentor. For example, a plot of labeled ligand binding versus enhancer concentration can be fit to the equation:

S([E])=S(0)+(S(0)*(D_{enhancer}−1)*[E]/(([E]+K_{enhancer})

where “K_{enhancer}” is the potency of the augmenting compound, and “D_{enhancer}” is the fold-increase in binding of the labeled ligand obtained with addition of saturating amounts of the enhancing compound, [E] is the concentration of the enhancer. It will be understood that saturating amounts are the amount of enhancer such that further addition does not significantly increase the binding signal. Knowledge of “K_{enhancer}” is useful because it describes a concentration of the augmenting compound in a target cell that will result in a biological effect due to dysregulation of the PDZ-PL interaction. Typical therapeutic concentrations are between about 0.1 and about 100 K_{enhancer}.

### V. Validation of Binding Assays

Compounds identified in the foregoing binding assays can be further analyzed using a variety of biological assays to confirm that the ability of the compound to inhibit a PDZ:PL protein interaction actually inhibits a cellular activity correlated with the PDZ:PL binding interaction. Alternatively, these assays can be used directly to assay the activity of a potential inhibitory compound without conducting a binding assay beforehand. These assays can be conducted using various in vitro assays, or in vivo assays using various appropriate animal model systems.

The PDZ:PL binding interactions described herein include those involved in various biological activities in neurons. As already noted, one set of cellular activities of interest are those associated with various types of neurological disorders or injury, such as cellular responses associated with stroke and ischemia. Because neurological injury is often associated with cell death, apoptosis and excitotoxicity responses, assays for each of these responses can be conducted to validate the inhibitory activity of a compound identified through a binding assay.

For example, a variety of different parameters can be monitored to assess toxicity. Examples of such parameters include, but are not limited to, cell proliferation, monitoring activation of cellular pathways for toxicological responses by gene or protein expression analysis, DNA fragmentation, changes in the composition of cellular membranes, membrane permeability, activation of components of death-receptors or downstream signaling pathways (e.g., caspases), generic stress responses, NF-kappaB activation and responses to mitogens. Related assays are used to assay for apoptosis (a programmed process of cell death) and necrosis, including cGMP formation and NO formation. The following are illustrative of the type of biological assays that can be conducted to assess whether a inhibitory agent has a protective effect against neuronal injury or disease.

A. Morphological Changes

Apoptosis in many cell types is correlated with altered morphological appearances. Examples of such alterations include, but are not limited to, plasma membrane blebbing, cell shape change, loss of substrate adhesion properties. Such changes are readily detectable with a light microscope. Cells undergoing apoptosis can also be detected by fragmentation and disintegration of chromosomes. These changes can be detected using light microscopy and/or DNA or chromatin specific dyes.

B. Altered Membrane Permeability

Often the membranes of cells undergoing apoptosis become increasingly permeable. This change in membrane properties can be readily detected using vital dyes (e.g., propidium iodide and trypan blue). Dyes can be used to detect the presence of necrotic cells. For example, certain methods utilize a green-fluorescent LIVE/DEAD Cytotoxicity Kit #2, available from Molecular Probes. The dye specifically reacts with cellular amine groups. In necrotic cells, the entire free amine content is available to react with the dye, thus resulting in intense fluorescent staining. In contrast, only the cell-surface amines of viable cells are available to react with the dye. Hence, the fluorescence intensity for viable cells is reduced significantly relative to necrotic cells (see, e.g., Haugland, 1996 Handbook of Fluorescent Probes and Research Chemicals, 6th ed., Molecular Probes, OR).

C. Dysfunction of Mitochondrial Membrane Potential

Mitochondria provide direct and indirect biochemical regulation of diverse cellular processes as the main energy source in cells of higher organisms. These process include the electron transport chain activity, which drives oxidative phosphorylation to produce metabolic energy in the form of adenosine triphosphate (i.e., ATP). Altered or defective mitochondrial activity can result in mitochondrial collapse called the “permeability transition” or mitochondrial permeability transition. Proper mitochondrial functioning requires maintenance of the membrane potential established across the membrane. Dissipation of the membrane potential prevents ATP synthesis and thus halts or restricts the production of a vital biochemical energy source.

Consequently, a variety of assays designed to assess toxicity and cell death involve monitoring the effect of a test agent on mitochondrial membrane potentials or on the mitochondrial permeability transition. One approach is to utilize fluorescent indicators (see, e.g., Haugland, 1996 Handbook of Fluorescent Probes and Research Chemicals, 6th ed., Molecular Probes, OR, pp. 266-274 and 589-594). Various non-fluorescent probes can also be utilized (see, e.g., Kamo et al. (1979) J. Membrane Biol. 49:105). Mitochondrial membrane potentials can also be determined indirectly from mitochondrial membrane permeability (see, e.g., Quinn (1976) The Molecular Biology of Cell Membranes, University Park Press, Baltimore, Md., pp. 200-217). Further guidance on methods for conducting such assays is provided in PCT publication WO 00/19200 to Dykens et al.

D. Caspase Activation

Apoptosis is the process of programmed cell death and involves the activation of a genetic program when cells are no longer needed or have become seriously damaged. Apoptosis involves a cascade of biochemical events and is under the regulation of a number of different genes. One group of genes act as effectors of apoptosis and are referred to as the interleukin-1.beta.converting enzyme (ICE) family of genes. These genes encode a family of cysteine proteases whose activity is increased in apoptosis. The ICE family of proteases is generically referred to as caspase enzymes. The “c” in the name reflects the fact that the enzymes are cysteine proteases, while “aspase” refers to the ability of these enzymes to cleave after aspartic acid residues.

Consequently, some assays for apoptosis are based upon the observation that caspases are induced during apoptosis. Induction of these enzymes can be detected by monitoring the cleavage of specifically-recognized substrates for these enzymes. A number of naturally occurring and synthetic protein substrates are known (see, e.g., Ellerby et al. (1997) J. Neurosci. 17:6165; Kluck, et al. (1997) Science 275:1132; Nicholson et al. (1995) Nature 376:37; and Rosen and Casciola-Rosen (1997) J. Cell Biochem. 64:50). Methods for preparing a number of different substrates that can be utilized in these assays are described in U.S. Pat. No. 5,976,822. This patent also describes assays that can be conducted using whole cells that are amendable to certain of the microfluidic devices described herein. Other methods using FRET techniques are discussed in Mahajan, et al. (1999) Chem. Biol. 6:401-9; and Xu, et al. (1998) Nucl. Acids. Res. 26:2034-5.

E. Cytochrome c Release

In healthy cells, the inner mitochondrial membrane is impermeable to macromolecules. Thus, one indicator of cell apoptosis is the release or leakage of cytochrome c from the mitochondria. Detection of cytochrome c can be performed using spectroscopic methods because of the inherent absorption properties of the protein. Thus, one detection option with the present devices is to place the cells within a holding space and monitor absorbance at a characteristic absorption wavelength for cytochrome c. Alternatively, the protein can be detected using standard immunological methods (e.g., ELISA assays) with an antibody that specifically binds to cytochrome c (see, e.g., Liu et al. (1996) Cell 86:147).

F. Assays for Cell Lysis

The final stage of cell death is typically lysis of the cell. When cells die they typically release a mixture of chemicals, including nucleotides, and a variety of other substances (e.g., proteins and carbohydrates) into their surroundings. Some of the substances released include ADP and ATP, as well as the enzyme adenylate cyclase, which catalyzes the conversion of ADP to ATP in the presence of excess ADP. Thus, certain assays involve providing sufficient ADP in the assay medium to drive the equilibrium towards the generation of ATP which can subsequently be detected via a number of different means. One such approach is to utilize a luciferin/luciferase system that is well known to those of ordinary skill in the art in which the enzyme luciferase utilizes ATP and the substrate luciferin to generate a photometrically detectable signal. Further details regarding certain cell lysis assays that can be performed are set forth in PCT publication WO 00/70082.

G. Ischemic Model Systems

Methods for assaying whether a compound can confer protective neurological effects against ischemia and stroke are discussed by Aarts, et al. (Science 298:846-850, 2002). In general, this assay involves subjecting rats to a middle cerebral artery occlusion (MCAO) for a relatively short period of time (e.g., about 90 minutes). MCAO can be induced using various methods, including an intraluminal suture method (see, e.g., Longa, E. Z. et al. (1989) Stroke 20:84; and Belayev, L., et al. (1996) Stroke 27:1616). A composition containing the putative inhibitor is introduced into the rat using conventional methods (e.g., via intravenous injection). To evaluate the compositions prophylactic effect, the composition is administered before performing MCAO. If the compound is to be evaluated for its ability to mitigate against an ischemic event that has already occurred, the composition with the compound is introduced after MCAO has been initiated. The extent of cerebral infarction is then evaluated using various measures of neurological function. Examples of such measures include the postural reflex test (Bederson, J. B. et al. (1986) Stroke 17:472) and the forelimb placing test (De Ryck, M. et al. (1989) Stroke 20:1383). Methods are also described in Aarts et al assessing the effects of NMDA-induced excitotoxicity using in vitro assays.

### VI. Global Analysis of PDZ-PL Interactions

As described supra, the present invention provides powerful methods for analysis of PDZ-ligand interactions, including high-throughput methods such as the “G” assay and affinity assays described supra. In one embodiment of the invention, the affinity is determined for a particular ligand and a plurality of PDZ proteins. Typically the plurality is at least 5, and often at least 25, or at least 40 different PDZ proteins. In a preferred embodiment, the plurality of different PDZ proteins are from a particular tissue (e.g., central nervous system) or a particular class or type of cell, (e.g., a neuron) and the like. In a most preferred embodiment, the plurality of different PDZ proteins represents a substantial fraction (e.g., typically a majority, more often at least 80%) of all of the PDZ proteins known to be, or suspected of being, expressed in the tissue or cell(s), e.g., all of the PDZ proteins known to be present in neuronal cells. In an embodiment, the plurality is at least 50%, usually at least 80%, at least 90% or all of the PDZ proteins disclosed herein as being expressed in neuronal cells.

In one embodiment of the invention, the binding of a ligand to the plurality of PDZ proteins is determined. Using this method, it is possible to identify a particular PDZ domain bound with particular specificity by the ligand. The binding may be designated as “specific” if the affinity of the ligand to the particular PDZ domain is at least 2-fold that of the binding to other PDZ domains in the plurality (e.g., present in that cell type). The binding is deemed “very specific” if the affinity is at least 10-fold higher than to any other PDZ in the plurality or, alternatively, at least 10-fold higher than to at least 90%, more often 95% of the other PDZs in a defined plurality. Similarly, the binding is deemed “exceedingly specific” if it is at least 100-fold higher. For example, a ligand could bind to 2 different PDZs with an affinity of 1 uM and to no other PDZs out of a set 40 with an affinity of less than 100 uM. This would constitute specific binding to those 2 PDZs. Similar measures of specificity are used to describe binding of a PDZ to a plurality of PLs.

It will be recognized that high specificity PDZ-PL interactions represent potentially more valuable targets for achieving a desired biological effect. The ability of an inhibitor or enhancer to act with high specificity is often desirable. In particular, the most specific PDZ-ligand interactions are also the best therapeutic targets, allowing specific inhibition of the interaction.

Thus, in one embodiment, the invention provides a method of identifying a high specificity interaction between a particular PDZ domain and a ligand known or suspected of binding at least one PDZ domain, by providing a plurality of different immobilized polypeptides, each of said polypeptides comprising a PDZ domain and a non-PDZ domain; determining the affinity of the ligand for each of said polypeptides, and comparing the affinity of binding of the ligand to each of said polypeptides, wherein an interaction between the ligand and a particular PDZ domain is deemed to have high specificity when the ligand binds an immobilized polypeptide comprising the particular PDZ domain with at least 2-fold higher affinity than to immobilized polypeptides not comprising the particular PDZ domain.

In a related aspect, the affinity of binding of a specific PDZ domain to a plurality of ligands (or suspected ligands) is determined. For example, in one embodiment, the invention provides a method of identifying a high specificity interaction between a PDZ domain and a particular ligand known or suspected of binding at least one PDZ domain, by providing an immobilized polypeptide comprising the PDZ domain and a non-PDZ domain; determining the affinity of each of a plurality of ligands for the polypeptide, and comparing the affinity of binding of each of the ligands to the polypeptide, wherein an interaction between a particular ligand and the PDZ domain is deemed to have high specificity when the ligand binds an immobilized polypeptide comprising the PDZ domain with at least 2-fold higher affinity than other ligands tested. Thus, the binding may be designated as “specific” if the affinity of the PDZ to the particular PL is at least 2-fold that of the binding to other PLs in the plurality (e.g., present in that cell type). The binding is deemed “very specific” if the affinity is at least 10-fold higher than to any other PL in the plurality or, alternatively, at least 10-fold higher than to at least 90%, more often 95% of the other PLs in a defined plurality. Similarly, the binding is deemed “exceedingly specific” if it is at least 100-fold higher. Typically the plurality is at least 5 different ligands, more often at least 10.

1. Use of Array for Global Predictions

One discovery of the present inventors relates to the important and extensive roles played by interactions between PDZ proteins and PL proteins, particularly in the biological function of neuronal cells. Further, it has been discovered that valuable information can be ascertained by analysis (e.g., simultaneous analysis) of a large number of PDZ-PL interactions. In a preferred embodiment, the analysis encompasses all of the PDZ proteins expressed in a particular tissue (e.g., brain) or type or class of cell (e.g., neuron). Alternatively, the analysis encompasses at least about 5, or at least about 10, or at least about 12, or at least about 15 and often at least 50 different polypeptides, up to about 60, about 80, about 100, about 150, about 200, or even more different polypeptides; or a substantial fraction (e.g., typically a majority, more often at least 80%) of all of the PDZ proteins known to be, or suspected of being, expressed in the tissue or cell(s), (e.g., all of the PDZ proteins known to be present in neurons).

It will be recognized that the arrays and methods of the invention are directed to analyze of PDZ and PL interactions, and involve selection of such proteins for analysis. While the devices and methods of the invention may include or involve a small number of control polypeptides, they typically do not include significant numbers of proteins or fusion proteins that do not include either PDZ or PL domains (e.g., typically, at least about 90% of the arrayed or immobilized polypeptides in a method or device of the invention is a PDZ or PL sequence protein, more often at least about 95%, or at least about 99%).

It will be apparent from this disclosure that analysis of the relatively large number of different interactions preferably takes place simultaneously. In this context, “simultaneously” means that the analysis of several different PDZ-PL interactions (or the effect of a test agent on such interactions) is assessed at the same time. Typically the analysis is carried out in a high throughput (e.g., robotic) fashion. One advantage of this method of simultaneous analysis is that it permits rigorous comparison of multiple different PDZ-PL interactions. For example, as explained in detail elsewhere herein, simultaneous analysis (and use of the arrays described infra) facilitates, for example, the direct comparison of the effect of an agent (e.g., an potential interaction inhibitor) on the interactions between a substantial portion of PDZs and/or PLs in a tissue or cell.

Accordingly, in one aspect, the invention provides an array of immobilized polypeptide comprising the PDZ domain and a non-PDZ domain on a surface. Typically, the array comprises at least about 5, or at least about 10, or at least about 12, or at least about 15 and often at least 50 different polypeptides. In one preferred embodiment, the different PDZ proteins are from a particular tissue (e.g., central nervous system) or a particular class or type of cell, (e.g., a neuron) and the like. In a most preferred embodiment, the plurality of different PDZ proteins represents a substantial fraction (e.g., typically a majority, more often at least 60%, 70% or 80%) of all of the PDZ proteins known to be, or suspected of being, expressed in the tissue or cell(s), (e.g., all of the PDZ proteins known to be present in neurons).

Certain embodiments are arrays which include a plurality, usually at least 5, 10, 25, 50 PDZ proteins present in a particular cell of interest. In this context, “array” refers to an ordered series of immobilized polypeptides in which the identity of each polypeptide is associated with its location. In some embodiments the plurality of polypeptides are arrayed in a “common” area such that they can be simultaneously exposed to a solution (e.g., containing a ligand or test agent). For example, the plurality of polypeptides can be on a slide, plate or similar surface, which may be plastic, glass, metal, silica, beads or other surface to which proteins can be immobilized. In a different embodiment, the different immobilized polypeptides are situated in separate areas, such as different wells of multi-well plate (e.g., a 24-well plate, a 96-well plate, a 384 well plate, and the like). It will be recognized that a similar advantage can be obtained by using multiple arrays in tandem.

2. Analysis of PDZ-PL Inhibition Profile

In one aspect, the invention provides a method for determining if a test compound inhibits any PDZ-ligand interaction in large set of PDZ-ligand interaction (e.g., a plurality of the PDZ-ligands interactions described in TABLE 3, 8 or 9; a majority of the PDZ-ligands identified in a particular cell or tissue as described supra (e.g., neurons) and the like. In one embodiment, the PDZ domains of interest are expressed as GST-PDZ fusion proteins and immobilized as described herein. For each PDZ domain, a labeled ligand that binds to the domain with a known affinity is identified as described herein.

For any known or suspected modulator (e.g., inhibitor) of a PDL-PL interaction(s), it is useful to know which interactions are inhibited (or augmented). For example, an agent that inhibits all PDZ-PL interactions in a cell (e.g., a neuron) will have different uses than an agent that inhibits only one, or a small number, of specific PDZ-PL interactions. The profile of PDZ interactions inhibited by a particular agent is referred to as the “inhibition profile” for the agent, and is described in detail below. The profile of PDZ interactions enhanced by a particular agent is referred to as the “enhancement profile” for the agent. It will be readily apparent to one of skill guided by the description of the inhibition profile how to determine the enhancement profile for an agent. The present invention provides methods for determining the PDZ interaction (inhibition/enhancement) profile of an agent in a single assay.

In one aspect, the invention provides a method for determining the PDZ-PL inhibition profile of a compound by providing (i) a plurality of different immobilized polypeptides, each of said polypeptides comprising a PDZ domain and a non-PDZ domain and (ii) a plurality of corresponding ligands, wherein each ligand binds at least one PDZ domain in (i), then contacting each of said immobilized polypeptides in (i) with a corresponding ligand in (ii) in the presence and absence of a test compound, and determining for each polypeptide-ligand pair whether the test compound inhibits binding between the immobilized polypeptide and the corresponding ligand.

Typically the plurality is at least 5, and often at least 25, or at least 40 different PDZ proteins. In a preferred embodiment, the plurality of different ligands and the plurality of different PDZ proteins are from the same tissue or a particular class or type of cell, (e.g., a neuron). In a most preferred embodiment, the plurality of different PDZs represents a substantial fraction (e.g., at least 80%) of all of the PDZs known to be, or suspected of being, expressed in the tissue or cell(s), e.g., all of the PDZs known to be present in neurons (for example, at least 80%, at least 90% or all of the PDZs disclosed herein as being expressed in neuronal cells).

In one embodiment, the inhibition profile is determined as follows: A plurality (e.g., all known) PDZ domains expressed in a cell (e.g., neurons) are expressed as GST-fusion proteins and immobilized without altering their ligand binding properties as described supra. For each PDZ domain, a labeled ligand that binds to this domain with a known affinity is identified. If the set of PDZ domains expressed in neurons is denoted by {P1 . . . Pn}, any given PDZ domain Pi binds a (labeled) ligand Li with affinity K_{d}i. To determine the inhibition profile for a test agent “compound X” the “G” assay (supra) can be performed as follows in 96-well plates with rows A-H and columns 1-12. Column 1 is coated with P1 and washed. The corresponding ligand L1 is added to each washed coated well of column 1 at a concentration 0.5 K_{d}1 with (rows B, D, F, H) or without (rows A, C, E, F) between about 1 and about 1000 uM) of test compound X. Column 2 is coated with P2, and L2 (at a concentration 0.5 K_{d}2) is added with or without inhibitor X. Additional PDZ domains and ligands are similarly tested.

Compound X is considered to inhibit the binding of Li to Pi if the average signal in the wells of column i containing X is less than half the signal in the equivalent wells of the column lacking X. Thus, in this single assay one determines the full set of neural PDZs that are inhibited by compound X.

In some embodiments, the test compound X is a mixture of compounds, such as the product of a combinatorial chemistry synthesis as described supra. In some embodiments, the test compound is known to have a desired biological effect, and the assay is used to determine the mechanism of action (i.e., if the biological effect is due to modulating a PDZ-PL interaction).

It will be apparent that an agent that modulates only one, or a few PDZ-PL interactions, in a panel (e.g., a panel of all known PDZs in neurons, a panel of at least 10, at least 20 or at least 50 PDZ domains) is a more specific modulator than an agent that modulate many or most interactions. Typically, an agent that modulates less than 20% of PDZ domains in a panel (e.g., TABLE 4) is deemed a “specific” inhibitor, less than 6% a “very specific” inhibitor, and a single PDZ domain a “maximally specific” inhibitor.

It will be recognized that high specificity modulators of PDZ-PL interactions represent potentially more valuable drug targets for achieving a desired biological effect. The ability of an inhibitor or enhancer to act with “maximal specificity” is most desirable.

In one embodiment, the assays of the invention can be used to determine a maximally specific modulator of the interaction between a NMDA receptor and a PDZ domain.

In a preferred embodiment, the assays of the invention are used to identify a maximally specific modulator of the interaction between NMDA receptor 2B (NMDAR2B) and PSD95.

It will also be appreciated that “compound X” may be a composition containing mixture of compounds (e.g., generated using combinatorial chemistry methods) rather than a single compound.

Several variations of this assay are contemplated:

In some alternative embodiments, the assay above is performed using varying concentrations of the test compound X, rather than fixed concentration. This allows determination of the Ki of the X for each PDZ as described above.

In an alternative embodiment, instead of pairing each PDZ Pi with a specific labeled ligand Li, a mixture of different labeled ligands is created that such that for every PDZ at least one of the ligands in the mixture binds to this PDZ sufficiently to detect the binding in the “G” assay. This mixture is then used for every PDZ domain.

In one embodiment, compound X is known to have a desired biological effect, but the chemical mechanism by which it has that effect is unknown. The assays of the invention can then be used to determine if compound X has its effect by binding to a PDZ domain.

In one embodiment, PDZ-domain containing proteins are classified in to groups based on their biological function, e.g. into those that regulate apoptosis versus those that regulate transcription. An optimal inhibitor of a particular function (e.g., including but not limited to an anti-apoptotic agent, an anti-T cell activation agent, cell-cycle control, vesicle transport, etc.) will inhibit multiple PDZ-ligand interactions involved in the function (e.g., apoptosis, activation) but few other interactions. Thus, the assay is used in one embodiment in screening and design of a drug that specifically blocks a particular function. For example, an agent designed to block apoptosis might be identified because, at a given concentration, the agent inhibits 2 or more PDZs involved in apoptosis but fewer than 3 other PDZs, or that inhibits PDZs involved in apoptosis with a Ki>10-fold better than for other PDZs. Thus, the invention provides a method for identifying an agent that inhibits a first selected PDZ-PL interaction or plurality of interactions but does not inhibit a second selected PDZ-PL interaction or plurality of interactions. The two (or more) sets of interactions can be selected on the basis of the known biological function of the PDZ proteins, the tissue specificity of the PDZ proteins, or any other criteria. Moreover, the assay can be used to determine effective doses (i.e., drug concentrations) that result in desired biological effects while avoiding undesirable effects.

3. Side Effects of PDZ-PL Modulator Interactions

In a related embodiment, the invention provides a method for determining likely side effects of a therapeutic that inhibits PDZ-ligand interactions. The method entails identifying those target tissues, organs or cell types that express PDZ proteins and ligands that are disrupted by a specified inhibitor. If, at a therapeutic dosage, a drug intended to have an effect in one organ system (e.g., central nervous system) disrupts PDZ-PL interactions in a different system (e.g., hematopoietic system) it can be predicted that the drug will have effects (“side effects”) on the second system. It will be apparent that the information obtained from this assay will be useful in the rational design and selection of drugs that do not have the side-effect.

In one embodiment, for example, a comprehensive PDZ protein set is obtained. A “perfectly comprehensive” PDZ protein set is defined as the set of all PDZ proteins expressed in the subject animal (e.g., humans). A comprehensive set may be obtained by analysis of, for example, the human genome sequence. However, a “perfectly comprehensive” set is not required and any reasonably large set of PDZ domain proteins (e.g., the set of all known PDZ proteins; or the set listed in TABLE 4) will provide valuable information.

In one embodiment, the method involves some of all of the following steps:

a) For each PDZ protein, determine the tissues in which it is highly expressed. This can be done experimentally although the information generally will be available in the scientific literature;

b) For each PDZ protein (or as many as possible), identify the cognate PL(s) bound by the PDZ protein;

c) Determine the Ki at which the test agent inhibits each PDZ-PL interaction, using the methods described supra;

d) From this information it is possible to calculate the pattern of PDZ-PL interactions disrupted at various concentrations of the test agent.

By correlating the set of PDZ-PL interactions disrupted with the expression pattern of the members of that set, it will be possible to identify the tissues likely affected by the agent.

Additional steps can also be carried out, including determining whether a specified tissue or cell type is exposed to an agent following a particular route of administration. This can be determined using basis pharmacokinetic methods and principles.

4. Modulation of Activities

The PDZ binding moieties and inhibitors described herein that disrupt PDZ:PL protein interactions can be used to modulate biological activities or functions of cells (e.g., neurons). These agents can also be utilized to treat diseases and conditions in human and nonhuman animals (e.g., experimental models). Exemplary biological activities are listed supra.

When administered to patients, the compounds of the invention (e.g., PL-PDZ interaction inhibitors) are useful for treating (ameliorating symptoms of) a variety of neurological disorders, including those associated with some type of injury to neuronal cells or the death of neurons. Such disorders include, but are not limited to, stroke, ischemia, brain traumas and chronic pain. Certain inhibitors can also be used to treat other types of neuorological disorders like Alzheimer's disease, epilepsy, Parkinson's disease, Huntington's disease, motor neuron diseases and inherited ataxias.

Some other inhibitors can be utilized to treat other disease types, including, for instance, inflammatory and humoral immune responses, e.g., inflammation, allergy (e.g., systemic anaphylaxis, hypersensitivity responses, drug allergies, insect sting allergies);

infectious diseases (e.g., viral infection, such as HIV, measles, parainfluenza, virus-mediated cell fusion,), and ischemia (e.g., post-myocardial infarction complications, joint injury, kidney, scleroderma).

### VII. Antagonists of PDZ-PL Interactions

As described herein, interactions between PDZ proteins and PL proteins in cells (e.g., neurons) may be disrupted or inhibited by the administration of inhibitors or antagonists. Inhibitors can be identified using screening assays described herein. In embodiment, the motifs disclosed herein are used to design inhibitors. In some embodiments, the antagonists of the invention have a structure (e.g., peptide sequence) based on the C-terminal residues of PL-domain proteins listed in TABLE 2. In some embodiments, the antagonists of the invention have a structure (e.g., peptide sequence) based on a PL motif disclosed herein.

The PDZ/PL antagonists and antagonists of the invention can be any of a large variety of compounds, both naturally occurring and synthetic, organic and inorganic, and including polymers (e.g., oligopeptides, polypeptides, oligonucleotides, and polynucleotides), small molecules, antibodies, sugars, fatty acids, nucleotides and nucleotide analogs, analogs of naturally occurring structures (e.g., peptide mimetics, nucleic acid analogs, and the like), and numerous other compounds. Although, for convenience, the present discussion primarily refers antagonists of PDZ-PL interactions, it will be recognized that PDZ-PL interaction agonists can also be use in the methods disclosed herein.

In one aspect, the peptides and peptide mimetics or analogues of the invention contain an amino acid sequence that binds a PDZ domain in a cell of interest. In one embodiment, the antagonists comprise a peptide that has a sequence corresponding to the carboxy-terminal sequence of a PL protein listed in TABLE 2, e.g., a peptide listed TABLE 2. Typically, the peptide comprises at least the C-terminal two (3), three (3) or four (4) residues of the PL protein, and often the inhibitory peptide comprises more than four residues (e.g., at least five, six, seven, eight, nine, ten, twelve or fifteen residues) from the PL protein C-terminus.

In some embodiments, the inhibitor is a peptide, e.g., having a sequence of a PL C-terminal protein sequence.

In some embodiments, the antagonist is a fusion protein comprising such a sequence. Fusion proteins containing a transmembrane transporter amino acid sequence can be used to facilitate transport of the inhibitor into a cell.

In some embodiments, the inhibitor is conserved variant of the PL C-terminal protein sequence having inhibitory activity.

In some embodiments, the antagonist is a peptide mimetic of a PL C-terminal sequence.

In some embodiments, the inhibitor is a small molecule (i.e., having a molecular weight less than 1 kD).

A. Polypeptide Antagonists

1. Inhibitors with a PL Sequence One class of inhibitors or antagonists that are provided comprise a peptide that has a sequence of a PL protein carboxy-terminus listed in TABLE 2. The PL protein carboxy-terminus sequences can be considered as the “core PDZ motif sequence” because of the ability of the short sequence from the carboxy terminus to interact with the PDZ domain. For example, in some inhibitors the “core PDZ motif sequence” or simply the “PL sequence” contains the last 2, 3 or 4 C-terminus amino acids. In other instances, however, the core PDZ motif comprises more than 2-4 residues (e.g., at least 5, 6, 7, 8, 9, 10, 11, 12, 13, 14, 15, 16, 17, 18, 19, or 20 residues) from the PL protein C-terminus. For some inhibitors, the PDZ motif sequence peptide is from 4-15 amino acids in length. Other inhibitors have a PDZ motif sequence that is 6-10 amino acids in length, or 3-8 amino acids in length, or 3-7 amino acids in length. Certain inhibitors have a PDZ motif sequence that is 8 amino acids in length. Although the residues shared by the inhibitory peptide and the PL protein are often found at the C-terminus of the peptide, some inhibitors incorporate a PL sequence that is located in an internal region of a PL protein. Similarly, in some cases, the inhibitory peptide comprises residues from a PL sequence that is near, but not at the C-terminus of a PL protein (see, Gee et al., 1998, J Biological Chem. 273:21980-87).

Another set of inhibitors are based upon the identification of amino acid sequences that specifically disrupt binding between NMDAR proteins and PSD-95. This particular class of inhibitors are polypeptides that share the following characteristics: 1) a size ranging from 3-20 amino acids in length (although somewhat longer polypeptides can be used), and 2) a C-terminal consensus sequence of X-T-X-V/L/A (the slash separates different amino acids that can appear at a given position).

Specific examples of polypeptides that were found to be able to inhibit NMDAR and PSD-95 interactions include:

1) peptides 3 amino acids in length: TEV and SDV;

2) peptides 4 amino acids in length: ETEV, ETQL, QTQV, ETAL, QTEV, ESEV, ETVA and FTDV;

3) 19 C-terminal amino acids from TAX (ISPGGLEPPSEKHFRETEV);

4) 19 C-terminal amino acids from modified HPV 16 E6 (TGRGMSGGRSSRTRRETQL); and

5) 20 C-terminal amino acids from TAX (QISPGGLEPPSEKHFRETEV).

These specific examples should not be considered as limiting but simply illustrative of inhibitors having the general characteristics listed above.

Yet another set of inhibitors are based upon the PL sequences that were identified as binding to the PDZ domain of nNOS (see Example 8 and TABLE 8). Inhibitors in this class can be polypeptides whose carboxy terminus comprises at least two contiguous amino acids from the C-terminus of one of the PL sequences. As with the other classes of inhibitors described above, the PL sequence/PDZ core sequence motif can be longer, such as 3-20 amino acids from the C-terminus of the PL sequences listed in TABLE 8.

A third group of inhibitors include a PL sequence from the list shown in TABLE 9. This table lists PL sequences that were identified as binding to the PDZ domain of PSD-95 (see Example 9). Like the other classes of inhibitors based upon PL sequences, these inhibitors generally include at least 2-3 continguous amino acids from the C-terminus of the sequences listed in this table. Typically, the PL sequence portion of these inhibitors is 3-20 amino acids in length. Inhibitors within this class can be utilized to disrupt binding between PL proteins containing these sequences can PDZ proteins such as PSD-95.

As described in greater detail below, short PL peptides, such as just described can be used in the rational design of other small molecules with similar properties according to established techniques.

Core PDZ motif sequences/PL sequences such as those just listed can optionally be joined to additional amino acids at their amino terminus to further increase binding affinity and/or stability and/or transportability into cells. These additional sequences located at the amino terminus can be from the natural sequence of a neuronal cell surface receptor or from other sources. The PDZ motif sequence and additional N-terminal sequences can optionally be joined by a linker. The additional amino acids can also be conservatively substituted. The total peptide length (i.e., core PDZ motif sequence plus optional N-terminal segment) can be of a variety of lengths (e.g., at least 2, 3, 4, 5, 6, 8, 10, 15, 20, 25, 30, 40, 50, 60, 70, 80, 90, 100 or more amino acids). Typically, the overall length is in the range of 30-40 amino acids. For those inhibitors in which additional sequences are attached at the N-terminus of the core PDZ motif sequence (PL sequence), the overall structure is thus: N-terminal segment—core PDZ motif sequence (PL sequence), or N-terminal segment—linker—core PDZ motif sequence (PL sequence). As discussed further below, one useful class of proteins that can be fused to the core PDZ motifs or PL sequences are transmembrane transporter peptides. These peptides can be fused to the inhibitory sequences to facilitate transport into a target cell (e.g., neuron). Further details are provided below. Purification tags that are known in the art can also optionally be fused to the N-terminus of the PL sequence.

2. Inhibitors with a PDZ-Domain Polypeptide

Some of the inhibitors that are provided rather than containing a PL sequence, instead contain all or a portion of a PDZ binding domain. The PDZ-domain sequence included in these inhibitors is selected to mimic (i.e., have similar binding characteristics) of the PDZ domain in the PDZ protein of interest (i.e., the PDZ protein whose binding interaction with a PL protein one seeks to disrupt). The PDZ-domain sequence is long enough to include at least enough of the PDZ domain such that the resulting polypeptide inhibitor can effectively bind to the cognate PL protein. This typically means that the PDZ-domain sequence is at least 50, 55, 60, 65, 70, 75, 80, 85, 90 or more amino acids long. But certain inhibitors can include the entire PDZ-domain, or even additional amino acids from the PDZ protein that extend beyond the PDZ-domain.

3. Optional Features of Inhibitors

Polypeptide inhibitors such as those just described can optionally be derivatized (e.g., acetylated, phosphorylated and/or glycoslylated) to improve the binding affinity of the inhibitor, to improve the ability of the inhibitor to be transported across a cell membrane or to improve stability. As a specific example, for inhibitors in which the third residue from the C-terminus is S, T or Y, this residue can be phosphorylated prior to the use of the peptide.

The polypeptide inhibitors can also optionally be linked directly or via a linker to a transmembrane transporter peptide. Specific examples of these sequences are described in the section on formulation and administration of the polypeptides of the invention. But certain polypeptide inhibitors do not include a transporter peptide.

B. Peptide Variants

Having identified PDZ binding peptides and PDZ-PL interaction inhibitory sequences, variations of these sequences can be made and the resulting peptide variants can be tested for PDZ domain binding or PDZ-PL inhibitory activity. In embodiments, the variants have the same or a different ability to bind a PDZ domain as the parent peptide. Typically, such amino acid substitutions are conservative, i.e., the amino acid residues are replaced with other amino acid residues having physical and/or chemical properties similar to the residues they are replacing. Preferably, conservative amino acid substitutions are those wherein an amino acid is replaced with another amino acid encompassed within the same designated class.

C. Peptide Mimetics

Having identified PDZ binding peptides and PDZ-PL interaction inhibitory sequences, peptide mimetics can be prepared using routine methods, and the inhibitory activity of the mimetics can be confirmed using the assays of the invention. Thus, in some embodiments, the antagonist is a peptide mimetic of a PL C-terminal sequence. The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

D. Small Molecules

In some embodiments, the inhibitor is a small molecule (i.e., having a molecular weight less than 1 kD). Methods for screening small molecules are well known in the art and include those described supra.

E. Binding Affinity

Regardless of type, the inhibitors generally have an EC₅₀ of less than 50 um. Some inhibitors have an EC₅₀ of less than 10 uM, others have an EC₅₀ of 1 uM, and still others an EC₅₀ of less than 100 nM. The inhibitors typically have an EC₅₀ value of 20-100 nM.

### VIII. Uses of PDZ Domain Binding and Antagonist Compounds

Because the inhibitors that are described herein are useful in interfering with binding between certain PDZ and PL proteins in neurons (e.g., the NMDAR/PSD-95 interaction, and the interaction between nNOS and various PL proteins), the inhibitors can be utilized in the treatment of a variety of biological processes in neuron cells. For instance, the inhibitors can be utilized to treat problems associated with excitotoxicity and apoptosis occasioned by neuronal damage. The inhibitors can also be utilized to treat various neurological diseases, including those associated with stroke and ischemia. Specific examples of neurological diseases that can be treated with certain inhibitors include, Alzheimer's disease, epilepsy, Parkinson's disease, Huntington's disease, motor neuron diseases and inherited ataxias.

Because PDZ proteins are involved in a number of biological functions besides involvement in excitotoxicity responses, some of the inhibitors that are provided can be used in the treatment of other conditions and activities correlated with the PDZ:PL protein interactions described herein. Examples of such activities include, but are not limited to, organization and regulation of multiprotein complexes, vesicular trafficking, tumor suppression, protein sorting, establishment of membrane polarity, apoptosis, regulation of immune response and organization of synapse formation. In general, PDZ proteins have a common function of facilitating the assembly of multi-protein complexes, often serving as a bridge between several proteins, or regulating the function of other proteins. Additionally, as also noted supra, these proteins are found in essentially all cell types.

Consequently, modulation of these interactions can be utilized to control a wide variety of biological conditions and physiological conditions. In particular, modulation of interactions such as those disclosed herein can be utilized to control movement of vesicles within a cell, inhibition of tumor formation, as well as in the treatment of immune disorders, neurological disorders, muscular disorders, and intestinal disorders.

Certain compounds which modulate binding of the PDZ proteins and PL proteins can be used to inhibit leukocyte activation, which is manifested in measurable events including but not limited to, cytokine production, cell adhesion, expansion of cell numbers, apoptosis and cytotoxicity. Thus, some compounds of the invention can be used to treat diverse conditions associated with undesirable leukocyte activation, including but not limited to, acute and chronic inflammation, graft-versus-host disease, transplantation rejection, hypersensitivities and autoimmunity such as multiple sclerosis, rheumatoid arthritis, peridontal disease, systemic lupus erythematosis, juvenile diabetes mellitis, non-insulin-dependent diabetes, and allergies, and other conditions listed herein.

Thus, the invention also relates to methods of using such compositions in modulating leukocyte activation as measured by, for example, cytotoxicity, cytokine production, cell proliferation, and apoptosis.

### IX. Formulation and Route of Administration

A. Introduction of Antagonists (e.g., Peptides and Fusion Proteins) into Cells

The inhibitors disclosed herein or identified using the screening methods that are provided can be used in the manufacture of a medicament or pharmaceutical composition. These can then be administered according to a number of different methods.

In one aspect, the PDZ-PL antagonists of the invention are introduced into a cell to modulate (i.e., increase or decrease) a biological function or activity of the cell. Many small organic molecules readily cross the cell membranes (or can be modified by one of skill using routine methods to increase the ability of compounds to enter cells, e.g., by reducing or eliminating charge, increasing lipophilicity, conjugating the molecule to a moiety targeting a cell surface receptor such that after interacting with the receptor). Methods for introducing larger molecules, e.g., peptides and fusion proteins are also well known, including, e.g., injection, liposome-mediated fusion, application of a hydrogel, conjugation to a targeting moiety conjugate endocytozed by the cell, electroporation, and the like).

In one embodiment, the antagonist or agent is a fusion polypeptide or derivatized polypeptide. A fusion or derivatized protein may include a targeting moiety that increases the ability of the polypeptide to traverse a cell membrane or causes the polypeptide to be delivered to a specified cell type (e.g., a neuron) preferentially or cell compartment (e.g., nuclear compartment) preferentially. Examples of targeting moieties include lipid tails, amino acid sequences such as antennapoedia peptide or a nuclear localization signal (NLS; e.g., Xenopus nucleoplasmin Robbins et al., 1991, Cell 64:615).

In one embodiment of the invention, a peptide sequence or peptide analog determined to inhibit a PDZ domain-PL protein binding interaction as described herein is introduced into a cell by linking the sequence to an amino acid sequence that facilitates its transport through the plasma membrane (a “transmembrane transporter sequence”). The peptides of the invention may be used directly or fused to a transmembrane transporter sequence to facilitate their entry into cells. In the case of such a fusion peptide, each peptide may be fused with a heterologous peptide at its amino terminus directly or by using a flexible polylinker such as the pentamer G-G-G-G-S repeated 1 to 3 times. Such linker has been used in constructing single chain antibodies (scFv) by being inserted between V_{H} and V_{L} (Bird et al., 1988, Science 242:423-426; Huston et al., 1988, Proc. Natl. Acad. Sci. U.S.A. 85:5979-5883). The linker is designed to enable the correct interaction between two beta-sheets forming the variable region of the single chain antibody. Other linkers which may be used include Glu-Gly-Lys-Ser-Ser-Gly-Ser-Gly-Ser-Glu-Ser-Lys-Val-Asp (Chaudhary et al., 1990, Proc. Natl. Acad. Sci. U.S.A. 87:1066-1070) and Lys-Glu-Ser-Gly-Ser-Val-Ser-Ser-Glu-Gln-Leu-Ala-Gln-Phe-Arg-Ser-Leu-Asp (Bird et al., 1988, Science 242:423-426). A number of peptide sequences have been described in the art as capable of facilitating the entry of a peptide linked to these sequences into a cell through the plasma membrane (Derossi et al., 1998, Trends in Cell Biol. 8:84). For the purpose of this invention, such peptides are collectively referred to as transmembrane transporter peptides. Examples of these peptide include, but are not limited to, tat derived from HIV (Vives et al., 1997, J. Biol. Chem. 272:16010; Nagahara et al., 1998, Nat. Med. 4:1449), antennapedia from Drosophila (Derossi et al., 1994, J. Biol. Chem. 261:10444), VP22 from herpes simplex virus (Elliot and D'Hare, 1997, Cell 88:223-233), complementarity-determining regions (CDR) 2 and 3 of anti-DNA antibodies (Avrameas et al., 1998, Proc. Nall Acad. Sci. U.S.A., 95:5601-5606), 70 KDa heat shock protein (Fujihara, 1999, EMBO J. 18:411-419) and transportan (Pooga et al., 1998, FASEB J. 12:67-77). In a preferred embodiment of the invention, a truncated HIV tat peptide having the sequence of GYGRKKRRQRRRG is used.

In some instances, a transmembrane transporter sequence is fused to a neuronal cell surface receptor carboxyl terminal sequence at its amino-terminus with or without a linker. Generally, the C-terminus of a PDZ motif sequence (PL sequence) is free to interact with a PDZ domain. The transmembrane transporter sequence can be used in whole or in part as long as it is capable of facilitating entry of the peptide into a cell.

In an alternate embodiment of the invention, a neuronal cell surface receptor C-terminal sequence can be used alone when it is delivered in a manner that allows its entry into cells in the absence of a transmembrane transporter sequence. For example, the peptide may be delivered in a liposome formulation or using a gene therapy approach by delivering a coding sequence for the PDZ motif alone or as a fusion molecule into a target cell.

The compounds of the of the invention can also be administered via liposomes, which serve to target the conjugates to a particular tissue, such as neural tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations, the peptide to be delivered is incorporated as part of a liposome, alone or in conjunction with a molecule which binds to, e.g., a receptor prevalent among neural cells, such as monoclonal antibodies which bind to the NMDA Receptor. Thus, liposomes filled with a desired peptide or conjugate of the invention can be directed to the site of neural cells, where the liposomes then deliver the selected inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Arm. Rev. Biophys. Bioeng. 9:467 (1980), U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to the neural cells, a ligand to be incorporated into the liposome can include, e.g., antibodies or fragments thereof specific for cell surface determinants of the desired nervous system cells. A liposome suspension containing a peptide or conjugate may be administered intravenously, locally, topically, etc. in a dose which varies according to, inter alia, the manner of administration, the conjugate being delivered, and the stage of the disease being treated.

In order to specifically deliver a PDZ motif sequence (PL sequence) peptide into a specific cell type, the peptide can be linked to a cell-specific targeting moiety, which include but are not limited to, ligands for diverse neuron surface molecules such as growth factors, hormones and cytokines, neuronal receptors, ion transporters, as well as antibodies or antigen-binding fragments thereof. Since a large number of cell surface receptors have been identified in neurons, ligands or antibodies specific for these receptors may be used as cell-specific targeting moieties.

Antibodies are the most versatile cell-specific targeting moieties because they can be generated against any cell surface antigen. Monoclonal antibodies have been generated against neuron-specific markers. Antibody variable region genes can be readily isolated from hybridoma cells by methods well known in the art. However, since antibodies are assembled between two heavy chains and two light chains, it is preferred that a scFv be used as a cell-specific targeting moiety in the present invention. Such scFv are comprised of V_{H} and V_{L} domains linked into a single polypeptide chain by a flexible linker peptide.

The PDZ motif sequence (PL sequence) may be linked to a transmembrane transporter sequence and a cell-specific targeting moiety to produce a tri-fusion molecule. This molecule can bind to a neuron surface molecule, passes through the membrane and targets PDZ domains. Alternatively, a PDZ motif sequence (PL sequence) may be linked to a cell-specific targeting moiety that binds to a surface molecule that internalizes the fusion peptide.

In an other approach, microspheres of artificial polymers of mixed amino acids (proteinoids) have been used to deliver pharmaceuticals. For example, U.S. Pat. No. 4,925,673 describes drug-containing proteinoid microsphere carriers as well as methods for their preparation and use. These proteinoid microspheres are useful for the delivery of a number of active agents. Also see, U.S. Pat. Nos. 5,907,030 and 6,033,884, which are incorporated herein by reference.

B. Introduction of Polynucleotides into Cells

By introducing gene sequences into cells, gene therapy can be used to treat diseased cells (e.g., neuron cells that are associated with apoptosis or an excitotoxic response due to a neuronal insult). In one embodiment, a polynucleotide that encodes a PL sequence peptide of the invention is introduced into a cell where it is expressed. The expressed peptide then inhibits the interaction of PDZ proteins and PL proteins in the cell.

Thus, in one embodiment, the polypeptides of the invention are expressed in a cell by introducing a nucleic acid (e.g., a DNA expression vector or mRNA) encoding the desired protein or peptide into the cell. Expression can be either constitutive or inducible depending on the vector and choice of promoter. Methods for introduction and expression of nucleic acids into a cell are well known in the art and described herein.

In a specific embodiment, nucleic acids comprising a sequence encoding a peptide disclosed herein, are administered to a human subject. In this embodiment of the invention, the nucleic acid produces its encoded product that mediates a therapeutic effect. Any of the methods for gene therapy available in the art can be used according to the present invention. Exemplary methods are described below.

For general reviews of the methods of gene therapy, see Goldspiel et al., 1993,

Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred embodiment of the invention, the therapeutic composition comprises a coding sequence that is part of an expression vector. In particular, such a nucleic acid has a promoter operably linked to the coding sequence, said promoter being inducible or constitutive, and, optionally, tissue-specific. In another specific embodiment, a nucleic acid molecule is used in which the coding sequence and any other desired sequences are flanked by regions that promote homologous recombination at a desired site in the genome, thus providing for intrachromosomal expression of the nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus, or by administering it in linkage to a ligand subject to receptor-mediated endocytosis (see e.g., Wu and Wu, 1987, J. Biol. Chem. 262:4429-4432) which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated April 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a preferred embodiment of the invention, adenoviruses as viral vectors can be used in gene therapy. Adenoviruses have the advantage of being capable of infecting non-dividing cells (Kozarsky and Wilson, 1993, Current Opinion in Genetics and Development 3:499-503). Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234. Furthermore, adenoviral vectors with modified tropism may be used for cell specific targeting (WO98/40508). Adeno-associated virus (AAV) has also been proposed for use in gene therapy (Walsh et al., 1993, Proc. Soc. Exp. Biol. Med. 204:289-300).

In addition, retroviral vectors (see Miller et al., 1993, Meth. Enzymol. 217:581-599) have been modified to delete retroviral sequences that are not necessary for packaging of the viral genome and integration into host cell DNA. The coding sequence to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a gene to cells in tissue culture. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, lipofection, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. In a preferred embodiment, the cell used for gene therapy is autologous to the patient.

In a specific embodiment, the nucleic acid to be introduced for purposes of gene therapy comprises an inducible promoter operably linked to the coding sequence, such that expression of the nucleic acid is controllable by controlling the presence or absence of the appropriate inducer of transcription.

Oligonucleotides such as anti-sense RNA and DNA molecules, and ribozymes that function to inhibit the translation of a targeted mRNA, especially its C-terminus are also within the scope of the invention. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a nucleotide sequence, are preferred.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of target RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

The anti-sense RNA and DNA molecules and ribozymes of the invention may be prepared by any method known in the art for the synthesis of nucleic acid molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the RNA molecule. Such DNA sequences may be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

C. Other Pharmaceutical Compositions

The compounds of the invention, may be administered to a subject per se or in the form of a sterile composition or a pharmaceutical composition. Pharmaceutical compositions comprising the compounds of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the active peptides or peptide analogues into preparations which can be used pharmaceutically. Proper formulation is dependent upon the route of administration chosen.

For topical administration the compounds of the invention can be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the compounds of the invention can be formulated in aqueous solutions, preferably in physiologically compatible buffers such as Hanks's solution, Ringer's solution, or physiological saline buffer. The solution can contain formulatory agents such as suspending, stabilizing and/or dispersing agents.

Alternatively, the compounds can be in powder form for constitution with a suitable vehicle, e.g., sterile pyrogen-free water, before use.

For transmucosal administration, penetrants appropriate to the barrier to be permeated are used in the formulation. Such penetrants are generally known in the art. This route of administration may be used to deliver the compounds to the nasal cavity.

For oral administration, the compounds can be readily formulated by combining the active peptides or peptide analogues with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compounds may take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Alternatively, other pharmaceutical delivery systems may be employed.

Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the compounds of the invention may contain charged side chains . or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

D. Effective Dosages

The compounds of the invention will generally be used in an amount effective to achieve the intended purpose (e.g., treatment of a neuronal injury). The compounds of the invention or pharmaceutical compositions thereof, are administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein. An “inhibitory amount” or “inhibitory concentration” of a PL-PDZ binding inhibitor is an amount that reduces binding by at least about 40%, preferably at least about 50%, often at least about 70%, and even as much as at least about 90%. Binding can as measured in vitro (e.g., in an A assay or G assay) or in situ.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day. For usual peptide therpaeutic treatment of stroke, acute administration of 0.03 nmol/g to 30 nmol/g within 6 hours of stroke or brain ischemia is typical. In other instances, 0.1 nmol/g to 20 nmol/g within 6 hours are administered. And in still other instances lnmoUg to 10 nmol/g is administered with in 6 hours.

In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs.

E. Toxicity

Preferably, a therapeutically effective dose of the compounds described herein will provide therapeutic benefit without causing substantial toxicity.

Toxicity of the compounds described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

### Example 1

### Generation of Eukaryotic Expression Constructs Bearing DNA Fragments that Encode PDZ Domain Containing Genes or Portions of PDZ Domain Genes

This example describes the cloning of PDZ domain containing genes or portions of PDZ domain containing genes into prokaryotic expression vectors in fusion with Glutathione S-Transferase (GST). Some PDZ proteins were also cloned into eukaryotic expression vectors in fusion with a number of protein tags, including but not limited to Enhanced Green Fluorescent Protein (EGFP) or Hemagglutinin (HA).

A. Strategy

DNA fragments corresponding to PDZ domain containing genes were generated by RT-PCR from RNA from a library of individual cell lines (CLONTECH Cat#K4000-1) derived RNA, using random (oligo-nucleotide) primers (Invitrogen Cat.#48190011). DNA fragments corresponding to PDZ domain containing genes or portions of PDZ domain containing genes were generated by standard PCR, using above purified cDNA fragments and specific primers (see TABLE 5). Primers used were designed to create restriction nuclease recognition sites at the PCR fragment's ends, to allow cloning of those fragments into appropriate expression vectors. Subsequent to PCR, DNA samples were submitted to agarose gel electrophoresis. Bands corresponding to the expected size were excised. DNA was extracted by Sephaglas Band Prep Kit (Amersham Pharmacia Cat#27-9285-01) and digested with appropriate restriction endonuclease. Digested DNA samples were purified once more by gel electrophoresis, according to the same protocol used above. Purified DNA fragments were coprecipitated and ligated with the appropriate linearized vector. After transformation into E. coli, bacterial colonies were screened by colony PCR and restriction digest for the presence and correct orientation of insert. Positive clones were innoculated in liquid culture for large scale DNA purification. Plasmid purification was done by mini, midi, or maxiprep (Quiagen or Mo Bio), according to the manufacturer's protocol. The insert and flanking vector sites from the purified plasmid DNA were sequenced to ensure correct sequence of fragments and junctions between the vectors and fusion proteins.

B. Vectors

All PDZ domain-containing genes were cloned into the vector pGEX-3X (Amersham Pharmacia #27-4803-01, Genemed Acc#U13852, GI#595717), containing a tac promoter, GST, Factor Xa, β-lactamase, and lac repressor.

The amino acid sequence of the pGEX-3X coding region including GST, Factor Xa, and the multiple cloning site is listed below. Note that linker sequences between the cloned inserts and GST-Factor Xa vary depending on the restriction endonuclease used for cloning. Amino acids in the translated region below that may change depending on the insertion used are indicated in small caps, and are included as changed in the construct sequence listed in (C).

aa 1-aa 232:
MSPILGYWKIKGLVQPTRLLLEYLEEKYEEHLYERDEGDKWRNKKFEL
GLEFPNLPYYIDGDVKLTQSMAIIRYIADKHNMLGGCPKERAEISMLE
GAVLDIRYGVSRIAYSKDFETLKVDFLSKLPEMLKMFEDRLCHKTYLN
GDHVTHPDFMLYDALDVVLYMDPMCLDAFPKLVCFKKRIEAIPQIDKY
LKSSKYIAWPLQGWQATFGGGDHPPKSDLIEGRgipgnss

C. Constructs

Primers used to generate DNA fragments by PCR are listed in TABLE 5. PCR primer combinations and restriction sites for insert and vector are listed below, along with amino acid translation for insert and restriction sites. Non-native amino acid sequences are shown in lower case.

TABLE 5
Primers used in cloning of PSD95 (all 3 domains), DLG 1 (domains 1 and 2), TIP2
(domain 1 of 1), and LIM (domain 1 of 1) into representative expression vectors.

| Primer | Primer | |
|---|---|---|
| Name | Sequence | Description |
| 1DF | TCGGATCCAGGTT | Forward (5′ to 3′) primer corresponding to DLG 1, |
| | AATGGCTCAGATG | nucleotide numbers 815-841. Generates a Bam H1 |
| | | site upstream (5′) of the PDZ 1 boundary. Used for |
| | | cloning into pGEX-3X. |
| 2DR | CGGAATTCGGTGC | Reverse (3′ to 5′) primer corresponding to DLG 1, |
| | ATAGCCATC | nucleotide numbers 1442-1421. Generates an |
| | | EcoR1 site downstream (3′) of the PDZ 2 boundary. |
| | | Used for cloning into pGEX-3X. |
| 8PSF | TCGGATCCTTGAG | Forward (5′ to 3′) primer corresponding to PSD95, |
| | GGGGAGATGGA | nucleotide numbers 1150-1173. Generates a |
| | | BamH1 site upstream (5′) of the PDZ 1 boundary. |
| | | Used for cloning into pGEX-3X. |
| 11PSR | TCGGAATTCGCTA | Reverse (3′ to 5′) primer corresponding to PSD95, |
| | TACTCTTCTGG | nucleotide numbers 2191-2168. Generates an |
| | | EcoR1 site downstream (3′) of the PDZ 3 boundary. |
| | | Used for cloning into pGEX-3X. |
| 182LF | TTAGGATCCTGAG | Forward (5′ to 3′) primer corresponding to LIM, |
| | CAAGTACAGTGTG | nucleutide numbers 86-115. Generates a Bam H1 |
| | TCAC | site upstream (5′) of the PDZ boundary. Used for |
| | | cloning into pGEX-3X. |
| 183LR | CTTGAATTCAGCA | Reverse (3′ to 5′) primer corresponding to LIM, |
| | GATGCTCTTTGCA | nucleotide numbers 350-320. Generates an EcoR1 |
| | GAGTC | site downstream (3′) of the PDZ boundary. Used for |
| | | cloning into pGEX-3X. |
| 197TF | AGGGGATCCGCAA | Forward (5′ to 3′) primer corresponding to TIP2. |
| | GGAGGTGGAGGTG | Generates a Bam H1 site upstream (5′) of the PDZ |
| | TTC | boundary. Used for cloning into pGEX-3X. |
| 198TR | TGTGGAATTCCTT | Reverse (3′ to 5′) primer corresponding to TIP2, |
| | GCGAGGCTCCGTG | nucleotide numbers 429-401. Generates an EcoR1 |
| | AGC | site downstream (3′) of the PDZ boundary. Used for |
| | | cloning into pGEX-3X. |

1. DLG 1, PDZ domains 1 and 2:

Acc#:U13897

Construct: DLG 1, PDZ domains 1 and 2-pGEX-3X

Primers: 1DF & 2DR
Vector Cloning Sites(5′/3′): Bam H1/EcoR1
Insert Cloning Sites(5′/3′): BamH1/EcoR1

aa 275-aa 477
qVNGTDADYEYEEITLERGNSGLGFSIAGGTDNPHIGDDSSIFITKIIT
GGAAAQDGRLRVNDCILRVNEVDVRDVTHSKAVEALKEAGSIVRLYVKR
RKPVSEKIMEIKLIKGPKGLGFSIAGGVGNQHIPGDNSIYVTKIIEGGA
AHKDGKLQIGDKLLAVNNVCLEEVTHEEAVTALKNTSDFVYLKVAK
PTSMYMNDGYApns

2. PSD95, PDZ domains 3 of 3:

Acc#:U83192

Construct: PSD95, PDZ domains 3 of 3-pGEX-3X
Primers: 8PSF & 11PSR
Vector Cloning Sites(5′/3′): Bam H1/EcoR1
Insert Cloning Sites(5′/3′): BamH1/EcoR1

aa 387-aa 724
legEGEMEYEEITLERGNSGLGFSIAGGTDNPHIGDDPSIFITKIIP
GGAAAQDGRLRVNDSILFVNEVDVREVTHSAAVEALKEAGSIVRLYV
MRRKPPAEKVMEIKLIKGPKGLGFSIAGGVGNQHLPGDNSIYVTKII
EGGAAHKDGRLQIGDKILAVNSVGLEDVMHEDAVAALICNTYDVVYL
KVAICPSNAYLSDSYAPPDITTSYSQHLDNEISHSSYLGTDYPTAMT
PTSPRRYSPVAKDLLGEEDIPREPRRIVIHRGSTGLGFNIVGGEDGE
GIFISFILAGGPADLSGELRKGDQILSVNGVDLRNASHEQAAIALKN
AGQTVTIIAQYKPEfiv

### 3. TAX Interacting Protein 2 (TIP2):

Acc#:AF028824

Construct: TIP2, PDZ domain 1 of 1-pGEX-3X
Primers: 197TF & 198TR
Vector Cloning Sites(5′/3′): Bam H1/EcoR1
Insert Cloning Sites(5′/3′): BamH1/EcoR1

aa 54-aa 140
RKEVEVFKSEDALGLTITDNGAGYAFIKRIKEGSVIDHIHLISVG
DMIEAINGQSLLGCRHYEVARLLKELPRGRTFTLKLTEPRKefiv
td

### 3. LIM Protein:

Acc#:AF061258

Construct: LIM-pGEX-3X
Primers: 182LF & 183LR
Vector Cloning Sites(5′/3′): Bam H1/Bam H1
Insert Cloning Sites(5′/3′): BamH1/Bam H1

aa 29-aa 112
lSNYSVSLVGPAPWGFRLQGGKDFNMPLTISSLKDGGKAAQA
NVRIGDVVLSIDGINAQGMTHLEAQNKIKGCTGSLNMTLQRA
Sc

D. GST Fusion Protein Production and Purification

The constructs using pGEX-3X expression vector were used to make fusion proteins according to the protocol outlined in the “GST Gene Fusion System”, Second Edition, Revision 2, Pharmacia Biotech. Method 11 was used, optimized for a 1 L LgPP.

In brief, a small culture (3-5 mls) containing a bacterial strain (DH5□, BL21 or JM109) with the fusion protein construct was grown overnight in 2XYT-media at 37° C. with the appropriate antibiotic selection (100 ug/ml ampicillin; a.k.a. LB-amp). The overnight culture was poured into a fresh preparation of 2XYT-amp (typically 250-500 mls) and grown until the optical density (OD) of the culture was between 0.5 and 0.9 (approximately 2.5 hours). IPTG was added to a final concentration of 1.0 mM to induce production of GST fusion protein, and culture was grown an additional 1-2 hours. Bacteria were collect by centrifugation (4500 g) and resuspended in Buffer A− (50 mM Tris, pH 8.0, 50 mM dextrose, 1 mM EDTA, 200 uM PMSF). An equal volume of Buffer A+ (Buffer A−, 4 mg/ml lysozyme) was added and incubated on ice for 3 min to lyse bacteria. An equal volume of Buffer B (10 mM Tris, pH 8.0, 50 mM KCl, 1 mM EDTA. 0.5% Tween-20, 0.5% NP40 (a.k.a. IGEPAL CA-630), 200 uM PMSF) was added and incubated for an additional 20 min. The bacterial cell lysate was centrifuged (×20,000 g), and supernatant was added to Glutathione Sepharose 4B (Pharmacia, cat no. 17-0765-01) previously swelled (rehydrated) in 1× phosphate-buffered saline (PBS). The supernatant-Sepharose slurry was poured into a column and washed with at least 20 bed volumes of 1× PBS. GST fusion protein was eluted off the glutathione sepharose by applying 0.5-1.0 ml aliquots of 5 mM glutathione and collected as separate fractions. Concentrations of fractions were determined using BioRad Protein Assay (cat no. 500-0006) according to manufacturer's specifications. Those fractions containing the highest concentration of fusion protein were pooled and glycerol was added to a final concentration of 35% glycerol. Fusion proteins were assayed for size-and quality by SDS gel electrophoresis (PAGE). Fusion protein aliquots were stored at minus 80° C.

Purified proteins were used for ELISA-based assays and antibody production.

### Example 2

### Identification of N-methyl-D-aspartate Receptor 2A (NMDAR2A) Interations with PSD95 TIP2, DLG1, and LIM in vitro

This example describes the binding of NMDAR2A to PSD95, TIP2, DLG1, and LIM, assessed using a modified ELISA. Briefly, a GST-PDZ fusion was produced that contained the entire PDZ domain of human LIM or TIP2, domains 1 and 2 of 3 in DLG1, or all 3 PDZ domains for PSD95 (see Example 1). In addition, biotinylated peptide corresponding to the C-terminal 20 amino acids of NMDAR2A was synthesized and purified by HPLC. Binding between these entities was detected through the “G” Assay, a colorimetric assay using avidin-HRP to bind the biotin and a peroxidase substrate.

### A. Peptide Purification

Peptide representing the C-terminal 20 amino acids of NMDAR2A, as shown in TABLES 2 and 3 was synthesized by standard FMOC chemistry and biotinylated if not used as an unlabeled competitor. Peptide was purified by reverse phase high performance liquid chromatography (HPLC) using a Vydac 218TP C18 Reversed Phase column having the dimensions of 10*25 mm, 5 um. Approximately 40 mg of peptide was dissolved in 2.0 ml of aqueous solution of 49.9% acetonitrile and 0.1% Tri-Fluoro acetic acid (TFA). This solution was then injected into the HPLC machine through a 25 micron syringe filter (Millipore). Buffers used to get a good separation are (A) distilled water with 0.1% TFA and (B) 0.1% TFA with Acetonitrile. Gradient Segment setup is listed in TABLE 6.

TABLE 6

| Time | A | B | C | Flow rate (ml/min) |
|---|---|---|---|---|
| 0 | 96% | 4% | 0 | 5.00 |
| 30 | 100% | 100% | 0 | 5.00 |
| 35 | 100% | 100% | 0 | 5.00 |
| 40 | 96% | 4% | 0 | 5.00 |

The separation occurs based on the nature of the peptides. A peptide of overall hydrophobic nature will elute off later than a peptide of a hydrophilic nature. Fractions containing the “pure” peptide were collected and checked by Mass Spectrometer (MS). Purified peptides are lyophilized for stability and later use.

### B. “G” Assay for Identification of Interactions Between Peptides and Fusion Proteins

### Reagents and Materials:

Nunc Polysorp 96 well Immuno-plate (Nunc cat#62409-005)
   (Maxisorp plates have been shown to have higher background signal)
PBS pH 7.4 (Gibco BRL cat#16777-148) or (AVC phosphate buffered saline, 8 gm NaCl, 0.29 gm KCl, 1.44 gm Na₂HPO₄, 0.24 gm KH₂PO₄, add H₂O to 1 L and pH 7.4; 0.2 μm filter
2% BSA/PBS (10 gm of bovine serum albumin, fraction V (ICN Biomedicals cat#IC15142983) into 500 ml PBS
Goat anti-GST mAb stock @ 5 mg/ml, store at 4° C., (Amersham Pharmacia cat#27-4577-01), dilute 1:1000 in PBS, final concentration 5 μg/ml
HRP-Streptavidin, 2.5 mg/2 ml stock stored at 4° C. (Zymed cat#43-4323), dilute 1:2000 into 2% BSA, final concentration at 0.5 μg/ml
Wash Buffer, 0.2% Tween 20 in 50 mM Tris pH 8.0
TMB ready to use (Dako cat#S 1600)
1M H₂SO₄
12 w multichannel pipettor,
50 ml reagent reservoirs,
15 ml polypropylene conical tubes

### Protocol

1) Coat plate with 100 μl of 5 μg/ml goat anti GST, O/N @ 4° C.
2) Dump coating antibodies out and tap dry
3) Blocking—Add 200 μl per well 2% BSA, 2 hrs at 4° C.
4) Prepare proteins in 2% BSA
   (2 ml per row or per two columns)
5) 3 washes with cold PBS (must be cold through entire experiment)
   (at last wash leave PBS in wells until immediately adding next step)
6) Add proteins at 50 μl per well on ice (1 to 2 hrs at 4° C.)
7) Prepare Peptides in 2% BSA (2 ml/row or /columns)
8) 3× wash with cold PBS
9) Add peptides at 50 μl per well on ice (time on/time off)
   a. keep on ice after last peptide has been added for 10 minutes exactly
   b. place at room temp for 20 minutes exactly
10) Prepare 12 ml/plate of HRP-Streptavidin (1:2000 dilution in 2% BSA)
11) 3× wash with cold PBS
12) Add HRP-Streptavidin at 100 ul per well on ice, 20 minutes at 4° C.
13) Turn on plate reader and prepare files
14) 5× washes, avoid bubbles
15) Using gloves, add TMB substrate at 100 μl per well
   a. incubate in dark at room temp
   b. check plate periodically (5, 10, & 20 minutes)
   c. take early readings, if necessary, at 650 nm (blue)
   d. at 20 minutes, stop reaction with 100 ul of 1M H2SO4
   e. take last reading at 450 nm (yellow)

### C. Results of Binding Experiments

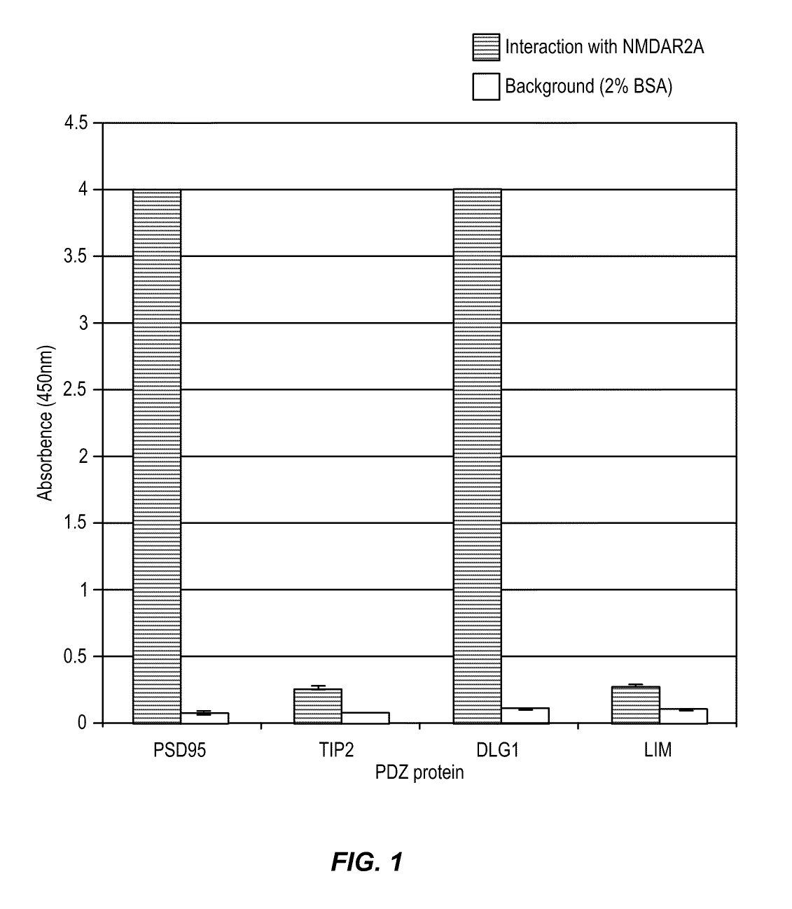

Results of peptides representing the carboxy-terminal 20 amino acids of NMDAR2A binding to PSD95, TIP2, DLG1, and LIM are shown in FIG. 1. NMDAR2A binds GST-PSD95 and GST-DLG1 with much higher affinity than it does to GST-LIM or GST-TIP2 at equivalent peptide concentrations and with an equivalent amount of GST-PDZ fusion protein. Because the interaction between NMDAR2A and LIM is not significantly higher than background, this particular experiment indicates that LIM PDZ's may not interact with NMDAR2A PL peptide.

### D. Conclusions and Summary

PSD-95 and DLG1 bind to NMDAR2A better than TIP2 and LIM bind to the same peptide. Thus, they are more likely to be in vivo interactions and binding of PDZ domains to the C-terminus of NMDA R2A will strongly favor PSD-95 and DLG1 over TIP2 or Lim.

### Example 3

### Treatment of Ischemic Brain Damage by Modulating NMDA-Receptor PSD-95 Interactions

Recent experiments performed by Aarts et al. (Science 298:846-850, 2002, which is incorporated herein by reference in its entirety) are consistent with the interactions identified herein, specifically the interactions between NMDAR and PSD-95.

Aarts et al. conducted studies with a fusion polypeptide in which the C-terminal 9 amino acids of NMDA Receptor 2B were fused to a Tat transmembrane transporter peptide and found that this fusion polypeptide could inhibit binding between NMDAR2 with domain 2 of PSD-95. The sequence of the inhibitory peptide was YGRKKRRQRRRKLSSIESDV. These researchers also demonstrated that this peptide, when labeled with dansyl chloride, could penetrate cells in the coronal section of the brain in a short amount of time. It was also found that administration of the polypeptide to rats either before or up to one hour after induction of transient middle cerebral artery occlusion (MCAO) significantly protected the rat brain from ischemic damage due to the occlusion. For example, in the presence of the inhibitory polypeptide, the infarct area in the cortical area of the brain following inducement of MCAO was reduced to below 20% of the infarct area of untreated rats.

### Example 4

### NMDA Receptor 2 Subunits Bind a Number of PDZ Domains

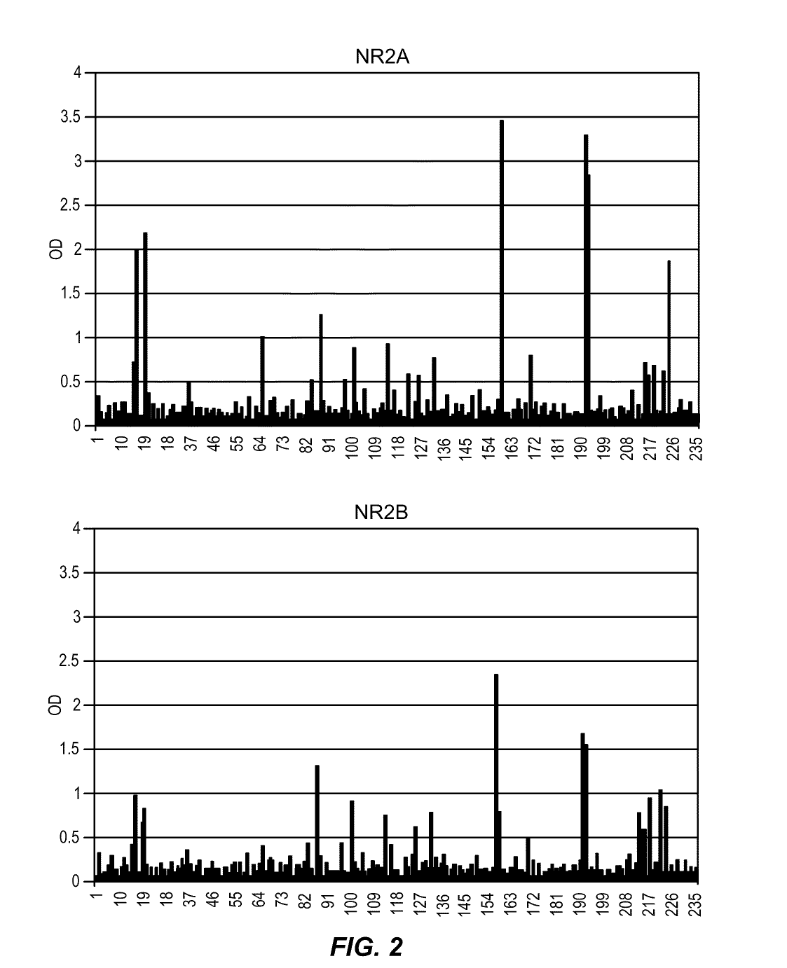

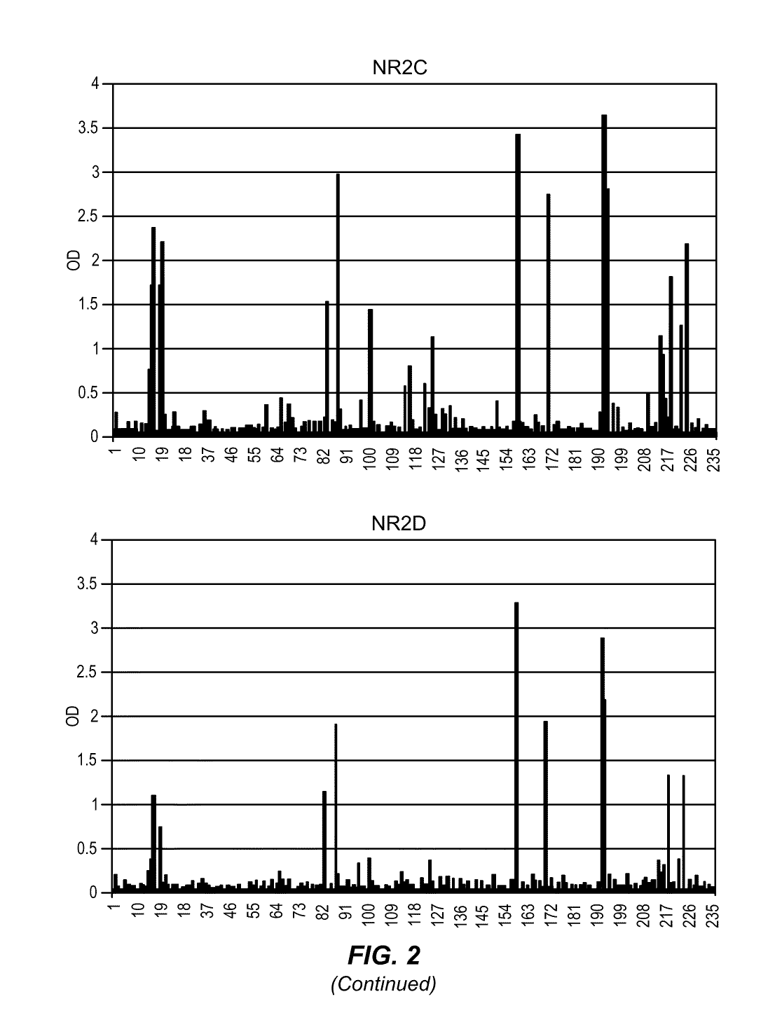

The selectivity of NMDA Receptor 2 (NR2) subunit binding to PDZ domains was assayed using the G assay described supra. Biotinylated peptides corresponding to the C-terminal 19 or 20 amino acids of NR2A, NR2B, NR2C, and NR2D were synthesized and tested for their ability to specifically interact with 238 independent PDZ domain constructs. FIG. 2 shows the results of these interactions. Each binds a similar subset of approximately 16 to 20 PDZ domains. PDZ interactions that are common to all NMDA R2 subunits or to only a subset are listed in TABLE 7.

TABLE 7
PDZ domains that interact strongly with NMDA R2 subunits

| PDZ domains that interact | PDZ domains that interact |
|---|---|
| with all NR2s | with a subset of NR2s |
| DLG1 d2 | DLG1 d1 |
| DLG2 d2 | INADL d8 |
| KIAA0973 | KIAA0807 |
| NeDLG | KIAA1634 d1 |
| Outermembrane Protein | Lim-Mystique |
| PSD-95 d2 | LIM-RIL |
| Syntrophin alpha 1 | MAGI1 d2, d4, d5 |
| TIP1 | MAGI2 d5 |
| TIP2 | PSD-95 d1 |
| | Syntrophin beta-1 |
| | Syntrophin gamma 1 |

Table 7 legend:
Domain numbers of PDZ proteins that contain multiple PDZ domains are indicated as d1 or d2 etc.

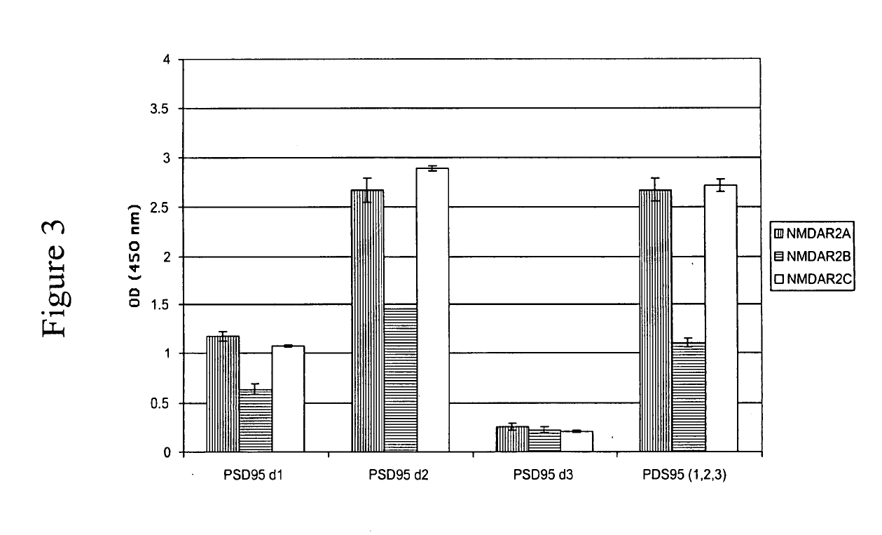

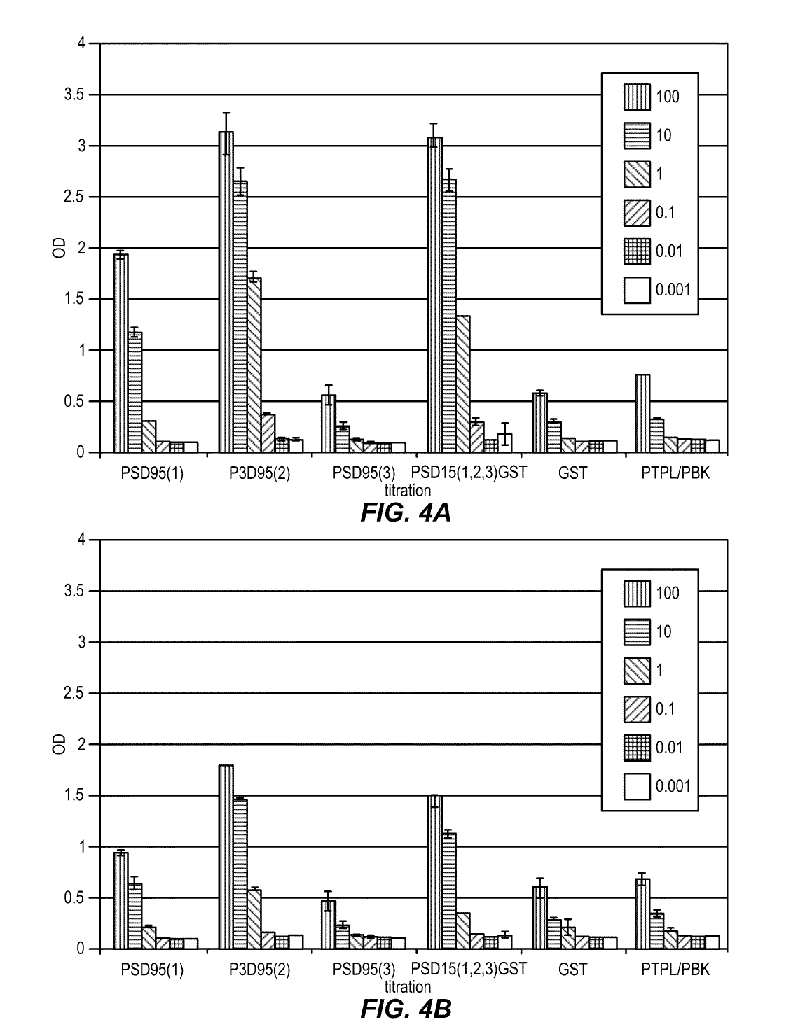

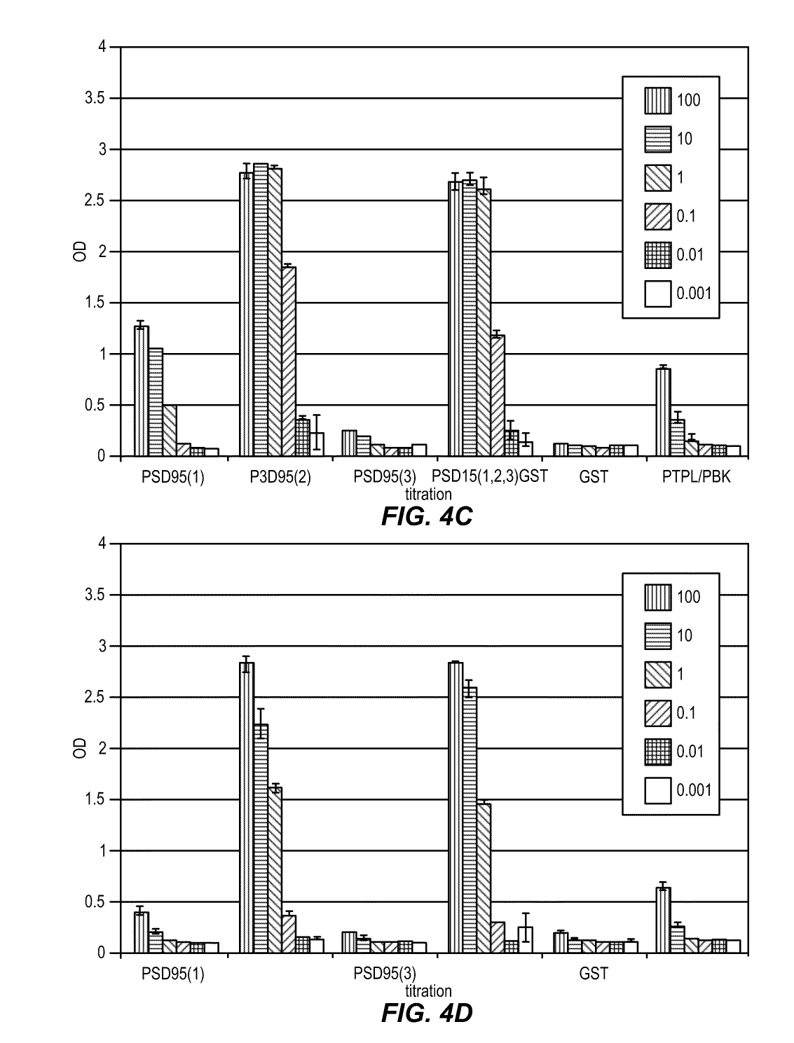

Concurrent binding tests were performed with the main R2 subunits indicated in neuroprotection (R2A, R2B, R2C) and the individual and complexed PDZ domains of PSD95 (FIG. 3). All three NR2 subunits bind PSD95 domains 1 and 2 but fail to bind PSD95 domain 3. Peptides corresponding to the C-termini of all 4 NR2 subunits were titrated against a constant amount of PSD95 PDZ domain proteins to determine relative binding affinities for the PL-mimicking peptides and each domain of PSD95. Results are shown in FIG. 4.

These experiments show that NR2 subunits can bind a number of different PDZ domains, and that the highest relative affinity interaction occurs between NR2C and PSD95 domain 2. Thus, peptides as described in Example 3 may inhibit a number of interactions. In addition to previous demonstrations that NMDA Receptor antagonists are neuroprotective, previous research has demonstrated that reduction of PSD95 protein in neuronal cells is neuroprotective. The methods for identifying inhibitors disclosed herein can be used to identify inhibitors that are specific for PSD-95 as well as inhibitors specific to other NMDA R2 PDZ interactions. Using such specific inhibitors, one can ascertain whether the neuroprotective effect of inhibitors is due wholly or partially to the NMDA R2 PSD95 interactions. Specific inhibitors that block only the necessary interaction(s) are extremely valuable in the reduction of side effects which often occur during clinical testing.

### Example 5

### Identification of 3,4 and 19 Amino Acid Inhibitors or NR2 Subunit/PSD95 PDZ Interactions

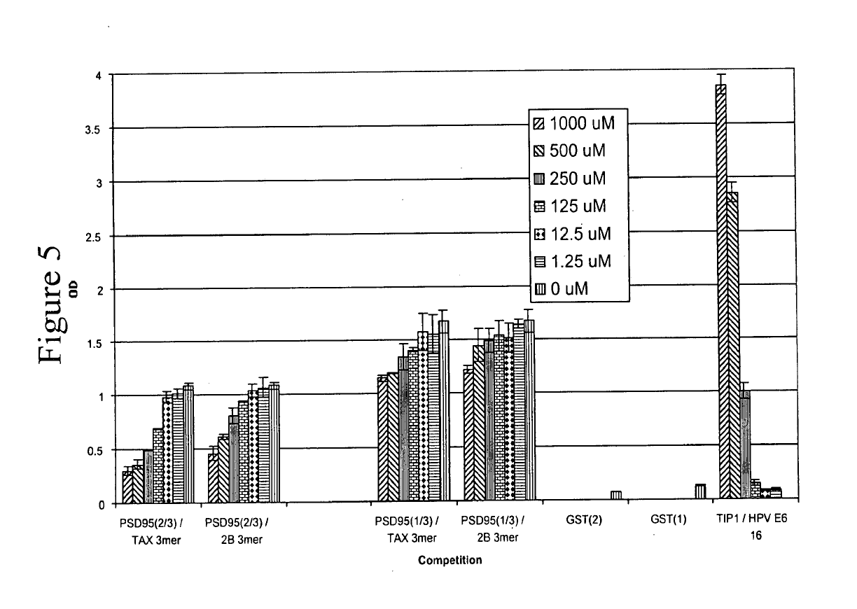

A number of peptides of different length were synthesized and tested for their ability to inhibit NMDA R2 interactions with PSD95 domain 1 or domain 2. These peptides were tested using the G assay as described supra and results are shown in FIGS. 5-9.

FIG. 5 shows the ability of N-terminal acetylated peptides corresponding to the C-terminal 3 amino acids of the TAX oncoprotein (Ac-TEV) and NMDA R2B (Ac-SDV) to inhibit the interaction between NMDA R2A and PSD95 domain 1 or domain 2. Both peptides are able to inhibit the interactions of NR2A and PSD95 domain 2, and only at the highest concentration (1 mM) is any inhibition seen with PSD95 domain 1 and NR2A.

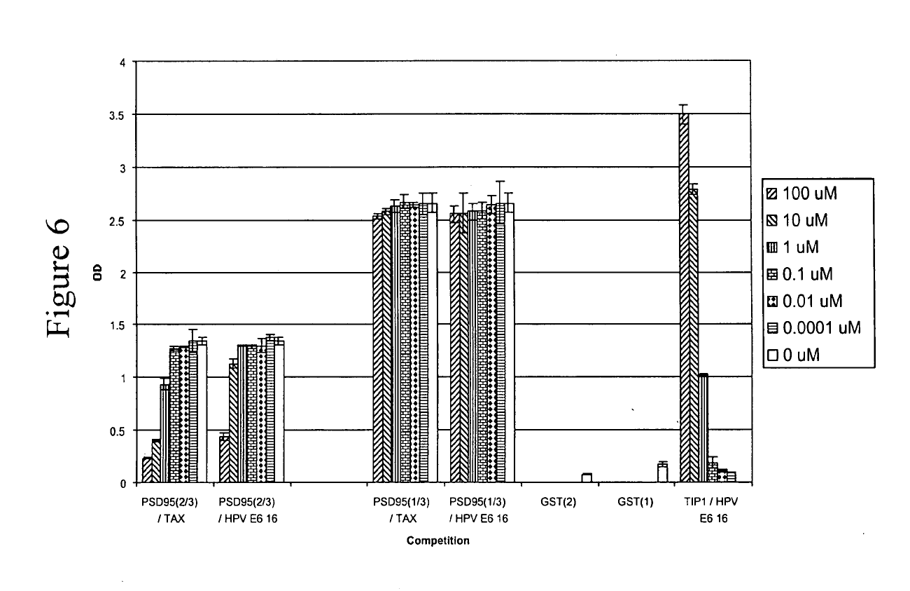

FIG. 6 shows the ability of N-terminal peptides corresponding to the C-terminal 19 or 20 amino acids of the TAX oncoprotein and HPV 16 E6 protein to inhibit the interaction between NMDA R2C and PSD95 domain 1 or domain 2. Both peptides are able to inhibit the interactions of NR2C and PSD95 domain 2, and no inhibition between PSD95 domain 1 and NR2C is seen in this concentration range (up to 100 uM). Peptides corresponding to the C-terminus of TAX (ending ETEV) show better inhibition that those of E6 (ending ETQL).

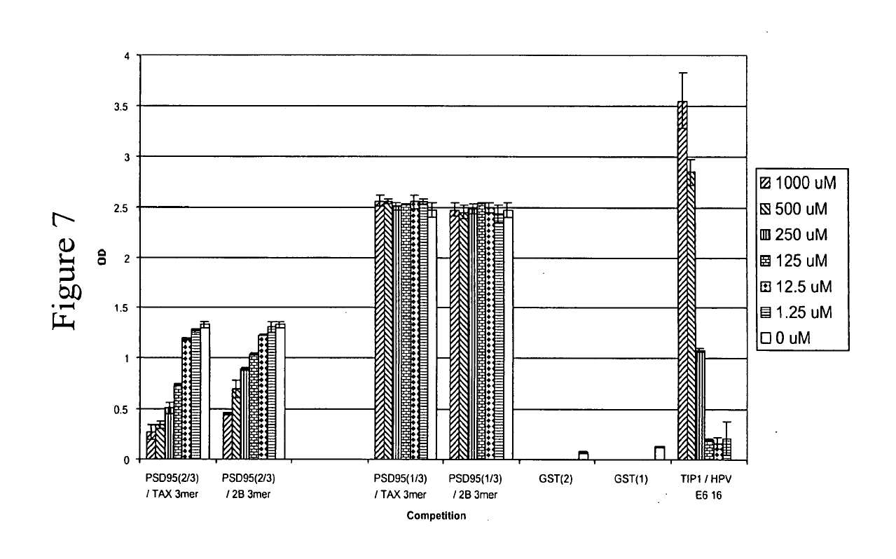

FIG. 7 shows the ability of N-terminal acetylated peptides corresponding to the C-terminal 3 amino acids of the TAX oncoprotein (Ac-TEV) and NMDA R2B (Ac-SDV) to inhibit the interaction between NMDA R2C and PSD95 domain 1 or domain 2. Both peptides are able to inhibit the interaction between NR2C and PSD95 domain 2, and no inhibition between PSD95 domain 1 and NR2C is seen in this concentration range (up to 1 mM).

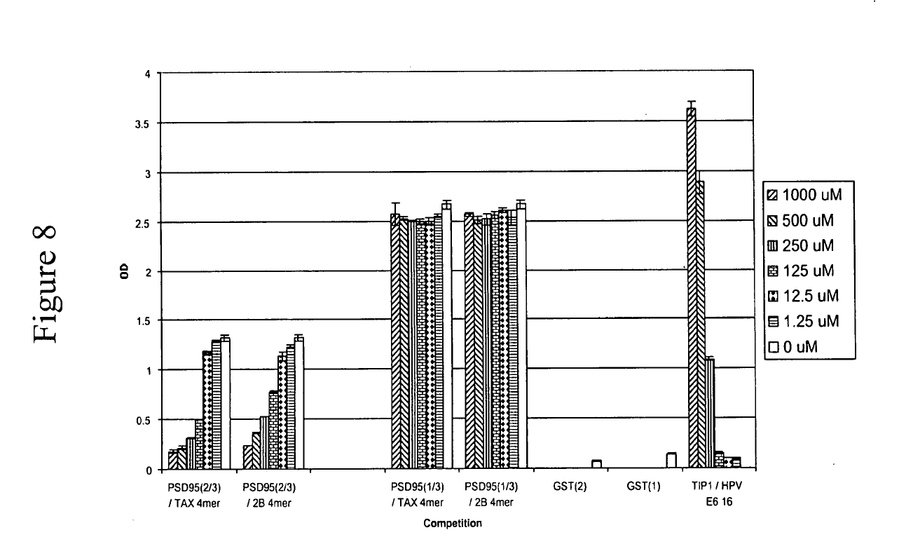

FIG. 8 shows the ability of N-terminal acetylated peptides corresponding to the C-terminal 4 amino acids of the TAX oncoprotein (Ac-ETEV) and NMDA R2B (Ac-ESDV) to inhibit the interaction between NMDA R2C and PSD95 domain 1 or domain 2. Both peptides are able to inhibit the interaction between NR2C and PSD95 domain 2, and no inhibition between PSD95 domain 1 and NR2C is seen in this concentration range (up to 1 mM). These 4 amino acid inhibitors both demonstrate a slightly better Ki than the 3 amino acid variants.

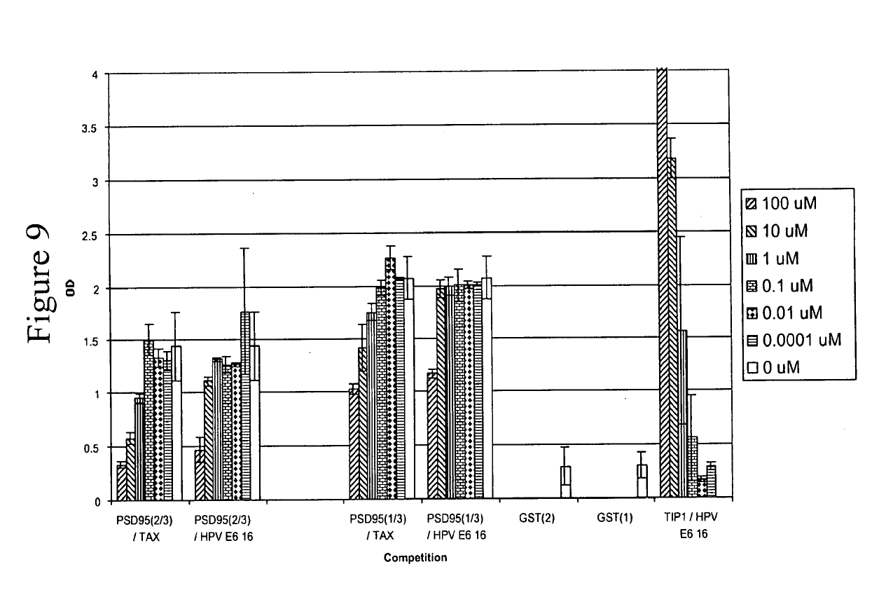

FIG. 9 shows the ability of N-terminal peptides corresponding to the C-terminal 19 or 20 amino acids of the TAX oncoprotein and HPV 16 E6 protein to inhibit the interaction between NMDA R2A and PSD95 domain 1 or domain 2. Both peptides are able to inhibit the interactions of NR2A and PSD95 domain 2, and for TAX sequences inhibition of the interaction between PSD95 domain 1 and NR2A is seen at 1 to 10 peptide concentration equivalents ([NR2A] for PSD95 domain 1=10 uM; inhibition seen at Tax concentrations of 10 uM and 100 uM). Peptides corresponding to the C-terminus of TAX (ending ETEV) show better inhibition that those of E6 (ending ETQL) for both domain 1 and 2 of PSD95.

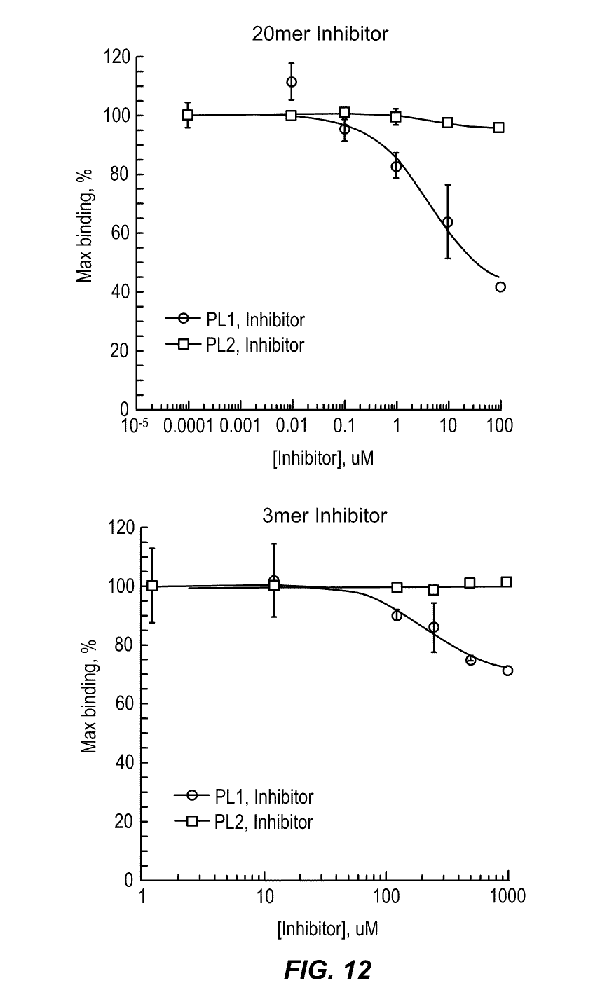

FIG. 12 shows that although both NMDA R2A and NMDA R2C can bind the 1st PDZ of PSD-95, either a 20 amino acid or a three amino acid inhibitor corresponding to the C-terminus of the TAX oncoprotein can selectively block the ability of NMDA R2A (PL1) to bind PSD-95 d1 without blocking the ability of NMDA R2C (PL2) to bind PSD-95 d1.

### Summary

Peptide inhibitors of NMDA Receptor 2 subunit interactions with PSD95 PDZ domains 1 and 2 have been identified. These inhibitors can function with as little as 3 amino acids to 20 amino acids with increasing affinity. Many more sequences were tested in this manner, and peptide inhibitors terminating in ETEV, ETQL, QTQV, ETAL, QTEV and ESEV showed the best ability to block interactions between NMDA R2′s and PSD95 domain 2 (with varying concurrent ability to inhibit PSD95 domain 1 interactions). Peptides sequences terminating in ETVA and FTDV had greater ability to inhibit PSD95 domain 1 interactions. Grouping these findings, peptides with consensus X-T-X-(V,L, or A) can inhibit PSD95 domain 1 and 2 interactions with NMDA Receptor 2 subunits. FIG. 12 shows the ability to achieve selective inhibition, where either the 20 amino acid or the three amino acid inhibitors corresponding to the C-terminus of Tax (TEV) are able to selectively inhibit the interaction of NR2A with PSD95 domain 1 without inhibiting the ability of NR2C to interact with domain 1. Thus, using approaches such as described herein, one can design inhibitors to selectively modulate interactions to treat specific phenotypes without disrupting all potential activities of the PDZ domain.

### Example 6

### Addition of Transporter Peptides to 9 Amino Acid Inhibitors Does Not Negatively Affect Inhibitory Ability

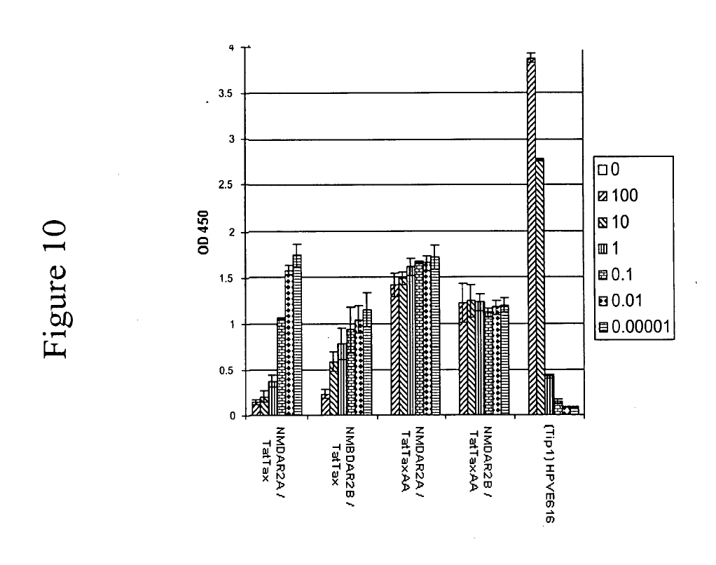

Since peptides corresponding to the TAX oncoprotein were effective inhibitors of NMDA R2 interactions with PSD95 PDZ domains, transporter peptide-coupled versions with native or disrupted PL sequences were synthesized. These peptides were assessed for their ability to inhibit interactions between PSD95 domain 2 and either NMDAR2A or R2B. The TatTAX construct with the native PL was able to show inhibition at concentrations as low as 10 nM (FIG. 10), with half maximal inhibition between 0.1 and 1.0 uM. When the PL was mutated by alanine substitution at residues 0 and −2, no inhibition was seen for NMDA R2A or R2B with PSD95 domain 2, indicating that the inhibition is dependent on the PL sequence and not the additional transporter sequences. In a similar manner, other transporters described supra were also effective.

### Example 7

### An Internal PDZ Ligand in nNOS Binds to the 2^{nd} PDZ Domain of PSD95 Without Binding the 1^{st} or 3^{rd} PDZS

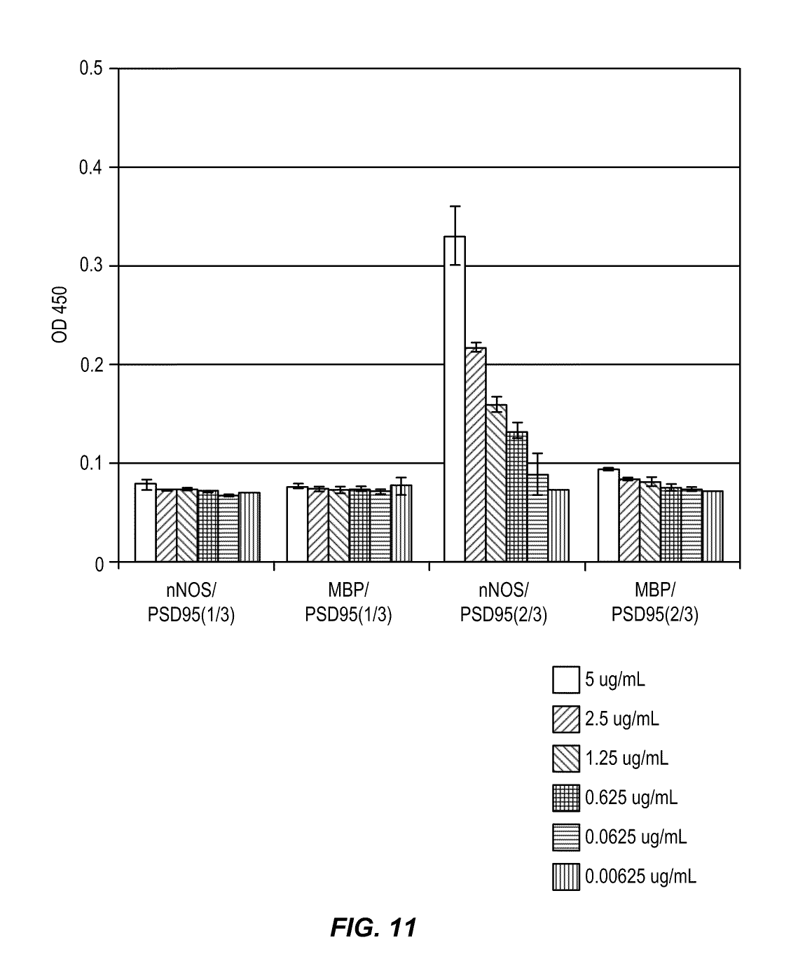

Excitotoxicity stemming from ischemia or neurotrauma is highly correlated with Nitric Oxide (NO) production. Inhibition of nNOS, the major neuronal enzyme producing NO, is neuroprotective, and nNOS itself has been shown to interact with PSD95. An expression construct was made comprising the Maltose Binding Protein (MBP) and amino acids 1-120 of nNOS (gi:10835173). This protein was used in the G assay in place of a labeled peptide to assess the interaction between PSD95 domains 1 and 2 and nNOS, and detection was performed using an HRP-conjugated antibody against MBP. FIG. 11 shows that the internal PL of MBP-nNOS can specfically recognize PDZ domain 2 of PSD95 but fails to interact with PSD95 domain 1. MBP/PSD95 indicates a negative control containing MBP without the nNOS sequences.

Since PSD95 and nNOS have each been implicated in neuroprotection, modulating of the interaction between these proteins provides an alternative therapeutic target for neuroprotection.

### Example 8

### The PDZ Domain of nNOS Binds a Number of Different PL Sequences and Can Recruit Additional Proteins to NMDAR/PSD95/nNOS Complexes

The G assay was used to identify PL sequences able to bind the PDZ domain of nNOS, and these sequences and binding values are shown in TABLE 8. These sequences can be used to identify similar sequences in the genome (methods described supra) that may be important nNOS interactions involved in neurotoxicity or neuroprotection. Designing inhibitors that block the PDZ domain of nNOS in neurons or neuronal tissues could provide and attractive alternative therapeutic target for neuroprotection.

TABLE 8
PL sequences that bind the PDZ domain of nNOS by G-assay.

| | Average | Relative | |
|---|---|---|---|
| PL Name | OD | STDV | Sequence |
| AdenoE4 typ9 | 4.00 | 0.0000 | VGTLLLERVIFPSVKIATLV |
| AdenoE4 typ9 | 4.00 | 0.0000 | VGTLLLERVIFPSVKIATLV |
| ephrin A2 | 4.00 | 0.0000 | RIAYSLLGLKDQVNTVGIPI |
| HPV-E6 #63 | 4.00 | 0.0000 | VHKVRNKFKAKCSLCRLYII |
| HPV-E6 #63 | 4.00 | 0.0000 | VHKVRNKFKAKCSLCRLYII |
| MINT-1 | 4.00 | 0.0000 | KTMPAAMYRLLTAQEQPVYI |
| MINT-1 | 4.00 | 0.0000 | KTMPAAMYRLLTAQEQPVYI |
| MINT-1 | 4.00 | 0.0163 | KTMPAAMYRLLTAQEQPVYI |
| MINT-1 | 3.76 | 0.0047 | KTMPAAMYRLLTAQEQPVYI |
| AdenoE4 typ9 | 3.64 | 0.0259 | VGTLLLERVIFPSVKIATLV |
| a-2B Adrenergic receptor | 3.54 | 0.1835 | QDFRRAFRRILARPWTQTAW |
| a-2C Adrenergic receptor | 3.02 | 0.4586 | DFRPSFKHILFRRARRGFRQ |
| AdenoE4 typ9 | 2.73 | 0.0005 | VGTLLLERVIFPSVKIATLV |
| HPV-E6 #63 | 2.55 | 0.0385 | VHKVRNKFKAKCSLCRLYII |
| a-2B Adrenergic receptor | 2.52 | 0.0208 | QDFRRAFRRILARPWTQTAW |
| CSPG4 (chondroitin sulfae | 2.43 | 0.0134 | ELLQFCRTPNPALKNGQYWV |
| proteoglycan 4, melanoma- | | | |
| associated) | | | |
| DNAM-1 | 2.40 | 0.0871 | TREDIYVNYPTFSRRPKTRV |
| HPV-E6 #18 | 2.14 | 0.1098 | HSCCNRARQERLQRRRETQV |
| catenin-delta 2 | 2.10 | 0.0967 | PYSELNYETSHYPASPDSWV |
| alpha-2C Adrenergic receptor | 2.09 | 0.0186 | DFRPSFKHILFRRARRGFRQ |
| Fas Ligand | 1.99 | 0.0515 | SSKSKSSEESQTFFGLYKL |
| Fas Ligand | 1.93 | 0.1362 | SSKSKSSEESQTFFGLYKL |
| ephrin A2 | 1.83 | 0.0186 | RIAYSLLGLKDQVNTVGIPI |
| presenilin-1 | 1.80 | 0.0173 | ATDYLVQPFMDQLAFHQFYI |
| ephrin B2 | 1.68 | 0.0644 | ILNSIQVMRAQMNQIQSVEV |
| DNAM-1 | 1.67 | 0.1086 | TREDIYVNYPTFSRRPKTRV |
| Dopamine transporter | 1.38 | 0.0471 | RELVDRGEVRQFTLRHWLKV |
| CSPG4 (chondroitin sulfae | 1.27 | 0.0627 | ELLQFCRTPNPALKNGQYWV |
| proteoglycan 4, melanoma- | | | |
| associated) | | | |
| Dopamine transporter | 1.27 | 0.0545 | RELVDRGEVRQFTLRHWLKV |
| Dopamine transporter | 1.10 | 0.0155 | RELVDRGEVRQFTLRHWLKV |
| noradrenaline transporter | 1.08 | 0.0864 | HHLVAQRDIRQFQLQHWLAI |
| presenilin-1 | 1.06 | 0.1494 | ATDYLVQPFMDQLAFHQFYI |
| Serotonin receptor 3a | 1.02 | 0.1510 | LAVLAYSITLVMLWSIWQYA |
| claudin 7 | 1.01 | 0.1490 | KAGYRAPRSYPKSNSSKEYV |
| alpha-2A Adrenergic receptor | 0.96 | 0.0471 | HDFRRAFKKILARGDRKRIV |
| DNAM-1 | 0.96 | 0.1079 | TREDIYVNYPTFSRRPKTRV |
| claudin 7 | 0.93 | 0.0053 | KAGYRAPRSYPKSNSSKEYV |
| claudin 1 | 0.90 | 0.1158 | SYPTPRPYPKPAPSSGKDYV |
| DNAM-1 | 0.86 | 0.0148 | TREDIYVNYPTFSRRPKTRV |
| beta-2 Adrenergic Receptor | 0.85 | 0.9946 | VPSDNIDSQGRNASINDSLL |
| LPAP | 0.77 | 0.1007 | AWDDSARAAGGQGLHVTAL |
| GLUR7 (metabotropic | 0.75 | 0.1655 | VDPNSPAAKKKYVSYNNLVI |
| glutamate receptor) | | | |
| HPV E6 #35 (cysteine-free) | 0.74 | 0.0411 | GRWTGRAMSAWKPTRRETEV |
| alpha-2A Adrenergic receptor | 0.73 | 0.1252 | HDFRRAFKKILARGDRKRIV |
| KIAA 1481 | 0.71 | 0.0961 | PIPAGGCTFSGIFPTLTSPL |
| claudin 2 | 0.69 | 0.1976 | PGQPPKVKSEFNSYSLTGYV |
| CD68 | 0.67 | 0.0242 | ALVLIAFCIIRRRPSAYQAL |
| KV1.3 | 0.67 | 0.0011 | TTNNNPNSAVNIKKIFTDV |
| GluR5-2 (rat) | 0.65 | 0.0186 | SFTSILTCHQRRTQRKETVA |
| GLUR7 (metabotropic | 0.64 | 0.0759 | VDPNSPAAKKKYVSYNNLVI |
| glutamate receptor) | | | |
| HPV E6 #35 (cysteine-free) | 0.62 | 0.0366 | GRWTGRAMSAWKPTRRETEV |
| CD6 | 0.59 | 0.0528 | SPQPDSTDNDDYDDISAA |
| HPV E6 58 (modified) | 0.59 | 0.0624 | AVGGRPARGGRLQGRRQTQV |
| GLUR7 (metabotropic | 0.59 | 0.4410 | VDPNSPAAKKKYVSYNNLVI |
| glutamate receptor) | | | |
| Nectin 2 | 0.54 | 0.1487 | SSPDSSYQGKGFVMSRAMYV |
| ephrin B2 | 0.54 | 0.0183 | ILNSIQVMRAQMNQIQSVEV |
| claudin 9 | 0.51 | 0.0498 | LGYSIPSRSGASGLDKRDYV |
| CD62E | 0.50 | 0.0225 | SSSQSLESDGSYQKPSYIL |
| NMDA Glutamate Receptor | 0.49 | 0.1629 | TQGFPGPATWRRISSLESEV |
| 2C (cysteine-free) | | | |

### Example 9

### The PDZ Domains of PSD-95 Bind a Number of Different PL Sequences and Can Recruit Additional Proteins to NMDAR/PSD95/nNOS Complexes

The G assay was used to identify PL sequences able to bind the three PDZ domains of PSD-95, and these sequences and binding values are shown in TABLE 9. These sequences can be used to identify similar sequences in the genome (methods described supra) that may be important PSD-95 interactions involved in neurotoxicity or neuroprotection. Inhibitors based on these sequences that block the PDZ domains of PSD-95 in neurons or neuronal tissues can provide an attractive alternative therapeutic target for neuroprotection. Sequences with higher OD binding to the 3 domains of PSD-95 are potentially stronger interactions.

TABLE 9
Peptide sequences that bind
the PDZ domains of PSD-95

| | Domain | Average | |
|---|---|---|---|
| Gene | String | OD | Sequence |
| PSD95 | 1 | 4.17 | QISPGGLEPPSEKHFRETEV |
| PSD95 | 1 | 4.07 | LNSCSNRRVYKKMPSIESDV |
| PSD95 | 1 | 4.06 | VGTLLLERVIFPSVKIATLV |
| PSD95 | 1 | 4.04 | VGTLLLERVIFPSVKIATLV |
| PSD95 | 1 | 4.00 | LNSCSNRRVYKKMPSIESDV |
| PSD95 | 1 | 4.00 | QISPGGLEPPSEKHFRETEV |
| PSD95 | 1 | 4.00 | VHKVRNKFKAKCSLCRLYII |
| PSD95 | 1 | 4.00 | VHKVRNKFKAKCSLCRLYII |
| PSD95 | 1 | 4.00 | GRWTGRAMSAWKPTRRETEV |
| PSD95 | 1 | 4.00 | GRWTGRAMSAWKPTRRETEV |
| PSD95 | 1 | 4.00 | AVGGRPARGGRLQGRRQTQV |
| PSD95 | 1 | 4.00 | GRWTGRAMSAWKPTRRETEV |
| PSD95 | 1 | 4.00 | TQGFPGPATWRRISSLESEV |
| PSD95 | 1 | 4.00 | AVGGRPARGGRLQGRRQTQV |
| PSD95 | 1 | 4.00 | VGTLLLERVIFPSVKIATLV |
| PSD95 | 1 | 3.87 | YGRKKRRQRRRKLSSIESDV |
| PSD95 | 1 | 3.86 | TGSALQAWRHTSRQATESTV |
| PSD95 | 1 | 3.15 | VGTLLLERVIFPSVKIATLV |
| PSD95 | 1 | 3.12 | SFTSILTCHQRRTQRKETVA |
| PSD95 | 1 | 3.08 | GRWTGRAMSAWKPTRRETEV |
| PSD95 | 1 | 2.81 | YGRKKRRQRRREKHFRETEV |
| PSD95 | 1 | 2.76 | AAGGRSARGGRLQGRRETAL |
| PSD95 | 1 | 2.67 | VHKVRNKFKAKCSLCRLYII |
| PSD95 | 1 | 2.66 | TQGFPGPATWRRISSLESEV |
| PSD95 | 1 | 2.66 | AAGGRSARGGRLQGRRETAL |
| PSD95 | 1 | 2.66 | TQGFPGPATWRRISSLESEV |
| PSD95 | 1 | 2.51 | AAGGRSARGGRLQGRRETAL |
| PSD95 | 1 | 2.47 | TQGFPGPATWRRISSLESEV |
| PSD95 | 1 | 2.28 | TTNNNPNSAVNIKKIFTDV |
| PSD95 | 1 | 2.26 | SFTSILTCHQRRTQRKETVA |
| PSD95 | 1 | 2.13 | YGRKKRRQRRRKLSSIESDV |
| PSD95 | 1 | 2.07 | LNSSSNRRVYKKMPSIESAV |
| PSD95 | 1 | 2.05 | DFRPSFKHILFRRARRGFRQ |
| PSD95 | 1 | 2.02 | LNSSSNRRVYKKMPSIESAV |
| PSD95 | 1 | 1.83 | AAGGRSARGGRLQGRRETAL |
| PSD95 | 1 | 1.67 | LNSSSNRRVYKKMPSIESAV |
| PSD95 | 1 | 1.59 | QDFRRAFRRILARPWTQTAW |
| PSD95 | 1 | 1.49 | AAGGRSARGGRLQGRRETAL |
| PSD95 | 1 | 1.40 | YGRKKRRQRRRKLSSIESDV |
| PSD95 | 1 | 1.28 | KAGYRAPRSYPKSNSSKEYV |
| PSD95 | 1 | 1.26 | DFRPSFKHILFRRARRGFRQ |
| PSD95 | 1 | 1.25 | DGGARTEDEVQSYPSKHDYV |
| PSD95 | 1 | 1.23 | SSKSKSSEESQTFFGLYKL |
| PSD95 | 1 | 1.18 | PYSELNYETSHYPASPDSWV |
| PSD95 | 1 | 1.17 | LNSSSNRRVYKKMPSIESAV |
| PSD95 | 1 | 1.12 | TTNNNPNSAVNIKKIFTDV |
| PSD95 | 1 | 1.08 | TQGFPGPATWRRISSLESEV |
| PSD95 | 1 | 1.08 | KAGYRAPRSYPKSNSSKEYV |
| PSD95 | 1 | 1.03 | QISPGGLEPPSEKHFRETEV |
| PSD95 | 1 | 1.03 | AVGGRPARGGRLQGRRQTQV |
| PSD95 | 1 | 1.03 | PGQPPKVKSEFNSYSLTGYV |
| PSD95 | 1 | 1.01 | SSPDSSYQGKGFVMSRAMYV |
| PSD95 | 1 | 0.98 | RNIEEVYVGGKQVVPFSSSV |
| PSD95 | 1 | 0.96 | AAGGRSARGGRLQGRRETAL |
| PSD95 | 1 | 0.95 | SYPTPRPYPKPAPSSGKDYV |
| PSD95 | 1 | 0.94 | |
| PSD95 | 1 | 0.87 | QDFRRAFRRILARPWTQTAW |
| PSD95 | 1 | 0.86 | GGDLGTRRGSAHFSSLESEV |
| PSD95 | 1 | 0.85 | VDPNSPAAKKKYVSYNNLVI |
| PSD95 | 1 | 0.85 | PGQPPKVKSEFNSYSLTGYV |
| PSD95 | 1 | 0.82 | LGYSIPSRSGASGLDKRDYV |
| PSD95 | 1 | 0.82 | LNSSSNRRVYKKMPSIESAV |
| PSD95 | 1 | 0.80 | RNIEEVYVGGKQVVPFSSSV |
| PSD95 | 1 | 0.77 | GGDLGTRRGSAHFSSLESEV |
| PSD95 | 2 | 4.18 | AAGGRSARGGRLQGRRETAL |
| PSD95 | 2 | 4.06 | YGRKKRRQRRRKLSSIESDV |
| PSD95 | 2 | 4.00 | VHKVRNKFKAKCSLCRLYII |
| PSD95 | 2 | 4.00 | VHKVRNKFKAKCSLCRLYII |
| PSD95 | 2 | 3.56 | TQGFPGPATWRRISSLESEV |
| PSD95 | 2 | 3.53 | YGRKKRRQRRRKLSSIESDV |
| PSD95 | 2 | 3.37 | LNSSSNRRVYKKMPSIESAV |
| PSD95 | 2 | 3.11 | GGDLGTRRGSAHFSSLESEV |
| PSD95 | 2 | 3.10 | YGRKKRRQRRRKLSSIESDV |
| PSD95 | 2 | 2.89 | TQGFPGPATWRRISSLESEV |
| PSD95 | 2 | 2.82 | GRWTGRAMSAWKPTRRETEV |
| PSD95 | 2 | 2.79 | YGRKKRRQRRREKHFRETEV |
| PSD95 | 2 | 2.67 | LNSSSNRRVYKKMPSIESAV |
| PSD95 | 2 | 2.62 | VHKVRNKFKAKCSLCRLYII |
| PSD95 | 2 | 2.39 | QISPGGLEPPSEKHFRETEV |
| PSD95 | 2 | 2.32 | FNGSSNGHVYEKLSSIESDV |
| PSD95 | 2 | 2.29 | LNSSSNRRVYKKMPSIESAV |
| PSD95 | 2 | 2.28 | FNGSSNGHVYEKLSSIESDV |
| PSD95 | 2 | 2.22 | GGDLGTRRGSAHFSSLESEV |
| PSD95 | 2 | 2.17 | AAGGRSARGGRLQGRRETAL |
| PSD95 | 2 | 2.07 | TQGFPGPATWRRISSLESEV |
| PSD95 | 2 | 2.04 | FNGSSNGHVYEKLSSIESDV |
| PSD95 | 2 | 1.86 | FNGSSNGHVYEKLSSIESDV |
| PSD95 | 2 | 1.85 | AVGGRPARGGRLQGRRQTQV |
| PSD95 | 2 | 1.59 | FNGSSNGHVYEKLSSIESDV |
| PSD95 | 2 | 1.35 | KDITSDSENSNFRNEIQSLV |
| PSD95 | 2 | 1.31 | GGDLGTRRGSAHFSSLESEV |
| PSD95 | 2 | 1.23 | RSGATIPLVGQDIIDLQTEV |
| PSD95 | 2 | 1.12 | FNGSSNGHVYEKLSSIESDV |
| PSD95 | 2 | 1.06 | TTNNNPNSAVNIKKIFTDV |
| PSD95 | 2 | 0.80 | YGRKKRRQRRREKHFREAEA |
| PSD95 | 3 | 4.00 | SFTSILTCHQRRTQRKETVA |
| PSD95 | 3 | 4.00 | SFTSILTCHQRRTQRKETVA |
| PSD95 | 3 | 4.00 | VHKVRNKFKAKCSLCRLYII |
| PSD95 | 3 | 4.00 | VHKVRNKFKAKCSLCRLYII |
| PSD95 | 3 | 4.00 | GRWTGRAMSAWKPTRRETEV |
| PSD95 | 3 | 4.00 | AAGGRSARGGRLQGRRETAL |
| PSD95 | 3 | 4.00 | AVGGRPARGGRLQGRRQTQV |
| PSD95 | 3 | 4.00 | SFTSILTCHQRRTQRKETVA |
| PSD95 | 3 | 4.00 | GRWTGRAMSAWKPTRRETEV |
| PSD95 | 3 | 4.00 | GRWTGRAMSAWKPTRRETEV |
| PSD95 | 3 | 4.00 | AAGGRSARGGRLQGRRETAL |
| PSD95 | 3 | 4.00 | AVGGRPARGGRLQGRRQTQV |
| PSD95 | 3 | 3.74 | QDFRRAFRRILARPWTQTAW |
| PSD95 | 3 | 2.98 | QDFRRAFRRILARPWTQTAW |
| PSD95 | 3 | 2.93 | YGRKKRRQRRREKHFRETEV |
| PSD95 | 3 | 1.72 | DFRPSFKHILFRRARRGFRQ |
| PSD95 | 3 | 1.46 | TQGFPGPATWRRISSLESEV |
| PSD95 | 3 | 1.19 | DFRPSFKHILFRRARRGFRQ |
| PSD95 | 3 | 1.17 | AGAVRTPLSQVNKVWDQSSV |
| PSD95 | 3 | 1.17 | TQGFPGPATWRRISSLESEV |
| PSD95 | 3 | 1.07 | SSKSKSSEESQTFFGLYKL |
| PSD95 | 3 | 1.05 | DGGARTEDEVQSYPSKHDYV |
| PSD95 | 3 | 1.00 | TTNNNPNSAVNIKKIFTDV |
| PSD95 | 3 | 0.92 | KAGYRAPRSYPKSNSSKEYV |
| PSD95 | 3 | 0.91 | SSPDSSYQGKGFVMSRAMYV |
| PSD95 | 3 | 0.89 | AGAVRTPLSQVNKVWDQSSV |
| PSD95 | 3 | 0.86 | KAGYRAPRSYPKSNSSKEYV |
| PSD95 | 3 | 0.85 | SYPTPRPYPKPAPSSGKDYV |
| PSD95 | 3 | 0.82 | VDPNSPAAKKKYVSYNNLVI |
| PSD95 | 3 | 0.82 | HHLVAQRDIRQFQLQHWLAI |
| PSD95 | 3 | 0.79 | PGQPPKVKSEFNSYSLTGYV |
| PSD95 | 3 | 0.79 | PGQPPKVKSEFNSYSLTGYV |
| PSD95 | 3 | 0.78 | TGSALQAWRHTSRQATESTV |
| PSD95 | 3 | 0.70 | HHLVAQRDIRQFQLQHWLAI |
| PSD95 | 3 | 0.70 | ESSGTQSPKRHSGSYLVTSV |

The present invention is not to be limited in scope by the exemplified embodiments which are intended as illustrations of single aspects of the invention and any sequences which are functionally equivalent are within the scope of the invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications cited herein are hereby incorporated by reference in their entirety for all purposes to the same extent as if each individual publication, patent or patent application were specifically and individually indicated to be so incorporated by reference.

TABLE 2
NMDA Receptors with PL Sequences

| | | | C-terminal | | internal PL |
|---|---|---|---|---|---|
| Name | GI# | C-terminal 20mer sequence | 4mer sequence | PL? | ID |
| NMDAR1-1 | 292282 | HPTDITGPLNLSDPSVSTVV | STVV | X | AA216 |
| NMDAR1-4 | 472845 | HPTDITGPLNLSDPSVSTVV | STVV | X | AA216 |
| NMDAR1-3b | 2343286 | HPTDITGPLNLSDPSVSTVV | STVV | X | AA216 |
| NMDAR1-4b | 2343288 | HPTDITGPLNLSDPSVSTVV | STVV | X | AA216 |
| NMDAR1-2 | 11038634 | RRAIEREEGQLQLCSRHRES | HRES | | |
| NMDAR1-3 | 11038636 | RRAIEREEGQLQLCSRHRES | HRES | | |
| NMDAR2C | 6006004 | TQGFPGPCTWRRISSLESEV | ESEV | X | AA180 |
| NMDAR3 | 560546 | FNGSSNGHVYEKLSSIESDV | ESDV | X | AA34.1 |
| NMDAR3A | 17530176 | AVSRKTELEEYQRTSRTCES | TCES | | |
| NMDAR2B | 4099612 | FNGSSNGHVYEKLSSIESDV | ESDV | X | |
| NMDAR2A | 558748 | LNSCSNRRVYKKMPSIESDV | ESDV | X | AA34.2 |
| NMDAR2D | 4504130 | GGDLGTRRGSAHFSSLESEV | ESEV | X | |

TABLE 3
PDZ Proteins that Interact with NMDA Receptor Proteins (a PL protein)

| internal | | | | PDZ | Binding | Assay |
|---|---|---|---|---|---|---|
| PL ID | PL Name | PL 20Mer Sequence | PDZ Name | Domain | Strength | Used |
| AA34.2 | NMDAR2A | LNSCSNRRVYKKMPSIESDV | MPP1 | 1 | | A |
| AA34.2 | NMDAR2A | LNSCSNRRVYKKMPSIESDV | DLG1 | 1, 2 | | A/G |
| AA34.2 | NMDAR2A | LNSCSNRRVYKKMPSIESDV | PSD95 | 1, 2, 3 | | A/G |
| AA34.2 | NMDAR2A | LNSCSNRRVYKKMPSIESDV | NeDLG | 1, 2 | | A/G |
| AA34.2 | NMDAR2A | LNSCSNRRVYKKMPSIESDV | Syntrophin 1 | 1 | | G |
| | | | alpha | | | |
| AA34.2 | NMDAR2A | LNSCSNRRVYKKMPSIESDV | T1P43 | 1 | | A |
| AA34.2 | NMDAR2A | LNSCSNRRVYKKMPSIESDV | LIMK1 | 1 | | A |
| AA34.2 | NMDAR2A | LNSCSNRRVYKKMPSIESDV | MPP2 | 1 | | G |
| AA34.2 | NMDAR2A | LNSCSNRRVYKKMPSIESDV | PTN4 | 1 | | A/G |
| AA34.2 | NMDAR2A | LNSCSNRRVYKKMPSIESDV | 41.8 | 1 | | A/G |
| AA34.2 | NMDAR2A | LNSCSNRRVYKKMPSIESDV | RGS12 | 1 | | A/G |
| AA34.2 | NMDAR2A | LNSCSNRRVYKKMPSIESDV | DVL1 | 1 | | A |
| AA34.2 | NMDAR2A | LNSCSNRRVYKKMPSIESDV | MINT1 | 1, 2 | | A/G |
| AA34.2 | NMDAR2A | LNSCSNRRVYKKMPSIESDV | TIP2 | 1 | | A |
| AA34.2 | NMDAR2A | LNSCSNRRVYKKMPSIESDV | KIAA561 | 1 | | G |
| AA180 | NMDAR2C | TQGFPGPATWRRISSLESEV | FLJ00011 | 1 | 2 | G |
| AA180 | NMDAR2C | TQGFPGPATWRRISSLESEV | Magi2 | 3 | 2 | G |
| AA180 | NMDAR2C | TQGFPGPATWRRISSLESEV | DLG1 | 1, 2 | 5 | G |
| AA180 | NMDAR2C | TQGFPGPATWRRISSLESEV | MAGI 1 | 2 | 4 | G |
| AA180 | NMDAR2C | TQGFPGPATWRRISSLESEV | MAGI 1 | 5 | 4 | G |
| AA180 | NMDAR2C | TQGFPGPATWRRISSLESEV | KIAA0807 | 1 | 4 | G |
| AA180 | NMDAR2C | TQGFPGPATWRRISSLESEV | DLG1 | 1 | 5 | G |
| AA180 | NMDAR2C | TQGFPGPATWRRISSLESEV | DLG1 | 2 | 5 | G |
| AA180 | NMDAR2C | TQGFPGPATWRRISSLESEV | DLG2 | 1 | 4 | G |
| AA180 | NMDAR2C | TQGFPGPATWRRISSLESEV | Erbin | 1 | 4 | G |
| AA180 | NMDAR2C | TQGFPGPATWRRISSLESEV | FLJ 11215 | 1 | 1 | G |
| AA180 | NMDAR2C | TQGFPGPATWRRISSLESEV | INADL | 3 | 2 | G |
| AA180 | NMDAR2C | TQGFPGPATWRRISSLESEV | INADL | 8 | 1 | G |
| AA180 | NMDAR2C | TQGFPGPATWRRISSLESEV | KIAA0147 | 2 | 1 | G |
| AA180 | NMDAR2C | TQGFPGPATWRRISSLESEV | KIAA0147 | 3 | 1 | G |
| AA180 | NMDAR2C | TQGFPGPATWRRISSLESEV | KIAA0380 | 1 | 1 | G |
| AA180 | NMDAR2C | TQGFPGPATWRRISSLESEV | KIAA0382 | 1 | 1 | G |
| AA180 | NMDAR2C | TQGFPGPATWRRISSLESEV | KIAA0973 | 1 | 1 | G |
| AA180 | NMDAR2C | TQGFPGPATWRRISSLESEV | KIAA1634 | 2 | 3 | G |
| AA180 | NMDAR2C | TQGFPGPATWRRISSLESEV | KIAA1634 | 5 | 1 | G |
| AA180 | NMDAR2C | TQGFPGPATWRRISSLESEV | M1NT1 | 1, 2 | 1 | G |
| AA180 | NMDAR2C | TQGFPGPATWRRISSLESEV | KIAA1634 | 1 | 5 | G |
| AA180 | NMDAR2C | TQGFPGPATWRRISSLESEV | LIMK1 | 1 | 1 | G |
| AA180 | NMDAR2C | TQGFPGPATWRRISSLESEV | NeDLG | 1, 2 | 5 | G |
| AA180 | NMDAR2C | TQGFPGPATWRRISSLESEV | PSD95 | 1, 2, 3 | 5 | G |
| AA180 | NMDAR2C | TQGFPGPATWRRISSLESEV | NeDLG | 3 | 1 | G |
| AA180 | NMDAR2C | TQGFPGPATWRRISSLESEV | NOS1 | 1 | 1 | G |
| AA180 | NMDAR2C | TQGFPGPATWRRISSLESEV | PSD95 | 3 | 2 | G |
| AA180 | NMDAR2C | TQGFPGPATWRRISSLESEV | Syntrophin 1 | 1 | 4 | G |
| | | | alpha | | | |
| AA180 | NMDAR2C | TQGFPGPATWRRISSLESEV | TIP1 | 1 | 5 | G |
| AA180 | NMDAR2C | TQGFPGPATWRRISSLESEV | Syntrophin | 1 | 2 | G |
| | | | gamma-2 | | | |
| AA180 | NMDAR2C | TQGFPGPATWRRISSLESEV | TAX IP 2 | 1 | 4 | G |
| AA180 | NMDAR2C | TQGFPGPATWRRISSLESEV | LIM RIL | 1 | 5 | G |
| AA180 | NMDAR2C | TQGFPGPATWRRISSLESEV | Syntrophin | 1 | 4 | G |
| | | | gamma-1 | | | |
| AA180 | NMDAR2C | TQGFPGPATWRRISSLESEV | PTPL1 | 2 | 5 | G |
| AA180 | NMDAR2C | TQGFPGPATWRRISSLESEV | AIPC | 1 | 3 | G |
| AA180 | NMDAR2C | TQGFPGPATWRRISSLESEV | NeDLG | 2 | 5 | G |
| AA180 | NMDAR2C | TQGFPGPATWRRISSLESEV | KIAA1526 | 1 | 3 | G |
| AA180 | NMDAR2C | TQGFPGPATWRRISSLESEV | MUPP1 | 13 | 5 | G |
| AA180 | NMDAR2C | TQGFPGPATWRRISSLESEV | LIM- | 1 | 4 | G |
| | | | Mystique | | | |
| AA180 | NMDAR2C | TQGFPGPATWRRISSLESEV | Outer | 1 | 5 | G |
| | | | Membrane | | | |
| AA180 | NMDAR2C | TQGFPGPATWRRISSLESEV | KIAA0807 | 1 | 5 | G |
| AA180 | NMDAR2C | TQGFPGPATWRRISSLESEV | Magi2 | 1 | 5 | G |
| AA180 | NMDAR2C | TQGFPGPATWRRISSLESEV | KIAA0147 | 1 | 5 | G |
| AA180 | NMDAR2C | TQGFPGPATWRRISSLESEV | PSD95 | 1 | 5 | G |
| AA180 | NMDAR2C | TQGFPGPATWRRISSLESEV | Magi2 | 5 | 5 | G |
| AA180 | NMDAR2C | TQGFPGPATWRRISSLESEV | DLG2 | 2 | 5 | G |
| AA180 | NMDAR2C | TQGFPGPATWRRISSLESEV | MAGI 1 | 4 | 3 | G |
| AA180 | NMDAR2C | TQGFPGPATWRRISSLESEV | MAGI 1 | 3 | 1 | G |
| AA180 | NMDAR2C | TQGFPGPATWRRISSLESEV | Mint 1 | 2 | 1 | G |
| AA180 | NMDAR2C | TQGFPGPATWRRISSLESEV | MUPP1 | 10 | 1 | G |
| AA180 | NMDAR2C | TQGFPGPATWRRISSLESEV | KIAA1634 | 4 | 1 | G |
| AA180 | NMDAR2C | TQGFPGPATWRRISSLESEV | MAGI 1 | 6 | 2 | G |
| AA180 | NMDAR2C | TQGFPGPATWRRISSLESEV | MUPP1 | 5 | 1 | G |
| AA180 | NMDAR2C | TQGFPGPATWRRISSLESEV | NeDLG | 1 | 2 | G |
| AA180 | NMDAR2C | TQGFPGPATWRRISSLESEV | APXL1 | 1 | 1 | G |
| AA180 | NMDAR2C | TQGFPGPATWRRISSLESEV | KIAA1095 | 1 | 1 | G |
| AA180 | NMDAR2C | TQGFPGPATWRRISSLESEV | INADL | 5 | 1 | G |
| AA180 | NMDAR2C | TQGFPGPATWRRISSLESEV | PTN-4 | 1 | 2 | G |
| AA216 | NMDAR2C | HPTDITGLPNLSDPSVSTVV | NeDLG | 1, 2 | 2 | G |
| AA216 | NMDAR1 | HPTDITGLPNLSDPSVSTVV | PTPL1 | 2 | 1 | G |
| AA216 | NMDAR1 | HPTDITGLPNLSDPSVSTVV | DLG1 | 1, 2 | 1 | G |
| AA216 | NMDAR1 | HPTDITGLPNLSDPSVSTVV | KIAA1634 | 2 | 1 | G |
| AA216 | NMDAR1 | HPTDITGLPNLSDPSVSTVV | PSD95 | 1, 2, 3 | 1 | G |

TABLE 4
Sequences of PDZ Domains Cloned to Produce GST-PDZ Fusions

| Gene | GI or | Domain | |
|---|---|---|---|
| Name | Acc# | # | Sequence fused to GST Construct |
| 26s subunit | 9184389 | 1 | RDMAEAHKEAMSRKLGQSESQGPPRAFAKVNSIS |
| p27 | | | PGSPSIAGLQVDDEIVEFGSVNTQNFQSLHNIGS |
| | | | VVQHSEGALAPTILLSVSM |
| AF6 | 430993 | 1 | LRKEPEIITVTLKKQNGMGLSIVAAKGAGQDKLG |
| | | | IYVKSVVKGGAADVDGRLAAGDQLLSVDGRSLVG |
| | | | LSQERAAELMTRTSSVVTLEVAKQG |
| AIPC | 12751451 | 1 | LIRPSVISIIGLYKEKGKGLGFSIAGGRDCIRGQ |
| | | | LMGFVKTIFPNGSAAEDGRLKEGDEILDVNGIPI |
| | | | KGLTFQEAIHTFKQIRSGLFVLTVRTKLVSPSLT |
| | | | NSS |
| AIPC | 12751451 | 2 | GISSLGRKTPGPKDRIVMEVTLNKEPRVGLGIGA |
| | | | CCLALENSPPGIYIHSLAPGSVAKMESNLSRGDQ |
| | | | ILEVNSVNVRHAALSKVHAILSKCPPGPVRLVIG |
| | | | RHPNPKVSEQEMDEVIARSTYQESKEANSS |
| AIPC | 12751451 | 3 | QSENEEDVCFIVLNRKEGSGLGFSVAGGTDVEPK |
| | | | SITVHRVFSQGAASQEGTMNRGDFLLSVNGASLA |
| | | | GLAHGNVLKVLHQAQLHKDALVVIKKGMDQPR |
| | | | PSNSS |
| AIPC | 12751451 | 4 | LGRSVAVHDALCVEVLKTSAGLGLSLDGGKSSVT |
| | | | GDGPLVIKRVYKGGAAEQAGIIEAGDEILAINGK |
| | | | PLVGLMHFDAWNIMKSVPEGPVQLLIRKHRNSS |
| alpha actinin-2 | 2773059 | 1 | QTVILPGPAAWGFRLSGGIDFNQPLVITRITPGS |
| associated LEVI | | | KAAAANLCPGDVILALDGFGTESMTHADGQDRIK |
| protein | | | AAEFIV |
| APXL-1 | 13651263 | 1 | ILVEVQLSGGAPWGFTLKGGREHGEPLVITKIEE |
| | | | GSKAAAVDKLLAGDEIVGINDIGLSGFRQEAICL |
| | | | VKGSHKTLKLVVKRNSS |
| Atrophin-1 | 2947231 | 1 | REKPLFTRDASQLKGTFLSTTLKKSNMGFGFTII |
| Interacting | | | GGDEPDEFLQVKSVIPDGPAAQDGKMETGDVIVY |
| Protein | | | INEVCVLGHTHADVVKLFQSVPIGQSVNLVLCRG |
| | | | YP |
| Atrophin-1 | 2947231 | 2 | LSGATQAELMTLTIVKGAQGFGFTIADSPTGQRV |
| Interacting | | | KQILDIQGCPGLCEGDLIVEINQQNVQNLSHTEV |
| Protein | | | VDILKDCPIGSETSLIIHRGGFF |
| Atrophin-1 | 2947231 | 3 | HYKELDVHLRRMESGFGFRILGGDEPGQPILIGA |
| Interacting | | | VIAMGSADRDGRLHPGDELVYVDGIPVAGKTHRY |
| Protein | | | VIDLMHHAARNGQVNLTVRRKVLCG |
| Atrophin-1 | 2947231 | 4 | EGRGISSHSLQTSDAVIHRKENEGFGFVIISSLN |
| Interacting | | | RPESGSTITVPHKIGRIIDGSPADRCAKLKVGDR |
| Protein | | | ILAVNGQSIINMPHADIVKLIKDAGLSVTLRIIP |
| | | | QEEL |
| Atrophin-1 | 2947231 | 5 | LSDYRQPQDFDYFTVDMEKGAKGFGFSIRGGREY |
| Interacting | | | KMDLYVLRLAEDGPAIRNGRMRVGDQIIEINGES |
| Protein | | | TRDMTHARAIELIKSGGRRVRLLLKRGTGQ |
| Atrophin-1 | 2947231 | 6 | HESVIGRNPEGQLGFELKGGAENGQFPYLGEVKP |
| Interacting | | | GKVAYESGSKLVSEELLLEVNETPVAGLTIRDVL |
| Protein | | | AVIKHCKDPLRLKCVKQGGIHR |
| CARD11 | 12382772 | 1 | NLMFRICFSLERPFRP SVTSVGHVRGPGPSVQH |
| | | | TTLNGDSLTSQLTLLGGNARGSFVHSVICPGSLA |
| | | | EICAGLREGHQLLLLEGCIRGERQSVPLDTCTKE |
| | | | EAHWTIQRCSGPVTLHYKVNHEGYRKLV |
| CARD14 | 13129123 | 1 | ILSQVTMLAFQGDALLEQISVIGGNLTGIFIHRV |
| | | | TPGSAADQMALRPGTQIVMVDYEASEPLFKAVLE |
| | | | DTTLEEAVGLLRRVDGFCCLSVKVNTDGYKRL |
| CASK | 3087815 | 1 | TRVRLVQFQICNTDEPMGITLKMNELNHCIVARI |
| | | | MHGGMIHRQGTLHVGDEIREINGISVANQTVEQL |
| | | | QKMLREMRGSITFKIVPSYRTQS |
| Connector | 3930780 | 1 | LEQKAVLEQVQLDSPLGLEIHTTSNCQHFVSQVD |
| Enhancer | | | TQVPTDSRLQIQPGDEVVQINEQVVVGWPRKNMV |
| | | | RELLREPAGLSLVLKKIPIP |
| Cytohesin | 3192908 | 1 | QRKLVTVEKQDNETFGFEIQSYRPQNQNACSSEM |
| Binding | | | FTLICKIQEDSPAHCAGLQAGDVLANINGVSTEG |
| Protein | | | FTYKQVVDLIRSSGNLLTIETLNG |
| Densin 180 | 16755892 | 1 | RCLIQTKGQRSMDGYPEQFCVRIEKNPGLGFSIS |
| | | | GGISGQGNPFKPSDKGIFVTRVQPDGPASNLLQP |
| | | | GDKILQANGHSFVHMEHEKAVLLLKSFQNTVDLV |
| | | | IQRELTV |
| DLG1 | 475816 | 1 | IQVNGTDADYEYEEITLERGNSGLGFSIAGGTDN |
| | | | PHIGDDSSIFITKIITGGAAAQDGRLRVNDCILQ |
| | | | VNEVDVRDVTHSKAVEALKEAGSIVRLYVKRRN |
| DLG1 | 475816 | 2 | IQLIKGPKGLGFSIAGGVGNQHIPGDNSIYVTKI |
| | | | IEGGAAHKDGKLQIGDKLLAVNNVCLEEVTHEEA |
| | | | VTALKNTSDFVYLKVAKPTSMYMNDGN |
| DLG1 | 475816 | 3 | ILHRGSTGLGFNIVGGEDGEGIFISFILAGGPAD |
| | | | GLSELRKGDRIISVNSVDLRAASHEQAAAALKNA |
| | | | GQAVTIVAQYRPEEYSR |
| DLG2 | 12736552 | 1 | ISYVNGTEIEYEFEEITLERGNSGLGFSIAGGTD |
| | | | NPHIGDDPGIFITKIIPGGAAAEDGRLRVNDCIL |
| | | | RVNEVDVSEVSHSICAVEALICEAGSIVRLYVRR |
| | | | R |
| DLG2 | 12736552 | 2 | ISVVEIKLFKGPKGLGFSIAGGVGNQHIPGDNSI |
| | | | YVTKIIDGGAAQKDGRLQVGDRLLMVNNYSLEEV |
| | | | THEEAVAILKNTSEVVYLKVGNPTTI |
| DLG2 | 12736552 | 3 | IWAVSLEGEPRKVVLHKGSTGLGFNIVGGEDGEG |
| | | | IFVSFILAGGPADLSGELQRGDQILSVNGIDLRG |
| | | | ASHEQAAAALKGAGQTVTIIAQYQPED |
| DLG5 | 3650451 | 1 | GIPYVEEPRHVKVQKGSEPLGISIVSGEKGGIYV |
| | | | SKVTVGSIAHQAGLEYGDQLLEFNGINLRSATEQ |
| | | | QARLIIGQQCDTITILAQYNPHVHQLRNSSZLTD |
| DLG5 | 3650451 | 2 | GILAGDANICKTLEPRVVFIICKSQLELGVHLCG |
| | | | GNLHGVFVAEVEDDSPAKGPDGLVPGDLILEYGS |
| | | | LDVRNKTVEEVYVEMLKPRDGVRLKVQYRPEEFI |
| | | | VTD |
| DLG6, splice | 14647140 | 1 | PTSPEIQELRQMLQAPHFKALLSAHDTIAQKDFE |
| variarft 1 | | | PLLPPLPDNIPESEEAMRIVCLVKNQQPLGATIK |
| | | | RHEMTGDILVARIIHGGLAERSGLLYAGDKLVEV |
| | | | NGVSVEGLDPEQVIHILAMSRGTIMFKVVPVSDP |
| | | | PVNSS |
| DLG6, splice | AB053303 | 1 | PTSPEIQELRQMLQAPHFKGATIKRHEMTGDILV |
| variant 2 | | | ARIIHGGLAERSGLLYAGDKLVEVNGVSVEGLDP |
| | | | EQVIHILAMSRGTIMFKVVPVSDPPVNSS |
| DVL1 | 2291005 | 1 | LNIVTVTLNMERHHFLGISIVGQSNDRGDGGIYI |
| | | | GSTMKGGAVAADGRIEPGDMLLQVNDVNFENMSN |
| | | | DDAVRVLREIVSQTGPISLTVAKCW |
| DVL2 | 2291007 | 1 | LNIITVTLNMEKYNFLGISIVGQSNERGDGGIYI |
| | | | GSIMKGGAVAADGRIEPGDMLLQVNDMNFENMSN |
| | | | DDAVRVLRDIVHKPGPIVLTVAKCWDPSPQNS |
| DVL3 | 6806886 | 1 | IITVTLNMEKYNFLGISIVGQSNERGDGGIYIGS |
| | | | IMKGGAVAADGRIEPGDMLLQVNEINFENMSNDD |
| | | | AVRVLREIVHKPGPITLTVAKCWDPSP |
| ELFIN 1 | 2957144 | 1 | TTQQIDLQGPGPWGFRLVGRKDFEQPLAISRVTP |
| | | | GSKAALANLCIGDVITAIDGENTSNMTHLEAQNR |
| | | | IKGCTQNLTLTVARSEHKVWSPLV |
| ENIGMA | 561636 | 1 | IFMDSFKVVLEGPAPWGFRLQGGKDFNVPLSISR |
| | | | LTPGGKAAQAGVAVGDWVLSIDGENAGSLTHIEA |
| | | | QNKIRACGERLSLGLSRAQPV |
| ERBIN | 8923908 | 1 | QGHELAKQEIRVRVEKDPELGFSISGGVGGRGNP |
| | | | FRPDDDGIFVTRVQPEGPASKLLQPGDKIIQANG |
| | | | YSFINIEHGQAVSLLKTFQNTVELIIVREVSS |
| EZRIN | 3220018 | 1 | ILCCLEKGPNGYGFHLHGEKGKLGQYIRLVEPGS |
| Binding | | | PAEKAGLLAGDRLVEVNGENVEKETHQQVVSRIR |
| Protein 50 | | | AALNAVRLLVVDPEFIVTD |
| EZRIN | 3220018 | 2 | IRLCTMKKGPSGYGFNLHSDKSKPGQFIRSVDPD |
| Binding | | | SPAEASGLRAQDRIVEVNGVCMEGKQHGDVVSAI |
| Protein 50 | | | RAGGDETKLLVVDRETDEFFMNSS |
| FLJ00011 | 10440352 | 1 | KNPSGELKTVTLSKMKQSLGISISGGIESKVQPM |
| | | | VKIEKIFPGGAAFLSGALQAGFELVAVDGENLEQ |
| | | | VTHQRAVDTIRRAYRNKAREPMELVVRVPGPSPR |
| | | | PSPSD |
| FLJ11215 | 11436365 | 1 | EGHSHPRVVELPKTEEGLGFNIMGGKEQNSPIYI |
| | | | RSIIPGGIADRHGGLKRGDQLLSVNGVSVEGEHH |
| | | | EKAVELLKAAQGKVKLVVRYTPKVLEEME |
| FLJ12428 | BC012040 | 1 | PGAPYARKTFTIVGDAVGWGFVVRGSKPCHIQAV |
| | | | DPSGPAAAAGMKVCQFVVSVNGLNVLHVDYRTVS |
| | | | NLILTGPRTIVMEVMEELEC |
| FLJ12615 | 10434209 | 1 | GQYGGETVKIVRIEKARDIPLGATVRNEMDSVII |
| | | | SRIVKGGAAEKSGLLHEGDEVLEINGIEIRGKDV |
| | | | NEVFDLLSDMHGTLTFVLIPSQQIKPPPA |
| FLJ20075 | 7019938 | 1 | ILAHVKGIEKEVNVYKSEDSLGLTITDNGVGYAF |
| | | | IKRIKDGGVIDSVKTICVGDHIESINGENIVGWR |
| | | | HYDVAKKLKELKKEELFTMKLIEPKKAFEI |
| FLJ21687 | 10437836 | 1 | KPSQASGHFSVELVRGYAGFGLTLGGGRDVAGDT |
| | | | PLAVRGLLKDGPAQRCGRLEVGDLVLHINGESTQ |
| | | | GLTHAQAVERIRAGGPQLHLVIRRPLETHPGKPR |
| | | | GV |
| FLJ31349 | AK055911 | 1 | PVMSQCACLEEVHLPNIKPGEGLGMYIKSTYDGL |
| | | | HVITGTTENSPADRSQKIHAGDEVIQVNQQTVVG |
| | | | WQLKNLVKKLRENPTGVVLLLKKRPTGSFNFTPE |
| | | | FIVTD |
| FLJ32798 | AK057360 | 1 | LDDEEDSVKIIRLVKNREPLGATIKKDEQTGAII |
| | | | VARIMRGGAADRSGLIHVGDELREVNGIPVEDKR |
| | | | PEEIIQILAQSQGAITFKIIPGSKEETPSNSS |
| GRIP 1 | 4539083 | 1 | VVELMKKEGTTLGLTVSGGIDKDGKPRVSNLRQG |
| | | | GIAARSDQLDVGDYIKAVNGINLAKFRHDEIISL |
| | | | LKNVGERVVLEVEYE |
| GRIP 1 | 4539083 | 2 | RSSVIFRTVEVTLHKEGNTFGFVIRGGAHDDRNK |
| | | | SRPVVITCVRPGGPADREGTIKPGDRLLSVDGIR |
| | | | LLGTTHAEAMSILKQCGQEAALLIEYDVSVMDSV |
| | | | ATASGNSS |
| GRIP 1 | 4539083 | 3 | HVATASGPLLVEVAKTPGASLGVALTTSMCCNKQ |
| | | | VIVIDKIKSASIADRCGALHVGDHILSIDGTSME |
| | | | YCTLAEATQFLANTTDQVKLEILPHHQTRLALKG |
| | | | PNSS |
| GRIP 1 | 4539083 | 4 | TETTEVVLTADPVTGFGIQLQGSVFATETLSSPP |
| | | | LISYIEADSPAERCGVLQIGDRVMAINGIPTEDS |
| | | | TFEEASQLLRDSSITSKVTLEIEFDVAES |
| GRIP 1 | 4539083 | 5 | AESVIPSSGTFHVKLPKKHNVELGITISSPSSRK |
| | | | PGDPLVISDIKKGSVAHRTGTLELGDKLLAIDNI |
| | | | RLDNCSMEDAVQILQQCEDLVKLKIRKDEDNSD |
| GRIP 1 | 4539083 | 6 | IYTVELKRYGGPLGITISGTEEPFDPIIISSLTK |
| | | | GGLAERTGAIHIGDRILAINSSSLKGKPLSEAIH |
| | | | LLQMAGETVTLKIKKQTDAQSA |
| GRIP 1 | 4539083 | 7 | IMSPTPVELHKVTLYKDSDMEDFGFSVADGLLEK |
| | | | GVYVKNIRPAGPGDLGGLKPYDRLLQVNHVRTRD |
| | | | FDCCLVVPLIAESGNKLDLVISRNPLA |
| GTPase | 2389008 | 1 | SRGCETRELALPRDGQGRLGFEVDAEGFVTHVER |
| Activating | | | FTFAETAGLRPGARLLRVCGQTLPSLRPEAAAQL |
| Enzyme | | | LRSAPKVCVTVLPPDESGRP |
| Guanine | 6650765 | 1 | AKAKWRQVVLQKASRESPLQFSLNGGSEKGFGIF |
| Exchange | | | VEGVEPGSKAADSGLKRGDQIMEVNGQNFENITF |
| Factor | | | MKAVEILRNNTHLALTVKTNIFVFKEL |
| HEMBA | 10436367 | 1 | LENVIAKSLLIKSNEGSYGFGLEDKNKVPIEKLV |
| 1000505 | | | EKGSNAEMAGMEVGKKIFAINGDLVFMRPFNEVD |
| | | | CFLKSCLNSRKPLRVLVSTKP |
| HEMBA | 104363672 | | PRETVKIPDSADGLGFQIRGFGPSVVHAVGRGTV |
| 1000505 | | | AAAAGLHPGQCIEKVNGINVSKETHASVIAHVTA |
| | | | CRKYRRPTKQDSIQ |
| HEMBA | 7022001 | 1 | EDFCYVFTVELERGPSGLGMGLIDGMHTHLGAPG |
| 1003117 | | | LYIQTLLPGSPAAADGRLSLGDRILEVNGSSLLG |
| | | | LGYLRAVDLIRHGGKKMRFLVAKSDVETAKKI |
| HTRA3 | AY040094 | 1 | LTEFQDKQIKDWICKRFIGIRMRTITPSLVDELK |
| | | | ASNPDFPEVSSGIYVQEVAPNSPSQRGGIQDGDI |
| | | | IVKVNGRPLVDSSELQEAVLTESPLLLEVRRGND |
| | | | DLLFSNSS |
| HTRA4 | AL576444 | 1 | HKKYLGLQMLSLTVPLSEELKMHYPDFPDVSSGV |
| | | | YVCKVVEGTAAQSSGLRDHDVIVNINGKPITTTT |
| | | | DVVKALDSDSLSMAVLRGKDNLLLTVNSS |
| INADL | 2370148 | 1 | IWQIEYIDIERPSTGGLGFSVVALRSQNLGKVDI |
| | | | FVKDVQPGSVADRDQRLKENDQILAINHTPLDQN |
| | | | ISHQQAIALLQQTTGSLRLIVAREPVHTKSSTSS |
| | | | SE |
| INADL | 2370148 | 2 | PGHVEEVELINDGSGLGFGIVGGKTSGVVVRTIV |
| | | | PGGLADRDGRLQTGDHILKIGGTNVQGMTSEQVA |
| | | | QVLRNCGNSS |
| INADL | 2370148 | 3 | PGSDSSLFETYNVELVRKDGQSLGIRIVGYVGTS |
| | | | HTGEASGIYVKSIIPGSAAYHNGHIQVNDKIVAV |
| | | | DGVNIQGFANHDVVEVLRNAGQVVHLTLVRRKTS |
| | | | SSTSRIHRD |
| INADL | 2370148 | 4 | NSDDAELQKYSKLLPIHTLRLGVEVDSFDGHHYI |
| | | | SSIVSGGPVDTLGLLQPEDELLEVNGMQLYGKSR |
| | | | REAVSFLKEVPPPFTLVCCRRLFDDEAS |
| INADL | 2370148 | 5 | LSSPEVKIVELVKDCKGLGFSILDYQDPLDPTRS |
| | | | VIVIRSLVADGVAERSGGLLPGDRLVSVNEYCLD |
| | | | NTSLAEAVEILKAVPPGLVHLGICKPLVEFIVTD |
| INADL | 2370148 | 6 | PNFSHWGPPRIVEIFREPNVSLGISIVVGQTVIK |
| | | | KRLNGEELKGIFIKQVLEDSPAGKTNALKTGDKI |
| | | | LEVSGVDLQNASHSEAVEAIKNAGNPVVFIVQSL |
| | | | SSTPRVIPNVHNKANSS |
| INADL | 2370148 | 7 | PGELHIIELEKDKNGLGLSLAGNKDRSRMSIFVV |
| | | | GINPEGPAAADGRMRIGDELLEINNQILYGRSHQ |
| | | | NASAIIKTAPSKVKLVFIRNEDAVNQMANSS |
| INADL | 2370148 | 8 | PATCPIVPGQEMIIEISKGRSGLGLSIVGGKDTP |
| | | | LNAIVIHEVYEEGAAARDGRLWAGDQILEVNGVD |
| | | | LRNSSHEEAITALRQTPQKVRLVVY |
| KIAA0147 | 1469875 | 1 | ILTLTILRQTGGLGISIAGGKGSTPYKGDDEGIF |
| | | | ISRVSEEGPAARAGVRVGDKLLEVNGVALQGAEH |
| | | | HEAVEALRGAGTAVQMRVWRERMVEPENAEFIVT |
| | | | D |
| KIAA0147 | 1469875 | 2 | PLRQRHVACLARSERGLGFSIAGGKGSTPYRAGD |
| | | | AGIFVSRIAEGGAAHRAGTLQVGDRVLSINGVDV |
| | | | TEARHDHAVSLLTAASPTIALLLEREAGG |
| KIAA0147 | 1469875 | 3 | ILEGPYPVEEIRLPRAGGPLGLSIVGGSDHSSHP |
| | | | FGVQEPGVFISKVLPRGLAARSGLRVGDRILAVN |
| | | | GQDVRDATHQEAVSALLRPCLELSLLVRRDPAEF |
| | | | IVTD |
| KIAA0147 | 1469875 | 4 | RELCIQKAPGERLGISIRGGARGHAGNPRDPTDE |
| | | | GIFISKVSPTGAAGRDGRLRVGLRLLEVNQQSLL |
| | | | GLTHGEAVQLLRSVGDTLTVLVCDGFEASTDAAL |
| | | | EVS |
| KIAA0303 | 2224546 | 1 | PHQPIVIFISSGKNYGFTIRAIRVYVGDSDIYTV |
| | | | HHIVWNVEEGSPACQAGL1CAGDLITHINGEPVH |
| | | | GLVHTEVIELLLKSGNKVSITTTPF |
| KIAA0313 | 7657260 | 1 | ILACAAKAKRRLMTLTKPSREAPLPFILLGGSEK |
| | | | GFGIFVDSVDSGSKATEAGLKRGDQILEVNGQNF |
| | | | ENIQLSKAMEILRNNTHLSITVKTNLFVFKELLT |
| | | | NSS |
| KIAA0316 | 6683123 | 1 | IPPAPRKVEMRRDPVLGFGFVAGSEKPVVVRSVT |
| | | | PGGPSEGKLIPGDQIVMINDEPVSAAPRERVIDL |
| | | | VRSCKESILLTVIQPYPSPK |
| KIAA0340 | 2224620 | 1 | LNKRTTMPKDSGALLGLKVVGGKMTDLGRLGAFI |
| | | | TKVKKGSLADVVGHLRAGDEVLEWNGKPLPGATN |
| | | | EEVYNIILESKSEPQVEIIVSRPIGDIPRIHRD |
| KIAA0380 | 2224700 | 1 | QRCVIIQKDQHGFGFTVSGDRIVLVQSVRPGGAA |
| | | | MKAGVKEGDRIIKVNGTMVTNSSHLEVVKLIKSG |
| | | | AYVALTLLGSS |
| KIAA0382 | 7662087 | 1 | ILVQRCVIIQKDDNGFGLTVSGDNPVFVQSVKED |
| | | | GAAMRAGVQTGDRIIKVNGTLVTHSNHLEVVKLI |
| | | | KSGSYVALTVQGRPPGNSS |
| KIAA0440 | 2662160 | 1 | SVEMTLRRNGLGQLGFHVNYEGIVADVEPYGYAW |
| | | | QAGLRQGSRLVEICKVAVATLSHEQMIDLLRTSV |
| | | | TVKVVIIPPHD |
| KIAA0545 | 14762850 | 1 | LKVMTSGWETVDMTLRRNGLGQLGFHVKYDGTVA |
| | | | EVEDYGFAWQAGLRQGSRLVEICKVAVVTLTHDQ |
| | | | MIDLLRTSVTVKVVIIPPFEDGTPRRGW |
| KIAA0559 | 3043641 | 1 | HYIFPHARIKITRDSKDHTVSGNGLGIRIVGGKE |
| | | | IPGHSGEIGAYIAKILPGGSAEQTGKLMEGMQVL |
| | | | EWNGIPLTSKTYEEVQSIISQQSGEAEICVRLDL |
| | | | NML |
| KIAA0561 | 3043645 | 1 | LCGSLRPPIVIHSSGKKYGFSLRAIRVYMGDSDV |
| | | | YTVHHVVWSVEDGSPAQEAGLRAGDLITHINGES |
| | | | VLGLVHMDVVELLLKSGNKISLRTTALENTSIKV |
| | | | G |
| KIAA0613 | 3327039 | 1 | SYSVTLTGPGPWGFRLQGGICDFNMPLTISRITP |
| | | | GSKAAQSQLSQGDLVVATDGVNTDTMTHLEAQNK |
| | | | IKSASYNLSLTLQKSKNSS |
| KIAA0751 | 12734165 | 1 | ISRDSGAMLGLKVVGGKMTESGRLCAFITKVKKG |
| | | | SLADTVGHLRPGDEVLEWNGRLLQGATFEEVYNI |
| | | | ILESKPEPQVELVVSRPIAIHRD |
| KIAA0807 | 3882334 | 1 | ISALGSMRPPIIIEHRAGKKYGFTLRAIRVYMGD |
| | | | SDVYTVHHMVWHVEDGGPASEAGLRQGDLITHVN |
| | | | GEPVHGLVHTEVVELILKSGNKVAISTTPLENSS |
| KIAA0858 | 4240204 | 1 | FSDMRISINQTPGKSLDFGFTIKWDIPGIEVASV |
| | | | EAGSPAEFSQLQVDDEIIAINNTKFSYNDSKEWE |
| | | | EAMAKAQETGHLVMDVRRYGKAGSPE |
| KIAA0902 | 4240292 | 1 | QSAHLEVIQLANIKPSEGLGMYIKSTYDGLHVIT |
| | | | GTTENSPADRCKKIHAGDEVIQVNHQTVVGWQLK |
| | | | NLVNALREDPSGVILTLKKRPQSMLTSAPA |
| KIAA0967 | 4589577 | 1 | ILTQTLIPVRHTVKIDKDTLLQDYGFHISESLPL |
| | | | TVVAVTAGGSAHGKLFPGDQILQMNNEPAEDLSW |
| | | | ERAVDILREAEDSLSITVVRCTSGVPKSSNSS |
| KIAA0973 | 4589589 | 1 | GLRSPITIQRSGKKYGFTLRAIRVYMGDTDVYSV |
| | | | HHIVWHVEEGGPAQEAGLCAGDLITHVNGEPVHG |
| | | | MVHPEVVELILKSGNKVAVTTTPFE |
| KIAA1095 | 5889526 | 1 | QGEETKSLTLVLHRD SGSLGFNIIGGRPSVDNH |
| | | | DGSSSEGIFVSKIVDSGPAAKEGGLQIHDRIIEV |
| | | | NGRDLSRATHDQAVEAFKTAICEPIVVQVLRRTP |
| | | | RTICMFTP |
| KIAA1095 | 5889526 | 2 | QEMDREELELEEVDLYRIVINSQDKLGLTVCYRT |
| | | | DDEDDIGIYISEIDPNSIAAKDGRIREGDRIIQI |
| | | | NGIEVQNREEAVALLTSEENKNFSLLIARPELQL |
| | | | D |
| KIAA1202 | 6330421 | 1 | RSFQYVPVQLQGGAPWGFTLKGGLEHCEPLTVSK |
| | | | IEDGGKAALSQKMRTGDELVNINGTPLYGSRQEA |
| | | | LILIKGSFRILKL1VRRRNAPVS |
| KIAA1222 | 6330610 | 1 | ILEKLELFPVELEKDEDGLGISIIGMGVGADAGL |
| | | | EKLGIFVKTVTEGGAAQRDGRIQVNDQIVEVDGI |
| | | | SLVGVTQNFAATVLRNTKGNVRFVIGREKPGQVS |
| KIAA1284 | 6331369 | 1 | ICDVNVYVNPKKLTVIKAKEQLKLLEVLVGIIHQ |
| | | | TKWSWRRTGKQGDGERLVVHGLLPGGSAMKSGQV |
| | | | LIGDVLVAVNDVDVTTENIERVLSCIPGPMQVKL |
| | | | TFENAYDVKRET |
| KIAA1389 | 7243158 | 1 | TRGCETVEMTLRRNGLGQLGFHVNFEGIVADVEP |
| | | | FGFAWKAGLRQGSRLVEICKVAVATLTHEQMIDL |
| | | | LRTSVTVKVVIIQPHDDGSPRR |
| KIAA1415 | 7243210 | 1 | VENILAKRLLILPQEEDYGFDIEEKNKAVVVKSV |
| | | | QRGSLAEVAGLQVGRKIYSINEDLVFLRPFSEVE |
| | | | SILNQSFCSRRPLRLLVATKAICEIIKIP |
| KIAA1526 | 5817166 | 1 | PDSAGPGEVRLVSLRRAKAHEGLGFSIRGGSEHG |
| | | | VGIYVSLVEPGSLAEKEGLRVGDQILRVNDKSLA |
| | | | RVTHAEAVICALKGSKKLVLSVYSAGRIPGGYVT |
| | | | NH |
| KIAA1526 | 5817166 | 2 | LQGGDEKKVNLVLGDGRSLGLTIRGGAEYGLGIY |
| | | | ITGVDPGSEAEGSGLKVGDQILEVNWRSFLNILH |
| | | | DEAVRLLKSSRHLILTVKDVGRLPHARTTVDE |
| KIAA1526 | 5817166 | 3 | WTSGAHVHSGPCEEKCGHPGHRQPLPRIVTIQRG |
| | | | GSAHNCGQLKVGHVILEVNGLTLRGKEHREAARI |
| | | | IAEAFKTKDRDYIDFLDSL |
| KIAA1620 | 10047316 | 1 | ELRRAELVEIIVETEAQTGVSGINVAGGGKEGIF |
| | | | VRELREDSPAARSLSLQEGDQLLSARVFFENFKY |
| | | | EDALRLLQCAEPYKVSFCLICRTVPTGDLALRP |
| KIAA1634 | 10047344 | 1 | PSQLKGVLVRASLKKSTMGFGFTIIGGDRPDEFL |
| | | | QVKNVLKDGPAAQDGKIAPGDVIVDINGNCVLGH |
| | | | THADVVQMFQLVPVNQYVNLTLCRGYPLPDDSED |
| KIAA1634 | 10047344 | 2 | ASSGSSQPELVTIPLIKGPKGFGFAIADSPTGQK |
| | | | VKMILDSQWCQGLQKGDIIKEIYHQNVQNLTHLQ |
| | | | VVEVLKQFPVGADVPLLILRGGPPSPTKTAKM |
| KIAA1634 | 10047344 | 3 | LYEDKPPLTNTFLISNPRTTADPRILYEDKPPNT |
| | | | KDLDVFLRKQESGFGFRVLGGDGPDQSIYIGAII |
| | | | PLGAAEKDGRLRAADELMCIDGIPVKGKSHKQVL |
| | | | DLMTTAARNGHVLLTVRRKIFYGEKQPEDDSGSP |
| | | | GIHRELT |
| KIAA1634 | 10047344 | 4 | PAPQEPYDVVLQRKENEGFGFVILTSKNKPPPGV |
| | | | IPHKIGRVIEGSPADRCGKLKVGDHISAVNGQSI |
| | | | LVESHDNIVQLIKDAGVTVTLTVIAEEEHHGPPS |
| KIAA1634 | 10047344 | 5 | QNLGCYPVELERGPRGFGFSLRGGKEYNIVIGLF |
| | | | ILRLAEDGPAIKDGRIHVGDQIVEINGEPTQGIT |
| | | | HTRAIELIQAGGNKVLLLLRPGTGLIPDHGLA |
| KIAA1719 | 1267982 | 0 | ITVVELIKKEGSTLGLTISGGTDKDGKPRVSNLR |
| | | | PGGLAARSDLLNIGDYIRSVNGIFILTRLRHDEH |
| | | | TLLKNVGERVVLEVEY |
| KIAA1719 | 1267982 | 1 | ILDVSLYKEGNSFGFVLRGGAHEDGHKSRPLVLT |
| | | | YVRPGGPADREGSLKVGDRLLSVDGIPLHGASHA |
| | | | TALATLRQCSHEALFQVEYDVATP |
| KIAA1719 | 1267982 | 2 | IHTVANASGPLMVEIVKTPGSALGISLTTTSLRN |
| | | | KSVITIDRIKPASVVDRSGALHPGDHILSIDGTS |
| | | | MEHCSLLEATKLLASISEKVRLEILPVPQSQRPL |
| KIAA1719 | 1267982 | 3 | IQIVHTETTEVVLCGDPLSGFGLQLQGGIFATET |
| | | | LSSPPLVCFIEPDSPAERCGLLQVGDRVLSINGI |
| | | | ATEDGTMEEANQLLRDAALAHKVVLEVEFDVAES |
| | | | V |
| KIAA1719 | 1267982 | 4 | IQFDVAESVIPSSGTFHVKLPKKRSVELGITISS |
| | | | KASRRGEPLIISDIKKGSVAHRTGTLEPGDKLLA |
| | | | IDNIRLDNCPMEDAVQILRQCEDLVKLKIRKDED |
| | | | N |
| KIAA1719 | 1267982 | 5 | IQTTGAVSYTVELKRYGGPLGITISGTEEPFDPI |
| | | | GVISLTKRGLAERTGAIHVGDRILAINNVSLKGR |
| | | | PLSEAIHLLQVAGETVTLKIKKQLDR |
| KIAA1719 | 1267982 | 6 | ILEMEELLLPTPLEMHKVTLHKDPMRHDFGFSVS |
| | | | DGLLEKGVYVHTVRPDGPAHRGGLQPFDRVLQVN |
| | | | HVRTRDFDCCLAVPLLAEAGDVLELIISRKPHTA |
| | | | HSS |
| LIM Mystique | 12734250 | 1 | MALTVDVAGPAPWGFRITGGRDFHTPIMVTKVAE |
| | | | RGKAKDADLRPGDIIVAINGESAEGMLHAEAQSK |
| | | | IRQSPSPLRLQLDRSQATSPGQT |
| LIM Protein | 3108092 | 1 | SNYSVSLVGPAPWGFRLQGGKDFNMPLTISSLKD |
| | | | GGKAAQANVRIGDVVLSIDGINAQGMTHLEAQNK |
| | | | IKGCTGSLNMTLQRAS |
| LIMK1 | 4587498 | 1 | TLVEHSKLYCGHCYYQTVVTPVIEQILPDSPGSH |
| | | | LPHTVTLVSIPASSHGKRGLSVSIDPPHGPPGCG |
| | | | TEHSHTVRVQGVDPGCMSPDVKNSIHVGDRILEI |
| | | | NGTPIRNVPLDEIDLLIQETSRLLQLTLEHD |
| LIMK2 | 1805593 | 1 | PYSVTLISMPATTEGRRGFSVSVESACSNYATTV |
| | | | QVKEVNRMHISPNNRNAIHPGDRILEINGTPVRT |
| | | | LRVEEVEDAISQTSQTLQLLIEHD |
| LIM-RIL | 1085021 | 1 | IHSVTLRGPSPWGFRLVGRDFSAPLTISRVHAGS |
| | | | KASLAALCPGDLIQAINGESTELMTHLEAQNRIK |
| | | | GCHDHLTLSVSRPE |
| LU-1 | U52111 | 1 | VCYRTDDEEDLGIYVGEVNPNSIAAKDGRIREGD |
| | | | RIIQINGVDVQNREEAVAILSQEENTNISLLVAR |
| | | | PESQLA |
| MAGI1 | 3370997 | 1 | IQKKNHWTSRVHECTVKRGPQGELGVTVLGGAEH |
| | | | GEFPYVGAVAAVEAAGLPGGGEGPRLGEGELLLE |
| | | | VQGVRVSGLPRYDVLGVIDSCKEAVTFKAVRQGG |
| | | | R |
| MAGI1 | 3370997 | 2 | PSELKGKFIHTKLRKSSRGFGFTVVGGDEPDEFL |
| | | | QIKSLVLDGPAALDGKMETGDVIVSVNDTCVLGH |
| | | | THAQVVKIFQSIPIGASVDLELCRGYPLPFDPDD |
| | | | PN |
| MAGI1 | 3370997 | 3 | PATQPELITVHIVKGPMGFGFTIADSPGGGGQRV |
| | | | KQIVDSPRCRGLKEGDLIVEVNKKNVQALTHNQV |
| | | | VDMLVECPKGSEVTLLVQRGGNLS |
| MAGI1 | 3370997 | 4 | PDYQEQDIFLWRKETGFGFRILGGNEPGEPIYIG |
| | | | HIVPLGAADTDGRLRSGDELICVDGTPVIGKSHQ |
| | | | LVVQLMQQAAKQGHVNLTVRRKVVFAVPKTENSS |
| MAGI1 | 3370997 | 5 | GVVSTVVQPYDVEIRRGENEGFGFVIVSSVSRPE |
| | | | AGTTFAGNACVAMPHKIGRIIEGSPADRCGKLKV |
| | | | GDRILAVNGCSITNKSHSDIVNLIKEAGNTVTLR |
| | | | IIPGDESSNA |
| MAGI1 | 3370997 | 6 | QATQEQDFYTVELERGAKGFGFSLRGGREYNMDL |
| | | | YVLRLAEDGPAERCGKMRIGDEILEINGETTKNM |
| | | | KHSRAIELIKNGGRRVRLFLKRG |
| MGC5395 | BC012477 | 1 | PAKMEKEETTRELLLPNWQGSGSHGLTIAQRDDG |
| | | | VFVQEVTQNSPAARTGVVKEGDQIVGATIYFDNL |
| | | | QSGEVTQLLNTMGHHTVGLKLHRKGDRSPNSS |
| MINT1 | 2625024 | 1 | SENCKdVFIEKQKGEILGVVIVESGWGSILPTVI |
| | | | IANMMHGGPAEKSGKLNIGDQIMSINGTSLVGLP |
| | | | LSTCQSIIKGLKNQSRVKLNIVRCPPVNSS |
| MINT1 | 2625024 | 2 | LRCPPVTTVLIRRPDLRYQLGFSVQNGIICSLMR |
| | | | GGIAERGGVRVGHRIIEINGQSVVATPHEKIVHI |
| | | | LSNAVGEIHMKTMPAAMYRLLNSS |
| MINT3 | 3169808 | 1 | LSNSDNCREVHLEKRRGEGLGVALVESGWGSLLP |
| | | | TAVIANLLHGGPAERSGALSIGDRLTAINGTSLV |
| | | | GLPLAACQAAVRETKSQTSVTLSIVHCPPVTTAI |
| | | | M |
| MINT3 | 3169808 | 2 | LVHCPPVTTAIIHRPHAREQLGFCVEDGIICSLL |
| | | | RGGIAERGGIRVGHRIIEINGQSVVATPHARIIE |
| | | | LLTEAYGEVHIKTMPAATYRLLTG |
| MPP1 | 189785 | 1 | RKVRLIQFEKVTEEPMGITLKLNEKQSCTVARIL |
| | | | HGGMIHRQGSLHVGDEILEINGTNVTNHSVDQLQ |
| | | | KAMKETKGMISLKVIPNQ |
| MPP2 | 939884 | 1 | PVPPDAVRMVGIRKTAGEHLGVTFRVEGGELVIA |
| | | | RILHGGMVAQQGLLHVGDIIKEVNGQPVGSDPRA |
| | | | LQELLRNASGSVILKILPNYQ |
| MUPP1 | 2104784 | 1 | QGRHVEVFELLKPPSGGLGFSVVGLRSENRGELG |
| | | | IFVQEIQEGSVAHRDGRLKETDQILAINGQALDQ |
| | | | TITHQQAISILQKAKDTVQLVIARGSLPQLV |
| MUPP1 | 2104784 | 2 | PVHWQHMETIELVNDGSGLGFGIIGGKATGVIVK |
| | | | TILPGGVADQHGRLCSGDHILKIGDTDLAGMSSE |
| | | | QVAQVLRQCGNRVKLMIARGAIEERTAPT |
| MUPP1 | 2104784 | 3 | QESETFDVELTKNVQGLGITIAGYIGDKKLEPSG |
| | | | IFVKSITKSSAVEHDGRIQIGDQIIAVDGTNLQG |
| | | | FTNQQAVEVLRHTGQTVLLTLMRRGMKQEA |
| MUPP1 | 2104784 | 4 | LNYEIVVAHVSKFSENSGLGISLEATVGHHFIRS |
| | | | VLPEGPVGHSGKLFSGDELLEVNGITLLGENHQD |
| | | | VVNILKELPIEVTMVCCRRTVPPT |
| MUPP1 | 2104784 | 5 | WEAGIQHIELEKGSKGLGFSILDYQDPIDPASTV |
| | | | IIIRSLVPGGIAEKDGRLLPGDRLMFVNDVNLEN |
| | | | SSLEEAVEALKGAPSGTVRIGVAKPLPLSPEE |
| MUPP1 | 2104784 | 6 | RNVSKESFERTINIAKGNSSLGMTVSANKDGLGM |
| | | | IVRSIIHGGAISRDGRIAIGDCILSINEESTISV |
| | | | TNAQARAMLRRHSLIGPDIKITYVPAEHLEE |
| MUPP1 | 2104784 | 7 | LNWNQPRRVELWREPSKSLGISIVGGRGMGSRLS |
| | | | NGEVMRGIFIKHVLEDSPAGKNGTLKPGDRIVEV |
| | | | DGMDLRDASHEQAVEAIRKAGNPVVFMVQSIINR |
| | | | PRKSPLPSLL |
| MUPP1 | 2104784 | 8 | LTGELHMIELEKGHSGLGLSLAGNKDRSRMSVFI |
| | | | VGIDPNGAAGKDGRLQIADELLEINGQILYGRSH |
| | | | QNASSIIKCAPSKVKIIFIRNKDAVNQ |
| MUPP1 | 2104784 | 9 | LSSFICNVQHLELPKDQGGLGIAISEEDTLSGVI |
| | | | IKSLTEHGVAATDGRLKVGDQILAVDDEIVVGYP |
| | | | IEKFISLLKTAKMTVKLTIHAENPDSQ |
| MUPP1 | 2104784 | 10 | LPGCETTIEISKGRTGLGLSIVGGSDTLLGAIII |
| | | | HEVYEEGAACKDGRLWAGDQILEVNGIDLRKATH |
| | | | DEAINVLRQTPQRVRLTLYRDEAPYKE |
| MUPP1 | 2104784 | 11 | KEEEVCDTLTIELQKKPGKGLGLSIVGKRNDTGV |
| | | | FVSDIVKGGIADADGRLMQGDQILMVNGEDVRNA |
| | | | TQEAVAALLKCSLGTVTLEVGRIKAGPFHS |
| MUPP1 | 2104784 | 12 | LQGLRTVEMICKGPTDSLGISIAGGVGSPLGDVP |
| | | | IFIAMMHPTGVAAQTQKLRVGDRIVTICGTSTEG |
| | | | MTHTQAVNLLKNASGSIEMQVVAGGDVSV |
| MUPP1 | 2104784 | 13 | LGPPQCKSITLERGPDGLGFSIVGGYGSPHGDLP |
| | | | IYVKTVFAKGAASEDGRLKRGDQIIAVNGQSLEG |
| | | | VTHEEAVAILKRTKGTVTLMVLS |
| NeDLG | 10863920 | 1 | IQYEEIVLERGNSGLGFSIAGGIDNPHVPDDPGI |
| | | | FITKIIPGGAAAIVIDGRLGVNDCVLRVNEVEVS |
| | | | EVVHSRAVEALKEAGPVVRLVVRRRQN |
| NeDLG | 10863920 | 2 | ITLLKGPKGLGFSIAGGIGNQHIPGDNSIYITKI |
| | | | IEGGAAQKDGRLQIGDRLLAVNNTNLQDVRHEEA |
| | | | VASLKNTSDMVYLKVAKPGSLE |
| NeDLG | 10863920 | 3 | ILLHKGSTGLGFNIVGGEDGEGIFVSFILAGGPA |
| | | | DLSGELRRGDRILSVNGVNLRNATHEQAAAALKR |
| | | | AGQSVTIVAQYRPEEYSRFESKIHDLREQMMNSS |
| | | | SMSGSGSLRTSEKRSLE |
| Neurabin II | AJ401189 | 1 | CVERLELFPVELEKDSEGLGISIIGMGAGADMGL |
| | | | EKLGIFVKTVTEGGAAHRDGRIQVNDLLVEVDGT |
| | | | SLVGVTQSFAASVLRNTKGRVRFMIGRERPGEQS |
| | | | EVAQRIHRD |
| NOS1 | 642525 | 1 | IQPNVISVRLFKRKVGGLGFLVKERVSKPPVIIS |
| | | | DLIRGGAAEQSGLIQAGDIILAVNGRPLVDLSYD |
| | | | SALEVLRGIASETHVVLILRGP |
| novel PDZ | 7228177 | 1 | QANSDESDIIHSVRVEKSPAGRLGFSVRGGSEHG |
| gene | | | LGIFVSKVEEGSSAERAGLCVGDKITEVNGLSLE |
| | | | STTMGSAVKVLTSSSRLHMMVRRMGRVPGIKFSK |
| | | | EKNSS |
| novel PDZ | 7228177 | 2 | PSDTSSEDGVRRIVHLYTTSDDFCLGFNIRGGKE |
| gene | | | FGLGIYVSKVDHGGLAEENGIKVGDQVLAANGVR |
| | | | FDDISHSQAVEVLKGQTHIMLTIKETGRYPAYKE |
| | | | MNSS |
| Novel Serine | 1621243 | 1 | KIKKFLTESHDRQAKGKAITKKKYIGIRMMSLTS |
| Protease | | | SKAKELKDRHRDFPDVISGAYIIEVIPDTPAEAG |
| | | | GLKENDVIISINGQSVVSANDVSDVIKRESTLNM |
| | | | VVRRGNEDIMITV |
| Numb Binding | AK056823 | 1 | PDGEITSIKINRVDPSESLSIRLVGGSETPLVHI |
| Protein | | | IIQHIYRDGVIARDGRLLPGDIILKVNGMDISNV |
| | | | PHNYAVRLLRQPCQVLWLTVMREQKFRSRNSS |
| Numb Binding | AK056823 | 2 | HRPRDDSFHVILNKSSPEEQLGIKLVRKVDEPGV |
| Protein | | | IFFNVLDGGVAYRHGQLEENDRVLAINGHDLRYG |
| | | | SPESAAHLIQASERRVHLVVSRQVRQRSPENSS |
| Numb Binding | AK056823 | 3 | PTITCHEKVVNIQKDPGESLGMTVAGGASHREWD |
| Protein | | | LPIYVISVEPGGVISRDGRIKTGDILLNVDGVEL |
| | | | TEVSRSEAVALLKRTSSSIVLKALEVKEYEPQEF |
| | | | IV |
| Numb Binding | AK056823 | 4 | PRCLYNCKDIVLRRNTAGSLGFCIVGGYEEYNGN |
| Protein | | | KPFFIKSIVEGTPAYNDGRIRCGDILLAVNGRST |
| | | | SGMIHACLARLLKELKGRITLTIVSWPGTFL |
| Outer | 7023825 | 1 | LLTEEEINLTRGPSGLGFNIVGGTDQQYVSNDSG |
| Membrane | | | IYVSRIKENGAAALDGRLQEGDKILSVNGQDLKN |
| | | | LLHQDAVDLFRNAGYAVSLRVQHRLQVQNGIHS |
| p55T | 12733367 | 1 | PVDAIRILGIHKRAGEPLGVTFRVENNDLVIARI |
| | | | LHGGMIDRQGLLHVGDIIKEVNGHEVGNNPKELQ |
| | | | ELLKNISGSVTLKILPSYRDTITPQQ |
| PAR3 | 8037914 | 1 | DDMVKLVEVPNDGGPLGIHVVPFSARGGRTLGLL |
| | | | VKRLEKGGKAEHENLFRENDCIVRINDGDLRNRR |
| | | | FEQAQHMFRQAMRTPIIWFHVVPAA |
| PAR3 | 8037914 | 2 | GKRLNIQLKKGTEGLGFSITSRDVTIGGSAPIYV |
| | | | KNILPRGAAIQDGRLKAGDRLIEVNGVDLVGKSQ |
| | | | EEVVSLLRSTKMEGTVSLLVFRQEDA |
| PAR3 | 8037914 | 3 | TPDGTREFLTFEVPLNDSGSAGLGVSVKGNRSKE |
| | | | NHADLGIFVKSIINGGAASKDGRLRVNDQLIAVN |
| | | | GESLLGKTNQDAMETLRRSMSTEGNKRGMIQLIV |
| | | | A |
| PAR6 | 2613011 | 1 | LPETHRRVRLHKHGSDRPLGFYIRDGMSVRVAPQ |
| | | | GLERVPGIFISRLVRGGLAESTGLLAVSDEILEV |
| | | | NGIEVAGKTLDQVTDMMVANSHNLIVTVKPANQR |
| PAR6 | 13537118 | 1 | IDVDLVPETHRRVRLHRHGCEKPLGFYIRDGASV |
| GAMMA | | | RVTPHGLEKVPGIFISRMVPGGLAESTGLLAVND |
| | | | EVLEVNGIEVAGKTLDQVTDMMIANSHNLIVTVK |
| | | | PANQRNNVV |
| PDZ-73 | 5031978 | 1 | RSRKLKEVRLDRLHPEGLGLSVRGGLEFGCGLFI |
| | | | SHLIKGGQADSVGLQVGDEIVRINGYSISSCTHE |
| | | | EVINLIRTICKTVSIKVRHIGLIPVKSSPDEFH |
| PDZ-73 | 5031978 | 2 | IPGNRENKEKKVFISLVGSRGLGCSISSGPIQKP |
| | | | GIFISHVKPGSLSAEVGLEIGDQIVEVNGVDFSN |
| | | | LDHKEAVNVLKSSRSLTISIVAAAGRELFMTDEF |
| PDZ-73 | 5031978 | 3 | PEQIMGKDVRLLRIKKEGSLDLALEGGVDSPIGK |
| | | | VVVSAVYERGAAERHGGIVKGDEIMAINGKIVTD |
| | | | YTLAEADAALQKAWNQGGDWIDLVVAVCPPKEYD |
| | | | D |
| PDZK1 | 2944188 | 1 | LTSTFNPRECKLSKQEGQNYGFFLRIEKDTEGHL |
| | | | VRVVEKCSPAEKAGLQDGDRVLRINGVFVDKEEH |
| | | | MQVVDLVRKSGNSVTLLVLDGDSYEKAGSPGIHR |
| | | | D |
| PDZK1 | 2944188 | 2 | RLCYLVKEGGSYGFSLKTVQGKKGVYMTDITPQG |
| | | | VAMRAGVLADDHLIEVNGENVEDASHEEVVEKVK |
| | | | KSGSRVMFLLVDKETDKREFIVTD |
| PDZK1 | 2944188 | 3 | QFKRETASLKLLPHQPRIVEMKKGSNGYGFYLRA |
| | | | GSEQKGQIIKDIDSGSPAEEAGLKNNDLVVAVNG |
| | | | ESVETLDHDSVVEMIRKGGDQTSLLVVDKETDNM |
| | | | YRLAEFIVTD |
| PDZK1 | 2944188 | 4 | PDTTEEVDHKPKLCRLAKGENGYGFHLNAIRGLP |
| | | | GSFIKEVQKGGPADLAGLEDEDVIIEVNGVNVLD |
| | | | EPYEKVVDRIQSSGKNVTLLVZGKNSS |
| PICK1 | 4678411 | 1 | PTVPGKVTLQKDAQNLIGISIGGGAQYCPCLYIV |
| | | | QVFDNTPAALDGTVAAGDEITGVNGRSIKGKTKV |
| | | | EVAKMIQEVKGEVTIHYNKLQ |
| PIST | 98374330 | 1 | SQGVGPIRKVLLLKEDHEGLGISITGGKEHGVPI |
| | | | LISEIHPGQPADRCGGLHVGDAILAVNGVNLRDT |
| | | | ICHKEAVTILSQQRGEIEFEVVYVAPEVDSD |
| prIL16 | 1478492 | 1 | IHVTILHKEEGAGLGFSLAGGADLENKVITVHRV |
| | | | FPNGLASQEGTIQKGNEVLSINGKSLKGTTHHDA |
| | | | LAILRQAREPRQAVIVTRKLTPEEFIVTD |
| prIL16 | 1478492 | 2 | TAEATVCTVTLEKMSAGLGFSLEGGKGSLHGDKP |
| | | | LTINRIFKGAASEQSETVQPGDEILQLGGTAMQG |
| | | | LTRFEAWNIIKALPDGPVTIV1RRKSLQSK |
| PSD95 | 3318652 | 1 | LEYEeITLERGNSGLGFSIAGGTDNPHIGDDPSI |
| | | | FITKIIPGGAAAQDGRLRVNDSILFVNEVDVREV |
| | | | THSAAVEALKEAGSIVRLYVMRRKPPAENSS |
| PSD95 | 3318652 | 2 | HVMRRKPPAEKVMEIKLIKGPKGLGFSIAGGVGN |
| | | | QHIPGDNSIYVTKIIEGGAAHKDGRLQIGDKILA |
| | | | VNSVGLEDVMHEDAVAALKNTYDVVYLKVAKPS |
| | | | NAYL |
| PSD95 | 3318652 | 3 | REDIPREPRRIVIHRGSTGLGFNIVGGEDGEGIF |
| | | | ISFILAGGPADLSGELRKGDQILSVNGVDLRNAS |
| | | | HEQAAIALKNAGQTVTIIAQYKPEFIVTD |
| PTN-3 | 179912 | 1 | LIRITPDEDGKFGFNLKGGVDQKMPLVVSRINPE |
| | | | SPADTCIPKLNEGDQIVLINGRDISEHTHDQVVM |
| | | | FIKASRESHSRELALVIRRR |
| PTN-4 | 190747 | 1 | IRMKPDENGRFGFNVKGGYDQKMPVIVSRVAPGT |
| | | | PADLCVPRLNEGDQVVLINGRDIAEHTHDQVVLF |
| | | | IKASCERHSGELMLLVRPNA |
| PTPL1 | 515030 | 1 | PEREITLVNLKKDAKYGLGFQIIGGEKMGRLDLG |
| | | | IFISSVAPGGPADFHGCLKPGDRLISVNSVSLEG |
| | | | VSHHAAIEILQNAPEDVTLVISQPKEKISKVPST |
| | | | PVHL |
| PTPL1 | 515030 | 2 | GDIFEVELAKNDNSLGISVTGGVNTSVRHGGIYV |
| | | | KAVIPQGAAESDGRIHKGDRVLAVNGVSLEGATH |
| | | | KQAVETLRNTGQVVHLLLEKGQSPTSK |
| PTPLI | 515030 | 3 | TEENTFEVKLFICNSSGLGFSFSREDNLIPEQIN |
| | | | ASIVRVKKLFAGQPAAESGKIDVGDVILKVNGAS |
| | | | LKGLSQQEVISALRGTAPEVFLLLCRPPPGVLPE |
| | | | IDT |
| PTPL1 | 515030 | 4 | ELEVELLITLIKSEKASLGFTVTKGNQRIGCYVH |
| | | | DVIQDPAKSDGRLKPGDRLIKVNDTDVTNMTHTD |
| | | | AVNLLRAASKTVRLVIGRVLELPRIPMLPH |
| PTPLI | 515030 | 5 | MLPHLLPDITLTCNKEELGFSLCGGHDSLYQVVY |
| | | | ISDINPRSVAAIEGNLQLLDVIHYVNGVSTQGMT |
| | | | LEEVNRALDMSLPSLVLKATRNDLPV |
| RGS12 | 3290015 | 1 | RPSPPRVRSVEVARGRAGYGFTLSGQAPCVLSCV |
| | | | MRGSPADFVGLRAGDQILAVNEINVKKASHEDVV |
| | | | KLIGKCSGVLHMVIAEGVGRFESCS |
| RGS3 | 18644735 | 1 | LCSERRYRQITIPRGKDGFGFTICCDSPVRVQAV |
| | | | DSGGPAERAGLQQLDTVLQLNERPVEHWKCVELA |
| | | | HEIRSCPSEIILLVWRMVPQVICPGIHRD |
| Rhophilin-like | 14279408 | 1 | ISFSANKRWTPPRSIRFTAEEGDLGFTLRGNAPV |
| | | | QVHFLDPYCSASVAGAREGDYIVSIQLVDCKWLT |
| | | | LSEVMKLLKSFGEDEIEMKVVSLLDSTSSMHNKS |
| | | | AT |
| Serine | 2738914 | 1 | RGEKKNSSSGISGSQRRYIGVMMLTLSPSILAEL |
| Protease | | | QLREPSFPDVQHGVLIFIKVILGSPAHRAGLRPG |
| | | | DVILAIGEQMVQNAEDVYEAVRTQSQLAVQIRRG |
| | | | RETLTLYV |
| Shank 1 | 6049185 | 1 | EEKTVVLQKKDNEGFGFVLRGAKADTPIEEFTPT |
| | | | PAFPALQYLESVDEGGVAWQAGLRTGDFLIEVNN |
| | | | ENVVKVGHRQVVNMIRQGGNHLVLKVVTVTRNLD |
| | | | PDDTARKKA |
| Shank 3 | * | 1 | SDYVIDDKVAVLQKRDHEGFGFVLRGAKAETPIE |
| | | | EFTPTPAFPALQYLESVDVEGVAWRAGLRTGDFL |
| | | | IEVNGVNVVKVGHKQVVALIRQGGNRLVMKVVSV |
| | | | TRKPEEDG |
| Shroom | 18652858 | 1 | IYLEAFLEGGAPWGFTLKGGLEHGEPLIISKVEE |
| | | | GGKADTLSSKLQAGDEVVHINEVTLSSSRKEAVS |
| | | | LVKGSYKTLRLVVRRDVCTDPGH |
| SIP1 | 2047327 | 1 | IRLCRLVRGEQGYGFHLHGEKGRRGQFIRRVEPG |
| | | | SPAEAAALRAGDRLVEVNGVNVEGETHHQVVQRI |
| | | | KAVEGQTRLLVVDQN |
| SIP1 | 2047327 | 2 | IRHLRKGPQGYGFNLHSDKSRPGQY1RSVDPGSP |
| | | | AARSGLRAQDRLIEVNGQNVEGLRHAEVVASIKA |
| | | | REDEARLLVVDPETDE |
| SITAC-18 | 8886071 | 1 | PGVREIHLCKDERGKTGLRLRKVDQGLFVQLVQA |
| | | | NTPASLVGLRFGDQLLQIDGRDCAGWSSHKAHQV |
| | | | VKKASGDKIVVVVRDRPFQRTVTM |
| SITAC-18 | 8886071 | 2 | PFQRTVTMHKDSMGHVGFVIKKGKIVSLVKGSSA |
| | | | ARNGLLTNHYVCEVDGQNVIGLKDKKIMEILATA |
| | | | GNVVTLTIIPSVIYEHIVEFIV |
| SSTRIP | 7025450 | 1 | LKEKTVLLQKKDSEGFGFVLRGAKAQTPIEEFTP |
| | | | TPAFPALQYLESVDEGGVAWRAGLRMGDFLIEVN |
| | | | GQNVVKVGHRQVVNMIRQGGNTLMVKVVMVTRHP |
| | | | DMDEAVQ |
| SYNTENIN | 2795862 | 1 | LEIKQGIREVILCKDQDGKIGLRLKSIDNGIFVQ |
| | | | LVQANSPASLVGLRFGDQVLQINGENCAGWSSDK |
| | | | AHKVLKQAFGEKITMRTHRD |
| SYNTENIN | 2795862 | 2 | RDRPFERTITMHKDSTGHVGFIFKNGKITSIVKD |
| | | | SSAARNGLLTEHNICEINGQNVIGLKDSQIADIL |
| | | | STSGNSS |
| Syntrophin 1 | 1145727 | 1 | QRRRVTVRKADAGGLGISIKGGRENKMPILISKI |
| alpha | | | FKGLAADQTEALFVGDAILSVNGEDLSSATHDEA |
| | | | VQVLKKTGKEVVLEVKYMKDVSPYFK |
| Syntrophin | 476700 | 1 | IRVVKQEAGGLGISLKGGRENRMPILISKIFPGL |
| beta 2 | | | AADQSRALRLGDAILSVNGTDLRQATHDQAVQAL |
| | | | KRAGKEVLLEVKFIREFIVTD |
| Syntrophin | 9507162 | 1 | EPFYSGERTVTIRRQTVGGFGLSIKGGAEHNIPV |
| gamma 1 | | | VVSKISKEQRAELSGLLFIGDAILQINGINVRKC |
| | | | RHEEVVQVLRNAGEEVTLTVSFLKRAPAFLKLP |
| Syntrophin | 9507164 | 1 | SHQGRNRRTVTLRRQPVGGLGLSIKGGSEHNVPV |
| gamma 2 | | | VISKIFEDQAADQTGMLFVGDAVLQVNGIHVENA |
| | | | THEEVVHLLRNAGDEVTITVEYLREAPAFLK |
| TAX2-like | 3253116 | 1 | RGETKEVEVTKTEDALGLTITDNGAGYAFIKRIK |
| protein | | | EGSIINRIEAVCVGDSIEAINDHSIVGCRHYEVA |
| | | | KMLRELPKSQPFTLRLVQPKRAF |
| TIAM 1 | 4507500 | 1 | HSTHIEKSDTAADTYGFSLSSVEEDGERRLYVNS |
| | | | VKETGLASKKGLKAGDEILEINNRAADALNSSML |
| | | | KDFLSQPSLGLLVRTYPELE |
| TIAM 2 | 6912703 | 1 | PLNVYDVQLTKTGSVCDFGFAVTAQVDERQHLSR |
| | | | IFISDVLPDGLAYGEGLRKGNEIMTLNGEAVSDL |
| | | | DLKQMEALFSEKSVGLTLIARPPDTKATL |
| TIP1 | 2613001 | 1 | QRVEIHKLRQGENLILGFSIGGGIDQDPSQNPFS |
| | | | EDKTDKGIYVTRVSEGGPAEIAGLQIGDKIMQVN |
| | | | GWDMTMVTHDQARKRLTKRSEEVVRLLVTRQSLQ |
| | | | K |
| TIP2 | 2613003 | 1 | RKEVEVFKSEDALGLTITDNGAGYAFIKRIKEGS |
| | | | VIDHIHLISVGDMIEAINGQSLLGCRHYEVARLL |
| | | | KELPRGRTFTLKLTEPRK |
| TIP33 | 2613007 | 1 | HSHPRVVELPKTDEGLGFNVMGGKEQNSPIYISR |
| | | | IIPGGVAERHGGLKRGDQLLSVNGVSVEGEHHEK |
| | | | AVELLKAAKDSVKLVVRYTPKVL |
| T1P43 | 2613011 | 1 | ISNQKRGVKVLKQELGGLGISIKGGKENKMPILI |
| | | | SKIFKGLAADQTQALYVGDAILSVNGADLRDATH |
| | | | DEAVQALKRAGKEVLLEVKYMREATPYV |
| X-11 beta | 3005559 | 1 | IHFSNSENCKELQLEKHKGEILGVVVVESGWGSI |
| | | | LPTVILANMMNGGPAARSGKLSIGDQIMSINGTS |
| | | | LVGLPLATCQGIIKGLKNQTQVICLNIVSCPPVT |
| | | | TVLIKRNSS |
| X-11 beta | 3005559 | 2 | IPPVTTVLIKRPDLKYQLGFSVQNGIICSLMRGG |
| | | | IAERGGVRVGHRIIEINGQSVVATAHEKIVQALS |
| | | | NSVGEIHMKTMPAAMFRLLTGQENSS |
| ZO-1 | 292937 | 1 | IWEQHTVTLHRAPGFGFGIAISGGRDNPHFQSGE |
| | | | TSIVISDVLKGGPAEGQLQENDRVAMVNGVSMDN |
| | | | VEHAFAVQQLRKSGKNAKITIRRKKKVQIPNSS |
| Z0-1 | 292937 | 2 | ISSQPAKPTKVTLVKSRKNEEYGLRLASHIFVKE |
| | | | ISQDSLAARDGNIQEGDVVLKINGTVTENMSLTD |
| | | | AKTLIERSKGKLKMVVQRDRATLLNSS |
| ZO-1 | 292937 | 3 | IRMKLVKFRKGDSVGLRLAGGNDVGIFVAGVLED |
| | | | SPAAKEGLEEGDQILRVNNVDFTNIIREEAVLFL |
| | | | LDLPKGEEVTILAQKKKDVFSN |
| ZO-2 | 12734763 | 1 | LIWEQYTVTLQKDSKRGFGIAVSGGRDNPHFENG |
| | | | ETSIVISDVLPGGPADGLLQENDRVVMVNGTPME |
| | | | DVLHSFAVQQLRKSGKVAAIVVKRPRKV |
| ZO-2 | 12734763 | 2 | RVLLMKSRANEEYGLRLGSQIFVKEMTRTGLATK |
| | | | DGNLHEGDIILKINGTVTENMSLTDARKLIEKSR |
| | | | GKLQLVVLRDS |
| ZO-2 | 12734763 | 3 | HAPNTKMVRFKKGDSVGLRLAGGNDVGIFVAGIQ |
| | | | EGTSAEQEGLQEGDQILKVNTQDFRGLVREDAVL |
| | | | YLLEIPKGEMVTILAQSRADVY |
| ZO-3 | 10092690 | 1 | IPGNSTIWEQHTATLSKDPRRGFGIAISGGRDRP |
| | | | GGSMVVSDVVPGGPAEGRLQTGDHIVMVNGVSME |
| | | | NATSAFAIQILKTCTKMANITVKRPRRIHLPAEF |
| | | | IVTD |
| ZO-3 | 10092690 | 2 | QDVQMKPVKSVLVKRRDSEEFGVKLGSQIFIKHI |
| | | | TDSGLAARHRGLQEGDLILQINGVSSQNLSLNDT |
| | | | RRLIEKSEGKLSLLVLRDRGQFLVNIPNSS |
| ZO-3 | 10092690 | 3 | RGYSPDTRVVRFLKGKSIGLRLAGGNDVGIFVSG |
| | | | VQAGSPADGQGIQEGDQILQVNDVPFQNLTREEA |
| | | | VQFLLGLPPGEEMELVTQRKQDIFWKMVQSEFIV |
| | | | TD |

*: No GI number for this PDZ domain containing protein - it was computer cloned by
J.S. using rat Shank3 seq against human genomic clone AC000036.
In silico spliced together nt6400-6496, 6985-7109, 7211-7400 to create hypothetical
human Shank3.

1-74. (canceled)

75. An isolated, recombinant or synthetic polypeptide comprising a C-terminal PDZ-binding sequence fused to a transmembrane transporter peptide sequence that facilitates transport of the polypeptide into a cell, wherein the C-terminal PDZ-binding sequence is ETVA (SEQ ID NO: 6).

76. The polypeptide of claim 75, wherein the polypeptide is at least 10 amino acids in length.

77. The polypeptide of claim 75, wherein the polypeptide is less than 40 amino acids in length.

78. The polypeptide of claim 77, wherein the polypeptide is at least 10 amino acids in length.

79. The polypeptide of claim 75, wherein the transmembrane transporter peptide has an amino acid sequence selected from the group consisting of HIV tat, Drosophila antennapedia, herpes simplex virus VP22 and anti-DNA CDR 2 and anti-DNA CDR3, or functional fragments thereof.

80. A pharmaceutical composition comprising a polypeptide of claim 75 and a physiologically acceptable carrier, diluent or excipient.

81. The polypeptide of claim 79, wherein the transmembrane transporter peptide sequence is 10-40 amino acids in length.

82. The polypeptide of claim 81, wherein the transmembrane transporter peptide sequence comprises a truncated HIV tat peptide.

83. The polypeptide of claim 82, wherein the truncated HIV tat peptide comprises YGRKKRRQRRR (SEQ ID NO: 305).

84. The polypeptide of claim 83, wherein the truncated HIV tat peptide comprises GYGRKKRRQRRRG (SEQ ID NO:17).


SEQUENCE LISTING (312 sequences)

SEQ ID NO: 1 - protein, 4 aa, Artificial Sequence
  synthetic C-terminus of polypeptide inhibiting NMDA receptor binding by PDZ protein
ETEV

SEQ ID NO: 2 - protein, 4 aa, Artificial Sequence
  synthetic C-terminus of polypeptide inhibiting NMDA receptor binding by PDX protein
ETQL

SEQ ID NO: 3 - protein, 4 aa, Artificial Sequence
  synthetic C-terminus of polypeptide inhibiting NMDA receptor binding by PDZ protein
QTQV

SEQ ID NO: 4 - protein, 4 aa, Artificial Sequence
  synthetic C-terminus of polypeptide inhibiting NMDA receptor binding by PDZ protein
ETAL

SEQ ID NO: 5 - protein, 4 aa, Artificial Sequence
  synthetic C-terminus of polypeptide inhibiting NMDA receptor binding by PDZ protein
QTEV

SEQ ID NO: 6 - protein, 4 aa, Artificial
  synthetic C-terminus of polypeptide inhibiting NMDA receptor binding by PDX protein
ETVA

SEQ ID NO: 7 - protein, 4 aa, Artificial Sequence
  synthetic C-terminus of polypeptide inhibiting NMDA receptor binding by PDX protein
FTDV

SEQ ID NO: 8 - protein, 8 aa, Artificial Sequence
  synthetic Mutated PDZ ligand motif
TATTAXAA

SEQ ID NO: 9 - protein, 6 aa, Artificial Sequence
  synthetic C-terminus PDZ ligand motif
TATTAX

SEQ ID NO: 10 - protein, 4 aa, Artificial Sequence
  synthetic C-terminus of polypeptide inhibiting NMDA receptor binding by PDZ protein
ESEV

SEQ ID NO: 11 - protein, 19 aa, Artificial Sequence
  synthetic C-terminus from TAX
ISPGGLEPPSEKHFRETEV

SEQ ID NO: 12 - protein, 19 aa, Artificial Sequence
  synthetic C-terminus from modified HPV 16 E6
TGRGMSGGRSSRTRRETQL

SEQ ID NO: 13 - protein, 20 aa, Artificial Sequence
  synthetic C-terminus from TAX
QISPGGLEPPSEKHFRETEV

SEQ ID NO: 14 - protein, 5 aa, Artificial Sequence
  synthetic Flexible polylinker
GGGGS

SEQ ID NO: 15 - protein, 14 aa, Artificial Sequence
  synthetic Linker sequence
EGKSSGSGSESKVD

SEQ ID NO: 16 - protein, 18 aa, Artificial Sequence
  synthetic Linker sequence
KESGSVSSEQLAQFRSLD

SEQ ID NO: 17 - protein, 13 aa, Human immunodeficiency virus
GYGRKKRRQRRRG

SEQ ID NO: 18 - protein, 232 aa, Artificial Sequence
  synthetic pGEX-3X translated region
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLIEGRGIPGNSS

SEQ ID NO: 19 - DNA, 26 bp, Artificial Sequence
  synthetic 1DF primer
tcggatccaggttaatggctcagatg

SEQ ID NO: 20 - DNA, 22 bp, Artificial Sequence
  synthetic 2DR primer
cggaattcggtgcatagccatc

SEQ ID NO: 21 - DNA, 24 bp, Artificial Sequence
  synthetic 8PSF primer
tcggatccttgagggggagatgga

SEQ ID NO: 22 - DNA, 24 bp, Artificial Sequence
  synthetic 11PSR primer
tcggaattcgctatactcttctgg

SEQ ID NO: 23 - DNA, 30 bp, Artificial Sequence
  synthetic 182LF primer
ttaggatcctgagcaagtacagtgtgtcac

SEQ ID NO: 24 - DNA, 31 bp, Artificial Sequence
  synthetic 183LR primer
cttgaattcagcagatgctctttgcagagtc

SEQ ID NO: 25 - DNA, 29 bp, Artificial Sequence
  synthetic 197TF primer
aggggatccgcaaggaggtggaggtgttc

SEQ ID NO: 26 - DNA, 29 bp, Artificial Sequence
  synthetic 198TR primer
tgtggaattccttgcgaggctccgtgagc

SEQ ID NO: 27 - protein, 207 aa, Artificial Sequence
  synthetic DLG1, PDZ domains 1 and 2-pGEX-3X
QVNGTDADYEYEEITLERGNSGLGFSIAGGTDNPHIGDDSSIFITKIITGGAAAQDGRLRVNDCILRVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYAPNS

SEQ ID NO: 28 - protein, 344 aa, Artificial Sequence
  synthetic PSD95, PDZ domains 3 of 3
LEGEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEFIV

SEQ ID NO: 29 - protein, 92 aa, Artificial Sequence
  synthetic TIP2, PDZ domain 1 of pGEX-3X
RKEVEVFKSEDALGLTITDNGAGYAFIKRIKEGSVIDHIHLISVGDMIEAINGQSLLGCRHYEVARLLKELPRGRTFTLKLTEPRKEFIVTD

SEQ ID NO: 30 - protein, 86 aa, Artificial Sequence
  synthetic LIM-pGEX-3X
LSNYSVSLVGPAPWGFRLQGGKDFNMPLTISSLKDGGKAAQANVRIGDVVLSIDGINAQGMTHLEAQNKIKGCTGSLNMTLQRASC

SEQ ID NO: 31 - protein, 20 aa, Artificial Sequence
  synthetic NMDAR2 binding inhibitor peptide
YGRKKRRQRRRKLSSIESDV

SEQ ID NO: 32 - protein, 20 aa, Artificial Sequence
  synthetic AdenoE4 type 9 peptide
VGTLLLERVIFPSVKIATLV

SEQ ID NO: 33 - protein, 20 aa, Artificial Sequence
  synthetic ephrin A2 peptide
RIAYSLLGLKDQVNTVGIPI

SEQ ID NO: 34 - protein, 20 aa, Artificial Sequence
  synthetic HPV-E6 #63 peptide
VHKVRNKFKAKCSLCRLYII

SEQ ID NO: 35 - protein, 20 aa, Artificial Sequence
  synthetic MINT-1 peptide
KTMPAAMYRLLTAQEQPVYI

SEQ ID NO: 36 - protein, 20 aa, Artificial Sequence [sequence omitted]

SEQ ID NO: 37 - protein, 20 aa, Artificial Sequence
  synthetic a-2C adrenergic receptor peptide
DFRPSFKHILFRRARRGFRQ

SEQ ID NO: 38 - protein, 20 aa, Artificial Sequence
  synthetic CSPG4 peptide
ELLQFCRTPNPALKNGQYWV

SEQ ID NO: 39 - protein, 20 aa, Artificial Sequence
  synthetic DNAM-1 peptide
TREDIYVNYPTFSRRPKTRV

SEQ ID NO: 40 - protein, 20 aa, Artificial Sequence
  synthetic HPV-E6 #18 peptide
HSCCNRARQERLQRRRETQV

SEQ ID NO: 41 - protein, 20 aa, Artificial Sequence
  synthetic catenin-delta 2 peptide
PYSELNYETSHYPASPDSWV

SEQ ID NO: 42 - protein, 19 aa, Artificial Sequence
  synthetic Fas ligand peptide
SSKSKSSEESQTFFGLYKL

SEQ ID NO: 43 - protein, 19 aa, Artificial Sequence
  synthetic presenilin-1 peptide
ATDYLVQPFMDQLAFHQFY

SEQ ID NO: 44 - protein, 20 aa, Artificial Sequence
  synthetic ephrin B2 peptide
ILNSIQVMRAQMNQIQSVEV

SEQ ID NO: 45 - protein, 20 aa, Artificial Sequence
  synthetic Dopamine transporter peptide
RELVDRGEVRQFTLRHWLKV

SEQ ID NO: 46 - protein, 20 aa, Artificial Sequence
  synthetic noradrenaline transporter peptide
HHLVAQRDIRQFQLQHWLAI

SEQ ID NO: 47 - protein, 20 aa, Artificial Sequence
  synthetic Serotonin receptor 3A peptide
LAVLAYSITLVMLWSIWQYA

SEQ ID NO: 48 - protein, 20 aa, Artificial Sequence
  synthetic claudin 7 peptide
KAGYRAPRSYPKSNSSKEYV

SEQ ID NO: 49 - protein, 20 aa, Artificial Sequence
  synthetic alpha-2A adrenergic receptor
HDFRRAFKKILARGDRKRIV

SEQ ID NO: 50 - protein, 20 aa, Artificial Sequence
  synthetic claudin 1 peptide
SYPTPRPYPKPAPSSGKDYV

SEQ ID NO: 51 - protein, 20 aa, Artificial Sequence
  synthetic beta-2 adrenergic receptor peptide
VPSDNIDSQGRNASINDSLL

SEQ ID NO: 52 - protein, 19 aa, Artificial Sequence
  synthetic LPAP peptide
AWDDSARAAGGQGLHVTAL

SEQ ID NO: 53 - protein, 20 aa, Artificial Sequence
  synthetic GLUR7 peptide
VDPNSPAAKKKYVSYNNLVI

SEQ ID NO: 54 - protein, 20 aa, Artificial Sequence
  synthetic HPV E6 #35 (cysteine-free) peptide
GRWTGRAMSAWKPTRRETEV

SEQ ID NO: 55 - protein, 20 aa, Artificial Sequence
  synthetic KIAA 1481 peptide
PIPAGGCTFSGIFPTLTSPL

SEQ ID NO: 56 - protein, 20 aa, Artificial Sequence
  synthetic Claudin 2 peptide
PGQPPKVKSEFNSYSLTGYV

SEQ ID NO: 57 - protein, 20 aa, Artificial Sequence
  synthetic CD68 peptide
ALVLIAFCIIRRRPSAYQAL

SEQ ID NO: 58 - protein, 19 aa, Artificial Sequence
  synthetic KV1.3 peptide
TTNNNPNSAVNIKKIFTDV

SEQ ID NO: 59 - protein, 20 aa, Artificial Sequence
  synthetic GluR5-2 (rat) peptide
SFTSILTCHQRRTQRKETVA

SEQ ID NO: 60 - protein, 18 aa, Artificial Sequence
  synthetic CD6 peptide
SPQPDSTDNDDYDDISAA

SEQ ID NO: 61 - protein, 20 aa, Artificial Sequence
  synthetic HPV E6 58 peptide
AVGGRPARGGRLQGRRQTQV

SEQ ID NO: 62 - protein, 20 aa, Artificial Sequence
  synthetic Nectin 2 peptide
SSPDSSYQGKGFVMSRAMYV

SEQ ID NO: 63 - protein, 20 aa, Artificial Sequence
  synthetic Claudin 9 peptide
LGYSIPSRSGASGLDKRDYV

SEQ ID NO: 64 - protein, 19 aa, Artificial Sequence [sequence omitted]

SEQ ID NO: 65 - protein, 20 aa, Artificial Sequence
  synthetic NMDA Glutamate Receptor 2C (cysteine-free) peptide
TQGFPGPATWRRISSLESEV

SEQ ID NO: 66 - protein, 20 aa, Artificial Sequence
  synthetic PSD95 peptide
LNSCSNRRVYKKMPSIESDV

SEQ ID NO: 67 - protein, 20 aa, Artificial Sequence
  synthetic PSD95 peptide
TGSALQAWRHTSRQATESTV

SEQ ID NO: 68 - protein, 20 aa, Artificial Sequence
  synthetic PSD95 peptide
YGRKKRRQRRREKHFRETEV

SEQ ID NO: 69 - protein, 20 aa, Artificial Sequence
  synthetic PSD95 peptide
AAGGRSARGGRLQGRRETAL

SEQ ID NO: 70 - protein, 20 aa, Artificial Sequence
  synthetic PSD95 peptide
LNSSSNRRVYKKMPSIESAV

SEQ ID NO: 71 - protein, 20 aa, Artificial Sequence
  synthetic PSD95 peptide
DGGARTEDEVQSYPSKHDYV

SEQ ID NO: 72 - protein, 20 aa, Artificial Sequence
  synthetic PSD95 peptide
RNIEEVYVGGKQVVPFSSSV

SEQ ID NO: 73 - protein, 20 aa, Artificial Sequence
  synthetic PSD95 peptide
GGDLGTRRGSAHFSSLESEV

SEQ ID NO: 74 - protein, 20 aa, Artificial Sequence
  synthetic PSD95 peptide
FNGSSNGHVYEKLSSIESDV

SEQ ID NO: 75 - protein, 20 aa, Artificial Sequence
  synthetic PSD95 peptide
KDITSDSENSNFRNEIQSLV

SEQ ID NO: 76 - protein, 20 aa, Artificial Sequence
  synthetic PSD95 peptide
RSGATIPLVGQDIIDLQTEV

SEQ ID NO: 77 - protein, 20 aa, Artificial Sequence
  synthetic PSD95 peptide
YGRKKRRQRRREKHFREAEA

SEQ ID NO: 78 - protein, 20 aa, Artificial Sequence
  synthetic PSD95 peptide
AGAVRTPLSQVNKVWDQSSV

SEQ ID NO: 79 - protein, 20 aa, Artificial Sequence
  synthetic PSD95 peptide
ESSGTQSPKRHSGSYLVTSV

SEQ ID NO: 80 - protein, 20 aa, Artificial Sequence
  synthetic C-terminus of NMDAR1 receptor
HPTDITGPLNLSDPSVSTVV

SEQ ID NO: 81 - protein, 20 aa, Artificial Sequence
  synthetic C-terminus of NMDAR1-2 receptor
RRAIEREEGQLQLCSRHRES

SEQ ID NO: 82 - protein, 20 aa, Artificial Sequence
  synthetic C-terminus of NMDAR2C receptor
TQGFPGPCTWRRISSLESEV

SEQ ID NO: 83 - protein, 20 aa, Artificial Sequence
  synthetic C-terminus of NMDAR3A receptor
AVSRKTELEEYQRTSRTCES

SEQ ID NO: 84 - protein, 20 aa, Artificial Sequence
  synthetic NMDAR2C fragment
HPTDITGLPNLSDPSVSTVV

SEQ ID NO: 85 - protein, 87 aa, Artificial Sequence
  synthetic 26s subunit p27 fragment
RDMAEAHKEAMSRKLGQSESQGPPRAFAKVNSISPGSPSIAGLQVDDEIVEFGSVNTQNFQSLHNIGSVVQHSEGALAPTILLSVSM

SEQ ID NO: 86 - protein, 93 aa, Artificial Sequence
  synthetic AF6 fragment
LRKEPEIITVTLKKQNGMGLSIVAAKGAGQDKLGIYVKSVVKGGAADVDGRLAAGDQLLSVDGRSLVGLSQERAAELMTRTSSVVTLEVAKQG

SEQ ID NO: 87 - protein, 105 aa, Artificial Sequence
  synthetic AIPC fragment
LIRPSVISIIGLYKEKGKGLGFSIAGGRDCIRGQMGIFVKTIFPNGSAAEDGRLKEGDEILDVNGIPIKGLTFQEAIHTFKQIRSGLFVLTVRTKLVSPSLTNSS

SEQ ID NO: 88 - protein, 132 aa, Artificial Sequence
  synthetic AIPC fragment
GISSLGRKTPGPKDRIVMEVTLNKEPRVGLGIGACCLALENSPPGIYIHSLAPGSVAKMESNLSRGDQILEVNSVNVRHAALSKVHAILSKCPPGPVRLVIGRHPNPKVSEQEMDEVIARSTYQESKEANSS

SEQ ID NO: 89 - protein, 105 aa, Artificial Sequence
  synthetic AIPC fragment
QSENEEDVCFIVLNRKEGSGLGFSVAGGTDVEPKSITVHRVFSQGAASQEGTMNRGDFLLSVNGASLAGLAHGNVLKVLHQAQLHKDALVVIKKGMDQPRPSNSS

SEQ ID NO: 90 - protein, 101 aa, Artificial Sequence
  synthetic AIPC fragment
LGRSVAVHDALCVEVLKTSAGLGLSLDGGKSSVTGDGPLVIKRVYKGGAAEQAGIIEAGDEILAINGKPLVGLMHFDAWNIMKSVPEGPVQLLIRKHRNSS

SEQ ID NO: 91 - protein, 74 aa, Artificial Sequence
  synthetic alpha actinin-2 associated LIM protein fragment
QTVILPGPAAWGFRLSGGIDFNQPLVITRITPGSKAAAANLCPGDVILAIDGFGTESMTHADGQDRIKAAEFIV

SEQ ID NO: 92 - protein, 85 aa, Artificial Sequence
  synthetic APXL-1 fragment
ILVEVQLSGGAPWGFTLKGGREHGEPLVITKIEEGSKAAAVDKLLAGDEIVGINDIGLSGFRQEAICLVKGSHKTLKLVVKRNSS

SEQ ID NO: 93 - protein, 104 aa, Artificial Sequence
  synthetic Atrophin-1 interacting protein fragment
REKPLFTRDASQLKGTFLSTTLKKSNMGFGFTIIGGDEPDEFLQVKSVIPDGPAAQDGKMETGDVIVYINEVCVLGHTHADVVKLFQSVPIGQSVNLVLCRGYP

SEQ ID NO: 94 - protein, 91 aa, Artificial Sequence
  synthetic Atrophin-1 interacting protein fragment
LSGATQAELMTLTIVKGAQGFGFTIADSPTGQRVKQILDIQGCPGLCEGDLIVEINQQNVQNLSHTEVVDILKDCPIGSETSLIIHRGGFF

SEQ ID NO: 95 - protein, 93 aa, Artificial Sequence
  synthetic Atrophin-1 interacting protein fragment
HYKELDVHLRRMESGFGFRILGGDEPGQPILIGAVIAMGSADRDGRLHPGDELVYVDGIPVAGKTHRYVIDLMHHAARNGQVNLTVRRKVLCG

SEQ ID NO: 96 - protein, 106 aa, Artificial Sequence
  synthetic Atrophin-1 interacting protein fragment
EGRGISSHSLQTSDAVIHRKENEGFGFVIISSLNRPESGSTITVPHKIGRIIDGSPADRCAKLKVGDRILAVNGQSIINMPHADIVKLIKDAGLSVTLRIIPQEEL

SEQ ID NO: 97 - protein, 98 aa, Artificial Sequence
  synthetic Atrophin-1 interacting protein fragment
LSDYRQPQDFDYFTVDMEKGAKGFGFSIRGGREYKMDLYVLRLAEDGPAIRNGRMRVGDQIIEINGESTRDMTHARAIELIKSGGRRVRLLLKRGTGQ

SEQ ID NO: 98 - protein, 90 aa, Artificial Sequence
  synthetic Atrophin-1 interacting protein fragment
HESVIGRNPEGQLGFELKGGAENGQFPYLGEVKPGKVAYESGSKLVSEELLLEVNETPVAGLTIRDVLAVIKHCKDPLRLKCVKQGGIHR

SEQ ID NO: 99 - protein, 126 aa, Artificial Sequence
  synthetic CARD11 protein fragment
NLMFRKFSLERPFRPSVTSVGHVRGPGPSVQHTTLNGDSLTSQLTLLGGNARGSFVHSVKPGSLAEKAGLREGHQLLLLEGCIRGERQSVPLDTCTKEEAHWTIQRCSGPVTLHYKVNHEGYRKLV

SEQ ID NO: 100 - protein, 100 aa, Artificial Sequence
  synthetic CARD14 protein fragment
ILSQVTMLAFQGDALLEQISVIGGNLTGIFIHRVTPGSAADQMALRPGTQIVMVDYEASEPLFKAVLEDTTLEEAVGLLRRVDGFCCLSVKVNTDGYKRL

SEQ ID NO: 101 - protein, 90 aa, Artificial Sequence
  synthetic CASK protein fragment
TRVRLVQFQKNTDEPMGITLKMNELNHCIVARIMHGGMIHRQGTLHVGDEIREINGISVANQTVEQLQKMLREMRGSITFKIVPSYRTQS

SEQ ID NO: 102 - protein, 88 aa, Artificial Sequence
  synthetic Connector enhancer protein fragment
LEQKAVLEQVQLDSPLGLEIHTTSNCQHFVSQVDTQVPTDSRLQIQPGDEVVQINEQVVVGWPRKNMVRELLREPAGLSLVLKKIPIP

SEQ ID NO: 103 - protein, 92 aa, Artificial Sequence
  synthetic Cytohesin binding protein fragment
QRKLVTVEKQDNETFGFEIQSYRPQNQNACSSEMFTLICKIQEDSPAHCAGLQAGDVLANINGVSTEGFTYKQVVDLIRSSGNLLTIETLNG

SEQ ID NO: 104 - protein, 109 aa, Artificial Sequence
  synthetic Densin 180 protein fragment
RCLIQTKGQRSMDGYPEQFCVRIEKNPGLGFSISGGISGQGNPFKPSDKGIFVTRVQPDGPASNLLQPGDKILQANGHSFVHMEHEKAVLLLKSFQNTVDLVIQRELTV

SEQ ID NO: 105 - protein, 101 aa, Artificial Sequence
  synthetic DLG1 protein fragment
IQVNGTDADYEYEEITLERGNSGLGFSIAGGTDNPHIGDDSSIFITKIITGGAAAQDGRLRVNDCILQVNEVDVRDVTHSKAVEALKEAGSIVRLYVKRRN

SEQ ID NO: 106 - protein, 95 aa, Artificial Sequence
  synthetic DLG1 protein fragment
IQLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGN

SEQ ID NO: 107 - protein, 85 aa, Artificial Sequence
  synthetic DLG1 protein fragment
ILHRGSTGLGFNIVGGEDGEGIFISFILAGGPADLSGELRKGDRIISVNSVDLRAASHEQAAAALKNAGQAVTIVAQYRPEEYSR

SEQ ID NO: 108 - protein, 101 aa, Artificial Sequence
  synthetic DLG2 protein fragment
ISYVNGTEIEYEFEEITLERGNSGLGFSIAGGTDNPHIGDDPGIFITKIIPGGAAAEDGRLRVNDCILRVNEVDVSEVSHSKAVEALKEAGSIVRLYVRRR

SEQ ID NO: 109 - protein, 94 aa, Artificial Sequence
  synthetic DLG2 protein fragment
ISVVEIKLFKGPKGLGFSIAGGVGNQHIPGDNSIYVTKIIDGGAAQKDGRLQVGDRLLMVNNYSLEEVTHEEAVAILKNTSEVVYLKVGNPTTI

SEQ ID NO: 110 - protein, 95 aa, Artificial Sequence
  synthetic DLG2 protein fragment
IWAVSLEGEPRKVVLHKGSTGLGFNIVGGEDGEGIFVSFILAGGPADLSGELQRGDQILSVNGIDLRGASHEQAAAALKGAGQTVTIIAQYQPED

SEQ ID NO: 111 - protein, 102 aa, Artificial Sequence
  synthetic DLG5 protein fragment
GIPYVEEPRHVKVQKGSEPLGISIVSGEKGGIYVSKVTVGSIAHQAGLEYGDQLLEFNGINLRSATEQQARLIIGQQCDTITILAQYNPHVHQLRNSSZLTD

SEQ ID NO: 112 - protein, 103 aa, Artificial Sequence
  synthetic DLG5 protein fragment
GILAGDANKKTLEPRVVFIKKSQLELGVHLCGGNLHGVFVAEVEDDSPAKGPDGLVPGDLILEYGSLDVRNKTVEEVYVEMLKPRDGVRLKVQYRPEEFIVTD

SEQ ID NO: 113 - protein, 141 aa, Artificial Sequence
  synthetic DLG6 splice variant 1 protein fragment
PTSPEIQELRQMLQAPHFKALLSAHDTIAQKDFEPLLPPLPDNIPESEEAMRIVCLVKNQQPLGATIKRHEMTGDILVARIIHGGLAERSGLLYAGDKLVEVNGVSVEGLDPEQVIHILAMSRGTIMFKVVPVSDPPVNSS

SEQ ID NO: 114 - protein, 97 aa, Artificial Sequence
  synthetic DLG6 splice variant 2 protein fragment
PTSPEIQELRQMLQAPHFKGATIKRHEMTGDILVARIIHGGLAERSGLLYAGDKLVEVNGVSVEGLDPEQVIHILAMSRGTIMFKVVPVSDPPVNSS

SEQ ID NO: 115 - protein, 93 aa, Artificial Sequence
  synthetic DVL1 protein fragment
LNIVTVTLNMERHHFLGISIVGQSNDRGDGGIYIGSIMKGGAVAADGRIEPGDMLLQVNDVNFENMSNDDAVRVLREIVSQTGPISLTVAKCW

SEQ ID NO: 116 - protein, 100 aa, Artificial Sequence
  synthetic DVL2 protein fragment
LNIITVTLNMEKYNFLGISIVGQSNERGDGGIYIGSIMKGGAVAADGRIEPGDMLLQVNDMNFENMSNDDAVRVLRDIVHKPGPIVLTVAKCWDPSPQNS

SEQ ID NO: 117 - protein, 95 aa, Artificial Sequence
  synthetic DVL3 protein fragment
IITVTLNMEKYNFLGISIVGQSNERGDGGIYIGSIMKGGAVAADGRIEPGDMLLQVNEINFENMSNDDAVRVLREIVHKPGPITLTVAKCWDPSP

SEQ ID NO: 118 - protein, 92 aa, Artificial Sequence
  synthetic ELFIN1 protein fragment
TTQQIDLQGPGPWGFRLVGRKDFEQPLAISRVTPGSKAALANLCIGDVITAIDGENTSNMTHLEAQNRIKGCTDNLTLTVARSEHKVWSPLV

SEQ ID NO: 119 - protein, 89 aa, Artificial Sequence
  synthetic ENIGMA protein fragment
IFMDSFKVVLEGPAPWGFRLQGGKDFNVPLSISRLTPGGKAAQAGVAVGDWVLSIDGENAGSLTHIEAQNKIRACGERLSLGLSRAQPV

SEQ ID NO: 120 - protein, 100 aa, Artificial Sequence
  synthetic ERBIN protein fragment
QGHELAKQEIRVRVEKDPELGFSISGGVGGRGNPFRPDDDGIFVTRVQPEGPASKLLQPGDKIIQANGYSFINIEHGQAVSLLKTFQNTVELIIVREVSS

SEQ ID NO: 121 - protein, 87 aa, Artificial Sequence
  synthetic EZRIN protein fragment
ILCCLEKGPNGYGFHLHGEKGKLGQYIRLVEPGSPAEKAGLLAGDRLVEVNGENVEKETHQQVVSRIRAALNAVRLLVVDPEFIVTD

SEQ ID NO: 122 - protein, 92 aa, Artificial Sequence
  synthetic EZRIN protein fragment
IRLCTMKKGPSGYGFNLHSDKSKPGQFIRSVDPDSPAEASGLRAQDRIVEVNGVCMEGKQHGDVVSAIRAGGDETKLLVVDRETDEFFMNSS

SEQ ID NO: 123 - protein, 107 aa, Artificial Sequence
  synthetic FLJ00011 protein fragment
KNPSGELKTVTLSKMKQSLGISISGGIESKVQPMVKIEKIFPGGAAFLSGALQAGFELVAVDGENLEQVTHQRAVDTIRRAYRNKAREPMELVVRVPGPSPRPSPSD

SEQ ID NO: 124 - protein, 97 aa, Artificial Sequence
  synthetic FLJ11215 protein fragment
EGHSHPRVVELPKTEEGLGFNIMGGKEQNSPIYISRIIPGGIADRHGGLKRGDQLLSVNGVSVEGEHHEKAVELLKAAQGKVKLVVRYTPKVLEEME

SEQ ID NO: 125 - protein, 88 aa, Artificial Sequence
  synthetic FLJ12428 protein fragment
PGAPYARKTFTIVGDAVGWGFVVRGSKPCHIQAVDPSGPAAAAGMKVCQFVVSVNGLNVLHVDYRTVSNLILTGPRTIVMEVMEELEC

SEQ ID NO: 126 - protein, 97 aa, Artificial Sequence
  synthetic FLJ12615 protein fragment
GQYGGETVKIVRIEKARDIPLGATVRNEMDSVIISRIVKGGAAEKSGLLHEGDEVLEINGIEIRGKDVNEVFDLLSDMHGTLTFVLIPSQQIKPPPA

SEQ ID NO: 127 - protein, 98 aa, Artificial Sequence
  synthetic FLJ20075 protein fragment
ILAHVKGIEKEVNVYKSEDSLGLTITDNGVGYAFIKRIKDGGVIDSVKTICVGDHIESINGENIVGWRHYDVAKKLKELKKEELFTMKLIEPKKAFEI

SEQ ID NO: 128 - protein, 104 aa, Artificial Sequence
  synthetic FLJ21687 protein fragment
KPSQASGHFSVELVRGYAGFGLTLGGGRDVAGDTPLAVRGLLKDGPAQRCGRLEVGDLVLHINGESTQGLTHAQAVERIRAGGPQLHLVIRRPLETHPGKPRGV

SEQ ID NO: 129 - protein, 107 aa, Artificial Sequence
  synthetic FLJ31349 protein fragment
PVMSQCACLEEVHLPNIKPGEGLGMYIKSTYDGLHVITGTTENSPADRSQKIHAGDEVIQVNQQTVVGWQLKNLVKKLRENPTGVVLLLKKRPTGSFNFTPEFIVTD

SEQ ID NO: 130 - protein, 100 aa, Artificial Sequence
  synthetic FLJ32798 protein fragment
LDDEEDSVKIIRLVKNREPLGATIKKDEQTGAIIVARIMRGGAADRSGLIHVGDELREVNGIPVEDKRPEEIIQILAQSQGAITFKIIPGSKEETPSNSS

SEQ ID NO: 131 - protein, 83 aa, Artificial Sequence
  synthetic GRIP1 protein fragment
VVELMKKEGTTLGLTVSGGIDKDGKPRVSNLRQGGIAARSDQLDVGDYIKAVNGINLAKFRHDEIISLLKNVGERVVLEVEYE

SEQ ID NO: 132 - protein, 110 aa, Artificial Sequence
  synthetic GRIP1 protein fragment
RSSVIFRTVEVTLHKEGNTFGFVIRGGAHDDRNKSRPVVITCVRPGGPADREGTIKPGDRLLSVDGIRLLGTTHAEAMSILKQCGQEAALLIEYDVSVMDSVATASGNSS

SEQ ID NO: 133 - protein, 106 aa, Artificial Sequence
  synthetic GRIP 1 protein fragment
HVATASGPLLVEVAKTPGASLGVALTTSMCCNKQVIVIDKIKSASIADRCGALHVGDHILSIDGTSMEYCTLAEATQFLANTTDQVKLEILPHHQTRLALKGPNSS

SEQ ID NO: 134 - protein, 97 aa, Artificial Sequence
  synthetic GRIP 1 protein fragment
TETTEVVLTADPVTGFGIQLQGSVFATETLSSPPLISYIEADSPAERCGVLQIGDRVMAINGIPTEDSTFEEASQLLRDSSITSKVTLEIEFDVAES

SEQ ID NO: 135 - protein, 101 aa, Artificial Sequence
  synthetic GRIP 1 protein fragment
AESVIPSSGTFHVKLPKKHNVELGITISSPSSRKPGDPLVISDIKKGSVAHRTGTLELGDKLLAIDNIRLDNCSMEDAVQILQQCEDLVKLKIRKDEDNSD

SEQ ID NO: 136 - protein, 90 aa, Artificial Sequence
  synthetic GRIP 1 protein fragment
IYTVELKRYGGPLGITISGTEEPFDPIIISSLTKGGLAERTGAIHIGDRILAINSSSLKGKPLSEAIHLLQMAGETVTLKIKKQTDAQSA

SEQ ID NO: 137 - protein, 95 aa, Artificial Sequence
  synthetic GRIP 1 protein fragment
IMSPTPVELHKVTLYKDSDMEDFGFSVADGLLEKGVYVKNIRPAGPGDLGGLKPYDRLLQVNHVRTRDFDCCLVVPLIAESGNKLDLVISRNPLA

SEQ ID NO: 138 - protein, 88 aa, Artificial Sequence
  synthetic GTPase activating enzyme protein fragment
SRGCETRELALPRDGQGRLGFEVDAEGFVTHVERFTFAETAGLRPGARLLRVCGQTLPSLRPEAAAQLLRSAPKVCVTVLPPDESGRP

SEQ ID NO: 139 - protein, 95 aa, Artificial Sequence
  synthetic Guanine exchange factor protein fragment
AKAKWRQVVLQKASRESPLQFSLNGGSEKGFGIFVEGVEPGSKAADSGLKRGDQIMEVNGQNFENITFMKAVEILRNNTHLALTVKTNIFVFKEL

SEQ ID NO: 140 - protein, 89 aa, Artificial Sequence
  synthetic HEMBA 100505 protein fragment
LENVIAKSLLIKSNEGSYGFGLEDKNKVPIIKLVEKGSNAEMAGMEVGKKIFAINGDLVFMRPFNEVDCFLKSCLNSRKPLRVLVSTKP

SEQ ID NO: 141 - protein, 82 aa, Artificial Sequence
  synthetic HEMBA 1000505 protein fragment
PRETVKIPDSADGLGFQIRGFGPSVVHAVGRGTVAAAAGLHPGQCIIKVNGINVSKETHASVIAHVTACRKYRRPTKQDSIQ

SEQ ID NO: 142 - protein, 100 aa, Artificial Sequence
  synthetic HEMBA 1003117 protein fragment
EDFCYVFTVELERGPSGLGMGLIDGMHTHLGAPGLYIQTLLPGSPAAADGRLSLGDRILEVNGSSLLGLGYLRAVDLIRHGGKKMRFLVAKSDVETAKKI

SEQ ID NO: 143 - protein, 109 aa, Artificial Sequence
  synthetic HTRA3 protein fragment
LTEFQDKQIKDWKKRFIGIRMRTITPSLVDELKASNPDFPEVSSGIYVQEVAPNSPSQRGGIQDGDIIVKVNGRPLVDSSELQEAVLTESPLLLEVRRGNDDLLFSNSS

SEQ ID NO: 144 - protein, 97 aa, Artificial Sequence
  synthetic HTRA4 protein fragment
HKKYLGLQMLSLTVPLSEELKMHYPDFPDVSSGVYVCKVVEGTAAQSSGLRDHDVIVNINGKPITTTTDVVKALDSDSLSMAVLRGKDNLLLTVNSS

SEQ ID NO: 145 - protein, 104 aa, Artificial Sequence
  synthetic INADL protein fragment
IWQIEYIDIERPSTGGLGFSVVALRSQNLGKVDIFVKDVQPGSVADRDQRLKENDQILAINHTPLDQNISHQQAIALLQQTTGSLRLIVAREPVHTKSSTSSSE

SEQ ID NO: 146 - protein, 78 aa, Artificial Sequence
  synthetic INADL protein fragment
PGHVEEVELINDGSGLGFGIVGGKTSGVVVRTIVPGGLADRDGRLQTGDHILKIGGTNVQGMTSEQVAQVLRNCGNSS

SEQ ID NO: 147 - protein, 111 aa, Artificial Sequence
  synthetic INADL protein fragment
PGSDSSLFETYNVELVRKDGQSLGIRIVGYVGTSHTGEASGIYVKSIIPGSAAYHNGHIQVNDKIVAVDGVNIQGFANHDVVEVLRNAGQVVHLTLVRRKTSSSTSRIHRD

SEQ ID NO: 148 - protein, 96 aa, Artificial Sequence
  synthetic INADL protein fragment
NSDDAELQKYSKLLPIHTLRLGVEVDSFDGHHYISSIVSGGPVDTLGLLQPEDELLEVNGMQLYGKSRREAVSFLKEVPPPFTLVCCRRLFDDEAS

SEQ ID NO: 149 - protein, 102 aa, Artificial Sequence
  synthetic INADL protein fragment
LSSPEVKIVELVKDCKGLGFSILDYQDPLDPTRSVIVIRSLVADGVAERSGGLLPGDRLVSVNEYCLDNTSLAEAVEILKAVPPGLVHLGICKPLVEFIVTD

SEQ ID NO: 150 - protein, 119 aa, Artificial Sequence
  synthetic INADL protein fragment
PNFSHWGPPRIVEIFREPNVSLGISIVVGQTVIKRLKNGEELKGIFIKQVLEDSPAGKTNALKTGDKILEVSGVDLQNASHSEAVEAIKNAGNPVVFIVQSLSSTPRVIPNVHNKANSS

SEQ ID NO: 151 - protein, 99 aa, Artificial Sequence
  synthetic INADL protein fragment
PGELHIIELEKDKNGLGLSLAGNKDRSRMSIFVVGINPEGPAAADGRMRIGDELLEINNQILYGRSHQNASAIIKTAPSKVKLVFIRNEDAVNQMANSS

SEQ ID NO: 152 - protein, 93 aa, Artificial Sequence
  synthetic INADL protein fragment
PATCPIVPGQEMIIEISKGRSGLGLSIVGGKDTPLNAIVIHEVYEEGAAARDGRLWAGDQILEVNGVDLRNSSHEEAITALRQTPQKVRLVVY

SEQ ID NO: 153 - protein, 103 aa, Artificial Sequence
  synthetic K1AA0147 protein fragment
ILTLTILRQTGGLGISIAGGKGSTPYKGDDEGIFISRVSEEGPAARAGVRVGDKLLEVNGVALQGAEHHEAVEALRGAGTAVQMRVWRERMVEPENAEFIVTD

SEQ ID NO: 154 - protein, 97 aa, Artificial Sequence
  synthetic K1AA0147 protein fragment
PLRQRHVACLARSERGLGFSIAGGKGSTPYRAGDAGIFVSRIAEGGAAHRAGTLQVGDRVLSINGVDVTEARHDHAVSLLTAASPTIALLLEREAGG

SEQ ID NO: 155 - protein, 106 aa, Artificial Sequence
  synthetic K1AA0147 protein fragment
ILEGPYPVEEIRLPRAGGPLGLSIVGGSDHSSHPFGVQEPGVFISKVLPRGLAARSGLRVGDRILAVNGQDVRDATHQEAVSALLRPCLELSLLVRRDPAEFIVTD

SEQ ID NO: 156 - protein, 105 aa, Artificial Sequence
  synthetic K1AA0147 protein fragment
RELCIQKAPGERLGISIRGGARGHAGNPRDPTDEGIFISKVSPTGAAGRDGRLRVGLRLLEVNQQSLLGLTHGEAVQLLRSVGDTLTVLVCDGFEASTDAALEVS

SEQ ID NO: 157 - protein, 91 aa, Artificial Sequence
  synthetic KIAA303 protein fragment
PHQPIVIHSSGKNYGFTIRAIRVYVGDSDIYTVHHIVWNVEEGSPACQAGLKAGDLITHINGEPVHGLVHTEVIELLLKSGNKVSITTTPF

SEQ ID NO: 158 - protein, 105 aa, Artificial Sequence
  synthetic KIAA0313 protein fragment
ILACAAKAKRRLMTLTKPSREAPLPFILLGGSEKGFGIFVDSVDSGSKATEAGLKRGDQILEVNGQNFENIQLSKAMEILRNNTHLSITVKTNLFVFKELLTNSS

SEQ ID NO: 159 - protein, 88 aa, Artificial Sequence
  synthetic KIAA0316 protein fragment
IPPAPRKVEMRRDPVLGFGFVAGSEKPVVVRSVTPGGPSEGKLIPGDQIVMINDEPVSAAPRERVIDLVRSCKESILLTVIQPYPSPK

SEQ ID NO: 160 - protein, 101 aa, Artificial Sequence
  synthetic KIAA0340 protein fragment
LNKRTTMPKDSGALLGLKVVGGKMTDLGRLGAFITKVKKGSLADVVGHLRAGDEVLEWNGKPLPGATNEEVYNIILESKSEPQVEIIVSRPIGDIPRIHRD

SEQ ID NO: 161 - protein, 79 aa, Artificial Sequence
  synthetic KIAA0380 protein fragment
QRCVIIQKDQHGFGFTVSGDRIVLVQSVRPGGAAMKAGVKEGDRIIKVNGTMVTNSSHLEVVKLIKSGAYVALTLLGSS

SEQ ID NO: 162 - protein, 87 aa, Artificial Sequence
  synthetic KIAA0382 protein fragment
ILVQRCVIIQKDDNGFGLTVSGDNPVFVQSVKEDGAAMRAGVQTGDRIIKVNGTLVTHSNHLEVVKLIKSGSYVALTVQGRPPGNSS

SEQ ID NO: 163 - protein, 79 aa, Artificial Sequence
  synthetic KIAA0440 protein fragment
SVEMTLRRNGLGQLGFHVNYEGIVADVEPYGYAWQAGLRQGSRLVEICKVAVATLSHEQMIDLLRTSVTVKVVIIPPHD

SEQ ID NO: 164 - protein, 96 aa, Artificial Sequence
  synthetic KIAA0545 protein fragment
LKVMTSGWETVDMTLRRNGLGQLGFHVKYDGTVAEVEDYGFAWQAGLRQGSRLVEICKVAVVTLTHDQMIDLLRTSVTVKVVIIPPFEDGTPRRGW

SEQ ID NO: 165 - protein, 105 aa, Artificial Sequence
  synthetic KIAA0559 protein fragment
HYIFPHARIKITRDSKDHTVSGNGLGIRIVGGKEIPGHSGEIGAYIAKILPGGSAEQTGKLMEGMQVLEWNGIPLTSKTYEEVQSIISQQSGEAEICVRLDLNML

SEQ ID NO: 166 - protein, 103 aa, Artificial Sequence
  synthetic KIAA0561 protein fragment
LCGSLRPPIVIHSSGKKYGFSLRAIRVYMGDSDVYTVHHVVWSVEDGSPAQEAGLRAGDLITHINGESVLGLVHMDVVELLLKSGNKISLRTTALENTSIKVG

SEQ ID NO: 167 - protein, 86 aa, Artificial Sequence
  synthetic KIAA0613 protein fragment
SYSVTLTGPGPWGFRLQGGKDFNMPLTISRITPGSKAAQSQLSQGDLVVAIDGVNTDTMTHLEAQNKIKSASYNLSLTLQKSKNSS

SEQ ID NO: 168 - protein, 91 aa, Artificial Sequence
  synthetic KIAA0751 protein fragment
ISRDSGAMLGLKVVGGKMTESGRLCAFITKVKKGSLADTVGHLRPGDEVLEWNGRLLQGATFEEVYNIILESKPEPQVELVVSRPIAIHRD

SEQ ID NO: 169 - protein, 101 aa, Artificial Sequence
  synthetic KIAA0807 protein fragment
ISALGSMRPPIIIHRAGKKYGFTLRAIRVYMGDSDVYTVHHMVWHVEDGGPASEAGLRQGDLITHVNGEPVHGLVHTEVVELILKSGNKVAISTTPLENSS

SEQ ID NO: 170 - protein, 94 aa, Artificial Sequence
  synthetic KIAA0858 protein fragment
FSDMRISINQTPGKSLDFGFTIKWDIPGIFVASVEAGSPAEFSQLQVDDEIIAINNTKFSYNDSKEWEEAMAKAQETGHLVMDVRRYGKAGSPE

SEQ ID NO: 171 - protein, 98 aa, Artificial Sequence
  synthetic KIAA0902 protein fragment
QSAHLEVIQLANIKPSEGLGMYIKSTYDGLHVITGTTENSPADRCKKIHAGDEVIQVNHQTVVGWQLKNLVNALREDPSGVILTLKKRPQSMLTSAPA

SEQ ID NO: 172 - protein, 100 aa, Artificial Sequence
  synthetic KIAA0967 protein fragment
ILTQTLIPVRHTVKIDKDTLLQDYGFHISESLPLTVVAVTAGGSAHGKLFPGDQILQMNNEPAEDLSWERAVDILREAEDSLSITVVRCTSGVPKSSNSS

SEQ ID NO: 173 - protein, 93 aa, Artificial Sequence
  synthetic KIAA0973 protein fragment
GLRSPITIQRSGKKYGFTLRAIRVYMGDTDVYSVHHIVWHVEEGGPAQEAGLCAGDLITHVNGEPVHGMVHPEVVELILKSGNKVAVTTTPFE

SEQ ID NO: 174 - protein, 107 aa, Artificial Sequence
  synthetic KIAA1095 protein fragment
QGEETKSLTLVLHRDSGSLGFNIIGGRPSVDNHDGSSSEGIFVSKIVDSGPAAKEGGLQIHDRIIEVNGRDLSRATHDQAVEAFKTAKEPIVVQVLRRTPRTKMFTP

SEQ ID NO: 175 - protein, 101 aa, Artificial Sequence
  synthetic KIAA1095 protein fragment
QEMDREELELEEVDLYRMNSQDKLGLTVCYRTDDEDDIGIYISEIDPNSIAAKDGRIREGDRIIQINGIEVQNREEAVALLTSEENKNFSLLIARPELQLD

SEQ ID NO: 176 - protein, 91 aa, Artificial Sequence
  synthetic KIAA1202 protein fragment
RSFQYVPVQLQGGAPWGFTLKGGLEHCEPLTVSKIEDGGKAALSQKMRTGDELVNINGTPLYGSRQEALILIKGSFRILKLIVRRRNAPVS

SEQ ID NO: 177 - protein, 102 aa, Artificial Sequence
  synthetic KIAA1222 protein fragment
ILEKLELFPVELEKDEDGLGISIIGMGVGADAGLEKLGIFVKTVTEGGAAQRDGRIQVNDQIVEVDGISLVGVTQNFAATVLRNTKGNVRFVIGREKPGQVS

SEQ ID NO: 178 - protein, 113 aa, Artificial Sequence
  synthetic KIAA1284 protein fragment
KDVNVYVNPKKLTVIKAKEQLKLLEVLVGIIHQTKWSWRRTGKQGDGERLVVHGLLPGGSAMKSGQVLIGDVLVAVNDVDVTTENIERVLSCIPGPMQVKLTFENAYDVKRET

SEQ ID NO: 179 - protein, 90 aa, Artificial Sequence
  synthetic KIAA1389 protein fragment
TRGCETVEMTLRRNGLGQLGFHVNFEGIVADVEPFGFAWKAGLRQGSRLVEICKVAVATLTHEQMIDLLRTSVTVKVVIIQPHDDGSPRR

SEQ ID NO: 180 - protein, 96 aa, Artificial Sequence
  synthetic KIAA1415 protein fragment
VENILAKRLLILPQEEDYGFDIEEKNKAVVVKSVQRGSLAEVAGLQVGRKIYSINEDLVFLRPFSEVESILNQSFCSRRPLRLLVATKAKEIIKIP

SEQ ID NO: 181 - protein, 103 aa, Artificial Sequence
  synthetic KIAA1526 protein fragment
PDSAGPGEVRLVSLRRAKAHEGLGFSIRGGSEHGVGIYVSLVEPGSLAEKEGLRVGDQILRVNDKSLARVTHAEAVKALKGSKKLVLSVYSAGRIPGGYVTNH

SEQ ID NO: 182 - protein, 100 aa, Artificial Sequence
  synthetic KIAA1526 protein fragment
LQGGDEKKVNLVLGDGRSLGLTIRGGAEYGLGIYITGVDPGSEAEGSGLKVGDQILEVNWRSFLNILHDEAVRLLKSSRHLILTVKDVGRLPHARTTVDE

SEQ ID NO: 183 - protein, 87 aa, Artificial Sequence
  synthetic KIAA1526 protein fragment
WTSGAHVHSGPCEEKCGHPGHRQPLPRIVTIQRGGSAHNCGQLKVGHVILEVNGLTLRGKEHREAARIIAEAFKTKDRDYIDFLDSL

SEQ ID NO: 184 - protein, 100 aa, Artificial Sequence
  synthetic KIAA1620 protein fragment
ELRRAELVEIIVETEAQTGVSGINVAGGGKEGIFVRELREDSPAARSLSLQEGDQLLSARVFFENFKYEDALRLLQCAEPYKVSFCLKRTVPTGDLALRP

SEQ ID NO: 185 - protein, 102 aa, Artificial Sequence
  synthetic KIAA1634 protein fragment
PSQLKGVLVRASLKKSTMGFGFTIIGGDRPDEFLQVKNVLKDGPAAQDGKIAPGDVIVDINGNCVLGHTHADVVQMFQLVPVNQYVNLTLCRGYPLPDDSED

SEQ ID NO: 186 - protein, 100 aa, Artificial Sequence
  synthetic KIAA1634 protein fragment
ASSGSSQPELVTIPLIKGPKGFGFAIADSPTGQKVKMILDSQWCQGLQKGDIIKEIYHQNVQNLTHLQVVEVLKQFPVGADVPLLILRGGPPSPTKTAKM

SEQ ID NO: 187 - protein, 143 aa, Artificial Sequence
  synthetic KIAA1634 protein fragment
LYEDKPPLTNTFLISNPRTTADPRILYEDKPPNTKDLDVFLRKQESGFGFRVLGGDGPDQSIYIGAIIPLGAAEKDGRLRAADELMCIDGIPVKGKSHKQVLDLMTTAARNGHVLLTVRRKIFYGEKQPEDDSGSPGIHRELT

SEQ ID NO: 188 - protein, 102 aa, Artificial Sequence
  synthetic KIAA1634 protein fragment
PAPQEPYDVVLQRKENEGFGFVILTSKNKPPPGVIPHKIGRVIEGSPADRCGKLKVGDHISAVNGQSIVELSHDNIVQLIKDAGVTVTLTVIAEEEHHGPPS

SEQ ID NO: 189 - protein, 98 aa, Artificial Sequence
  synthetic KIAA1634 protein fragment
QNLGCYPVELERGPRGFGFSLRGGKEYNMGLFILRLAEDGPAIKDGRIHVGDQIVEINGEPTQGITHTRAIELIQAGGNKVLLLLRPGTGLIPDHGLA

SEQ ID NO: 190 - protein, 84 aa, Artificial Sequence
  synthetic KIAA1719 protein fragment
ITVVELIKKEGSTLGLTISGGTDKDGKPRVSNLRPGGLAARSDLLNIGDYIRSVNGIHLTRLRHDEIITLLKNVGERVVLEVEY

SEQ ID NO: 191 - protein, 92 aa, Artificial Sequence
  synthetic KIAA1719 protein fragment
ILDVSLYKEGNSFGFVLRGGAHEDGHKSRPLVLTYVRPGGPADREGSLKVGDRLLSVDGIPLHGASHATALATLRQCSHEALFQVEYDVATP

SEQ ID NO: 192 - protein, 102 aa, Artificial Sequence
  synthetic KIAA1719 protein fragment
IHTVANASGPLMVEIVKTPGSALGISLTTTSLRNKSVITIDRIKPASVVDRSGALHPGDHILSIDGTSMEHCSLLEATKLLASISEKVRLEILPVPQSQRPL

SEQ ID NO: 193 - protein, 103 aa, Artificial Sequence
  synthetic KIAA1719 protein fragment
IQIVHTETTEVVLCGDPLSGFGLQLQGGIFATETLSSPPLVCFIEPDSPAERCGLLQVGDRVLSINGIATEDGTMEEANQLLRDAALAHKVVLEVEFDVAESV

SEQ ID NO: 194 - protein, 103 aa, Artificial Sequence
  synthetic KIAA1719 protein fragment
IQFDVAESVIPSSGTFHVKLPKKRSVELGITISSASRKRGEPLIISDIKKGSVAHRTGTLEPGDKLLAIDNIRLDNCPMEDAVQILRQCEDLVKLKIRKDEDN

SEQ ID NO: 195 - protein, 94 aa, Artificial Sequence
  synthetic KIAA1719 protein fragment
IQTTGAVSYTVELKRYGGPLGITISGTEEPFDPIVISGLTKRGLAERTGAIHVGDRILAINNVSLKGRPLSEAIHLLQVAGETVTLKIKKQLDR

SEQ ID NO: 196 - protein, 105 aa, Artificial Sequence
  synthetic KIAA1719 protein fragment
ILEMEELLLPTPLEMHKVTLHKDPMRHDFGFSVSDGLLEKGVYVHTVRPDGPAHRGGLQPFDRVLQVNHVRTRDFDCCLAVPLLAEAGDVLELIISRKPHTAHSS

SEQ ID NO: 197 - protein, 91 aa, Artificial Sequence
  synthetic LIM Mystique protein fragment
MALTVDVAGPAPWGFRITGGRDFHTPIMVTKVAERGKAKDADLRPGDIIVAINGESAEGMLHAEAQSKIRQSPSPLRLQLDRSQATSPGQT

SEQ ID NO: 198 - protein, 84 aa, Artificial Sequence
  synthetic LIM protein fragment
SNYSVSLVGPAPWGFRLQGGKDFNMPLTISSLKDGGKAAQANVRIGDVVLSIDGINAQGMTHLEAQNKIKGCTGSLNMTLQRAS

SEQ ID NO: 199 - protein, 133 aa, Artificial Sequence
  synthetic LIMK1 protein fragment
TLVEHSKLYCGHCYYQTVVTPVIEQILPDSPGSHLPHTVTLVSIPASSHGKRGLSVSIDPPHGPPGCGTEHSHTVRVQGVDPGCMSPDVKNSIHVGDRILEINGTPIRNVPLDEIDLLIQETSRLLQLTLEHD

SEQ ID NO: 200 - protein, 92 aa, Artificial Sequence
  synthetic LIMK2 protein fragment
PYSVTLISMPATTEGRRGFSVSVESACSNYATTVQVKEVNRMHISPNNRNAIHPGDRILEINGTPVRTLRVEEVEDAISQTSQTLQLLIEHD

SEQ ID NO: 201 - protein, 82 aa, Artificial Sequence
  synthetic LIM-RIL protein fragment
IHSVTLRGPSPWGFRLVGRDFSAPLTISRVHAGSKASLAALCPGDLIQAINGESTELMTHLEAQNRIKGCHDHLTLSVSRPE

SEQ ID NO: 202 - protein, 74 aa, Artificial Sequence
  synthetic LU-1 protein fragment
VCYRTDDEEDLGIYVGEVNPNSIAAKDGRIREGDRIIQINGVDVQNREEAVAILSQEENTNISLLVARPESQLA

SEQ ID NO: 203 - protein, 103 aa, Artificial Sequence
  synthetic MAGII protein fragment
IQKKNHWTSRVHECTVKRGPQGELGVTVLGGAEHGEFPYVGAVAAVEAAGLPGGGEGPRLGEGELLLEVQGVRVSGLPRYDVLGVIDSCKEAVTFKAVRQGGR

SEQ ID NO: 204 - protein, 104 aa, Artificial Sequence
  synthetic MAGII protein fragment
PSELKGKFIHTKLRKSSRGFGFTVVGGDEPDEFLQIKSLVLDGPAALDGKMETGDVIVSVNDTCVLGHTHAQVVKIFQSIPIGASVDLELCRGYPLPFDPDDPN

SEQ ID NO: 205 - protein, 92 aa, Artificial Sequence
  synthetic MAGII protein fragment
PATQPELITVHIVKGPMGFGFTIADSPGGGGQRVKQIVDSPRCRGLKEGDLIVEVNKKNVQALTHNQVVDMLVECPKGSEVTLLVQRGGNLS

SEQ ID NO: 206 - protein, 102 aa, Artificial Sequence
  synthetic MAGII protein fragment
PDYQEQDIFLWRKETGFGFRILGGNEPGEPIYIGHIVPLGAADTDGRLRSGDELICVDGTPVIGKSHQLVVQLMQQAAKQGHVNLTVRRKVVFAVPKTENSS

SEQ ID NO: 207 - protein, 112 aa, Artificial Sequence
  synthetic MAGII protein fragment
GVVSTVVQPYDVEIRRGENEGFGFVIVSSVSRPEAGTTFAGNACVAMPHKIGRIIEGSPADRCGKLKVGDRILAVNGCSITNKSHSDIVNLIKEAGNTVTLRIIPGDESSNA

SEQ ID NO: 208 - protein, 91 aa, Artificial Sequence
  synthetic MAGII protein fragment
QATQEQDFYTVELERGAKGFGFSLRGGREYNMDLYVLRLAEDGPAERCGKMRIGDEILEINGETTKNMKHSRAIELIKNGGRRVRLFLKRG

SEQ ID NO: 209 - protein, 100 aa, Artificial Sequence
  synthetic MGC5395 protein fragment
PAKMEKEETTRELLLPNWQGSGSHGLTIAQRDDGVFVQEVTQNSPAARTGVVKEGDQIVGATIYFDNLQSGEVTQLLNTMGHHTVGLKLHRKGDRSPNSS

SEQ ID NO: 210 - protein, 98 aa, Artificial Sequence
  synthetic MINT1 protein fragment
SENCKDVFIEKQKGEILGVVIVESGWGSILPTVIIANMMHGGPAEKSGKLNIGDQIMSINGTSLVGLPLSTCQSIIKGLKNQSRVKLNIVRCPPVNSS

SEQ ID NO: 211 - protein, 92 aa, Artificial Sequence
  synthetic MINT1 protein fragment
LRCPPVTTVLIRRPDLRYQLGFSVQNGIICSLMRGGIAERGGVRVGHRIIEINGQSVVATPHEKIVHILSNAVGEIHMKTMPAAMYRLLNSS

SEQ ID NO: 212 - protein, 103 aa, Artificial Sequence
  synthetic MINT3 protein fragment
LSNSDNCREVHLEKRRGEGLGVALVESGWGSLLPTAVIANLLHGGPAERSGALSIGDRLTAINGTSLVGLPLAACQAAVRETKSQTSVTLSIVHCPPVTTAIM

SEQ ID NO: 213 - protein, 92 aa, Artificial Sequence
  synthetic MINT3 protein fragment
LVHCPPVTTAIIHRPHAREQLGFCVEDGIICSLLRGGIAERGGIRVGHRIIEINGQSVVATPHARIIELLTEAYGEVHIKTMPAATYRLLTG

SEQ ID NO: 214 - protein, 86 aa, Artificial Sequence
  synthetic MPP1 protein fragment
RKVRLIQFEKVTEEPMGITLKLNEKQSCTVARILHGGMIHRQGSLHVGDEILEINGTNVTNHSVDQLQKAMKETKGMISLKVIPNQ

SEQ ID NO: 215 - protein, 89 aa, Artificial Sequence
  synthetic MPP2 protein fragment
PVPPDAVRMVGIRKTAGEHLGVTFRVEGGELVIARILHGGMVAQQGLLHVGDIIKEVNGQPVGSDPRALQELLRNASGSVILKILPNYQ

SEQ ID NO: 216 - protein, 99 aa, Artificial Sequence
  synthetic MUPP1 protein fragment
QGRHVEVFELLKPPSGGLGFSVVGLRSENRGELGIFVQEIQEGSVAHRDGRLKETDQILAINGQALDQTITHQQAISILQKAKDTVQLVIARGSLPQLV

SEQ ID NO: 217 - protein, 97 aa, Artificial Sequence
  synthetic MUPP1 protein fragment
PVHWQHMETIELVNDGSGLGFGIIGGKATGVIVKTILPGGVADQHGRLCSGDHILKIGDTDLAGMSSEQVAQVLRQCGNRVKLMIARGAIEERTAPT

SEQ ID NO: 218 - protein, 98 aa, Artificial Sequence
  synthetic MUPP1 protein fragment
QESETFDVELTKNVQGLGITIAGYIGDKKLEPSGIFVKSITKSSAVEHDGRIQIGDQIIAVDGTNLQGFTNQQAVEVLRHTGQTVLLTLMRRGMKQEA

SEQ ID NO: 219 - protein, 92 aa, Artificial Sequence
  synthetic MUPP1 protein fragment
LNYEIVVAHVSKFSENSGLGISLEATVGHHFIRSVLPEGPVGHSGKLFSGDELLEVNGITLLGENHQDVVNILKELPIEVTMVCCRRTVPPT

SEQ ID NO: 220 - protein, 100 aa, Artificial Sequence
  synthetic MUPP1 protein fragment
WEAGIQHIELEKGSKGLGFSILDYQDPIDPASTVIIIRSLVPGGIAEKDGRLLPGDRLMFVNDVNLENSSLEEAVEALKGAPSGTVRIGVAKPLPLSPEE

SEQ ID NO: 221 - protein, 99 aa, Artificial Sequence
  synthetic MUPP1 protein fragment
RNVSKESFERTINIAKGNSSLGMTVSANKDGLGMIVRSIIHGGAISRDGRIAIGDCILSINEESTISVTNAQARAMLRRHSLIGPDIKITYVPAEHLEE

SEQ ID NO: 222 - protein, 112 aa, Artificial Sequence
  synthetic MUPP1 protein fragment
LNWNQPRRVELWREPSKSLGISIVGGRGMGSRLSNGEVMRGIFIKHVLEDSPAGKNGTLKPGDRIVEVDGMDLRDASHEQAVEAIRKAGNPVVFMVQSIINRPRKSPLPSLL

SEQ ID NO: 223 - protein, 95 aa, Artificial Sequence
  synthetic MUPP1 protein fragment
LTGELHMIELEKGHSGLGLSLAGNKDRSRMSVFIVGIDPNGAAGKDGRLQIADELLEINGQILYGRSHQNASSIIKCAPSKVKIIFIRNKDAVNQ

SEQ ID NO: 224 - protein, 94 aa, Artificial Sequence
  synthetic MUPP1 protein fragment
LSSFKNVQHLELPKDQGGLGIAISEEDTLSGVIIKSLTEHGVAATDGRLKVGDQILAVDDEIVVGYPIEKFISLLKTAKMTVKLTIHAENPDSQ

SEQ ID NO: 225 - protein, 95 aa, Artificial Sequence
  synthetic MUPP1 protein fragment
LPGCETTIEISKGRTGLGLSIVGGSDTLLGAIIIHEVYEEGAACKDGRLWAGDQILEVNGIDLRKATHDEAINVLRQTPQRVRLTLYRDEAPYKE

SEQ ID NO: 226 - protein, 98 aa, Artificial Sequence
  synthetic MUPP1 protein fragment
KEEEVCDTLTIELQKKPGKGLGLSIVGKRNDTGVFVSDIVKGGIADADGRLMQGDQILMVNGEDVRNATQEAVAALLKCSLGTVTLEVGRIKAGPFHS

SEQ ID NO: 227 - protein, 96 aa, Artificial Sequence
  synthetic MUPP1 protein fragment
LQGLRTVEMKKGPTDSLGISIAGGVGSPLGDVPIFIAMMHPTGVAAQTQKLRVGDRIVTICGTSTEGMTHTQAVNLLKNASGSIEMQVVAGGDVSV

SEQ ID NO: 228 - protein, 91 aa, Artificial Sequence
  synthetic MUPP1 protein fragment
LGPPQCKSITLERGPDGLGFSIVGGYGSPHGDLPIYVKTVFAKGAASEDGRLKRGDQIIAVNGQSLEGVTHEEAVAILKRTKGTVTLMVLS

SEQ ID NO: 229 - protein, 93 aa, Artificial Sequence
  synthetic NeDLG protein fragment
IQYEEIVLERGNSGLGFSIAGGIDNPHVPDDPGIFITKIIPGGAAAMDGRLGVNDCVLRVNEVEVSEVVHSRAVEALKEAGPVVRLVVRRRQN

SEQ ID NO: 230 - protein, 90 aa, Artificial Sequence
  synthetic NeDLG protein fragment
ITLLKGPKGLGFSIAGGIGNQHIPGDNSIYITKIIEGGAAQKDGRLQIGDRLLAVNNTNLQDVRHEEAVASLKNTSDMVYLKVAKPGSLE

SEQ ID NO: 231 - protein, 119 aa, Artificial Sequence
  synthetic NeDLG protein fragment
ILLHKGSTGLGFNIVGGEDGEGIFVSFILAGGPADLSGELRRGDRILSVNGVNLRNATHEQAAAALKRAGQSVTIVAQYRPEEYSRFESKIHDLREQMMNSSMSSGSGSLRTSEKRSLE

SEQ ID NO: 232 - protein, 111 aa, Artificial Sequence
  synthetic Neurabin II protein fragment
CVERLELFPVELEKDSEGLGISIIGMGAGADMGLEKLGIFVKTVTEGGAAHRDGRIQVNDLLVEVDGTSLVGVTQSFAASVLRNTKGRVRFMIGRERPGEQSEVAQRIHRD

SEQ ID NO: 233 - protein, 90 aa, Artificial Sequence
  synthetic NOS1 protein fragment
IQPNVISVRLFKRKVGGLGFLVKERVSKPPVIISDLIRGGAAEQSGLIQAGDIILAVNGRPLVDLSYDSALEVLRGIASETHVVLILRGP

SEQ ID NO: 234 - protein, 107 aa, Artificial Sequence
  synthetic Novel PDZ gene protein fragment
QANSDESDIIHSVRVEKSPAGRLGFSVRGGSEHGLGIFVSKVEEGSSAERAGLCVGDKITEVNGLSLESTTMGSAVKVLTSSSRLHMMVRRMGRVPGIKFSKEKNSS

SEQ ID NO: 235 - protein, 106 aa, Artificial Sequence
  synthetic Novel PDZ gene protein fragment
PSDTSSEDGVRRIVHLYTTSDDFCLGFNIRGGKEFGLGIYVSKVDHGGLAEENGIKVGDQVLAANGVRFDDISHSQAVEVLKGQTHIMLTIKETGRYPAYKEMNSS

SEQ ID NO: 236 - protein, 115 aa, Artificial Sequence
  synthetic Novel serine protease protein fragment
KIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITV

SEQ ID NO: 237 - protein, 100 aa, Artificial Sequence
  synthetic Numb Binding Protein protein fragment
PDGEITSIKINRVDPSESLSIRLVGGSETPLVHIIIQHIYRDGVIARDGRLLPGDIILKVNGMDISNVPHNYAVRLLRQPCQVLWLTVMREQKFRSRNSS

SEQ ID NO: 238 - protein, 101 aa, Artificial Sequence
  synthetic Numb Binding Protein protein fragment
HRPRDDSFHVILNKSSPEEQLGIKLVRKVDEPGVFIFNVLDGGVAYRHGQLEENDRVLAINGHDLRYGSPESAAHLIQASERRVHLVVSRQVRQRSPENSS

SEQ ID NO: 239 - protein, 104 aa, Artificial Sequence
  synthetic Numb Binding Protein protein fragment
PTITCHEKVVNIQKDPGESLGMTVAGGASHREWDLPIYVISVEPGGVISRDGRIKTGDILLNVDGVELTEVSRSEAVALLKRTSSSIVLKALEVKEYEPQEFIV

SEQ ID NO: 240 - protein, 99 aa, Artificial Sequence
  synthetic Numb Binding Protein protein fragment
PRCLYNCKDIVLRRNTAGSLGFCIVGGYEEYNGNKPFFIKSIVEGTPAYNDGRIRCGDILLAVNGRSTSGMIHACLARLLKELKGRITLTIVSWPGTFL

SEQ ID NO: 241 - protein, 101 aa, Artificial Sequence
  synthetic Outer membrane protein fragment
LLTEEEINLTRGPSGLGFNIVGGTDQQYVSNDSGIYVSRIKENGAAALDGRLQEGDKILSVNGQDLKNLLHQDAVDLFRNAGYAVSLRVQHRLQVQNGIHS

SEQ ID NO: 242 - protein, 94 aa, Artificial Sequence
  synthetic p55T protein fragment
PVDAIRILGIHKRAGEPLGVTFRVENNDLVIARILHGGMIDRQGLLHVGDIIKEVNGHEVGNNPKELQELLKNISGSVTLKILPSYRDTITPQQ

SEQ ID NO: 243 - protein, 93 aa, Artificial Sequence
  synthetic PAR3 protein fragment
DDMVKLVEVPNDGGPLGIHVVPFSARGGRTLGLLVKRLEKGGKAEHENLFRENDCIVRINDGDLRNRRFEQAQHMFRQAMRTPIIWFHVVPAA

SEQ ID NO: 244 - protein, 94 aa, Artificial Sequence
  synthetic PAR3 protein fragment
GKRLNIQLKKGTEGLGFSITSRDVTIGGSAPIYVKNILPRGAAIQDGRLKAGDRLIEVNGVDLVGKSQEEVVSLLRSTKMEGTVSLLVFRQEDA

SEQ ID NO: 245 - protein, 103 aa, Artificial Sequence
  synthetic PAR3 protein fragment
TPDGTREFLTFEVPLNDSGSAGLGVSVKGNRSKENHADLGIFVKSIINGGAASKDGRLRVNDQLIAVNGESLLGKTNQDAMETLRRSMSTEGNKRGMIQLIVA

SEQ ID NO: 246 - protein, 102 aa, Artificial Sequence
  synthetic PAR6 protein fragment
LPETHRRVRLHKHGSDRPLGFYIRDGMSVRVAPQGLERVPGIFISRLVRGGLAESTGLLAVSDEILEVNGIEVAGKTLDQVTDMMVANSHNLIVTVKPANQR

SEQ ID NO: 247 - protein, 111 aa, Artificial Sequence
  synthetic PAR6 GAMMA protein fragment
IDVDLVPETHRRVRLHRHGCEKPLGFYIRDGASVRVTPHGLEKVPGIFISRMVPGGLAESTGLLAVNDEVLEVNGIEVAGKTLDQVTDMMIANSHNLIVTVKPANQRNNVV

SEQ ID NO: 248 - protein, 100 aa, Artificial Sequence
  synthetic PDZ-73 protein fragment
RSRKLKEVRLDRLHPEGLGLSVRGGLEFGCGLFISHLIKGGQADSVGLQVGDEIVRINGYSISSCTHEEVINLIRTKKTVSIKVRHIGLIPVKSSPDEFH

SEQ ID NO: 249 - protein, 102 aa, Artificial Sequence
  synthetic PDZ-73 protein fragment
IPGNRENKEKKVFISLVGSRGLGCSISSGPIQKPGIFISHVKPGSLSAEVGLEIGDQIVEVNGVDFSNLDHKEAVNVLKSSRSLTISIVAAAGRELFMTDEF

SEQ ID NO: 250 - protein, 103 aa, Artificial Sequence
  synthetic PDZ-73 protein fragment
PEQIMGKDVRLLRIKKEGSLDLALEGGVDSPIGKVVVSAVYERGAAERHGGIVKGDEIMAINGKIVTDYTLAEADAALQKAWNQGGDWIDLVVAVCPPKEYDD

SEQ ID NO: 251 - protein, 103 aa, Artificial Sequence
  synthetic PDZK1 protein fragment
LTSTFNPRECKLSKQEGQNYGFFLRIEKDTEGHLVRVVEKCSPAEKAGLQDGDRVLRINGVFVDKEEHMQVVDLVRKSGNSVTLLVLDGDSYEKAGSPGIHRD

SEQ ID NO: 252 - protein, 92 aa, Artificial Sequence
  synthetic PDZK1 protein fragment
RLCYLVKEGGSYGFSLKTVQGKKGVYMTDITPQGVAMRAGVLADDHLIEVNGENVEDASHEEVVEKVKKSGSRVMFLLVDKETDKREFIVTD

SEQ ID NO: 253 - protein, 112 aa, Artificial Sequence
  synthetic PDZK1 protein fragment
QFKRETASLKLLPHQPRIVEMKKGSNGYGFYLRAGSEQKGQIIKDIDSGSPAEEAGLKNNDLVVAVNGESVETLDHDSVVEMIRKGGDQTSLLVVDKETDNMYRLAEFIVTD

SEQ ID NO: 254 - protein, 95 aa, Artificial Sequence
  synthetic PDZK1 protein fragment
PDTTEEVDHKPKLCRLAKGENGYGFHLNAIRGLPGSFIKEVQKGGPADLAGLEDEDVIIEVNGVNVLDEPYEKVVDRIQSSGKNVTLLVZGKNSS

SEQ ID NO: 255 - protein, 89 aa, Artificial Sequence
  synthetic PICK1 protein fragment
PTVPGKVTLQKDAQNLIGISIGGGAQYCPCLYIVQVFDNTPAALDGTVAAGDEITGVNGRSIKGKTKVEVAKMIQEVKGEVTIHYNKLQ

SEQ ID NO: 256 - protein, 98 aa, Artificial Sequence
  synthetic PIST protein fragment
SQGVGPIRKVLLLKEDHEGLGISITGGKEHGVPILISEIHPGQPADRCGGLHVGDAILAVNGVNLRDTKHKEAVTILSQQRGEIEFEVVYVAPEVDSD

SEQ ID NO: 257 - protein, 97 aa, Artificial Sequence
  synthetic prIL16 protein fragment
IHVTILHKEEGAGLGFSLAGGADLENKVITVHRVFPNGLASQEGTIQKGNEVLSINGKSLKGTTHHDALAILRQAREPRQAVIVTRKLTPEEFIVTD

SEQ ID NO: 258 - protein, 98 aa, Artificial Sequence
  synthetic prIL16 protein fragment
TAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSK

SEQ ID NO: 259 - protein, 99 aa, Artificial Sequence
  synthetic PSD95 protein fragment
LEYEEITLERGNSGLGFSIAGGTDNPHIGDDPSIFITKIIPGGAAAQDGRLRVNDSILFVNEVDVREVTHSAAVEALKEAGSIVRLYVMRRKPPAENSS

SEQ ID NO: 260 - protein, 105 aa, Artificial Sequence
  synthetic PSD95 protein fragment
HVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYL

SEQ ID NO: 261 - protein, 97 aa, Artificial Sequence
  synthetic PSD95 protein fragment
REDIPREPRRIVIHRGSTGLGFNIVGGEDGEGIFISFILAGGPADLSGELRKGDQILSVNGVDLRNASHEQAAIALKNAGQTVTIIAQYKPEFIVTD

SEQ ID NO: 262 - protein, 88 aa, Artificial Sequence
  synthetic PTN-3 protein fragment
LIRITPDEDGKFGFNLKGGVDQKMPLVVSRINPESPADTCIPKLNEGDQIVLINGRDISEHTHDQVVMFIKASRESHSRELALVIRRR

SEQ ID NO: 263 - protein, 88 aa, Artificial Sequence
  synthetic PTN-4 protein fragment
IRMKPDENGRFGFNVKGGYDQKMPVIVSRVAPGTPADLCVPRLNEGDQVVLINGRDIAEHTHDQVVLFIKASCERHSGELMLLVRPNA

SEQ ID NO: 264 - protein, 106 aa, Artificial Sequence
  synthetic PTPL1 protein fragment
PEREITLVNLKKDAKYGLGFQIIGGEKMGRLDLGIFISSVAPGGPADFHGCLKPGDRLISVNSVSLEGVSHHAAIEILQNAPEDVTLVISQPKEKISKVPSTPVHL

SEQ ID NO: 265 - protein, 95 aa, Artificial Sequence
  synthetic PTPL1 protein fragment
GDIFEVELAKNDNSLGISVTGGVNTSVRHGGIYVKAVIPQGAAESDGRIHKGDRVLAVNGVSLEGATHKQAVETLRNTGQVVHLLLEKGQSPTSK

SEQ ID NO: 266 - protein, 104 aa, Artificial Sequence
  synthetic PTPL1 protein fragment
TEENTFEVKLFKNSSGLGFSFSREDNLIPEQINASIVRVKKLFAGQPAAESGKIDVGDVILKVNGASLKGLSQQEVISALRGTAPEVFLLLCRPPPGVLPEIDT

SEQ ID NO: 267 - protein, 98 aa, Artificial Sequence
  synthetic PTPL1 protein fragment
ELEVELLITLIKSEKASLGFTVTKGNQRIGCYVHDVIQDPAKSDGRLKPGDRLIKVNDTDVTNMTHTDAVNLLRAASKTVRLVIGRVLELPRIPMLPH

SEQ ID NO: 268 - protein, 94 aa, Artificial Sequence
  synthetic PTPL1 protein fragment
MLPHLLPDITLTCNKEELGFSLCGGHDSLYQVVYISDINPRSVAAIEGNLQLLDVIHYVNGVSTQGMTLEEVNRALDMSLPSLVLKATRNDLPV

SEQ ID NO: 269 - protein, 93 aa, Artificial Sequence
  synthetic RGS12 protein fragment
RPSPPRVRSVEVARGRAGYGFTLSGQAPCVLSCVMRGSPADFVGLRAGDQILAVNEINVKKASHEDVVKLIGKCSGVLHMVIAEGVGRFESCS

SEQ ID NO: 270 - protein, 96 aa, Artificial Sequence
  synthetic RGS3 protein fragment
LCSERRYRQITIPRGKDGFGFTICCDSPVRVQAVDSGGPAERAGLQQLDTVLQLNERPVEHWKCVELAHEIRSCPSEIILLVWRMVPQVKPGIHRD

SEQ ID NO: 271 - protein, 104 aa, Artificial Sequence
  synthetic Rhophilin-like protein fragment
ISFSANKRWTPPRSIRFTAEEGDLGFTLRGNAPVQVHFLDPYCSASVAGAREGDYIVSIQLVDCKWLTLSEVMKLLKSFGEDEIEMKVVSLLDSTSSMHNKSAT

SEQ ID NO: 272 - protein, 109 aa, Artificial Sequence
  synthetic Serine protease protein fragment
RGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYV

SEQ ID NO: 273 - protein, 111 aa, Artificial Sequence
  synthetic Shank 1 protein fragment
EEKTVVLQKKDNEGFGFVLRGAKADTPIEEFTPTPAFPALQYLESVDEGGVAWQAGLRTGDFLIEVNNENVVKVGHRQVVNMIRQGGNHLVLKVVTVTRNLDPDDTARKKA

SEQ ID NO: 274 - protein, 110 aa, Artificial Sequence
  synthetic Shank 3 protein fragment
SDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDG

SEQ ID NO: 275 - protein, 91 aa, Artificial Sequence
  synthetic Shroom protein fragment
IYLEAFLEGGAPWGFTLKGGLEHGEPLIISKVEEGGKADTLSSKLQAGDEVVHINEVTLSSSRKEAVSLVKGSYKTLRLVVRRDVCTDPGH

SEQ ID NO: 276 - protein, 83 aa, Artificial Sequence
  synthetic SIPI protein fragment
IRLCRLVRGEQGYGFHLHGEKGRRGQFIRRVEPGSPAEAAALRAGDRLVEVNGVNVEGETHHQVVQRIKAVEGQTRLLVVDQN

SEQ ID NO: 277 - protein, 84 aa, Artificial Sequence
  synthetic SIPI protein fragment
IRHLRKGPQGYGFNLHSDKSRPGQYIRSVDPGSPAARSGLRAQDRLIEVNGQNVEGLRHAEVVASIKAREDEARLLVVDPETDE

SEQ ID NO: 278 - protein, 92 aa, Artificial Sequence
  synthetic SITAC-18 protein fragment
PGVREIHLCKDERGKTGLRLRKVDQGLFVQLVQANTPASLVGLRFGDQLLQIDGRDCAGWSSHKAHQVVKKASGDKIVVVVRDRPFQRTVTM

SEQ ID NO: 279 - protein, 90 aa, Artificial Sequence
  synthetic SITAC-18 protein fragment
PFQRTVTMHKDSMGHVGFVIKKGKIVSLVKGSSAARNGLLTNHYVCEVDGQNVIGLKDKKIMEILATAGNVVTLTIIPSVIYEHIVEFIV

SEQ ID NO: 280 - protein, 109 aa, Artificial Sequence
  synthetic SSTRIP protein fragment
LKEKTVLLQKKDSEGFGFVLRGAKAQTPIEEFTPTPAFPALQYLESVDEGGVAWRAGLRMGDFLIEVNGQNVVKVGHRQVVNMIRQGGNTLMVKVVMVTRHPDMDEAVQ

SEQ ID NO: 281 - protein, 88 aa, Artificial Sequence
  synthetic SYNTENIN protein fragment
LEIKQGIREVILCKDQDGKIGLRLKSIDNGIFVQLVQANSPASLVGLRFGDQVLQINGENCAGWSSDKAHKVLKQAFGEKITMRIHRD

SEQ ID NO: 282 - protein, 75 aa, Artificial Sequence
  synthetic SYNTENIN protein fragment
RDRPFERTITMHKDSTGHVGFIFKNGKITSIVKDSSAARNGLLTEHNICEINGQNVIGLKDSQIADILSTSGNSS

SEQ ID NO: 283 - protein, 94 aa, Artificial Sequence
  synthetic Syntrophin 1 alpha protein fragment
QRRRVTVRKADAGGLGISIKGGRENKMPILISKIFKGLAADQTEALFVGDAILSVNGEDLSSATHDEAVQVLKKTGKEVVLEVKYMKDVSPYFK

SEQ ID NO: 284 - protein, 89 aa, Artificial Sequence
  synthetic Syntrophin beta 2 protein fragment
IRVVKQEAGGLGISIKGGRENRMPILISKIFPGLAADQSRALRLGDAILSVNGTDLRQATHDQAVQALKRAGKEVLLEVKFIREFIVTD

SEQ ID NO: 285 - protein, 101 aa, Artificial Sequence
  synthetic Syntrophin gamma 1 protein fragment
EPFYSGERTVTIRRQTVGGFGLSIKGGAEHNIPVVVSKISKEQRAELSGLLFIGDAILQINGINVRKCRHEEVVQVLRNAGEEVTLTVSFLKRAPAFLKLP

SEQ ID NO: 286 - protein, 99 aa, Artificial Sequence
  synthetic Syntrophin gamma 2 protein fragment
SHQGRNRRTVTLRRQPVGGLGLSIKGGSEHNVPVVISKIFEDQAADQTGMLFVGDAVLQVNGIHVENATHEEVVHLLRNAGDEVTITVEYLREAPAFLK

SEQ ID NO: 287 - protein, 91 aa, Artificial Sequence
  synthetic TAX2-like Protein protein fragment
RGETKEVEVTKTEDALGLTITDNGAGYAFIKRIKEGSIINRIEAVCVGDSIEAINDHSIVGCRHYEVAKMLRELPKSQPFTLRLVQPKRAF

SEQ ID NO: 288 - protein, 88 aa, Artificial Sequence
  synthetic TIAM 1 protein fragment
HSIHIEKSDTAADTYGFSLSSVEEDGIRRLYVNSVKETGLASKKGLKAGDEILEINNRAADALNSSMLKDFLSQPSLGLLVRTYPELE

SEQ ID NO: 289 - protein, 97 aa, Artificial Sequence
  synthetic TIAM 2 protein fragment
PLNVYDVQLTKTGSVCDFGFAVTAQVDERQHLSRIFISDVLPDGLAYGEGLRKGNEIMTLNGEAVSDLDLKQMEALFSEKSVGLTLIARPPDTKATL

SEQ ID NO: 290 - protein, 103 aa, Artificial Sequence
  synthetic TIP1 protein fragment
QRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQK

SEQ ID NO: 291 - protein, 86 aa, Artificial Sequence
  synthetic TIP2 protein fragment
RKEVEVFKSEDALGLTITDNGAGYAFIKRIKEGSVIDHIHLISVGDMIEAINGQSLLGCRHYEVARLLKELPRGRTFTLKLTEPRK

SEQ ID NO: 292 - protein, 91 aa, Artificial Sequence
  synthetic TIP33 protein fragment
HSHPRVVELPKTDEGLGFNVMGGKEQNSPIYISRIIPGGVAERHGGLKRGDQLLSVNGVSVEGEHHEKAVELLKAAKDSVKLVVRYTPKVL

SEQ ID NO: 293 - protein, 96 aa, Artificial Sequence
  synthetic TIP43 protein fragment
ISNQKRGVKVLKQELGGLGISIKGGKENKMPILISKIFKGLAADQTQALYVGDAILSVNGADLRDATHDEAVQALKRAGKEVLLEVKYMREATPYV

SEQ ID NO: 294 - protein, 110 aa, Artificial Sequence
  synthetic X-11 beta protein fragment
IHFSNSENCKELQLEKHKGEILGVVVVESGWGSILPTVILANMMNGGPAARSGKLSIGDQIMSINGTSLVGLPLATCQGIIKGLKNQTQVKLNIVSCPPVTTVLIKRNSS

SEQ ID NO: 295 - protein, 94 aa, Artificial Sequence
  synthetic X-11 beta protein fragment
IPPVTTVLIKRPDLKYQLGFSVQNGIICSLMRGGIAERGGVRVGHRIIEINGQSVVATAHEKIVQALSNSVGEIHMKTMPAAMFRLLTGQENSS

SEQ ID NO: 296 - protein, 101 aa, Artificial Sequence
  synthetic ZO-1 protein fragment
IWEQHTVTLHRAPGFGFGIAISGGRDNPHFQSGETSIVISDVLKGGPAEGQLQENDRVAMVNGVSMDNVEHAFAVQQLRKSGKNAKITIRRKKKVQIPNSS

SEQ ID NO: 297 - protein, 95 aa, Artificial Sequence
  synthetic ZO-1 protein fragment
ISSQPAKPTKVTLVKSRKNEEYGLRLASHIFVKEISQDSLAARDGNIQEGDVVLKINGTVTENMSLTDAKTLIERSKGKLKMVVQRDRATLLNSS

SEQ ID NO: 298 - protein, 90 aa, Artificial Sequence
  synthetic ZO-1 protein fragment
IRMKLVKFRKGDSVGLRLAGGNDVGIFVAGVLEDSPAAKEGLEEGDQILRVNNVDFTNIIREEAVLFLLDLPKGEEVTILAQKKKDVFSN

SEQ ID NO: 299 - protein, 96 aa, Artificial Sequence
  synthetic ZO-2 protein fragment
LIWEQYTVTLQKDSKRGFGIAVSGGRDNPHFENGETSIVISDVLPGGPADGLLQENDRVVMVNGTPMEDVLHSFAVQQLRKSGKVAAIVVKRPRKV

SEQ ID NO: 300 - protein, 79 aa, Artificial Sequence
  synthetic ZO-2 protein fragment
RVLLMKSRANEEYGLRLGSQIFVKEMTRTGLATKDGNLHEGDIILKINGTVTENMSLTDARKLIEKSRGKLQLVVLRDS

SEQ ID NO: 301 - protein, 90 aa, Artificial Sequence
  synthetic ZO-2 protein fragment
HAPNTKMVRFKKGDSVGLRLAGGNDVGIFVAGIQEGTSAEQEGLQEGDQILKVNTQDFRGLVREDAVLYLLEIPKGEMVTILAQSRADVY

SEQ ID NO: 302 - protein, 106 aa, Artificial Sequence
  synthetic ZO-3 protein fragment
IPGNSTIWEQHTATLSKDPRRGFGIAISGGRDRPGGSMVVSDVVPGGPAEGRLQTGDHIVMVNGVSMENATSAFAIQILKTCTKMANITVKRPRRIHLPAEFIVTD

SEQ ID NO: 303 - protein, 98 aa, Artificial Sequence
  synthetic ZO-3 protein fragment
QDVQMKPVKSVLVKRRDSEEFGVKLGSQIFIKHITDSGLAARHRGLQEGDLILQINGVSSQNLSLNDTRRLIEKSEGKLSLLVLRDRGQFLVNIPNSS

SEQ ID NO: 304 - protein, 104 aa, Artificial Sequence
  synthetic ZO3 protein fragment
RGYSPDTRVVRFLKGKSIGLRLAGGNDVGIFVSGVQAGSPADGQGIQEGDQILQVNDVPFQNLTREEAVQFLLGLPPGEEMELVTQRKQDIFWKMVQSEFIVTD

SEQ ID NO: 305 - protein, 11 aa, Artificial Sequence
  synthetic Transmembrane transporter sequence
YGRKKRRQRRR

SEQ ID NO: 306 - protein, 4 aa, Artificial Sequence
  synthetic C-terminus of NMDAR1 receptor
STVV

SEQ ID NO: 307 - protein, 4 aa, Artificial Sequence
  synthetic C-terminus of NMDAR1-2 receptor
HRES

SEQ ID NO: 308 - protein, 4 aa, Artificial Sequence
  synthetic C-terminus of NMDAR3 receptor
ESDV

SEQ ID NO: 309 - protein, 4 aa, Artificial Sequence
  synthetic C-terminus of NMDAR3A receptor
TCES

SEQ ID NO: 310 - protein, 4 aa, Artificial Sequence
  synthetic C-terminus of TAX oncoprotein
ETEV

SEQ ID NO: 311 - protein, 4 aa, Artificial Sequence
  synthetic C-terminus of NMDA R2B
ESDV

SEQ ID NO: 312 - protein, 4 aa, Artificial Sequence
  synthetic PDZ protein domain
GLGF